(19) United States
(12) Patent Application Publication
(10) Pub. No.: US 2020/0095621 A1
(43) Pub. Date: Mar. 26, 2020

(54) METHODS AND COMPOSITIONS FOR 3-HYDROXYPROPIONATE PRODUCTION

(71) Applicant: The Regents of the University of California, Oakland, CA (US)

(72) Inventors: Yasuo YOSHIKUNI, Orinda, CA (US); Justin B. SIEGEL, Davis, CA (US); Youtian CUI, Davis, CA (US); Wai Shun MAK, Sacramento, CA (US)

(73) Assignee: The Regents of the University of California, Oakland, CA (US)

(21) Appl. No.: 16/612,304

(22) PCT Filed: May 15, 2018
(86) PCT No.: PCT/US18/32830

§ 371 Date: Nov. 8, 2019

Related U.S. Application Data
(60) Provisional application No. 62/507,019, filed on May 16, 2017.

(51) Int. Cl. C12P 7/42; C12N 9/88; C12N 9/04; C12N 1/20; C12N 1/16; C12N 15/70

(52) U.S. Cl. CPC C12P 7/42; C12N 9/88; C12Y 401/01003; C12N 9/0006; C12Y 101/01059; C12N 2533/70; C12N 1/16; C12N 15/70; C12N 2510/02; C12N 2533/20; C12N 1/20

(57) ABSTRACT

Provided herein, inter alia, are methods, host cells, and vectors for producing 3-hydroxypropionate (3-HP). In some embodiments, the host cells include a recombinant polynucleotide encoding an oxaloacetate decarboxylase (OAADC) and a polynucleotide encoding a 3-hydroxypropionate dehydrogenase (3-HPDH). In some embodiments, the methods include culturing said host cell(s) in a culture medium comprising a substrate under conditions suitable for the recombinant host cell to convert the substrate to 3-HP. Expression of the OAADC and the 3-HPDH results in increased production of 3-HP, as compared to production by a host cell lacking expression of the OAADC and the 3-HPDH.

### CROSS-REFERENCE TO RELATED APPLICATIONS

This application claims the priority benefit of U.S. Provisional Application Ser. No. 62/507,019, filed May 16, 2017, which is incorporated herein by reference in its entirety.

### STATEMENT OF GOVERNMENT SUPPORT

This invention was made with Government support under Grant No. DE-AC02-05CH11231 awarded by the Department of Energy. The Government has certain rights in this invention.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 220032001640SEQLIST.TXT, date recorded: May 11, 2018, size: 484 KB).

### FIELD

The present disclosure relates, inter alia, to methods, host cells, and vectors for producing 3-hydroxypropionate (3-HP) using an oxaloacetate decarboxylase (OAADC) and a 3-hydroxypropionate dehydrogenase (3-HPDH).

### BACKGROUND

Acrylate is an important industrial building block for polymers utilized in diapers, plastic additives, surface coatings, water treatment, adhesives, textiles, surfactants, and others. The market size for acrylate is estimated to expand to 8.2 MMT, $20Bi by 2020. 3-hydroxypropionate (3-HP) was identified as one of the top 12 value-added chemicals from biomass in 2004 (Werpy. T. et al “Top Value Added Chemicals from Biomass” US Department of Energy Report, Vol: 1. 2004), because 3-HP can be converted into acrylic acid, and several other commodity chemicals, in one step (FIG. 1).

There are more than 7 metabolic pathways proposed for 3-HP production (Kumar, V. et al. (2013) Biotech. Adv. 31:945-961; FIG. 2A), however none of them is efficient enough for industrial scale production. 3-HP could in theory be produced by a simplified metabolic pathway from glucose using an oxaloacetate decarboxylase to convert oxaloacetate into 3-oxopropanoate (FIG. 2B) with extremely high efficiency (e.g., 100% wt. 3-HP/wt. glucose); however, an enzyme that efficiently catalyzes this reaction has not been found (see U.S. Pat. Nos. 8,048,624 and 8,809,027).

Therefore, a need exists for methods, host cells, and vectors that allow for the efficient production of 3-HP, e.g., on an industrial scale. The use of an oxaloacetate decarboxylase would result in reduced costs and optimized processes as compared to existing methods.

### SUMMARY

To meet these and other demands, provided herein are methods, host cells, and vectors for producing 3-hydroxypropionate (3-HP), e.g., using an oxaloacetate decarboxylase (OAADC) and a 3-hydroxypropionate dehydrogenase (3-HPDH).

Accordingly, certain aspects of the present disclosure relate to a method for producing 3-hydroxypropionate (3-HP), the method comprising: providing a recombinant host cell, wherein the recombinant host cell comprises a recombinant polynucleotide encoding an oxaloacetate decarboxylase (OAADC) and a polynucleotide encoding a 3-hydroxypropionate dehydrogenase (3-HPDH), and wherein the OAADC has a ratio of activity against pyruvate to activity against oxaloacetate that is less than or equal to about 5:1; and culturing the recombinant host cell in a culture medium comprising a substrate under conditions suitable for the recombinant host cell to convert the substrate to 3-HP, wherein expression of the OAADC and the 3-HPDH results in increased production of 3-HP, as compared to production by a host cell lacking expression of the OAADC and the 3-HPDH. Other aspects of the present disclosure relate to a method for producing 3-hydroxypropionate (3-HP), the method comprising: providing a recombinant host cell, wherein the recombinant host cell comprises a recombinant polynucleotide encoding an oxaloacetate decarboxylase (OAADC) and a polynucleotide encoding a 3-hydroxypropionate dehydrogenase (3-HPDH), wherein the OAADC has a specific activity of at least 0.1 μmol/min/mg against oxaloacetate, and culturing the recombinant host cell in a culture medium comprising a substrate under conditions suitable for the recombinant host cell to convert the substrate to 3-HP, wherein expression of the OAADC and the 3-HPDH results in increased production of 3-HP, as compared to production by a host cell lacking expression of the OAADC and the 3-HPDH.

In some embodiments, the recombinant host cell is a recombinant prokaryotic cell. In some embodiments, the prokaryotic cell is an Escherichia coli cell. In some embodiments, the host cell is selected from the group consisting of Acetobacter aceti, Achromobacter, Acidiphilium, Acinetobacter, Actinomadura, Actinoplanes, Aeropyrum pemix, Agrobacterium, Alcaligenes, Ananas comosus (M), Arthrobacter, Bacillus alcalophilus, Bacillus amyloliquefaciens, Bacillus brevis, Bacillus circulans, Bacillus clausii, Bacillus lentus, Bacillus licheniformis, Bacillus macerans, Bacillus stearothermophilus Bacillus subtilis, Bifidobacterium, Brevibacillus brevis, Burkholderia cepacia, Candida cylindracea, Carica papaya (L), Cellulosimicrobium, Cephalosporium, Chaetomium erraticum, Chaetomium gracile, Clostridium, Clostridium butyricum, Clostridium acetobutylicum, Clostridium thermocellum, Corynebacterium (glutamicum), Corynebacterium efficiens, Escherichia coli, Enterococcus, Erwina chrysanthemi, Gliconobacter, Gluconacetobacter, Haloarcula, Humicola insolens, Kitacsatospora setae, Klebsiella, Klebsiella oxytoca, Kocuria, Lactlactis, Lactobacillus, Lactobacillus fermentum, Lactobacillus sake, Lactococcus, Lactococcus lactis, Leuconostoc, Methylocystis, Methanolobus siciliae, Methanogenium organophilum, Methanobacterium bryantii, Microbacterium imperiale, Micrococcus lysodeikticus, Microlunatus, Mucor javanicus, Mycobacterium, Myrothecium, Nitrobacter, Nitrosomonas, Nocardia, Papaya carica, Pediococcus, Pediococcus halophilus, Paracoccus pantotrophus, Propionibacterium, Pseudomonas, Pseudomonas fluorescens, Pseudomonas denitrificans, Pyrococcus, Pyrococcus firiosus, Pyrococcus horikoshii, Rhizobium, Rhizomucor miehei, Rhizomucor pusillus Lindt, Rhizopus, Rhizopus delemar, Rhizopus japonicus, Rhizopus niveus, Rhizopus oryzae, Rhizopus oligosporus, Rhodococcus, Sclerotina libertina, Sphingobacterium multivorum, Sphingohium, Sphingomonas, Streptococcus, Streptococcus thermophilus Y-1, Streptomyces, Streptomyces griseus, Streptomyces lividans, Streptomyces murinus, Streptomyces rubiginosus, Streptomyces violaceoruber, Streptoverticillium mobaraense, Tetragenococcus, Thermus, Thiosphaera pantotropha, Trametes, Vibrio alginolyticus, Xanthomonas, Zymomonas, and Zymomonus mobilis. In some embodiments, the recombinant host cell is a recombinant fungal cell.

Other aspects of the present disclosure relate to a method for producing 3-hydroxypropionate (3-HP), the method comprising: providing a recombinant host cell, wherein the recombinant host cell comprises a recombinant polynucleotide encoding an oxaloacetate decarboxylase (OAADC) and a polynucleotide encoding a 3-hydroxypropionate dehydrogenase (3-HPDH), and wherein the recombinant host cell is a recombinant fungal cell; and culturing the recombinant host cell in a culture medium comprising a substrate under conditions suitable for the recombinant host cell to convert the substrate to 3-HP, wherein expression of the OAADC and the 3-HPDH results in increased production of 3-HP, as compared to production by a host cell lacking expression of the OAADC and the 3-HPDH. In some embodiments, the OAADC has a ratio of activity against pyruvate to activity against oxaloacetate that is less than or equal to about 5:1. In some embodiments, the OAADC has a specific activity of at least 0.1 μmol/min/mg against oxaloacetate.

In some embodiments of any of the above embodiments, the OAADC has a specific activity of at least 10 μmol/min/mg against oxaloacetate. In some embodiments, the OAADC has a specific activity of at least 100 μmol/min/mg against oxaloacetate. In some embodiments of any of the above embodiments, the OAADC has a catalytic efficiency (k_{cat}/K_{M}) for oxaloacetate that is greater than about 2000 M⁻¹s⁻¹. In some embodiments, the recombinant host cell (e.g., a fungal host cell) is capable of producing 3-HP at a pH lower than 6. In some embodiments, the recombinant host cell is capable of producing 3-HP below the pKa of 3-HP. In some embodiments, the fungal cell is a yeast cell. In some embodiments, the fungal cell is of a genus or species selected from the group consisting of Aspergillus, Aspergillus nidulans, Aspargillus niger, Aspargillus oryze, Aspergillus melleus, Aspergillus pulverulentus, Aspergillus saitoi, Aspergillus sojea, Aspergillus terreus, Aspergillus pseudoterreus, Aspergillus usamii, Candida rugosa, Issatchenkia orientalis, Kluyveromyces, Kluyveromes fragilis, Kluyveromyces lactis, Kluyveromyces marxianas, Penicillium, Penicillium camemberti, Penicillium citrinum, Penicillium emersonii, Penicillium roqueforti, Penicillum lilactinum, Penicillum multicolor, Rhodosporidium toruloides, Saccharomyces cerevisiae, Schizosaccharomyces pombe, Trichoderma, Trichoderma longibrachiatum, Trichoderma reesei, Trichoderma viride, Trichosporon penicillatum, Yarrowia lipolytica, and Zygosaccharomyces rouxii.

In some embodiments of any of the above embodiments, the OAADC comprises an amino acid sequence shown in Table 2 or Table 5A. In some embodiments of any of the above embodiments, the OAADC comprises the amino acid sequence of a polypeptide selected from the group consisting of 4COK (SEQ ID NO:1), A0A0F6SDN1_9DELT (SEQ ID NO:3), 4K9Q (SEQ ID NO:5), 1JSC (SEQ ID NO:15), 3L84_3M34 (SEQ ID NO:19), A0A0F2PQV5_9FIRM (SEQ ID NO:25). A0A0R2PY37_9ACTN (SEQ ID NO:41), X1WK73_ACYPI (SEQ ID NO:43), F4RJP4_MELLP (SEQ ID NO:51), A0A081BQW3_9BACT (SEQ ID NO:53), CAK95977 (SEQ ID NO:55), YP_831380 (SEQ ID NO:57). ZP_06846103 (SEQ ID NO:61), ZP_08570611 (SEQ ID NO:65), WP_010764607.1 (SEQ ID NO:77), YP_005756646.1 (SEQ ID NO:81), WP_018535238.1 (SEQ ID NO:85), YP_006485164.1 (SEQ ID NO:112), YP_005461458.1 (SEQ ID NO: 113), YP_006991301.1 (SEQ ID NO:114), WP_003075272.1 (SEQ ID NO:115), WP_020634527.1 (SEQ ID NO:116), 10VM (SEQ ID NO:117), 2Q5Q (SEQ ID NO:118), 2VBG (SEQ ID NO:119), 2VBI (SEQ ID NO:120), and 3FZN (SEQ ID NO:121). In some embodiments of any of the above embodiments, the OAADC comprises an amino acid sequence at least 80% identical to SEQ ID NO:1. In some embodiments, the OAADC comprises the amino acid sequence of SEQ ID NO:1. In some embodiments of any of the above embodiments, the OAADC comprises an amino acid sequence at least 80% identical to a sequence selected from the group consisting of SEQ ID NOs:145, 146, 148, and 166. In some embodiments, the OAADC comprises an amino acid sequence selected from the group consisting of SEQ ID NOs:145, 146, 148, and 166.

In some embodiments of any of the above embodiments, the recombinant polynucleotide is stably integrated into a chromosome of the recombinant host cell. In some embodiments of any of the above embodiments, the recombinant polynucleotide is maintained in the recombinant host cell on an extra-chromosomal plasmid. In some embodiments of any of the above embodiments, the polynucleotide encoding the 3-HPDH is an endogenous polynucleotide. In some embodiments of any of the above embodiments, the polynucleotide encoding the 3-HPDH is a recombinant polynucleotide. In some embodiments of any of the above embodiments, the 3-HPDH comprises an amino acid sequence selected from the group consisting of SEQ ID NOs:122-130. In some embodiments of any of the above embodiments, the 3-HPDH comprises the amino acid sequence of SEQ ID NO:154 or 159. In some embodiments of any of the above embodiments, the recombinant host cell is cultured under anaerobic conditions suitable for the recombinant host cell to convert the substrate to 3-HP. In some embodiments of any of the above embodiments, the substrate comprises glucose. In some embodiments, at least 95% of the glucose metabolized by the recombinant host cell is converted to 3-HP. In some embodiments, 100% of the glucose metabolized by the recombinant host cell is converted to 3-HP. In some embodiments of any of the above embodiments, the substrate is selected from the group consisting of sucrose, fructose, xylose, arabinose, cellobiose, cellulose, alginate, mannitol, laminarin, galactose, and galactan. In some embodiments of any of the above embodiments, the recombinant host cell further comprises a recombinant polynucleotide encoding a phosphoenolpyruvate carboxykinase (PEPCK). In some embodiments, the PEPCK comprises the amino acid sequence of SEQ ID NO:162 or 163. In some embodiments of any of the above embodiments, the recombinant host cell further comprises a modification resulting in decreased production of pyruvate from phosphoenolpyruvate, as compared to a host cell lacking the modification. In some embodiments, the modification results in decreased pyruvate kinase (PK) activity, as compared to a host cell lacking the modification. In some embodiments, the modification results in decreased pyruvate kinase (PK) expression, as compared to a host cell lacking the modification. In some embodiments, the modification comprises an exogenous promoter in operable linkage with an endogenous pyruvate kinase (PK) coding sequence, wherein the exogenous promoter results in decreased endogenous PK coding sequence expression, as compared to expression of the endogenous PK coding sequence in operable linkage with an endogenous PK promoter. In some embodiments, the exogenous promoter is a MET3, CTR1, or CTR3 promoter. In some embodiments, the exogenous promoter comprises a polynucleotide sequence selected from the group consisting of SEQ ID NOs:131-133. In some embodiments, the recombinant host cell further comprises a second modification resulting in increased expression or activity of phosphoenolpyruvate carboxykinase (PEPCK), as compared to a host cell lacking the second modification. In some embodiments of any of the above embodiments, the method further comprises substantially purifying the 3-HP. In some embodiments of any of the above embodiments, the method further comprises converting the 3-HP to acrylic acid.

Other aspects of the present disclosure relate to a recombinant host cell comprising a recombinant polynucleotide encoding an oxaloacetate decarboxylase (OAADC), wherein the OAADC has a ratio of activity against pyruvate to activity against oxaloacetate that is less than or equal to about 5:1. Other aspects of the present disclosure relate to a recombinant host cell comprising a recombinant polynucleotide encoding an oxaloacetate decarboxylase (OAADC), wherein the OAADC has a specific activity of at least 0.1 μmol/min/mg against oxaloacetate. In some embodiments, the recombinant host cell is a recombinant prokaryotic cell. In some embodiments, the prokaryotic cell is an Escherichia cot cell. In some embodiments, the host cell is selected from the group consisting of Acetobacter aceti, Achromobacter, Acidiphilium, Acinetobacter, Actinonadura, Actinoplanes, Aeropyrum pernix, Agrobacterium, Alcaligenes, Ananas comosus (M), Arthrobacter, Bacillus alcalophilus, Bacillus amyloliquefaciens, Bacillus brews, Bacillus circulans, Bacillus clausii, Bacillus lentus, Bacillus licheniformis, Bacillus macerans, Bacillus stearothermophilus, Bacillus subtilis, Bifidobacterium, Brevibacillus brevis, Burkholderia cepacia, Candida cylindracea, Carica papaya (L), Cellulosimicrobium, Cephalosporium, Chaetomium erraticum, Chaetomium gracile, Clostridium, Clostridium butyricum, Clostridium acelobutylicum, Clostridium thermocellum, Corynebacterium (glutamicum), Corynebacterium efficiens, Escherichia coli, Enterococcus, Erwina chrysanthemi, Gliconobacter, Gluconacetobacter, Haloarcula, Humicola insolens, Kitasatospora setae, Klebsiella, Klebsiella oxytoca, Kocuria, Lactlactis, Lactobacillus, Lactobacillus fermentum, Lactobacillus sake, Lactococcus, Lactococcus lactis, Leuconostoc, Methylocystis, Methanolobus siciliae, Methanogenium organophilum, Methanobacterium bryantii, Microbacterium imperiale, Micrococcus lysodeikticus, Microlunatus, Mucor javanicus, Mycobacterium, Myrothecium, Nitrobacter, Nitrosomonas, Nocardia, Papaya carica, Pediococcus, Pediococcus halophilus, Paracoccus pantotrophus, Propionibacterium, Pseudomonas, Pseudomonas fluorescens, Pseudomonas denitrificans, Pyrococcus, Pyrococcus furiosus, Pyrococcus horikoshii, Rhizobium, Rhizomucor miehei, Rhizomucor pusillus Lindt, Rhizopus, Rhizopus delemar, Rhizopus japonicus, Rhizopus niveus, Rhizopus oryzae, Rhizopus oligosporus, Rhodococcus, Sclerotina libertina, Sphingobacterium multivorum, Sphingobium, Sphingomonas, Streptococcus, Streptococcus thermophilus Y-1, Streptomyces, Streptomyces griseus, Streptomyces lividans, Streptomyces murinus, Streptomyces rubiginosus, Streptomyces violaceoruber, Streptoverticillium mobaraense, Tetragenococcus, Thermus, Thiosphaera pantotropha, Trametes, Vibrio alginolyticus, Xanthomonas, Zymomonas, and Zymomonus mobilis. In some embodiments, the recombinant host cell is a recombinant fungal host cell.

Other aspects of the present disclosure relate to a recombinant fungal host cell comprising a recombinant polynucleotide encoding an oxaloacetate decarboxylase (OAADC). In some embodiments, the OAADC has a ratio of activity against pyruvate to activity against oxaloacetate that is less than or equal to about 5:1. In some embodiments, the OAADC has a specific activity of at least 0.1 μmol/min/mg against oxaloacetate.

In some embodiments of any of the above embodiments, the OAADC has a specific activity of at least 10 mol/min/mg against oxaloacetate. In some embodiments, the OAADC has a specific activity of at least 10 μmol/min/mg against oxaloacetate. In some embodiments of any of the above embodiments, the OAADC has a catalytic efficiency (k_{cat}/K_{M}) for oxaloacetate that is greater than about 2000 M⁻¹s⁻¹. In some embodiments of any of the above embodiments, the host cell further comprises a polynucleotide encoding a 3-hydroxypropionate dehydrogenase (3-HPDH). In some embodiments, the polynucleotide encoding the 3-HPDH is an endogenous polynucleotide. In some embodiments, the polynucleotide encoding the 3-HPDH is a recombinant polynucleotide. In some embodiments, the 3-HPDH comprises an amino acid sequence selected from the group consisting of SEQ ID NOs:122-130. In some embodiments, the 3-HPDH comprises the amino acid sequence of SEQ ID NO:154 or 159.

In some embodiments of any of the above embodiments, the recombinant fungal host cell is capable of producing 3-HP at a pH lower than 6. In some embodiments, the recombinant host cell is capable of producing 3-HP below the pKa of 3-HP. In some embodiments, the fungal cell is a yeast cell. In some embodiments, the fungal cell is of a genus or species selected from the group consisting of Aspergillus, Aspergillus nidulans, Aspargillus niger, Aspargillus oryze, Aspergillus melleus, Aspergillus pulverulentus, Aspergillus saitoi, Aspergillus sojea, Aspergillus terreus, Aspergillus pseudoterreus, Aspergillus usamii, Candida rugosa, Issatchenkia orientalis, Kluyveromyces, Kluyveromyces fragilis, Kluyveromyces lactis, Kluyveromyces marxianas, Penicillium, Penicillium camemberti, Penicillium citrinum, Penicillium emersonii, Penicillium roqueforti, Penicillum lilactinum, Penicillum multicolor, Rhodosporidium toruloides, Saccharomyces cerevisiae, Schizosaccharomyces pombe, Trichoderma, Trichoderma longibrachiatum, Trichoderma reesei, Trichoderma viride, Trichosporon penicillatum, Yarrowia lipolytica, and Zygosaccharomyces rouxii.

In some embodiments of any of the above embodiments, the OAADC comprises an amino acid sequence shown in Table 2 or Table 5A. In some embodiments of any of the above embodiments, the OAADC comprises the amino acid sequence of a polypeptide selected from the group consisting of 4COK (SEQ ID NO:1), A0A0F6SDN1_9DELT (SEQ ID NO:3), 4K9Q (SEQ ID NO:5), 1JSC (SEQ ID NO:15), 3L84_3M34 (SEQ ID NO:19), A0A0F2PQV5_9FIRM (SEQ ID NO:25), A0A0R2PY37_9ACTN (SEQ ID NO:41), X1WK73_ACYPI (SEQ ID NO:43), F4RJP4_MELLP (SEQ ID NO:51), A0A081BQW3_9BACT (SEQ ID NO:53), CAK95977 (SEQ ID NO:55), YP_831380 (SEQ ID NO:57), ZP_06846103 (SEQ ID NO:61), ZP_08570611 (SEQ ID NO:65), WP_010764607.1 (SEQ ID NO:77), YP_005756646.1 (SEQ ID NO:81), WP_018535238.1 (SEQ ID NO:85), YP_006485164.1 (SEQ ID NO:112), YP_005461458.1 (SEQ ID NO:113), YP_006991301.1 (SEQ ID NO:114), WP_003075272.1 (SEQ ID NO:115), WP_020634527.1 (SEQ ID NO:116), 1OVM (SEQ ID NO:117), 2Q5Q (SEQ ID NO:18), 2VBG (SEQ ID NO:119), 2VBI (SEQ ID NO:120), and 3FZN (SEQ ID NO:121). In some embodiments of any of the above embodiments, the OAADC comprises an amino acid sequence at least 80% identical to SEQ ID NO:1. In some embodiments of any of the above embodiments, the OAADC comprises the amino acid sequence of SEQ ID NO:1. In some embodiments of any of the above embodiments, the OAADC comprises an amino acid sequence at least 80% identical to a sequence selected from the group consisting of SEQ ID NOs:145, 146, 148, and 166. In some embodiments, the OAADC comprises an amino acid sequence selected from the group consisting of SEQ ID NOs:145, 146, 148, and 166.

In some embodiments of any of the above embodiments, the recombinant polynucleotide is stably integrated into a chromosome of the recombinant host cell. In some embodiments of any of the above embodiments, the recombinant polynucleotide is maintained in the recombinant host cell on an extra-chromosomal plasmid. In some embodiments of any of the above embodiments, the recombinant host cell is capable of producing 3-HP under anaerobic conditions. In some embodiments of any of the above embodiments, the recombinant host cell further comprises a recombinant polynucleotide encoding a phosphoenolpyruvate carboxykinase (PEPCK). In some embodiments, the PEPCK comprises the amino acid sequence of SEQ ID NO:162 or 163. In some embodiments of any of the above embodiments, the recombinant host cell further comprises a modification resulting in decreased production of pyruvate from phosphoenolpyruvate, as compared to a host cell lacking the modification. In some embodiments, the modification results in decreased pyruvate kinase (PK) activity, as compared to a host cell lacking the modification. In some embodiments, the modification results in decreased pyruvate kinase (PK) expression, as compared to a host cell lacking the modification. In some embodiments, the modification comprises an exogenous promoter in operable linkage with an endogenous pyruvate kinase (PK) coding sequence, wherein the exogenous promoter results in decreased endogenous PK coding sequence expression, as compared to expression of the endogenous PK coding sequence in operable linkage with an endogenous PK promoter. In some embodiments, the exogenous promoter is a MET3, CTR1, or CTR3 promoter. In some embodiments, the exogenous promoter comprises a polynucleotide sequence selected from the group consisting of SEQ ID NOs:131-133. In some embodiments, the recombinant host cell further comprises a second modification resulting in increased expression or activity of phosphoenolpyruvate carboxykinase (PEPCK), as compared to a host cell lacking the second modification.

Other aspects of the present disclosure relate to a vector comprising a polynucleotide that encodes an amino acid sequence at least 80% identical to a sequence selected from the group consisting of SEQ ID NOs:1, 145, 146, 148, and 166. In some embodiments, the polynucleotide encodes the amino acid sequence of SEQ ID NO:1. In some embodiments, the polynucleotide comprises the polynucleotide sequence of SEQ ID NO:2. In some embodiments, the polynucleotide encodes an amino acid sequence selected from the group consisting of SEQ ID NOs:145, 146, 148, and 166. In some embodiments, the vector further comprises a promoter operably linked to the polynucleotide. In some embodiments, the promoter is exogenous with respect to the polynucleotide that encodes the amino acid sequence at least 80% identical to SEQ ID NO:1. In some embodiments, the promoter is a T7 promoter. In some embodiments, the promoter is a TDH or FBA promoter. In some embodiments, the promoter comprises the polynucleotide sequence of SEQ ID NO:135 or 136. In some embodiments, the vector further comprises a polynucleotide encoding a 3-hydroxypropionate dehydrogenase (3-HPDH). In some embodiments, the 3-HPDH comprises an amino acid sequence selected from the group consisting of SEQ ID NOs:122-130. In some embodiments, the amino acid sequence of SEQ ID NO:154 or 159.

In some embodiments, the polynucleotide that encodes the sequence selected from the group consisting of SEQ ID NOs:1, 145, 146, 148, and 166 and the polynucleotide encoding the 3-hydroxypropionate dehydrogenase (3-HPDH) are arranged in an operon operably linked to the same promoter. In some embodiments, the promoter is a T7 or phage promoter. In some embodiments, an operon of the present disclosure comprises (a) a polynucleotide that encodes an amino acid sequence at least 80% identical to SEQ ID NO:1 (e.g., SEQ ID NO:2), (b) a polynucleotide encoding a 3-hydroxypropionate dehydrogenase (3-HPDH) (e.g., a polynucleotide encoding a 3-HPDH listed in Table 1 or Table 7A) or a polynucleotide encoding an alcohol dehydrogenase (e.g., comprising the sequence of NCBI GenBank Ref. No. ABX13006 or a polynucleotide encoding an alcohol dehydrogenase listed in Table 7A), and (c) a polynucleotide encoding a phosphoenolpyruvate carboxykinase (e.g., comprising a polynucleotide encoding a phosphoenolpyruvate carboxykinase listed in Table 9A). In some embodiments, the phosphoenolpyruvate carboxykinase is selected from the group consisting of E. coli Pck. NCBI Ref. Seq. No. WP_011201442, NCBI Ref. Seq. No. WP_011978877, NCBI Ref. Seq. No. WP_027939345, NCBI Ref. Seq. No. WP_074832324, and NCBI Ref. Seq. No. WP_074838421. In some embodiments, the 3-HPDH comprises the amino acid sequence of SEQ ID NO:154 or 159. In some embodiments, the vector further comprises a polynucleotide encoding a phosphoenolpyruvate carboxykinase (PEPCK). In some embodiments, the PEPCK comprises the amino acid sequence of SEQ ID NO:162 or 163. In some embodiments, the polynucleotide that encodes the sequence selected from the group consisting of SEQ ID NOs:1, 145, 146, 148, and 166; the polynucleotide encoding the 3-hydroxypropionate dehydrogenase (3-HPDH); and the polynucleotide encoding the phosphoenolpyruvate carboxykinase (PEPCK) are arranged in an operon operably linked to the same promoter (e.g., a T7 or phage promoter).

It is to be understood that one, some, or all of the properties of the various embodiments described above and herein may be combined to form other embodiments of the present invention. These and other aspects of the present disclosure will become apparent to one of skill in the art. These and other embodiments of the present disclosure are further described by the detailed description that follows.

### BRIEF DESCRIPTION OF THE DRAWINGS

FIG. 1 shows the chemical structure of 3-Hydroxypropionic acid (3-HP) and commodity/specialty chemicals that can be derived from 3-HP. The dehydration reaction of 3-HP into acrylic acid is indicated by a box. Adapted from Werpy, T. et al. “Top Value Added Chemicals from Biomass.” US Department of Energy Report, Vol. 1, 2004.

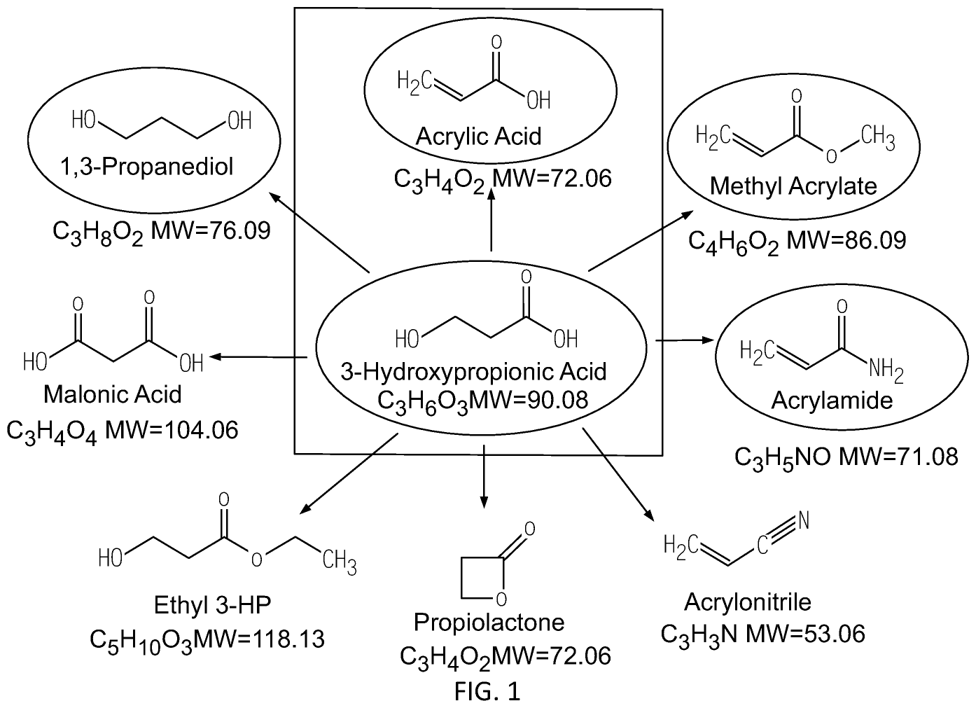

FIG. 2A shows the seven known, complex synthesis pathways involving combinations of 19 different metabolic enzymes for the production of 3-HP from glucose. Adapted from Kumar, V. et al. (2013) Biotech. Adv. 31:945-961.

FIG. 2B shows a simplified metabolic pathway for the production of 3-HP from glucose using a 3-oxopropanoate intermediate produced directly from oxaloacetate. The oval indicates a novel enzyme capable of efficiently catalyzing the decarboxylation of oxaloacetate to 3-oxopropanoate.

FIG. 3 depicts the scheme for genomic enzyme mining to identify active oxaloacetate decarboxylases.

FIG. 4 shows log specific activity towards oxaloacetate for 56 candidate enzymes identified by genomic enzyme mining.

FIG. 5 shows the kinetic characterization of the top candidate enzyme identified by genomic enzyme mining, 4COK, on substrates pyruvate (squares) and oxaloacetate (diamonds).

FIG. 6 shows the results of a second round of genomic mining centered around the sequence space of 4COK to identify other candidate OAADCs. A phylogenetic tree of candidate enzymes is shown, along with the corresponding OAADC activity measured for each enzyme (log scale). A clade containing enzymes with the highest measured OAADC activity is indicated.

FIG. 7 shows the activity of candidate 3-hydroxypropionate dehydrogenase (3-HPDH) enzymes towards 3-HP using either NAD+ or NADP+ as a co-factor.

FIG. 8A shows the activity of the candidate 3-HPDH enzyme 2CVZ towards 3-HP using either NAD+ or NADP+ as a co-factor.

FIG. 8B shows the activity of the candidate 3-HPDH enzyme A4YI81 towards 3-HP using either NAD+ or NADP+ as a co-factor.

FIG. 9 shows the activities of the candidate 3-HPDH enzymes 2CVZ and A4YI81 towards 3-HP using NAD+ as a co-factor.

FIG. 10 shows the activities of candidate phosphoenolpyruvate carboxykinase (PEPCK) enzymes from E. coli and A. succinogenes towards PEP.

### DETAILED DESCRIPTION

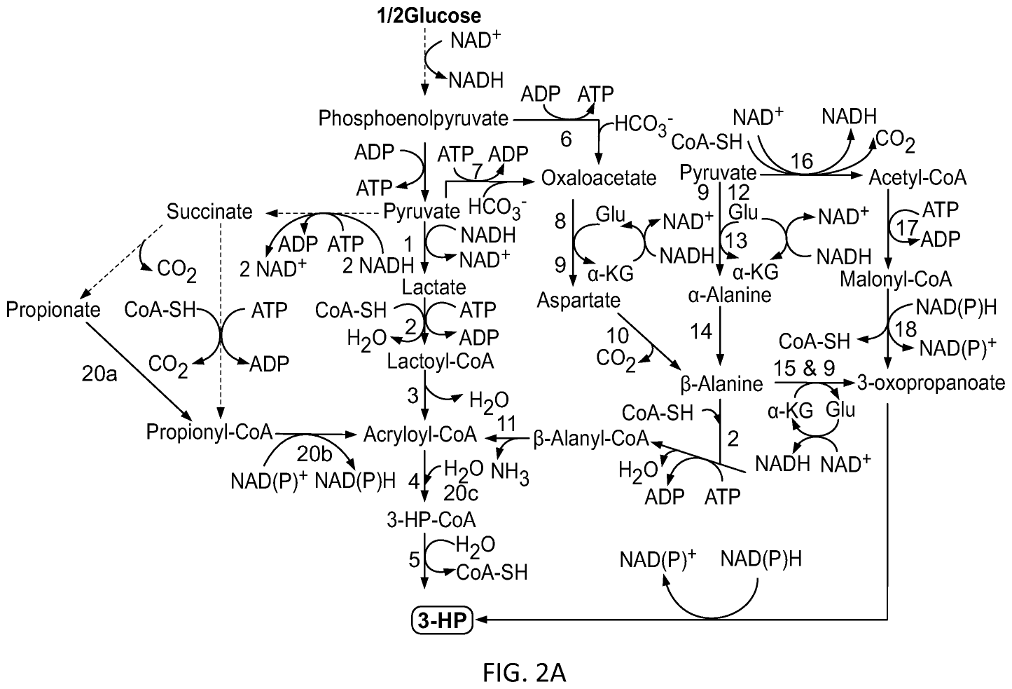

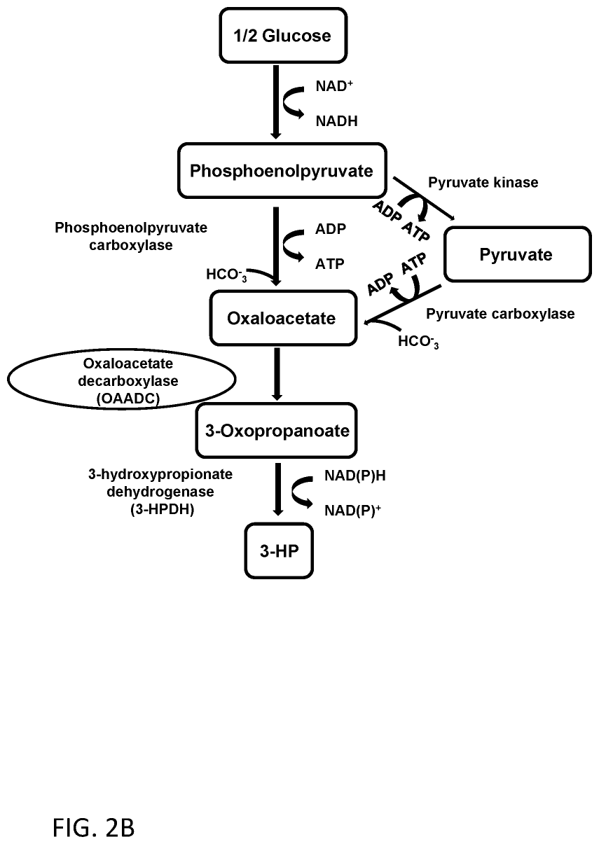

The present disclosure relates generally to methods, host cells, and vectors for producing 3-hydroxypropionate (3-HP). In some embodiments, the methods, host cells, and vectors comprise a recombinant polynucleotide encoding an oxaloacetate decarboxylase (OAADC) and a polynucleotide encoding a 3-hydroxypropionate dehydrogenase (3-HPDH). Without wishing to be bound to theory, it is thought that a simplified metabolic pathway using an OAADC to convert oxaloacetate into 3-oxopropanoate and a 3-HPDH to convert 3-oxopropanoate into 3-HP (FIG. 2B) would allow for more efficient production of 3-HP than existing pathways (FIG. 2A). For example, it is thought that utilizing this simplified metabolic pathway can result in approximately 100% conversion of glucose into 3-HP. Moreover, this metabolic pathway is active under anaerobic conditions such that host cells can grow and produce 3-HP without aeration, enabling an increased yield and increased scale of production (e.g., larger fermenter size) with lower operating costs (e.g., by eliminating the need for aeration). Finally, this pathway can be carried out using fungal cells, which are typically more tolerant of low pH than bacterial cells. For example, it is thought that using E. coli for large-scale production of 3-HP would lead to acidification of the culture medium, thereby requiring more complicated purification and pH neutralization processes to maintain the pH of the culture within a viable range for E. coli (which can also lead to undesirable waste products, such as gypsum, that raise environmental concerns).

In particular, the present disclosure is based, at least in part, on the demonstration described herein of a method for identifying enzymes with OAADC activity. As one example, 4COK from Gluconacetobacter diazotrophicus was found to have efficient OAADC activity with a particularly strong specific activity using oxaloacetate as a substrate (e.g., as compared to pyruvate and/or 2-ketoisovalerate). Additional enzymes having OAADC activity similar to that of 4COK were also identified, such as A0A0J7KM68_LASNI (SEQ ID NO:145), 5EUJ (SEQ ID NO:146). C7JF72_ACEP3 (SEQ ID NO:148), and A0A0D6NFJ6_9PROT (SEQ ID NO:166). Moreover, enzymes particularly suitable for catalyzing the other steps of the 3-HP biosynthesis pathway (e.g., PEPCK and 3-HPDH) were also characterized, such as the 3-HPDHs A4YI81 (SEQ ID NO: 154) and 2CVZ (SEQ ID NO:159) and the PEPCKs from E. coli (SEQ ID NO:162) and A. succinogenes (SEQ ID NO:163).

Methods and Host Cells for Producing 3-hydroxypropionate (3-HP)

Certain aspects of the present disclosure relate to methods of producing 3-HP. In some embodiments, the methods comprise providing a recombinant host cell that comprises a recombinant polynucleotide encoding an oxaloacetate decarboxylase (OAADC) and a polynucleotide encoding a 3-hydroxypropionate dehydrogenase (3-HPDH), wherein the OAADC has a ratio of activity against pyruvate to activity against oxaloacetate that is less than or equal to about 5:1, and culturing the recombinant host cell in a culture medium comprising a substrate under conditions suitable for the recombinant host cell to convert the substrate to 3-HP. In some embodiments, the methods comprise providing a recombinant host cell that comprises a recombinant polynucleotide encoding an oxaloacetate decarboxylase (OAADC) and a polynucleotide encoding a 3-hydroxypropionate dehydrogenase (3-HPDH), wherein the OAADC has a specific activity of at least 0.1 μmol/min/mg against oxaloacetate; and culturing the recombinant host cell in a culture medium comprising a substrate under conditions suitable for the recombinant host cell to convert the substrate to 3-HP. In some embodiments, the methods comprise providing a recombinant fungal host cell that comprises a recombinant polynucleotide encoding an oxaloacetate decarboxylase (OAADC) and a polynucleotide encoding a 3-hydroxypropionate dehydrogenase (3-HPDH); and culturing the recombinant fungal host cell in a culture medium comprising a substrate under conditions suitable for the recombinant host cell to convert the substrate to 3-HP. Expression of the OAADC and the 3-HPDH results in increased production of 3-HP, as compared to production by a host cell lacking expression of the OAADC and the 3-HPDH.

As used herein, “recombinant” or “exogenous” refer to a polynucleotide wherein the exact nucleotide sequence of the polynucleotide is not naturally found in a given host cell, e.g., as the host cell is found in nature. These terms may also refer to a polynucleotide sequence that may be naturally found in (e.g., “endogenous” with respect to) a given host, but in an unnatural (e.g., greater than or less than expected) amount, or additionally if the sequence of a polynucleotide comprises two or more subsequences that are not found in the same relationship to each other in nature. For example, regarding the latter, a recombinant polynucleotide can have two or more sequences from unrelated polynucleotides or from homologous nucleotides arranged to make a new polynucleotide, or a promoter sequence in operable linkage with a coding sequence in an unnatural combination. Specifically, the present disclosure describes the introduction of a recombinant vector into a host cell, wherein the vector contains a polynucleotide coding for a polypeptide that is not normally found in the host cell or contains a foreign polynucleotide coding for a substantially homologous polypeptide that is normally found in the host cell. With reference to the host cell's genome, the polynucleotide sequence that encodes the polypeptide is recombinant or exogenous. “Recombinant” may also be used to refer to a host cell that contains one or more exogenous or recombinant polynucleotides.

The terms “derived from” or “from” when used in reference to a polynucleotide or polypeptide indicate that its sequence is identical or substantially identical to that of an organism of interest. For instance, a 3-HPDH from Saccharomyces cerevisiae refers to a 3-HPDH enzyme having a sequence identical or substantially identical to a native 3-HPDH of Saccharomyces cerevisiae. The terms “derived from” and “from” when used in reference to a polynucleotide or polypeptide do not indicate that the polynucleotide or polypeptide in question was necessarily directly purified, isolated, or otherwise obtained from an organism of interest. By way of example, an isolated polynucleotide containing a 3-HPDH coding sequence of Saccharomyces cerevisiae need not be obtained directly from a Saccharomyces cerevisiae cell. Instead, the isolated polynucleotide may be prepared synthetically using methods known to one of skill in the art, including but not limited to polymerase chain reaction (PCR) and/or standard recombinant cloning techniques.

“Percent (%) amino acid sequence identity” with respect to a reference polypeptide sequence refers to the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. When comparing two sequences for identity, it is not necessary that the sequences be contiguous, but any gap would carry with it a penalty that would reduce the overall percent identity. For blastn, the default parameters are Gap opening penalty=5 and Gap extension penalty=2. For blastp, the default parameters are Gap opening penalty=11 and Gap extension penalty=1. Alignment for purposes of determining percent amino acid sequence identity can be achieved in various ways that are within the skill in the art, for instance, by the local homology algorithm of Smith and Waterman, Adv Appl Math, 2:482, 1981; by the homology alignment algorithm of Needleman and Wunsch, J Mol Biol, 48:443, 1970; by the search for similarity method of Pearson and Lipman, Proc Natl Acad Sci USA, 85:2444, 1988; by computerized implementations of these algorithms FASTDB (Intelligenetics), by the BLAST or BLAST 2.0 algorithms (Altschul et al., Nuc Acids Res, 25:3389-3402, 1977; and Altschul et al., J Mol Biol, 215:403-410, 1990, respectively), GAP, BESTFIT, FASTA, and TFASTA in the Wisconsin Genetics Software Package (Genetics Computer Group, Madison, Wis.), PILEUP (Feng and Doolittle. J Mol Evol, 35:351-360, 1987), the CLUSTALW program (Thompson et al., Nucl Acids. Res, 22:4673-4680, 1994), or by manual alignment and visual inspection. Suitable parameters for any of these exemplary algorithms, such as gap open and gap extension penalties, scoring matrices (see. e.g., the BLOSUM62 scoring matrix of Henikoff and Henikoff, Proc Natl Acad Sci USA, 89:10915, 1989), and the like can be selected by one of ordinary skill in the art.

The terms “coding sequence” and “open reading frame (ORF)” refer to a sequence of codons extending from an initiator codon (ATG) to a terminator codon (TAG, TAA or TGA), which can be translated into a polypeptide.

The terms “decrease,” “reduce” and “reduction” as used in reference to biological function (e.g., enzymatic activity, production of compound, expression of a protein, etc.) refer to a measurable lessening in the function by at least 10%, at least 50%, at least 75%, or at least 90%. Depending upon the function, the reduction may be from 10% to 100%. The term “substantial reduction” and the like refer to a reduction of at least 50%, 75%, 90%, 95%, or 100%.

The terms “increase,” “elevate” and “enhance” as used in reference to biological function (e.g., enzymatic activity, production of compound, expression of a protein, etc.) refer to a measurable augmentation in the function by at least 10%, at least 50%, at least 75%, or at least 90%. Depending upon the function, the elevation may be from 10% to 100%; or at least 10-fold, 100-fold, or 1000-fold up to 100-fold, 1000-fold or 10,000-fold or more. The term “substantial elevation” and the like refer to an elevation of at least 50%, 75%, 90%, 95%, or 100%.

### Oxaloacetate Decarboxylases

Certain aspects of the present disclosure relate to oxaloacetate decarboxylase (OAADC) enzymes and recombinant polynucleotides related thereto. As used herein, an oxaloacetate decarboxylase (OAADC) is capable of catalyzing the reaction converting oxaloacetate to 3-oxopropanoate (also known as malonate semialdehyde). The discovery of enzymes capable of catalyzing this reaction with sufficient efficiency for enabling large-scale processes (e.g., production of 3-HP) is described and demonstrated herein.

In some embodiments, the OAADC has a ratio of activity against pyruvate to activity against oxaloacetate that is less than or equal to about 5:1. In some embodiments, the OAADC has at least about 20% activity using oxaloacetate as a substrate as compared to its activity using pyruvate as a substrate. Exemplary assays for determining enzymatic activity against pyruvate or oxaloacetate (e.g., using pyruvate or oxaloacetate as a substrate) are described in greater detail in Examples 1 and 2 below.

In some embodiments, an OAADC of the present disclosure has a ratio of activity against oxaloacetate to activity against 2-ketoisovalerate that is greater than or equal to about 5, about 10, about 25, about 50, about 75, about 100, about 150, about 200, about 250, about 300, or about 350. For example, as described herein, 4COK from Gluconoacetobacter diazotrophicus was demonstrated to possess approximately 390-fold greater activity towards oxaloacetate than 2-ketoisovalerate. Additional OAADCs with similar enzymatic activity to that of 4COK were also identified, such as A0A0J7KM68_LASNI (SEQ ID NO:145), 5EUJ (SEQ ID NO:146), C7JF72_ACEP3 (SEQ ID NO:148), and A0A0D6NFJ6_9PROT (SEQ ID NO:166), as described in greater detail in Example 2 below. In some embodiments, an OAADC of the present disclosure has a ratio of activity against oxaloacetate to activity against 2-ketoisovalerate that is greater than or equal to about 5, about 10, about 25, about 50, about 75, about 100, about 150, about 200, about 250, about 300, or about 350 and a ratio of activity against pyruvate to activity against oxaloacetate that is less than or equal to about 5:1. Exemplary assays for determining enzymatic activity against pyruvate, 2-ketoisovalerate, or oxaloacetate (e.g., using pyruvate, 2-ketoisovalerate, or oxaloacetate as a substrate) are described in greater detail in Examples 1 and 2 below.

In some embodiments, an OAADC of the present disclosure has a ratio of activity against oxaloacetate to activity against 4-methyl-2-oxovaleric acid that is greater than or equal to about 5, about 10, about 25, about 50, about 75, about 100, about 150, about 200, about 250, about 300, or about 350. In some embodiments, an OAADC of the present disclosure has a ratio of activity against oxaloacetate to activity against 4-methyl-2-oxovaleric acid that is greater than or equal to about 5, about 10, about 25, about 50, about 75, about 100, about 150, about 200, about 250, about 300, or about 350 and a ratio of activity against pyruvate to activity against oxaloacetate that is less than or equal to about 5:1. The exemplary assays for determining enzymatic activity against pyruvate, 2-ketoisovalerate, or oxaloacetate (e.g., using pyruvate, 2-ketoisovalerate, or oxaloacetate as a substrate) described in Example 1 below can readily be modified to measure activity against 4-methyl-2-oxovaleric acid by one of skill in the art.

In some embodiments, an OAADC of the present disclosure has a specific activity of at least 0.1 μmol/min/mg, at least 10 μmol/min/mg, or at least 100 μmol/min/mg against oxaloacetate. In some embodiments, an OAADC of the present disclosure has a specific activity against oxaloacetate of at least about 0.1, at least about 0.5, at least about 1, at least about 5, at least about 10, at least about 25, at least about 50, at least about 75, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 μmol/min/mg. For example, as described herein, 4COK from Gluconoacetobacter diazotrophicus was demonstrated to possess a specific activity against oxaloacetate of approximately 5500 μmol/min/mg. Additional OAADCs with similar enzymatic activity to that of 4COK were also identified, such as A0A0J7KM68_LASNI (SEQ ID NO:145), 5EUJ (SEQ ID NO:146), C7JF72_ACEP3 (SEQ ID NO:148), and A0A0D6NFJ6_9PROT (SEQ ID NO:166), as described in greater detail in Example 2 below. In some embodiments, an OAADC of the present disclosure has a specific activity of at least 0.1 μmol/min/mg, at least 10 μmol/min/mg, or at least 100 mol/min/mg against oxaloacetate and a ratio of activity against pyruvate to activity against oxaloacetate that is less than or equal to about 5:1. In some embodiments, an OAADC of the present disclosure has a specific activity of at least 0.1 μmol/min/mg, at least 10 μmol/min/mg, or at least 100 mol/min/mg against oxaloacetate and a ratio of activity against oxaloacetate to activity against 2-ketoisovalerate that is greater than or equal to about 5, about 10, about 25, about 50, about 75, about 100, about 150, about 200, about 250, about 300, or about 350. In some embodiments, an OAADC of the present disclosure has a specific activity of at least 0.1 μmol/min/mg, at least 10 μmol/min/mg, or at least 100 μmol/min/mg against oxaloacetate, a ratio of activity against pyruvate to activity against oxaloacetate that is less than or equal to about 5:1, and a ratio of activity against oxaloacetate to activity against 2-ketoisovalerate that is greater than or equal to about 5, about 10, about 25, about 50, about 75, about 100, about 150, about 200, about 250, about 300, or about 350. Exemplary assays for determining specific activity against oxaloacetate (e.g., using oxaloacetate as a substrate) are described in greater detail in Example 1 below. In some embodiments, specific activity refers to enzymatic conversion of oxaloacetate into 3-oxopropanoate.

In some embodiments, an OAADC of the present disclosure is expressed in a host cell at up to 1% of total protein. In some embodiments, an OAADC and a 3-HPDH of the present disclosure have a combined expression in a host cell of up to 1% of total protein.

In some embodiments, an OAADC of the present disclosure has a catalytic efficiency (k_{cat}/K_{M}) for oxaloacetate that is greater than about 500, 1000, or 2000 (M⁻¹s⁻¹). For example, as described herein, 4COK from Gluconoacetobacter diazotrophicus was demonstrated to possess a catalytic efficiency for oxaloacetate of approximately 2296.4. Exemplary assays for determining catalytic efficiency and other rate constants using oxaloacetate as a substrate are described in greater detail in Example 1 below. Additional OAADCs with similar enzymatic activity to that of 4COK were also identified, such as A0A0J7KM68_LASNI (SEQ ID NO:145). 5EUJ (SEQ ID NO:146). C7JF72_ACEP3 (SEQ ID NO:148), and A0A0D6NFJ6_9PROT (SEQ ID NO:166), as described in greater detail in Example 2 below.

In some embodiments, an OAADC of the present disclosure comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to an amino acid sequence shown in Table 2. In some embodiments, an OAADC of the present disclosure is encoded by a polynucleotide sequence shown in Table 2.

In some embodiments, an OAADC of the present disclosure comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to MTYTVGRYLADRLAQIGLKHHFAVAGDYNLVLLDQLLLNTDMQQIYCSNELNCG FSAEGYARANGAAAAIVTFSVGALSAFNALGGAYAENLPVILISGAPNANDHGTGH ILHHTLGTTIDYGYQLEMARHITCAAESIVAAEDAPAKIDHVIRTALREKKPAYLEIA CNVAGAPCVRPGGIDALLSPPAPDEASLKAAVDAALAFIEQRGSVTMLVGSRIRAA GAQAQAVALADALGCAVTTMAAAKSFFPEDHPGYRGHYWGEVSSPGAQQAVEG ADGVICLAPVFNDYATVGWSAWPKGDNVMLVERHAVTVGGVAYAGIDMRDFLT RLAAHTVRRDATARGGAYVTPQTPAAAPTAPLNNAEMARQIGALLTPRTTLTAET GDSWFNAVRMKLPHGARVELEMQWGHIGWSVPAAFGNALAAPERQHVLMVGD GSFQLTAQEVAQMIRHDLPVIIFLINNHGYTIEVMIHDGPYNNVKNWDYAGLMEVF NAGEGNGLGLRARTGGELAAAIEQARANRNGPTLIECTLDRDDCTQELVTWGKRV AAANARPPRAG (SEQ ID NO:1). In some embodiments, an OAADC of the present disclosure comprises the amino acid sequence MTYTVGRYLADRLAQIGLKHHFAVAGDYNLVLLDQLLLNTDMQQIYCSNELNCG FSAEGYARANGAAAAIVTFSVGALSAFNALGGAYAENLPVILISGAPNANDHGTGH ILHHTLGTITDYGYQLEMARHITCAAESIVAAEDAPAKIDHVIRTALREKKPAYLEIA CNVAGAPCVRPGGIDALLSPPAPDEASLKAAVDAALAFIEQRGSVTMLVGSRIRAA GAQAQAVALADALGCAVITMAAAKSFFPEDHPGYRGHYWGEVSSPGAQQAVEG ADGVICLAPVFNDYATVGWSAWPKGDNVMLVERHAVTVGGVAYAGIDMRDFLT RLAAHTVRRDATARGGAYVTPQTPAAAPTAPLNNAEMARQIGALLTPRTTLTAET GDSWFNAVRMKLPHGARVELEMQWGHIGWSVPAAFGNALAAPERQHVLMVGD GSFQLTAQEVAQMIRHDLPVIIFLINNHGYTIEVMIHDGPYNNVKNWDYAGLMEVF NAGEGNGLGLRARTGGELAAAIEQARANRNGPTLIECTLDRDDCTQELVTWGKRV AAANARPPRAG (SEQ ID NO:1). In some embodiments, an OAADC of the present disclosure comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97% at, at least 98%, at least 99%, or 100% identical to the amino acid sequence of GenBank/NCBI RefSeq Accession Nos. AIG13066, WP_012554212, and/or WP_012222411.

In some embodiments, an OAADC of the present disclosure is encoded by the polynucleotide sequence of SEQ ID NO:2.

In some embodiments, an OAADC of the present disclosure has a specific activity against oxaloacetate of at least about 10 gμmol/min/mg. In some embodiments, an OAADC of the present disclosure comprises the amino acid sequence of a polypeptide selected from the group consisting of 4COK (SEQ ID NO:1), A0A0F6SDN1_9DELT (SEQ ID NO:3), 4K9Q (SEQ ID NO:5), 1JSC (SEQ ID NO:15). 3L84_3M34 (SEQ ID NO:19). A0A0F2PQV5_9FIRM (SEQ ID NO:25), A0A0R2PY37_9ACTN (SEQ ID NO:41), X1WK73_ACYPI (SEQ ID NO:43), F4RJP4_MELLP (SEQ ID NO:51), A0A081BQW3_9BACT (SEQ ID NO:53), CAK95977 (SEQ ID NO:55), YP_831380 (SEQ ID NO:57), ZP_06846103 (SEQ ID NO:61), ZP_08570611 (SEQ ID NO:65), WP_010764607.1 (SEQ ID NO:77), YP_005756646.1 (SEQ ID NO:81), WP_018535238.1 (SEQ ID NO:85), YP_006485164.1 (SEQ ID NO:112), YP_005461458.1 (SEQ ID NO: 113), YP_006991301.1 (SEQ ID NO:114), WP_003075272.1 (SEQ ID NO:115), WP_020634527.1 (SEQ ID NO:116), 1OVM (SEQ ID NO:117), 2Q5Q (SEQ ID NO:118), 2VBG (SEQ ID NO:119), 2VBI (SEQ ID NO:120), and 3FZN (SEQ ID NO:121). Additional OAADCs with similar enzymatic activity to that of 4COK were also identified, such as A0A0J7KM68_LASNI (SEQ ID NO:145), 5EUJ (SEQ ID NO:146), C7JF72_ACEP3 (SEQ ID NO:148), and A0A0D6NFJ6_9PROT (SEQ ID NO:166).

In some embodiments, an OAADC of the present disclosure comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence of A0A0J7KM68_LASNI, 5EUJ, or C7JF72_ACEP3 (see Table 5A). In some embodiments, an OAADC of the present disclosure comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence selected from the group consisting of SEQ ID NOs:1, 145, 146, 148, and 166. In some embodiments, an OAADC of the present disclosure comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence selected from the group consisting of SEQ ID NOs:145, 146, 148, and 166. In some embodiments, an OAADC of the present disclosure comprises the sequence of A0A0J7KM68_LASNI, 5EUJ, C7JF72_ACEP3, or A0A0D6NFJ6_9PROT (see Table 5A). In some embodiments, an OAADC of the present disclosure comprises a sequence selected from the group consisting of SEQ ID NOs:1, 145, 146, 148, and 166. In some embodiments, an OAADC of the present disclosure comprises a sequence selected from the group consisting of SEQ ID NOs:145, 146, 148, and 166.

In some embodiments, an OAADC of the present disclosure has a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence shown in Table 5A.

TABLE 5A
Candidate OAADC sequences.

| Enzyme name | Amino acid seqence |
|---|---|
| G6EYP0 9PROT | MEYTVGQYLATRLAQLGLNHVFAVAGDYNLTLLDEMAKAKDLEQVYCCNEL |
| | NCGFAGEGYARARIMGASVVTFSVGAFSAFNAVGGAFAENLPLLLISGAPNNN |
| | DYGSGHILHHTMGYSDYRYQMEMAKKITCEAVSVAHADEAPCLIDHAIRSAIR |
| | NRKPAYIEISCNVANQPCTEPGPISSITNSLISDDESLKAAAKACVEALEKAKNPV |
| | VIIGGKIRSAGCAVSKQVAELTKKLGCAVATMAQAKGLSPEEEAEYVGTFWGD |
| | ISSPGVEDLVRDSDCRIYIGAVFNDYSTVGWTCKLVSDNDILISSHHTRVGKKEF |
| | SGVYLKDFIPVLASSVKKNTTSLEQFKAKKLPAKETPVADGNAALTTVELCRQI |
| | QGAINKDTTLFLETGDSWFHGMHFNLPNGARVESEMQWGHIGWSIPSMFGYAV |
| | SEPNRRNIIMVGDGSFQLTAQEVCQMIRRNMPVIIILINNSGYTIEVKIHDGPYNRI |
| | KNWDYAGLIDVFNAEDGKGLGLKAKNGAELEKAMKTALAHKDGPTLIEVDID |
| | AQDCSPDLVVWGKKVAKANGRAPRKAGGSG (SEQ ID NO: 137) |
| W7DU13 9PROT | MKYTVGQYLATRLAQLGLNHVFAVAGDYNLTLLDEMAKVEDLEQVYCCNEL |
| | NCGFAGEGYARSRVMGASVVTFSVGAFSAFNAVGGAFAENLPLLLISGAPNNN |
| | DYGSGHILHHTMGYSDYRYQMDMAKQITCEAVSVAHADEAPCLIDHAIRSALR |
| | NRKPAYIEISCNVANQPCTEPGPISSITNSLISDDESLKAAAKACLDALEKAKSPV |
| | VIIGGKIRSAGCAVSKKVAELTKKLGCAVATMAQAKGLSPEEEAEYVGTFWGEI |
| | SSPGVEELVRESDCRIYIGAVFNDYSTVGWTCKLNGENDILISSHHTRVGHKEFS |
| | GVYLKDFIPVLTSCVKKNTTSLDQFKAKKIPVKQVPVADGKAPLTTVELCRQIQ |
| | GAINKDTTIYLETGDSWFHGMHFKLPNGARVESEMQWGHIGWSIPSMFGYAVS |
| | EPNRRNIIMVGDGSFQLTAQEVCQMIRRNIPIIIILINNSGYTIEVKIHDGPYNRIKN |
| | WDYAGLINVFNAEDGKGLGLKAKNGAELEKAMQTALAHKDGPTLIEVDIDAQ |
| | DCSPDLVVWGKKVAKANGRAPRKFQTFGGSG (SEQ ID NO: 138) |
| I4H6Y9 MICAE_1 | MSNYNVGTYLAERLVQIGVKHHFVVPGDYNLVLLDQFLKNQNLLQVGCCNEL |
| | NCGFAAEGYARANGLGVAVVTYSVGALSALNAIGGAYAENLPVILVSGAPNTN |
| | DYSTGHLLHHTMGTQDLTYVLEIARKLTCAAVSITSAEDAPEQIDHVIRTALREQ |
| | KPAYIEIACNIAAAPCASPGPVSAIINEVPSDAETLAAAVSAAAEFLDSKQKPVLL |
| | IGSQLRAAKAEQEAIELAEALGCSVAVMAAAKSFFPEEHPQYVGTYWGEISSPG |
| | TSAIVDWSDAVVCLGAVFNDYSTVGWTAMPSGPTVLNANKDSVKFDGYHFSGI |
| | HLRDFLSCLARKVEKRDATMAEFARFRSTSVPVEPARSEAKLSRIEMLRQIGPLV |
| | TAKTTVFAETGDSWFNGMKLQLPTGARFEIEMQWGHIGWSIPAAFGYALGAPE |
| | RQIICMIGDGSFQLTAQEVAQMIRQKLPIIIFLVNNHGYTIEVEIHDGPYNNIKNW |
| | DYAGLIKVFNAEDGAGQGLLATTAGELAQAIEVALENREGPTLIECVIDRDDAT |
| | ADLISWGRAVAVANARPHRGGSG (SEQ ID NO: 139) |
| A0A094IGF4 9PEZI | MATFTVGDYLAERLAQIGIRHHFVVPGDYNLILLDKLQSHPDLSELGCANELNC |
| | SLAAEGYARAQGVAACIVTYSVGAFSAFNGTGSAYAENLPLILVSGSPNTNDSA |
| | KFHLLHHTLGTNDFTYQFEMAKKITCCAVAVGRAQDAPRLIDQAIRAALLAKK |
| | PAYIEIPTNLSGAMCVRPGPISAVVEPVLSDKASLTAAVDRAVQYLCGKQKPAIL |
| | VGPKLRRAGAEMALLQVAEAIGCAVAVQPAAKGFFPEDHKQFAGVFWGQVST |
| | LAADSILNWADTILCVGTIFTDYSTVGWTALPNVPLMIAEMDHVMFPGATFGR |
| | VRLNDFLSGLAKTVGRNESTMVEYGYIRPDPPLVHAAAPDELLNRKETARQVQ |
| | MLLTPETTVFVDTGDSWFNGIRMKLPRGASFEIEMQWGHIGWSIPAAFGYAMG |
| | KPERKVITMVGDGSFQMTAQEVSQMVRYKVPIIIFLINNKGYTIEVEIHDGLYNR |
| | IKNWDYALLVRAFNSNDGQAIGFRASTGRELAEAIEKAKAHKDGPTLIECVIDQ |
| | DDCSRELITWGHYVAAANARPPVQTGGSG (SEQ ID NO: 140) |
| A0A0D2CX28 | MSWTVGSYLAERLAQIGIEHHFVVPGDYNLVLLDKLQAHPKLSEIGCANELNCS |
| 9EURO | FAAEGYARAKGVAAAVVTFSVGAFSAFNGVGGAYAENLPVILISGAPNTSDSG |
| | AFHLLHHTLGTHDFGYQLEMAKKITCAAVAIRRAQDAPRLIDHAIRSAMSAKKP |
| | AYIEIPTNLSIANCPAPGPISAVIAPERSDEITLAMAVNAALDWLKSKQKPVLLAG |
| | PKLRAAGAEAAFLQLADALGCAVAVLPGAKSFFPEDHKQFVGVYWGQVSTMG |
| | ADAIVDWSDGIFGAGVVFTDYSTVGWTALPPDSITLTADLDHMSFTGAEFNRV |
| | QLAELLSALAERATRNSSTMVEYAHLRPDVLFPHIEEPKLPLHRNEIARQIQQLL |
| | QPKTTLFVETGDSWFNGVQMRLPRSCRFEIEMQWGHIGWSVPASFGYAVGSPE |
| | RQIILMVGDGSFQMTVQEVSQMVRARLPIIIFLMNNRGYTIEVEIHDGLYNRIKN |
| | WNYASLIEAFNAEDGHAKGIKASNPEQLAQAIKLATSNSDGPTLIECVIDQDDCT |
| | RELITWGHYVASANARPPAHKGGSG (SEQ ID NO: 141) |
| H6C7K9 EXODN | MRCMSVPSMTFSRHTLRSCATSSDRMTGAPRKPFITSIKRQHQQPWHSICPNVTI |
| | IMSWTVGSYLAERLSQIGIEHHFVVPGDYNLVLLDQLQAHPKLSEIGCANELNC |
| | SFAAEGYARAKGVAAAVVTFSVGAFSAFNGLGGAYAENLPVILISGSPNTNDAG |
| | AFHLLHHTLGTHDFEYORQIAEKITCAAVAVRRAQDAPRLIDHAIRSALLAKKP |
| | SYIEIPTNLSNVTCPAPGPISAVIAPEPSDEPTLAAAVHAATNWLKAKQKPILLAG |
| | PKLRAAGGEAGFLQLAEAIGCAVAVMPGAKSFFPEDHKQFVGVYWGQASTMG |
| | ADAIVDWADGIFGAGLVFTDYSTVGWTAIPSESITLNADLDNMSFPGATFNRVR |
| | LADLLSALAKEATPNPSTMVEYARLRPDILPPHHEQPKLPLHRVEIARQIQELLH |
| | PKTTLFAETGDSWFNAMQMNLPRDCRFEIEMQWGHIGWSVPASFGYAVGAPE |
| | RQVLLMIGDGSFQMTAQEVSQMVRSKVPIIIFLMNNGGYTIEVEIHDGLYNRIKN |
| | WNYAAMMEVFNAGDGHAKGIKASNPEQLAQAIKLAKSNSEGPTLIECIIDQDD |
| | CTKELITWGHYVATANGRPPAHTGGSG (SEQ ID NO: 142) |
| PDC2 SCHPO | MTKDAESTMTVGTYLAQRLVEIGIKNHFVVPGDYNLRLLDFLEYYPGLSEIGCC |
| | NELNCAFAAEGYARSNGIACAVVTYSVGALTAFDGIGGAYAENLPVILVSGSPN |
| | TNDLSSGHLLHHTLGTHDFEYQMEIAKKLTCAAVAIKRAEDAPVMIDHAIRQAI |
| | LQHKPVYIEIPTNMANQPCPVPGPISAVISPEISDKESLEKATDIAAELISKKEKPIL |
| | LAGPKLRAAGAESAFVKLAEALNCAAFIMPAAKGFYSEEHKNYAGVYWGEVS |
| | SSETTKAVYESSDLVIGAGVLFNDYSTVGWRAAPNPNILLNSDYTSVSIPGYVFS |
| | RVYMAEFLELLAKKVSKKPATLEAYNKARPQTVVPKAAEPKAALNRVEVMRQ |
| | IQGLVDSNTTLYAETGDSWFNGLQMKLPAGAKFEVEMQWGHIGWSVPSAMGY |
| | AVAAPERRTIVMVGDGSFQLTGQEISQMIRHKLPVLIFLLNNRGYTIEIQIHDGPY |
| | NRIQNWDFAAFCESLNGETGKAKGLHAKTGEELTSAIKVALQNKEGPTLIECAI |
| | DTDDCTQELVDWGKAVRSANARPPTADNGGSG (SEQ ID NO: 143) |
| IZPD | MSYTVGTYLAERLVQIGLKHHFAVAGDYNLVLLDNLLLNKNMEQVYCCNELN |
| | CGFSAEGYARAKGAAAAVVTYSVGALSAFDAIGGAYAENLPVILISGAPNNND |
| | HAAGHVLHHALGKTDYHYQLEMAKNITAAAEAIYTPEEAPAKIDHVIKTALRE |
| | KKPVVLEIACNIASMPCAAPGPASALFNDEASDEASLNAAVDETLKFIANRDKV |
| | AVLVGSKLRAAGAEEAAVKFTDALGGAVATMAAAKSFFPEENALYIGTSWGE |
| | VSYPGVEKTMKEADAVIALAPVFNDYSTTGWTDIPDPKKLVLAEPRSVVVNGIR |
| | FPSVHLKDYLTRLAQKVSKKTGSLDFFKSLNAGELKKAAPADPSAPLVNAEIAR |
| | QVEALLTPNTTVIAETGDSWFNAQRMKLPNGARVEYEMQWGHIGWSVPAAFG |
| | YAVGAPERRNILMVGDGSFQLTAQEVAQMVRLKLPVIIFLINNYGYTIEVMIHD |
| | GPYNNIKNWDYAGLMEVFNGNGGYDSGAAKGLKAKTGGELAEAIKVALANT |
| | DGPTLIECFIGREDCTEELVKWGKRVAAANSRKPVNKVV (SEQ ID NO: 144) |
| 4COK | MTYTVGRYLADRLAQIGLKHHFAVAGDYNLVLLDQLLLNTDMQQIYCSNELN |
| | CGFSAEGYARANGAAAAIVTFSVGALSAFNALGGAYAENLPVILISGAPNANDH |
| | GTGHILHHTLGTTDYGYQLEMARHITCAAESIVAAEDAPAKIDHVIRTALREKK |
| | PAYLEIACNVAGAPCVRPGGIDALLSPPAPDEASLKAAVDAALAFIEQRGSVTM |
| | LVGSRIRAAGAQAQAVALADALGCAVTTMAAAKSFFPEDHPGYRGHYWGEVS |
| | SPGAOQAVEGADGVICLAPVFNDYATVGWSAWPKGDNVMLVERHAVTVGGV |
| | AYAGIDMRDFLTRLAAHTVRRDATARGGAYVTPQTPAAAPTAPLNNAEMARQI |
| | GALLTPRTTLTAETGDSWFNAVRMKLPHGARVELEMQWGHIGWSVPAAFGNA |
| | LAAPERQHVLMVGDGSFQLTAQEVAQMIRHDLPVIIFLINNHGYTIEVMIHDGP |
| | YNNVKNWDYAGLMEVFNAGEGNGLGLRARTGGELAAAIEQARANRNGPTLIE |
| | CTLDRDDCTQELVTWGKRVAAANARPPRAG (SEQ ID NO: 1) |
| A0A0J7KM68 | MSYTVGQYLADRLVQIGLKDHFAIAGDYNLVLLDQFLKNKNWNQIYDCNELN |
| LASNI | CGFAAEGYARANGAAACVVTYTVGAISAMNSALAGAYAENLPVLCISGAPNC |
| | NDYGSGRILHHTIGKPEFTQQLDMVKHVTCAAESVVQASEAPAKIDHVIRTMLL |
| | EQRPAYIDIACNISGLECPRPGPIEDLLPQYAADNKSLTSAIDAIAKKIEASQKVTL |
| | YVGPKVRPGKAKEASVKLADALGCAVTVGPASMSFFPAKHPGFRGTYWGIVST |
| | GDANKVVEEAETLIVLGPNWNDYATVGWKAWPKGPRVVTIDEKAAQVDGQV |
| | FSGLSMKALVEGLAKKVSKKPATAEGTKAPHFEYTVAKPDAKLTNAEMARQIN |
| | AILDDNTTLHAETGDSWFNVKNMNWPNGLRIESEMQYGHIGWSIPSGFGGAIGS |
| | PERKHIIMCGDGSFQLTCQEVSQMIRYKLPVTIFLIDNHGYGIEIAIHDGPYNYIQ |
| | NWNFTKLMEVFNGEGEECPYSHNKNGKSGLGLKATTPAELADAIKQAEANKE |
| | GPTLIQVVIDQDDCTKDLLTWGKEVAKTNARSPVVTDKAGGSG (SEQ ID |
| | NO: 145) |
| 5EUJ | MYTVGMYLAERLAQIGLKHHFAVAGDYNLVLLDQLLLNKDMEQVYCCNELN |
| | CGFSAEGYARARGAAAAIVTFSVGAISAMNAIGGAYAENLPVILISGSPNTNDY |
| | GTGHILHHTIGTTDYNYQLEMVKHVTCAAESIVSAEEAPAKIDHVIRTALRERKP |
| | AYLEIACNVAGAECVRPGPINSLLRELEVDQTSVTAAVDAAVEWLQDRQNVV |
| | MLVGSKLRAAAAEKQAVALADRLGCAVTIMAAAKGFFPEDHPNFRGLYWGEV |
| | SSEGAQELVENADAILCLAPVFNDYATVGWNSWPKGDNVMVMDTDRVTFAG |
| | QSFEGLSLSTFAAALAEKAPSRPATTQGTQAPVLGIEAAEPNAPLTNDEMTRQIQ |
| | SLITSDTTLTAETGDSWFNASRMPIPGGARVELEMQWGHIGWSVPSAFGNAVGS |
| | PERRHIMMVGDGSFQLTAQEVAQMIRYEIPVIIFLINNRGYVIEIAIHDGPYNYIK |
| | NWNYAGLIDVFNDEDGHGLGLKASTGAELEGAIKKALDNRRGPTLIECNIAQD |
| | DCTETLIAWGKRVAATNSRKPQAGGSG (SEQ ID NO: 146) |
| 2584327140 | MAYTVGMYLAERLAQIGLKHHFAVAGDYNLVLLDQLLLNKDMEQIYCCNELN |
| EU61DRAFT | CGFSAEGYARAHGAAAAVVTFSVGAISAMNAIGGAYAENLPVILISGSPNSNDY |
| | GSGHILHHTLGTTDYGYQLEMARHVTCAAESITDAASAPAKIDHVIRTALRERK |
| | PAYLEIACNVSSAECPRPGPVSSLLAEPATDPVSLKAALEASLSALNKAERVVML |
| | VGSKIRAADAQAQAVELADRLGCAVTIMSAAKGFFPEDHPGFRGLYWGEVSSP |
| | GAQELVENADAVLCLAPVFNDYSTVGWNAWPKGDKVLLAEPNRVTVGGQSFE |
| | GFALRDFLKGLTDRAPSKPATAQGTHAPKLEIKPAARDARLTNDEMARQINAM |
| | LTPNTTLAAETGDSWFNAMRMNLPGGARVEVEMQWGHIGWSVPSTFGNAMG |
| | SKDRQHIMMVGDGSFQLTAQEVAQMWYELPVIIFLVNNKGYVIEIAIHDGPYN |
| | YIKNWDYAGLMEVFNAGEGHGIGLHAKTAGELEDAIKKAQANKRGPTIIECSLE |
| | RTDCTETLIKWGKRVAAANSRKPQAVGGSG (SEQ ED NO: 147) |
| C7JF72 ACEP3 | MTYTVGMYLAERLSQIGLKHHFAVAGDFNLVLLDQLLVNKEMEQVYCCNELN |
| | CGFSAEGYARAHGAAAAVVTFSVGAISAMNAIAGAYAENLPVILISGSPNSNDY |
| | GTGHILHHTLGTNDYTYQLEMMRHVTCAAESITDAASAPAKIDHVIRTALRERK |
| | PAYVEIACNVSDAECVRPGPVSSLLAELRADDVSLKAAVEASLALLEKSQRVTM |
| | IVGSKVRAAHAQTQTEHLADKLGCAVTIMAAAKSFFPEDHKGFRGLYWGDVSS |
| | PGAQELVEKSDALICVAPVFNDYSTVGWTAWPKGDNVLLAEPNRVTVGGKTY |
| | EGFTLREFLEELAKKAPSRPLTAQESKKHTPVIEASKGDARLTNDEMTRQINAM |
| | LTSDTTLVAETGDSWFNATRMDLPRGARVELEMQWGHIGWSVPSAFGNAMGS |
| | QERQHILMVGDGSFQLTAQEMAQMVRYKLPVIIFLVNNRGYVIEIAIHDGPYNY |
| | IKNWDYAGLMEVFNAEDGHGLGLKATTAGELEEAIKKAKTNREGPTIIECQIER |
| | SDCTKTLVEWGKKVAAANSRKPQVSGGSG (SEQ ID NO: 148) |
| A0A0D6NFJ6 | MTYTVGMYLADRLAQIGLKHHFAVAGDYNLVLLDQLLTNKDMQQIYCCNELN |
| 9PROT | CGFSAEGYARAHGAAAAVVTFSVGAISAMNAIGGAYAENLPVILISGSPNSNDY |
| | GSGHILHHTIGSTDYGYQMEMVKHVTCAAESITDAASAPAKIDHVIRTALRESK |
| | PAYLEIACNVSAQECPRPGPVSSLLSEPAPDKTSLDAAVAAAVKLIEGAENTVIL |
| | VGSKLRAARAQAEAEKLADKLECAVTIMAAAKGFFPEDHAGFRGLYWGEVSS |
| | PGTQELVEKADAIICLAPVFNDYSTVGWTAWPKGDKVLLAEPNRVTIKGQTFEG |
| | FALRDFLTALAAKAPARPASAKASSHTPTAFPKADAKAPLTNDEMARQINAML |
| | TSDTTLVAETGDSWFNAMRMTLPRGARVELEMQWGHIGWSVPSSFGNAMGSQ |
| | DRQHVVMVGDGSFQLTAQEVAQMVRYELPVIIFLVNRGYVIEIAIHDGPYNYI |
| | KNWDYAGLMEVFNAGEGHGLGLHATTAEELEDAIKKAQANRRGPTIIECKIDR |
| | QDCTDTLVQWGKKVASANSRKPQAVGGSG (SEQ ID NO: 166) |

3-hydroxypropionate Dehydrogenases

Certain aspects of the present disclosure relate to 3-hydroxypropionate dehydrogenase (3-HPDH) enzymes and polynucleotides related thereto. In some embodiments, a 3-HPDH of the present disclosure refers to an enzyme that catalyzes the conversion of 3-oxopropanoate into 3-HP. Any enzyme capable of catalyzing the conversion of 3-oxopropanoate into 3-HP, e.g., known or predicted to have the enzymatic activity described by EC 1.1.1.59 and/or Gene Ontology (GO) ID 0047565, can be suitably used in the methods and host cells of the present disclosure.

In some embodiments, a 3-HPDH of the present disclosure refers to a polypeptide having the enzymatic activity of a polypeptide shown in Table 1 below. In some embodiments, a 3-HPDH of the present disclosure refers to a polypeptide that is at least 90%, at least 91%, at least 92%, at least 93%, at least 94%, at least 95%, at least 96%, at least 97%, at least 98%, at least 99%, or 100% identical to a polypeptide shown in Table 1 below. In some embodiments, a 3-HPDH of the present disclosure is derived from a source organism shown in Table 1 below. In some embodiments, a 3-HPDH of the present disclosure comprises an amino acid sequence selected from the group consisting of SEQ ID NOs:122-130.

In some embodiments, a 3-HPDH of the present disclosure refers to a polypeptide having the enzymatic activity of a polypeptide shown in Table 7A below. In some embodiments, a 3-HPDH of the present disclosure refers to a polypeptide that is at least 90%, at least 91%, at least 92%, at least 93%, at least 94%, at least 95%, at least 96%, at least 97%, at least 98%, at least 99%, or 100% identical to a polypeptide shown in Table 7A below. In some embodiments, a 3-HPDH of the present disclosure comprises a polypeptide sequence that is at least 90%, at least 91%, at least 92%, at least 93%, at least 94%, at least 95%, at least 96%, at least 97%, at least 98%, at least 99%, or 100% identical to the amino acid sequence of SEQ ID NO:154 or 159. In some embodiments, a 3-HPDH of the present disclosure comprises the amino acid sequence of SEQ ID NO:154 or 159.

In some embodiments, a 3-HPDH of the present disclosure is an endogenous 3-HPDH. A variety of host cells contemplated for use herein include endogenous genes encoding 3-HPDH enzymes; see. e.g., Table 1 below. In some embodiments, a 3-HPDH of the present disclosure is a recombinant 3-HPDH. For example, a polynucleotide encoding a 3-HPDH of the present disclosure can be introduced into a host cell that lacks endogenous 3-HPDH activity, or a polynucleotide encoding a 3-HPDH of the present disclosure can be introduced into a host cell with endogenous 3-HPDH activity in order to supplement, enhance, or supply said activity under different regulation than the endogenous activity.

TABLE 1
Exemplary 3-HPDH polypeptides.

| Sequence Name | Amino Acid Sequence | Source Organism |
|---|---|---|
| A4YI81_METS5 | MTEKVSVVGAGVIGVGWATLFASKGYSVSLYTEKKETL | Metallosphaera sedula |
| | DKGIEKLRNYVQVMKNNSQITEDVNTVISRVSPTTNLDE | |
| | AVRGANFVIEAVIEDYDAKKKIFGYLDSVLDKEVILASST | |
| | SGLLITEVQKAMSKHPERAVIAHPWNPPHLLPLVEIVPGE | |
| | KTSMEVVERTKSLMEKLDRIVVVLKKEIPGFIGNRLAFAL | |
| | FREAVYLVDEGVATVEDIDKVMTAAIGLRWAFMGPFLT | |
| | YHLGGGEGGLEYFFNRGFGYGANEWMHTLAKYDKFPY | |
| | TGVTKAIQQMKEYSFIKGKTFQEISKWRDEKLLKVYKLV | |
| | WEK (SEQ ED NO: 122) | |
| Q819E3_BACCR | MEHKTLSIGFIGIGVMGKSMVYHLMQDGHKVYVYNRTK | Bacillus cereus |
| | AKTDSLVQDGANWCNTPKELVKQVDIVMTMVGYPHDV | |
| | EEVYFGIEGIIEHAKEGTIAIDFTTSTPTLAKRINEVAKRK | |
| | NIYTLDAPVSGGDVGAKEAKLAIMVGGEKEIYDRCLPLL | |
| | EKLGTNIQLQGPAGSGQHTKMCNQIAIASNMIGVCEAVA | |
| | YAKKAGLNPDKVLESISTGAAGSWSLSNLAPRMLKGDF | |
| | EPGFYVKHFMKDMKIALEEAERLQLPVPGLSLAKELYEE | |
| | LIKDGEENSGTQVLYKKYIRG (SEQ ED NO: 123) | |
| 5JE8 | MKKIGFIGLGNMGLPMSKNLVKSGYTVYGVDLNKEAEA | Bacillus cereus |
| | SFEKEGGIIGLSISKLAETCDVVFTSLPSPRAYEAVYFGAE | |
| | GLFENGHSNVVFIDTSTVSPQLNKQLEEAAKEKKVDFLA | |
| | APVSGGVIGAENRTLTFMVGGSKDVYEKTESIMGVLGA | |
| | NIFHVSEQIDSGTTVKLINNLLIGFYTAGVSEALTLAKKN | |
| | NMDLDKMFDILNVSYGQSRIYERNYKSFIAPENYEPGFT | |
| | VNLLKKDLGFAVDLAKESELHLPVSEMLLNVYDEASQA | |
| | GYGENDMAALYKKVSEQLISNQK (SEQ ID NO: 124) | |
| SERDH_PSEAE | MKQIAFIGLGHMGAPMATNLLKAGYLLNVFDLVQSAVD | Psendomonas |
| | GLVAAGASAARSARDAVQGADVVISMLPASQHVEGLYL | aeruginosa |
| | DDDGLLAHIAPGTLVLECSTIAPTSARKIHAAARERGLA | |
| | MLDAPVSGGTAGAAAGTLTFMVGGDAEALEKARPLFEA | |
| | MGRNIFHAGPDGAGQVAKVCNNQLLAVLMIGTAEAMA | |
| | LGVANGLEAKVLAEIMRRSSGGNWALEVYNPWPGVME | |
| | NAPASRDYSGGFMAQLMAKDLGLAQEAAQASASSTPM | |
| | GSLALSLYRLLLKQGYAERDFSVVQKLFDPTQGQ (SEQ | |
| | ID NO: 125) | |
| E7KSY9_YEASL | MSQGRKAAERLAKKTVLITGASAGIGKATALEYLEASNG | Saccharomyces |
| | DMKLILAARRLEKLEELKKTIDQEFPNAKVHVAQLDITQ | cerevisiae |
| | AEKIKPFIENLPQEFKDIDILVNNAGKALGSDRVGQIATE | |
| | DIQDVFDTNVTALINITQAVLPIFQAKNSGDIVNTLGSIAGR | |
| | DAYPTGSIYCASKFAVGAFTDSLRKELINTKIRVILIAPGL | |
| | VETEFSLVRYRGNEEQAKNVYKDTTPLMADDVADLIVY | |
| | ATSRKQNTVIADTLIFPTNQASPHHIFRG (SEQ ID NO: 126) | |
| Q5FQ06_GLUOX | MSSPKIGFIGYGAMAQRMGANLRKAGYPVVAYAPSGGK | Gluconobacter oxydans |
| | DETEMLPSPRAIAEAAEIIIFCVPNDAAENESLHGENGAL | |
| | AALTPGKLVLDTSTVSPDQADAFASLAVEHGFSLLDAPM | |
| | SGSTPEAETGDLVMLVGGDEAVVKRAQPVLDVIGKLTIH | |
| | AGPAGSAARLKLVVNGVMGATLNVIAEGVSYGLAAGL | |
| | DRDVVFDTLQQVAVVSPHHKRKLKMGQNREFPSQFPTR | |
| | LMSKDMGLLLDAGRKVGAFMPGMAVADQALALSNRLH | |
| | ANEDYSALIGAMEHSVANLPHK (SEQ ID NO: 127) | |
| A9A4M8_NITMS | MHTVRIPKVINFGEDALGQTEYPKNALVVTTVPPELSDK | Nitrosopumilus |
| | WLAKMGIQDYMLYDKVKPEPSIDDVNTLISEFKEKKPSV | maritimus |
| | LIGLGGGSSMDVVKYAAQDFGVEKILIPTTFGTGAEMTT | |
| | YCVLKFDGKKKLLREDRFLADMAVVDSYFMDGTPEQVI | |
| | KNSVCDACAQATEGYDSKLGNDLTRTLCKQAFEILYDAI | |
| | MNDKPENYPYGSMLSGMGFGNCSTTLGHALSYVFSNEG | |
| | VPHGYSLSSCTTVAHKHNKSIFYDRFKEAMDKLGFDKLE | |
| | LKADVSEAADVVMTDKGHLDPNPIPISKDDVVKCLEDIK | |
| | AGNL (SEQ ID NO: 128) | |
| YDFG_ECOLI | MIVLVTGATAGFGECITRRFIQQGHKVIATGRRQERLQEL | Escherichia coli |
| | KDELGDNLYIAQLDVRNRAAIEEMLASLPAEWCNIDILV | |
| | NNAGLALGMEPAHKASVEDWETMIDTNNKGLVYMTRA | |
| | VLPGMVERNHGHIINIGSTAGSWPYAGGNVYGATKAFV | |
| | RQFSLNLRTDLHGTAVRVTDIEPGLVGGTEFSNVRFKGD | |
| | DGKAEKTYQNTVALTPEDVSEAVWWVSTLPAHVNINTL | |
| | EMMPVTQSYAGLNVHRQ (SEQ ID NO: 129) | |
| Q5SLQ6_THET8 | MEKVAFIGLGAMGYPMAGHLARRFPTLVWNRTFEKALR | Thermus thermophilus |
| | HQEEFGSEAVPLERVAEARVIFTCLPTTREVYEVAEALYP | |
| | YLREGTYWVDATSGEPEASRRLAERLREKGVTYLDAPV | |
| | SGGTSGAEAGTLTVMLGGPEEAVERVRPFLAYAKKVVH | |
| | VGPVGAGHAVKAINNALLAVNLWAAGEGLLALVKQGV | |
| | SAEKALEVINASSGRSNATENLIPQRVLTRAFPKTFALGL | |
| | LVKDLGIAMGVLDGEKAPSPLLRLAREVYEMAKRELGP | |
| | DADHVEALRLLERWGGVEIR (SEQ ID NO: 130) | |

TABLE 7A
Candidate 3-HPDH sequences.

| Enzyme name | Amino acid sequence |
|---|---|
| ADH6_YEAST | MSYPEKFEGIAIQSHEDWKNPKKTKYDPKPFYDHDIDIKIEACGVCGSDIHCAAG |
| | HWGNMKMPLVVGHEIVGKVVKLGPKSNSGLKVGQRVGVGAQVFSCLECDRCK |
| | NDNEPYCTKFVTTYSQPYEDGYVSQGGYANYVRVHEHFVVPIPENIPSHLAAPLL |
| | CGGLTVYSPLVRNGCGPGKKVGIVGLGGIGSMGTLISKAMGAETYVISRSSRKRE |
| | DAMKMGADHYIATLEEGDWGEKYFDTFDLIVVCASSLTDIDFNIMPKAMKVGG |
| | RIVSISIPEQHEMLSLKPYGLKAVSISYSALGSIKELNQLLKLVSEKDIKIWVETLPV |
| | GEAGVHEAFERMEKGDVRYRFTLVGYDKEFSD (SEQ ID NO: 149) |
| YQHD_ECOLI | MNNFNLHTPTRILFGKGAIAGLREQIPHDARVLITYGGGSVKKTGVLDQVLDALK |
| | GMDVLEFGGIEPNPAYETLMNAVKLVREQKVTFLLAVGGGSVLDGTKFIAAAA |
| | NYPENIDPWHILQTGGKEIKSAIPMGCVLTLPATGSESNAGAVISRKTTGDKQAF |
| | HSAHVQPVFAVLDPVYTYTLPPRQVANGVVDAFVHTVEQYVTKPVDAKIQDRF |
| | AEGILLTLIEDGPKALKEPENYDVRANVMWAATQALNGLIGAGVPQDWATHML |
| | GHELTAMHGLDHAQTLAIVLPALWNEKRDTKRAKLLQYAERVWNITEGSDDER |
| | IDAAIAATRNFFEQLGVPTHLSDYGLDGSSIPALLKKLEEHGMTQLGENHDITLD |
| | VSRRIYEAAR (SEQ ID NO: 150) |
| ADH2_YEAST_Alcohol_dehydrogenase_2 | MSIPETQKAIIFYESNGKLEHKDIPVPKPKPNELLINVKYSGVCHTDLHAWHGDW |
| | PLPTKLPLVGGHEGAGVVVGMGENVKGWKIGDYAGIKWLNGSCMACEYCELG |
| | NESNCPHADLSGYTHDGSFQEYATADAVQAAHIPQGTDLAEVAPILCAGITVYK |
| | ALKSANLRAGHWAAISGAAGGLGSLAVQYAKAMGYRVLGIDGGPGKEELFTSL |
| | GGEVFIDFTKEKDIVSAVVKATNGGAHGIINVSVSEAAIEASTRYCRANGTVVLV |
| | GLPAGAKCSSDVFNHVVKSISIVGSYVGNRADTREALDFFARGLVKSPIKVVGLS |
| | SLPEIYEKMEKGQIAGRYVVDTSK (SEQ ID NO: 151) |
| YdfG | MIVLVTGATAGFGECITRRFIQQGHKVIATGRRQERLQELKDELGDNLYIAQLDV |
| | RNRAAIEEMLASLPAEWCNIDILVNNAGLALGMEPAHKASVEDWETMIDTNNK |
| | GLVYMTRAVLPGMVERNHGHIINIGSTAGSWPYAGGNVYGATKAFVRQFSLNL |
| | RTDLHGTAVRVTDIEPGLVGGTEFSNVRFKGDDGKAEKTYQNTVALTPEDVSEA |
| | VWWVSTLPAHVNINTLEMMPVTQSYAGLNVHRQ (SEQ ID NO: 152) |
| A9A4M8 | MHTYRIPKVINFGEDALGQTEYPKNALVVTTVPPELSDKWLAKMGIQDYMLYD |
| | KVKPEPSIDDVNTLISEFKEKKPSVLIGLGGGSSMDVVKYAAQDFGVEKILIPTTF |
| | GTGAEMTTYCVLKFDGKKKLLREDRFLADMAVVDSYFMDGTPEQVIKNSVCDA |
| | CAQATEGYDSKLGNDLTRTLCKQAFEILYDAIMNDKPENYPYGSMLSGMGFGN |
| | CSTTLGHALSYVFSNEGVPHGYSLSSCTTVAHKHNKSIFYDRFKEAMDKLGFDK |
| | LELKADVSEAADVVMTDKGHLDPNPIPISKDDVVKCLEDIKAGNL (SEQ ID |
| | NO: 153) |
| A4YI81 | MTEKVSVVGAGVIGVGWATLFASKGYSVSLYTEKKETLDKGIEKLRNYVQVMK |
| | NNSQITEDVNTVISRVSPTTNLDEAVRGANFVIEAVIEDYDAKKKIFGYLDSVLDK |
| | EVILASSTSGLLITEVQKAMSKHPERAVIAHPWNPPHLLPLVEIVPGEKTSMEVVE |
| | RTKSLMEKLDRIVVVLKKEIPGFIGNRLAFALFREAVYLVDEGVATVEDIDKVMT |
| | AAIGLRWAFMGPFLTYHLGGGEGGLEYFFNRGFGYGANEWMHTLAKYDKFPYT |
| | GVTKAIQQMKEYSFIKGKTFQEISKWRDEKLLKVYKLVWEK (SEQ ID NO: 154) |
| 3OBB | MKQIAFIGLGHMGAPMATNLLKAGYLLNVFDLVQSAVDGLVAAGASAARSARD |
| | AVQGADVVISMLPASQHVEGLYLDDDGLLAHIAPGTLVLECSTIAPTSARKIHAA |
| | ARERGLAMLDAPVSGGTAGAAAGTLTFMVGGDAEALEKARPLFEAMGRNIFHA |
| | GPDGAGQVAKVCNNQLLAVLMIGTAEAMALGVANGLEAKVLAEIMRRSSGGN |
| | WALEVYNPWPGVMENAPASRDYSGGFMAQLMAKDLGLAQEAAQASASSTPM |
| | GSLALSLYRLLLKQGYAERDFSVVQKLFDPTQGQ (SEQ ID NO: 155) |
| 5JE8 | MKKIGFIGLGNMGLPMSKNLVKSGYTVYGVDLNKEAEASFEKEGGIIGLSISKLA |
| | ETCDVVFTSLPSPRAVEAVYFGAEGLFENGHSNVVFIDTSTVSPQLNKQLEEAAK |
| | EKKVDFLAAPVSGGVIGAENRTLTFMVGGSKDVVEKTESIMGVLGANIFHVSEQI |
| | DSGTTVKLINNLLIGFYTAGVSEALTLAKKNNMDLDKMFDILNVSYGQSRIYERN |
| | YKSFIAPENYEPGFTVNLLKKDLGFAVDLAKESELHLPVSEMLLNVYDEASQAG |
| | YGENDMAALYKKVSEQLISNQK (SEQ ID NO: 156) |
| Q819E3 | MEHKTLSIGFIGIGVMGKSMVYHLMQDGHKVYVYNRTKAKTDSLVQDGANWC |
| | NTPKELVKQVDIVMTMVGYPHDVEEVYFGIGIIEHAKEGTIAIDFTTSTPTLAKR |
| | INEVAKRKNIYTLDAPVSGGDVGAKEAKLAIMVGGEKEIYDRCLPLLEKLGTNIQ |
| | LQGPAGSGQHTKMCNQIAIASNMIGVCEAVAYAKKAGLNPDKVLESISTGAAGS |
| | WSLSNLAPRMLKGDFEPGFYVKHFMKDMKIALEEAERLQLPVPGLSLAKELYEE |
| | LIKDGEENSGTQVLYKKYIRG (SEQ ID NO: 157) |
| Q5FQ06 | MSSPKIGFIGYGAMAQRMGANLRKAGYPVVAYAPSGGKDETEMLPSPRAIAEAA |
| | EIIIFCVPNDAAENESLHGENGALAALTPGKLVLDTSTVSPDQADAFASLAVEHGF |
| | SLLDAPMSGSTPEAETGDLVMLVGGDEAVVKRAQPVLDVIGKLTIHAGPAGSAA |
| | RLKLVVNGVMGATLNVIAEGVSYGLAAGLDRDVVFDTLQQVAVVSPHHKRKL |
| | KMGQNREFPSQFPTRLMSKDMGLLLDAGRKVGAFMPGMAVADQALALSNRLH |
| | ANEDYSALIGAMEHSVANLPHK (SEQ ID NO: 158) |
| 2CVZ | MEKVAFIGLGAMGYPMAGHLARRFPTLVWNRTFEKALRHQEEFGSEAVPLERV |
| | AEARVIFTCLPTTREVYEVAEALYPYLREGTYWVDATSGEPEASRRLAERLREKG |
| | VTYLDAPVSGGTSGAEAGTLTVMLGGPEEAVERVRPFLAYAKKVVHVGPVGAG |
| | HAVKAINNALLAVNLWAAGEGLLALVKQGVSAEKALEVINASSGRSNATENLIP |
| | QRVLTRAFPKTFALGLLVKDLGIAMGVLDGEKAPSPLLRLAREVYEMAKRELGP |
| | DADHVEALRLLERWGGVEIR (SEQ ID NO: 159) |
| Q05016 | MSQGRKAAERLAKKTVLITGASAGIGKATALEYLEASNGDMKLILAARRLEKLE |
| | ELKKTIDQEFPNAKVHVAQLDITQAEKIKPFIENLPQEFKDIDILYNNAGKALGSD |
| | RVGQIATEDIODVFDTNVTALINITQAVLPIFQAKNSGDIVNLGSIAGRDAYPTGSI |
| | YCASKFAVGAFTDSLRKELDINTKIRVILIAPGLVETEFSLVRYRGNEEQAKNVYKD |
| | TTPLMADDVADLIVYATSRKQNTVIADTLIFPTNQASPHHIFRG (SEQ ID |
| | NO: 160) |

3-hydroxypropionate Metabolic Pathways

In some embodiments, a host cell of the present disclosure comprises one or more additional polynucleotides (e.g., encoding one or more additional polypeptides) whose activity promotes the synthesis or uptake of oxaloacetate into the host cell. As is known in the art, host cells are able to convert glucose into phosphoenolpyruvate through a series of metabolic reactions known as glycolysis. See. e.g., Alberts, B., Johnson, A., and Lewis. J. et al. Molecular Biology of the Cell. 4^{th} ed. New York: Garland Science: 2002. In some embodiments, a host cell of the present disclosure comprises polynucleotides encoding the following metabolic enzymes: hexokinase, phosphoglucose isomerase, phosphofructokinase, aldolase, triose phosphate isomerase, glyceraldehyde 3-phosphate dehydrogenase, phosphoglycerate kinase, phosphoglycerate mutase, and enolase. Suitable enzymes from a variety of host cells are well known in the art. In some embodiments, a host cell of the present disclosure comprises polynucleotides encoding one or more polypeptides active in the oxidative pentose phosphate or Entner-Doudoroff pathway. These pathways are also known to break down sugars (e.g., into glyceraldehyde-3-phosphate), see, e.g., Chen, X. et al. (2016) Proc. Natl. Acad. Sci. 113:5441-5446. The metabolic enzymes catalyzing steps in these pathways are known in the art.

Metabolic pathways that produce oxaloacetate are known, such as the tricarboxylic acid (TCA) cycle. Phosphoenolpyruvate (e.g., originating from the breakdown of glucose as described above) can be converted into oxaloacetate through multiple chemical reactions. See Sauer, U. and Eikmanns, B. J. (2005) FEMS Microbiol. Rev. 29:765-794. In some embodiments, a host cell of the present disclosure comprises a polynucleotide encoding a phosphoenolpyruvate carboxylase. In some embodiments, a phosphoenolpyruvate carboxylase refers to an enzyme that catalyzes the conversion of phosphoenolpyruvate into oxaloacetate. Any enzyme capable of catalyzing the conversion of phosphoenolpyruvate into oxaloacetate, e.g., known or predicted to have the enzymatic activity described by EC 4.1.1.31 and/or Gene Ontology (GO) ID 0008964, can be suitably used in the methods and host cells of the present disclosure. In some embodiments, the phosphoenolpyruvate carboxylase is an endogenous phosphoenolpyruvate carboxylase. In some embodiments, the phosphoenolpyruvate carboxylase is a recombinant phosphoenolpyruvate carboxylase. Phosphoenolpyruvate carboxylases are known in the art and include, without limitation. NP_312912, NP_252377, NP_232274, WP_001393487, WP_001863724, and WP_002230956 (see www.genome.jp/dbget-bin/get_linkdb?-t+refpep+ec:4.1.1.31 for additional enzymes).

In some embodiments, a host cell of the present disclosure comprises polynucleotides encoding a pyruvate kinase and a pyruvate carboxylase. In some embodiments, a pyruvate kinase refers to an enzyme that catalyzes the conversion of phosphoenolpyruvate into pyruvate. Any enzyme capable of catalyzing the conversion of phosphoenolpyruvate into pyruvate, e.g., known or predicted to have the enzymatic activity described by EC 2.7.1.40 and/or Gene Ontology (GO) ID 0004743, can be suitably used in the methods and host cells of the present disclosure. In some embodiments, the pyruvate kinase is an endogenous pyruvate kinase. In some embodiments, the pyruvate kinase is a recombinant pyruvate kinase. Pyruvate kinases are known in the art and include, without limitation, S. cerevisiae Pyk1 and Pyk2, NP_014992, NP_250189, NP_310410, NP_358391, NP_390796, and NP_465095 (see www.genome.jp/dbget-bin/get_linkdb?-t+refpep+ec:2.7.1.40 for additional enzymes). In some embodiments, a pyruvate carboxylase refers to an enzyme that catalyzes the conversion of pyruvate into oxaloacetate. Any enzyme capable of catalyzing the conversion of pyruvate into oxaloacetate, e.g., known or predicted to have the enzymatic activity described by EC 6.4.1.1 and/or Gene Ontology (GO) ID 0071734, can be suitably used in the methods and host cells of the present disclosure. In some embodiments, the pyruvate carboxylase is an endogenous pyruvate carboxylase. In some embodiments, the pyruvate carboxylase is a recombinant pyruvate carboxylase. Pyruvate carboxylases are known in the art and include, without limitation, NP_009777, NP_011453, NP_266825, NP_349267, and NP_464597 (see www.genome.jp/dbget-bin/get_linkdb?-t+refpep+ec:6.4.1.1 for additional enzymes).

In some embodiments, a host cell of the present disclosure comprises one or more modifications resulting in decreased production of pyruvate from phosphoenolpyruvate, e.g., as compared to a host cell (e.g., of the same species and grown under similar conditions) lacking the modification. Without wishing to be bound to theory, it is thought that decreasing production of pyruvate from phosphoenolpyruvate may favor the conversion of phosphoenolpyruvate into oxaloacetate, e.g., using a phosphoenolpyruvate carboxylase of the present disclosure.

In some embodiments, a host cell of the present disclosure comprises a polynucleotide encoding a phosphoenolpyruvate carboxykinase (PEPCK). In some embodiments, a host cell of the present disclosure comprises a polynucleotide encoding a recombinant phosphoenolpyruvate carboxykinase (PEPCK). In some embodiments, a PEPCK of the present disclosure refers to a polypeptide having the enzymatic activity of a polypeptide shown in Table 9A below. In some embodiments, a PEPCK of the present disclosure comprises a polypeptide that is at least 90%, at least 91%, at least 92%, at least 93%, at least 94%, at least 95%, at least 96%, at least 97%, at least 98%, at least 99%, or 100% identical to a polypeptide shown in Table 9A below. In some embodiments, a PEPCK of the present disclosure comprises a polypeptide sequence that is at least 90%, at least 91%, at least 92%, at least 93%, at least 94%, at least 95%, at least 96%, at least 97%, at least 98%, at least 99%, or 100% identical to the amino acid sequence of SEQ ID NO: 162 or 163. In some embodiments, a PEPCK of the present disclosure comprises the amino acid sequence of SEQ ID NO:162 or 163.

TABLE 9A
Candidate PEPCK sequences.

| Enzyme name | Amino acid sequence |
|---|---|
| Q7XAU8 | MASPNGLAKIDTQGKTEVYDGDTAAPVRAQTIDELHLLQRKRSA |
| | PTTPIKDGATSAFAAAISEEDRSQQQLQSISASLTSLARETGPKLVK |
| | GDPSDPAPHKHYQPAAPTIVATDSSLKFTHVLYNLSPAELYEQAF |
| | GQKKSSFITSTGALATLSGAKTGRSPIRDKRVVKDEATAQELWWG |
| | KGSPNIEMDERQFVINRERALDYLNSLDKVYVNDQFLNWDPENRI |
| | KVRIITSRAYHALFMHNMCIRPTDEELESFGTPDFTIYNAGEFPAN |
| | RYANYMTSSTSINISLARREMVILGTQYAGEMKKGLFGVMHYLM |
| | PKRGILSLHSGCNMGKDGDVALFFGLSGTGKTTLSTDHNRLLIGD |
| | DEHCWSDNGVSNIEGGCYAKCIDLSQEKEPDIWNAIKFGTVLENV |
| | VFNERTREVDYSDKSITENTRAAYPIEFIPNAKIPCVGPHPKNVILL |
| | ACDAFGVLPPVSKLNLAQTMYHFISGYTALVAGTVDGITEPTATF |
| | SACFGAAFIMYHPTKYAAMLAEKMQKYGATGWLVNTGWSGGR |
| | YGVGKRIRLPHTRKIIDAIHSGELLTANYKKTEVFGLEIPTEINGVP |
| | SEILDPINTWTDKAAYKENLLNLAGLFKKNFEVFASYKIGDDSSLT |
| | DEILAAGPNF (SEQ ID NO: 161) |
| PCKA_Ecoli | MRVNNGLTPQELEAYGISDVHDIVYNPSYDLLYQEELDPSLTGYE |
| | RGVLTNLGAVAVDTGIFTGRSPKDKYIVRDDTTRDTFWWADKGK |
| | GKNDNKPLSPETWQHLKGLVTRQLSGKRLFVVDAFCGANPDTRL |
| | SVRFITEVAWQAHFVKNMFIRPSDEELAGFKPDFIVMNGAKCTNP |
| | QWKEQGLNSENFVAFNLTERMQLIGGTWYGGEMKKGMFSMMN |
| | YLLPLKGIASMHCSANVGEKGDVAVFFGLSGTGKTTLSTDPKRRL |
| | IGDDEHGWDDDGVFNFEGGCYAKTIKLSKEAEPEIYNAIRRDALL |
| | ENVTVREDGTIDFDDGSKTENTRVSYPIYHIDNIVKPVSKAGHATK |
| | VIFLTADAFGVLPPVSRLTADQTQYHFLSGFTAKLAGTERGITEPT |
| | PTFSACFGAAFLSLHPTQYAEVLVKRMQAAGAQAYLVNTGWNG |
| | TGKRISIKDTRAIIDAILNGSIDNAETFTLPMFNLAIPTELPGVDTKI |
| | LDPRNTYASPEQWQEKAETLAKLFIDNFDKYTDTPAGAALVAAG |
| | PKL (SEQ ID NO: 162) |
| PCK from | MTDLNKLVKELNDLGLTDVKEIVYNPSYEQLFEEETKPGLEGFDK |
| Actinobaccilus_succinogenes | GTLTTLGAVAVDTGIFTGRSPKDKYIVCDETTKDTVWWNSEAAK |
| | NDNKPMTQETWKSLRELVAKQLSGKRLFVVEGYCGASEKHRIGV |
| | RMVTEVAWQAHFVKNMFIRPTDEELKNFKADFTVLNGAKCTNP |
| | NWKEQGLNSENFVAFNITEGIQLIGGTWYGGEMKKGMFSMMNY |
| | FLPLKGVASMHCSANVGKDGDVAIFFGLSGTGKTTLSTDPKRQLI |
| | GDDEHGWDESGVFNFEGGCYAKTINLSQENEPDIYGAIRRDALLE |
| | NVVVRADGSVDFDDGSKTENTRVSYPIYHIDNIVRPVSKAGHATK |
| | VIFLTADAFGVLPPVSKLTPEQTEYYFLSGFTAKLAGTERGVTEPT |
| | PTFSACFGAAFLSLHPIQYADVLVERMKASGAEAYLVNTGWNGT |
| | GKRISIKDTRGIIDAILDGSIEKAEMGELPIFNLAIPKALPGVDPAIL |
| | DPRDTYADKAQWQVKAEDLANRFVKNFVKYTANPEAAKLVGA |
| | GPKA(SEQ ID NO: 163) |
| 1J3B | MQRLEALGIHPKKRVFWNTVSPVLVEHTLLRGEGLLAHHGPLVV |
| | DTTPYTGRSPKDKFVVREPEVEGEIWWGEVNQPFAPEAFEALYQR |
| | VVQYTSERDLYVQDLYAGADRRYRLAVRVVTESPWHALFARNM |
| | FILPRRFGNDDEVEAFVPGFTVVHAPYFQAVPERDGTRSEVFVGIS |
| | FQRRLVLIVGTKYAGEIKKSIFTVMNYLMPKRGVFPMHASANVG |
| | KEGDVAVFFGLSGTGKTTLSTDPERPLIGDDEHGWSEDGVFNFEG |
| | GCYAKVIRLSPEHEPLIYKASNQFEAILENVVVNPESRRVQWDDD |
| | SKTENTRSSYPIAHLENVVESGVAGHPRAIFFLSADAYGVLPPIAR |
| | LSPEEAMYYFLSGYTARVAGTERGVTEPRATFSACFGAPFLPMHP |
| | GVYARMLGEKIRKHAPRVYLVNTGWTGGPYGVGYRFPLPVTRA |
| | LLKAALSGALENVPYRRDPVFGFEVPLEAPGVPQELLNPRETWAD |
| | KEAYDQQARKLARLFQENFQKYASGVAKEVAEAGPRTE (SEQ ID |
| | NO. 164) |
| 1YTM | MSLSESLAKYGITGATNIVHNPSHEELFAAETQASLEGFEKGTVTE |
| | MGAVNVMTGVYTGRSPKDKFIVKNEASKEIWWTSDEFKNDNKP |
| | VTEEAWAQLKALAGKELSNKPLYVVDLFCGANENTRLKIRFVME |
| | VAWQAHFVTNMFIRPTEEELKGFEPDFVVLNASKAKVENFKELG |
| | LNSETAVVFNLAEKMQIILNTWYGGEMKKGMFSMMNFYLPLQGI |
| | AAMHCSANTDLEGKNTAIFFGLSGTGKTTLSTDPKRLLIGDDEHG |
| | WDDDGVFNFEGGCYAKVINLSKENEPDIWGAIKRNALLENVTVD |
| | ANGKVDFADKSVTENTRVSYPIFHIKNIVKPVSKAPAAKRVIFLSA |
| | DAFGVLPPVSILSKEQTKYYFLSGFTAKLAGTERGITEPTPTFSSCF |
| | GAAFLTLPPTKYAEVLVKRMEASGAKAYLVNTGWNGTGKRISIK |
| | DTRGIIDAILDGSIDTANTATIPYFNFTVPTELKGVDTKILDPRNTY |
| | ADASEWEVKAKDLAERFQKNFKKFESLGGDLVKAGPQL (SEQ ID |
| | NO: 165) |

In some embodiments, the modification results in decreased pyruvate kinase (PK) activity, e.g., as compared to a host cell (e.g., of the same species and grown under similar conditions) lacking the modification. For example, the host cell may comprise one or more mutations in an endogenous PK enzyme, resulting in decreased PK activity.

In some embodiments, the modification results in decreased pyruvate kinase (PK) expression, e.g., as compared to a host cell (e.g., of the same species and grown under similar conditions) lacking the modification. Various methods for decreasing gene expression may be used and include, without limitation, homologous recombination or other mutagenesis techniques (e.g., transposon-mediated mutagenesis) to remove and/or replace part or all of the coding sequence or regulatory sequence(s); CRISPR/Cas9-mediated gene editing; CRISPR interference (CRISPRi; see Qi, L. S. et al. (2013) Cell 152:1173-1183); heterochromatin formation; RNA interference (RNAi), morpholinos, or other antisense nucleic acids; and the like.

As one example, PK expression can be decreased by placing a PK coding sequence (e.g., an endogenous PK coding sequence) under the control of a promoter (e.g., an exogenous promoter) that results in decreased PK coding sequence expression. For example, an endogenous PK coding sequence can be operably linked to an exogenous promoter that results in decreased expression of the endogenous PK coding sequence, e.g., as compared to endogenous PK expression (e.g., of the same species and grown under similar conditions).

In some embodiments, a PK coding sequence (e.g., an endogenous PK coding sequence) of the present disclosure is operably linked to an inducible promoter, such as the MET3, CTR1, and CTR3 promoters. The MET3 promoter is an inducible promoter commonly used in the art to regulate gene transcription in response to methionine levels, e.g., in the cell culture medium. See, e.g., Mao, X. et al. (2002) Curr. Microbiol. 45:37-40 and Asadollahi, M. A. et al. (2008) Biotechnol. Bioeng. 99:666-677. The CTR1 and CTR3 promoters are copper-repressible promoters commonly used in the art to regulate gene transcription in response to copper levels, e.g., in the cell culture medium. See. e.g., Labbe, S. et al. (1997) J. Biol. Chem. 272:15951-15958.

In some embodiments, a PK coding sequence (e.g., an endogenous PK coding sequence) of the present disclosure is operably linked to a promoter (e.g., a MET promoter) comprising the polynucleotide sequence of TGTGAAGATGAATGTATTGAATATAAAATTATTTCTTGATATCCATATATCCCA TAAACAAGAAATTACTACTTCCGGAAAAACGTAAACACAGTGGAAAATTTACG ATACCAATCACGTGATCAAATTACAAGGAAAGCACGTGACTTAAGGCTTCCTA AACTAGAAATTGTGGCTGTCAGGATCAATTGAAAATGGCGCCACACTTTCTTCT CTTATGGTTAGGAGTAGACCCCGAAGACAGAGGATTCCGGCAATCGGAGCACA GTACAACTTTATACTTTCGTTCACTGCATGGAGAGTGAAATTTTTCAAGCTGAT GCAATTGATATAAATATAACCCATTTACAGGATATGTCCCTCCAAAGGTTGATC CGTTATTGCTATAATGAATATTGOTTCACTATTTATGCCTCTTGATTTGTAAT CCGGGCCTTTGCTTTTGTACTTGACCTTAGACCTTAATCCACCCCAATAGTAAC TAATCAGAACACAAA (SEQ ID NO:131). In some embodiments, a PK coding sequence (e.g., an endogenous PK coding sequence) of the present disclosure is operably linked to a promoter (e.g., a CTR3 promoter) comprising the polynucleotide sequence of ATTCAACTAGAAAGTTGCAAGTAAAGCAACTAACTGCGGGACCAAACAAATTT AAACAAACCCGTGAATATTGTCTACCTATCCTATCCTATGCTTCGAAAAAATGAGC AAATATTAACGACAGTTTACTACTGTCGTAGCTTTTACTTCAAATAGAAGGAAA ACTGATGAATTTGCATACATGAGCAATTTATTAGAAATTATTACCTAAAAAGG CAAGAAAGCAGAGATAATTTTCTCATGCCCCCAACTACTTACTrATATCTACAA TTAAAACTTAATAATATGCTCTTTTGCAGTATGAACCTTTTCTTTAAATAACAG AGTACTGCCGCTTCAAACGATGTATTCTACATTGACTAAACGAAAATACTACAA GCTGTCTTACTTTTAAACAAAC (SEQ ID NO:132). In some embodiments, a PK coding sequence (e.g., an endogenous PK coding sequence) of the present disclosure is operably linked to a promoter (e.g., a CTR1 promoter) comprising the polynucleotide sequence of TTGCGTAAGATAGATTCAAACCAAGTGATGGACCTGTCACTGCTTAGTGTTGAT GAACAAACATATCTTCGAGGCCATTCCGCAATGAAAAATCAATTTCTGACTAGC TTTGCTGGAGAGGAGCCATCGATACCAGAGTCAGATCCTGACAACGAATCGTG TCACATTTTGTCCGTGCCCAAGCACCGTTTCCCTTCCGAGATGAAGATACCAT GCAAGTAGGTGATGTTCGTGTTGCTAAATGGAAAGACGTGGCGCATGGTGTAG CAGAGGGAGCTTTACACGTGATATAAACAGCATGCGCCTCATTGAGCAAATTA ACTACTAACGGTTTCCGAAATAGGTAATTGAGCAAATAAGAATTTCAGCACTT ATGAAGAAGGGTCAAGCGTATATAAAGGACACCTCTTACTTTGAGGTTGTAAG TTTGTCTCTAGCCTTATCAATGGTCTTTATTTTrTCTGCTACCTTGATTGGGAAAT AATCCAATCTTCAATA (SEQ ID NO:133).

In some embodiments, a host cell of the present disclosure comprises a modification resulting in increased expression or activity of phosphoenolpyruvate carboxykinase (PEPCK), e.g., as compared to a host cell (e.g., of the same species and grown under similar conditions) lacking the modification. As one example, an exogenous PEPCK coding sequence can be introduced into a host cell (e.g., operably linked to a constitutive or inducible promoter as described herein), or an endogenous PEPCK coding sequence can be operably linked to an exogenous promoter (e.g., a constitutive or inducible promoter as described herein). In some embodiments, a host cell of the present disclosure comprises a modification resulting in increased expression or activity of phosphoenolpyruvate carboxykinase (PEPCK) and a modification resulting in decreased pyruvate kinase (PK) expression and/or activity. In some embodiments, a PEPCK refers to an enzyme that catalyzes the conversion of phosphoenolpyruvate into oxaloacetate. Any enzyme capable of catalyzing the conversion of phosphoenolpyruvate into oxaloacetate, e.g., known or predicted to have the enzymatic activity described by EC 4.1.1.49 and/or Gene Ontology (GO) ID 0004611, can be suitably used in the methods and host cells of the present disclosure. Exemplary PEPCKs are also described supra and in Example 2 below.

### Host Cells

Certain aspects of the present disclosure relate to recombinant host cells. In some embodiments, a recombinant host cell of the present disclosure comprises a recombinant polynucleotide encoding an oxaloacetate decarboxylase (OAADC) of the present disclosure. For example, in some embodiments, the OAADC has a ratio of activity against pyruvate to activity against oxaloacetate that is less than or equal to about 5:1 and/or a specific activity of at least 0.1 μmol/min/mg against oxaloacetate. In some embodiments, the recombinant host cell further comprises a polynucleotide encoding a 3-hydroxypropionate dehydrogenase (3-HPDH) of the present disclosure. A host cell of the present disclosure can comprise one or more of the genetic modifications described supra in any number or combination.

Any microorganism may be utilized according to the present disclosure by one of ordinary skill in the art. In certain aspects, the microorganism is a prokaryotic microorganism, e.g., a recombinant prokaryotic host cell. In certain aspects, a microorganism is a bacterium, such as gram-positive bacteria or gram-negative bacteria. Given its rapid growth rate, well-understood genetics, variety of available genetic tools, and its capability in producing heterologous proteins, in some embodiments, a host cell of the present disclosure is an E. coli cell (e.g., a recombinant E. coli cell).

Other microorganisms may be used according to the present disclosure, e.g., based at least in part on the compatibility of enzymes and metabolites to host organisms. For example, other suitable organisms can include, without limitation: Acetobacter aceti, Achromobacter, Acidiphilium, Acinetobacter, Actinomadura, Actinoplanes, Aeropyrum pernix, Agrobacterium, Alcaligenes, Ananas comosus (M), Arthrobacter, Bacillus alcalophilus, Bacillus amyloliquefaciens, Bacillus brevis, Bacillus circulans, Bacillus clausii, Bacillus lentus, Bacillus licheniformis, Bacillus macerans, Bacillus stearothermophilus, Bacillus subtilis, Bifidobacterium, Brevibacillus brevis, Burkholderia cepacia, Candida cylindracea, Carica papaya (L), Cellulosimicrobium, Cephalosporium, Chaetomium erraticum, Chaetomium gracile, Clostridium, Clostridium butyricum, Clostridium acetobutylicum, Clostridium thermocellum, Corynebacterium (glutamicum), Corynebacterium efficiens, Escherichia coli, Enterococcus, Erwina chrysanthemi, Gliconobacter, Gluconacetobacter, Haloarcula, Humicola insolens, Kitasatospora setae, Klebsiella, Klebsiella oxytoca, Kocuria, Lactlactis, Lactobacillus, Lactobacillus fermentum, Lactobacillus sake, Lactococcus, Lactococcus lactis, Leuconostoc, Methylocystis, Methanolobus siciliae, Methanogenium organophilum, Methanobacterium bryantii, Microbacterium imperiale, Micrococcus lysodeikticus, Microlunatus, Mucor javanicus, Mycobacterium, Myrothecium, Nitrobacter, Nitrosomonas, Nocardia, Papaya carica, Pediococcus, Pediococcus halophilus, Paracoccus pantotrophus, Propionibacterium, Pseudomonas, Pseudomonas fluorescens, Pseudomonas denitrificans, Pyrococcus, Pyrococcus furiosus, Pyrococcus horikoshii, Rhizobium, Rhizomucor miehei, Rhizomucor pusillus Lindt, Rhizopus, Rhizopus delemar, Rhizopus japonicus, Rhizopus niveus, Rhizopus oryzae, Rhizopus oligosporus, Rhodococcus, Sclerotina libertina, Sphingobacterium multivorum, Sphingobium, Sphingomonas, Streptococcus, Streptococcus thermophilus Y-1, Streptomyces, Streptomyces griseus, Streptomyces lividans, Streptomyces murinus, Streptomyces rubiginosus, Streptomyces violaceoruber, Streptoverticillium mobaraense, Tetragenococcus, Thermus, Thiosphaera pantotropha, Trametes, Vibrio alginolyticus, Xanthomonas, Zymomonas, and Zymomonus mobilis. Any of these cells may suitably be selected by one of ordinary skill in the art as a recombinant host cell based on the present disclosure, e.g., for use in any of the methods of the present disclosure.

In some embodiments, a host cell of the present disclosure is a fungal host cell. In some embodiments, a recombinant fungal host cell of the present disclosure comprises a recombinant polynucleotide encoding an oxaloacetate decarboxylase (OAADC). In some embodiments, the recombinant fungal host cell further comprises a polynucleotide encoding a 3-hydroxypropionate dehydrogenase (3-HPDH). In some embodiments, the recombinant fungal host cell further comprises a polynucleotide encoding a phosphoenolpyruvate carboxykinase (PEPCK). Without wishing to be bound to theory, it is thought that fungal host cells are particularly advantageous for production of 3-HP, which can lead to acidification of a cell culture medium, since they can be more acid-tolerant than certain bacterial host cells. In some embodiments, a host cell of the present disclosure is a non-human host cell. In some embodiments, a host cell of the present disclosure is a yeast host cell.

A variety of fungal host cells are known in the art and contemplated for use as a host cell of the present disclosure. Non-limiting examples of fungal cells are any host cells (e.g., recombinant host cells) of a genus or species selected from Aspergillus, Aspergillus nidulans, Aspargillus niger, Aspargillus oryze, Aspergillus melleus, Aspergillus pulverulentus, Aspergillus saitoi, Aspergillus sojea, Aspergillus terreus, Aspergillus pseudoterreus, Aspergillus usamii, Candida rugosa, Issatchenkia orientalis, Kluyveromyces, Kluyveromyces fragilis, Kluyveromyces lactis, Kluyveromyces marxianas, Penicillium, Penicillium camemberti, Penicillium citrinum, Penicillium emersonii, Penicillium roqueforti, Penicillum lilactinum, Penicillum multicolor, Rhodosporidium toruloides, Saccharomyces cerevisiae, Schizosaccharomyces pombe, Trichoderma, Trichoderma longibrachiatum, Trichoderma reesei, Trichoderma viride, Trichosporon penicillatum, Yarrowia lipolytica, and Zygosaccharomyces rouxii.

Without wishing to be bound to theory, it is thought that the ability to tolerate and grow (e.g., be cultured in a culture medium/conditions characterized by) acidic pH is particularly advantageous for the methods described herein, since 3-HP production acidifies cell culture media. In some embodiments, a host cell of the present disclosure is capable of producing 3-HP at a pH (e.g., in a cell culture having a pH) lower than 4, lower than 4.5, lower than 5, lower than 5.5, lower than 6, or lower than 6.5. In some embodiments, a host cell of the present disclosure is capable of producing 3-HP at a pH (e.g., in a cell culture having a pH) lower than the pKa of 3-HP, i.e., 4.5 (e.g., at a temperature between about 20° C. and about 37° C., such as 20° C., 25° C., 30° C., or 37° C.).

### Recombinant Techniques

Many recombinant techniques commonly known in the art may be used to introduce one or more genes of the present disclosure (e.g., an OAADC, 3-HPDH, and/or PEPCK of the present disclosure) into a host cell, including without limitation protoplast fusion, transfection, transformation, conjugation, and transduction.

Unless otherwise indicated, the practice of the present disclosure employs conventional molecular biology techniques (e.g., recombinant techniques), microbiology, cell biology, and biochemistry, which are within the skill of the art. Such techniques are well known in the art; see. e.g., Molecular Cloning: A Laboratory Manual, second edition (Sambrook et al., 1989); Oligonucleotide Synthesis (Gait, ed., 1984); Animal Cell Culture (Freshney, ed., 1987): Gene Transfer Vectors for Mammalian Cells (Miller & Calos, eds., 1987); Current Protocols in Molecular Biology (Ausubel et al., eds., 1987): PCR: The Polymerase Chain Reaction, (Mullis et al., eds., 1994); and Current Protocols in Immunology (Coligan et al., eds., 1991).

In some embodiments, one or more recombinant polynucleotides are stably integrated into a host cell chromosome. In some embodiments, one or more recombinant polynucleotides are stably integrated into a host cell chromosome using homologous recombination, transposition-based chromosomal integration, recombinase-mediated cassette exchange (RMCE; e.g., using a Cre-lox system), or an integrating plasmid (e.g., a yeast integrating plasmid). A variety of integration techniques suitable for a range of host cells are known in the art (see. e.g., US PG Pub No. US20120329115; Daly, R. and Heam, M. T. (2005) J. Mol. Recognit. 18:119-138; and Griffiths, A. J. F., Miller, J. H., Suzuki, D. T. et al. An Introduction to Genetic Analysis. 7^{th} ed. New York: W.H. Freeman: 2000). See also PCT/US2017/014788, which is incorporated by reference in its entirety.

In some embodiments, one or more recombinant polynucleotides are maintained in a recombinant host cell of the present disclosure on an extra-chromosomal plasmid (e.g., an expression plasmid or vector). A variety of extra-chromosomal plasmids suitable for a range of host cells are known in the art, including without limitation replicating plasmids (e.g., yeast replicating plasmids that include an autonomously replicating sequence, ARS), centromere plasmids (e.g., yeast centromere plasmids that include an autonomously replicating sequence, CEN), episomal plasmids (e.g., 2-μm plasmids), and/or artificial chromosomes (e.g., yeast artificial chromosomes, YACs, or bacterial artificial chromosomes, BACs). See. e.g., Actis, L. A. et al. (1999) Front. Biosci. 4:D43-62; and Gunge, N. (1983) Annu. Rev. Microbiol. 37:253-276.

### Vectors

Certain aspects of the present disclosure relate to vectors comprising polynucleotide(s) encoding an OAADC of the present disclosure, a 3-HPDH of the present disclosure, and/or a PEPCK of the present disclosure.

As used herein, the term “vector” refers to a polynucleotide construct designed to introduce nucleic acids into one or more host cell(s). Vectors include cloning vectors, expression vectors, shuttle vectors, plasmids, cassettes, and the like. As used herein, the term “plasmid” refers to a circular double-stranded DNA construct used as a cloning and/or expression vector. Some plasmids take the form of an extrachromosomal self-replicating genetic element (episomal plasmid) when introduced into a host cell. Other plasmids integrate into a host cell chromosome when introduced into the host cell. Certain vectors are capable of directing the expression of coding regions to which they are operatively linked, e.g., “expression vectors.” Thus expression vectors cause host cells to express polynucleotides and/or polypeptides other than those native to the host cells, or in a non-naturally occurring manner in the host cells. Some vectors may result in the integration of one or more polynucleotides (e.g., recombinant polynucleotides) into the genome of a host cell.

In some embodiments, a vector of the present disclosure (e.g., an expression vector) comprises a polynucleotide sequence that encodes an OAADC of the present disclosure. For example, in some embodiments, a vector of the present disclosure (e.g., an expression vector) comprises a polynucleotide sequence that encodes an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to MTYTVGRYLADRLAQIGLKHHFAVAGDYNLVLLDQLLLNTDMQQIYCSNELNCG FSAEGYARANGAAAAIVTFSVGALSAFNALGGAYAENLPVILISGAPNANDHGTGH ILHHTLGTTDYGYQLEMARHITCAAESIVAAEDAPAKIDHVIRTALREKKPAYLEIA CNVAGAPCVRPGGIDALLSPPAPDEASLKAAVDAALAFIEQRGSVTMLVGSRIRAA GAQAQAVALADALGCAVTTMAAAKSFFPEDHPGYRGHYWGEVSSPGAQQAVEG ADGVICLAPVFNDYATVGWSAWPKGDNVMLVERHAVTVGGVAYAGIDMRDFLT RLAAHTVRRDATARGGAYVTPQTPAAAPTAPLNNAEMARQIGALLTPRTTLTAET GDSWFNAVRMKLPHGARVELEMQWGHIGWSVPAAFGNALAAPERQHVLMVGD GSFQLTAQEVAQMIRHDLPVIIFLINNHGYTIEVMIHDGPYNNVKNWDYAGLMEVF NAGEGNGLGLRARTGGELAAAIEQARANRNGPTLIECTLDRDDCTQELVTWGKRV AAANARPPRAG (SEQ ID NO:1). In some embodiments, a vector of the present disclosure (e.g., an expression vector) comprises the polynucleotide sequence of SEQ ID NO:2. In some embodiments, a vector of the present disclosure (e.g., an expression vector) comprises a polynucleotide sequence that encodes an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence selected from the group consisting of SEQ ID NOs:1, 145, 146, 148, and 166. In some embodiments, a vector of the present disclosure (e.g., an expression vector) comprises a polynucleotide sequence that encodes an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence selected from the group consisting of SEQ ID NOs:145, 146, 148, and 166. In some embodiments, a vector of the present disclosure (e.g., an expression vector) comprises a polynucleotide sequence that encodes a sequence selected from the group consisting of SEQ ID NOs:1, 145, 146, 148, and 166. In some embodiments, a vector of the present disclosure (e.g., an expression vector) comprises a polynucleotide sequence that encodes a sequence selected from the group consisting of SEQ ID NOs:145, 146, 148, and 166.

In some embodiments, a vector of the present disclosure (e.g., an expression vector) comprises a polynucleotide sequence that encodes a 3-HPDH of the present disclosure. For example, in some embodiments, a vector of the present disclosure (e.g., an expression vector) comprises a polynucleotide sequence that encodes a polypeptide that is at least 90%, at least 91%, at least 92%, at least 93%, at least 94%, at least 95%, at least 96%, at least 97%, at least 98%, at least 99%, or 100% identical to a polypeptide shown in Table 1. In some embodiments, a vector of the present disclosure (e.g., an expression vector) comprises a polynucleotide sequence that encodes an amino acid sequence selected from the group consisting of SEQ ID NOs:122-130. In some embodiments, a vector of the present disclosure (e.g., an expression vector) comprises a polynucleotide sequence that encodes a polypeptide that is at least 90%, at least 91%, at least 92%, at least 93%, at least 94%, at least 95%, at least 96%, at least 97%, at least 98%, at least 99%, or 100% identical to a polypeptide shown in Table 7A. In some embodiments, a vector of the present disclosure (e.g., an expression vector) comprises a polynucleotide sequence that encodes the amino acid sequence of SEQ ID NO:154 or 159.

In some embodiments, a vector of the present disclosure (e.g., an expression vector) comprises a polynucleotide sequence that encodes an OAADC of the present disclosure (e.g., as described supra) and a polynucleotide sequence that encodes a 3-HPDH of the present disclosure (e.g., as described supra).

In some embodiments, a vector of the present disclosure (e.g., an expression vector) comprises a polynucleotide sequence that encodes a PEPCK of the present disclosure. For example, in some embodiments, a vector of the present disclosure (e.g., an expression vector) comprises a polynucleotide sequence that encodes a polypeptide that is at least 90%, at least 91%, at least 92%, at least 93%, at least 94%, at least 95%, at least 96%, at least 97%, at least 98%, at least 99%, or 100% identical to a polypeptide shown in Table 9A. In some embodiments, a vector of the present disclosure (e.g., an expression vector) comprises a polynucleotide sequence that encodes the amino acid sequence of SEQ ID NO:162 or 163.

In some embodiments, a vector of the present disclosure (e.g., an expression vector) comprises a polynucleotide sequence that encodes an OAADC of the present disclosure (e.g., as described supra), a polynucleotide sequence that encodes a 3-HPDH of the present disclosure (e.g., as described supra), and a polynucleotide sequence that encodes a PEPCK of the present disclosure (e.g., as described supra).

In some embodiments, a vector of the present disclosure (e.g., an expression vector) comprises one or more of the promoters described infra, e.g., in operable linkage with a coding sequence or polynucleotide described herein. In some embodiments, a vector of the present disclosure (e.g., an expression vector) comprises a polynucleotide sequence that encodes an OAADC of the present disclosure operably linked to a promoter, where the promoter is not an endogenous OAADC promoter (e.g., the promoter is not operably linked to the polynucleotide as the polynucleotide is found in nature). In some embodiments, the vector is a bacterial or prokaryotic expression vector. In some embodiments, the vector is a yeast or fungal cell expression vector.

### Promoters

In some embodiments, a coding sequence of interest is placed under control of one or more promoters. “Under the control” refers to a recombinant nucleic acid that is operably linked to a control sequence, enhancer, or promoter. The term “operably linked” as used herein refers to a configuration in which a control sequence, enhancer, or promoter is placed at an appropriate position relative to the coding sequence of the nucleic acid sequence such that the control sequence, enhancer, or promoter directs the expression of a polypeptide.

“Promoter” is used herein to refer to any nucleic acid sequence that regulates the initiation of transcription for a particular coding sequence under its control. A promoter does not typically include nucleic acids that are transcribed, but it rather serves to coordinate the assembly of components that initiate the transcription of other nucleic acid sequences under its control. A promoter may further serve to limit this assembly and subsequent transcription to specific prerequisite conditions. Prerequisite conditions may include expression in response to one or more environmental, temporal, or developmental cues; these cues may be from outside stimuli or internal functions of the cell. Bacterial and fungal cells possess a multitude of proteins that sense external or internal conditions and initiate signaling cascades ending in the binding of proteins to specific promoters and subsequent initiation of transcription of nucleic acid(s) under the control of the promoters. When transcription of a nucleic acid(s) is actively occurring downstream of a promoter, the promoter can be said to “drive” expression of the nucleic acid(s). A promoter minimally includes the genetic elements necessary for the initiation of transcription, and may further include one or more genetic elements that serve to specify the prerequisite conditions for transcriptional initiation. A promoter may be encoded by the endogenous genome of a host cell, or it may be introduced as part of a recombinant, engineered polynucleotide. A promoter sequence may be taken from one host species and used to drive expression of a gene in a host cell of a different species. A promoter sequence may also be artificially designed for a particular mode of expression in a particular species, through random mutation or rational design. In recombinant engineering applications, specific promoters are used to express a recombinant gene under a desired set of physiological or temporal conditions or to modulate the amount of expression of a recombinant nucleic acid. In some embodiments, the promoters described herein are functional in a wide range of host cells.

In some embodiments, one or more genes of the present disclosure (e.g., polynucleotides encoding an OAADC, 3-HPDH, pyruvate kinase, phosphoenolpyruvate carboxylase, or pyruvate carboxylase) is operably linked to a promoter, e.g., a constitutive or inducible promoter. In some embodiments, the promoter is exogenous with respect to the polynucleotide that encodes the OAADC. For example, in some embodiments, the promoter is derived from a different source organism than the polynucleotide that encodes the OAADC and/or is not naturally found in operable linkage with the polynucleotide that encodes the OAADC (e.g., in the source organism of the OAADC).

Various promoters suitable for prokaryotic and/or yeast/fungal host cells are known. In some embodiments, a vector of the present disclosure (e.g., an expression vector) comprises a polynucleotide sequence that encodes an OAADC of the present disclosure, and a polynucleotide sequence that encodes a 3-HPDH of the present disclosure and/or a polynucleotide sequence that encodes a PEPCK of the present disclosure in a single operon. In some embodiments, the operon is operably linked to a T7 or phage promoter. In some embodiments, the T7 promoter comprises the polynucleotide sequence TAATACGACTCACTATAGGGAGA (SEQ ID NO:134). In some embodiments, an operon of the present disclosure comprises (a) a polynucleotide that encodes an amino acid sequence at least 80% identical to SEQ ID NO:1 (e.g., SEQ ID NO:2), (b) a polynucleotide encoding a 3-hydroxypropionate dehydrogenase (3-HPDH) (e.g., a polynucleotide encoding a 3-HPDH listed in Table 1 or Table 7A) or a polynucleotide encoding an alcohol dehydrogenase (e.g., comprising the sequence of NCBI GenBank Ref. No. ABX13006 or a polynucleotide encoding an alcohol dehydrogenase listed in Table 7A), and (c) a polynucleotide encoding a phosphoenolpyruvate carboxykinase (e.g., comprising a polynucleotide encoding a phosphoenolpyruvate carboxykinase listed in Table 9A). In some embodiments, the phosphoenolpyruvate carboxykinase is selected from the group consisting of E. coli Pck, NCBI Ref. Seq. No. WP_011201442, NCBI Ref. Seq. No. WP_011978877, NCBI Ref. Seq. No. WP_027939345, NCBI Ref. Seq. No. WP_074832324, and NCBI Ref. Seq. No. WP_074838421. In some embodiments, the 3-HPDH comprises the amino acid sequence of SEQ ID NO:154 or 159. In some embodiments, the PEPCK comprises the amino acid sequence of SEQ ID NO:162 or 163. In some embodiments, the OAADC comprises a sequence selected from the group consisting of SEQ ID NOs:1, 145, 146, 148, and 166.

In some embodiments, a vector of the present disclosure (e.g., an expression vector) comprises a polynucleotide sequence that encodes an OAADC of the present disclosure and a polynucleotide sequence that encodes a 3-HPDH of the present disclosure, both operably linked to the same promoter. In some embodiments, a vector of the present disclosure (e.g., an expression vector) comprises a polynucleotide sequence that encodes an OAADC of the present disclosure, a polynucleotide sequence that encodes a 3-HPDH of the present disclosure, and a polynucleotide sequence that encodes a PEPCK of the present disclosure, all operably linked to the same promoter. In some embodiments, a vector of the present disclosure (e.g., an expression vector) comprises a polynucleotide sequence that encodes an OAADC of the present disclosure and a polynucleotide sequence that encodes a 3-HPDH of the present disclosure operably linked to different promoters. In some embodiments, a vector of the present disclosure (e.g., an expression vector) comprises a polynucleotide sequence that encodes an OAADC of the present disclosure, a polynucleotide sequence that encodes a 3-HPDH of the present disclosure, and a polynucleotide sequence that encodes a PEPCK of the present disclosure operably linked to different promoters. In some embodiments, a vector of the present disclosure (e.g., an expression vector) comprises a polynucleotide sequence that encodes an OAADC of the present disclosure, a polynucleotide sequence that encodes a 3-HPDH of the present disclosure, and/or a polynucleotide sequence that encodes a PEPCK of the present disclosure operably linked to a TDH promoter or an FBA promoter. In some embodiments, the TDH promoter comprises the polynucleotide sequence TTGATTTAACCTGATCCAAAAGGGGTATGTCTATTTAGAGAGTGTTTTGTG TCAAATTATGGTAGAATGTGTAAAGTAGTATAAACTTCCTCTCAAATGACGAG GTTTAAAACACCCCCCGGGTGAGCCGAGCCGAGAATGGGGCAATTGTTCAATG TGAAATAGAAGTATCGAGTGAGAAACTTTGGGTGTTGGCCAGCCAAGGGGGGGG GGGGAAGGAAAATGGCGCGAATGCTCAGGTGAGATFGTTTGGAATTGGGTG AAGCGAGGAAATGAGCGACCCGGAGGTTGTGACTTTAGTGGCGGAGGAGGAC GGAGGAAAAGCCAAGAGGGAAGTGTATATAAGGGGAGCAATTTGCCACCAAGG ATAGAATTTGGATGAGTTATAATTCTACTGTATTTATTGTATAATTTATTTCTCCT TTTGTATCAAACACATTACAAAACACACAAAACACACAAACAAACACAATTAC AAAAA (SEQ ID NO:135). In some embodiments, the FBA promoter comprises the polynucleotide sequence TATCGTATTTATTAATCCCCTTCCCCCCAGCGCAGATCGTCCCGTCGATTCTAT TGTTGGGCATTATCAGCGACGCGACGGCGACGCGACGGCGATAATGGGCGAC GGTCACAAGATGGAACGAGAAAACAGTTTTTCGGATAGGACTCATTTTCCAG GTGAGAATGGGGTGACCCCGGGGAGAAACCTCCGCGAGTGGAGTGCGAGTGG AGTGGGAAATGTGGCCCCCCCCCCCCTTGTGGGCCATGAGGTTGACAAATACC GTGTGGCCCGGTGATGGAGTGAGAAAGAGAGGGAAATGATAATGGGAAAACA AGGAGAGGCCCGTTTCCCGGGATTTATATAAAGAGGTGTCTCTATCCCAGTTGA AGTAGAGATTTGTTGATGTAGTTTGTCCTTCCAATAAATTTGTTCAATCAGTACA CAGCTAATACTATTATTACAGCTACTACTAATACTACTACTACTATTACTACCAC CCCCAACACAAACACA (SEQ ID NO:136).

In some embodiments, a constitutive promoter is defined herein as a promoter that drives the expression of nucleic acid(s) continuously and without interruption in response to internal or external cues. Constitutive promoters are commonly used in recombinant engineering to ensure continuous expression of desired recombinant nucleic acid(s). Constitutive promoters often result in a robust amount of nucleic acid expression, and, as such, are used in many recombinant engineering applications to achieve a high level of recombinant protein and enzymatic activity.

Many constitutive promoters are known and characterized in the art. Exemplary bacterial constitutive promoters include without limitation the E. coli promoters Pspc, Pbla, PRNAI, PRNAII, P1 and P2 from rrnB, and the lambda phage promoter PL (Liang, S. T. et al. J Mol. Biol. 292(1): 19-37 (1999)). In some embodiments, the constitutive promoter is functional in a wide range of host cells.

An inducible promoter is defined herein as a promoter that drives the expression of nucleic acid(s) selectively and reliably in response to a specific stimulus. An ideal inducible promoter will drive no nucleic acid expression in the absence of its specific stimulus but drive robust nucleic acid expression rapidly upon exposure to its specific stimulus. Additionally, some inducible promoters induce a graded level of expression that is tightly correlated with the amount of stimulus received. Stimuli for known inducible promoters include, for example, heat shock, exogenous compounds or a lack thereof (e.g., a sugar, metal, drug, or phosphate), salts or osmotic shock, oxygen, and biological stimuli (e.g., a growth factor or pheromone).

Inducible promoters are often used in recombinant engineering applications to limit the expression of recombinant nucleic acid(s) to desired circumstances. For example, since high levels of recombinant protein expression may sometimes slow the growth of a host cell, the host cell may be grown in the absence of recombinant nucleic acid expression, and then the promoter may be induced when the host cells have reached a desired density. Many inducible promoters are known and characterized in the art. Exemplary bacterial inducible promoters include without limitation the E. coli promoters P_{lac}, Pₜᵣₚ, P_{lac}, P_{T7}, P_{BAD}, and P_{lacUV5} (Nocadello, S. and Swennen, E. F. Microb Cell Fact, 11:3 (2012)). In some preferred embodiments, the inducible promoter is a promoter that functions in a wide range of host cells. Inducible promoters that functional in a wide variety of host bacterial and yeast cells are well known in the art.

### Genetic Markers

Certain aspects of the present invention related to genetic markers that allow selection of host cells that have one or more desired polynucleotides. In some embodiments, the genetic marker is a positive selection marker that confers a selective advantage to the host organisms. Examples of positive markers are genes that complement a metabolic defect (autotrophic markers) and antibiotic resistance markers.

In some embodiments, the genetic marker is an antibiotic resistance marker such as Apramycin resistance, Ampicillin resistance, Kanamycin resistance, Spectinomycin resistance, Tetracyclin resistance, Neomycin resistance, Chloramphenicol resistance, Gentamycin resistance, Erythromycin resistance, Carbenicillin resistance, Actinomycin D resistance, Neomycin resistance, Polymyxin resistance, Zeocin resistance and Streptomycin resistance. In some embodiments, the genetic marker includes a coding sequence of an antibiotic resistance protein (e.g., a beta-lactamase for certain Ampicillin resistance markers) and a promoter or enhancer element that drives expression of the coding sequence in a host cell of the present disclosure. In some embodiments, a host cell of the present disclosure is grown under conditions in which an antibiotic resistance marker is expressed and confers resistance to the host cell, thereby selected for the host cell with a successful integration of the marker. Exemplary culture conditions and media are described herein.

In some embodiments, the genetic marker is an auxotrophic marker, such that marker complements a nutritional mutation in the host cell. In some embodiments, the auxotrophic marker is a gene involved in vitamin, amino acid, fatty acid synthesis, or carbohydrate metabolism; suitable auxotrophic markers for these nutrients are well known in the art. In some embodiments, the auxotrophic marker is a gene for synthesizing an amino acid. In some embodiments, the amino acid is any of the 20 essential amino acids. In some embodiments, the auxotrophic marker is a gene for synthesizing glycine, alanine, valine, leucine, isoleucine, proline, phenylalanine, tyrosine, tryptophan, serine, threonine, cysteine, methionine, asparagine, glutamine, lysine, arginine, histidine, aspartate or glutamate. In some embodiments, the auxotrophic marker is a gene for synthesizing adenosine, biotin, thiamine, leucine, glucose, lactose, or maltose. In some embodiments, a host cell of the present disclosure is grown under conditions in which an auxotrophic resistance marker is expressed in an environment or medium lacking the corresponding nutrient and confers growth to the host cell (lacking an endogenous ability to produce the nutrient), thereby selected for the host cell with a successful integration of the marker. Exemplary culture conditions and media are described herein.

### Cell Culture Media and Methods

Certain aspects of the present disclosure relate to methods of culturing a cell. As used herein, “culturing” a cell refers to introducing an appropriate culture medium, under appropriate conditions, to promote the growth of a cell. Methods of culturing various types of cells are known in the art. Culturing may be performed using a liquid or solid growth medium. Culturing may be performed under aerobic or anaerobic conditions where aerobic, anoxic, or anaerobic conditions are preferred based on the requirements of the microorganism and desired metabolic state of the microorganism. In addition to oxygen levels, other important conditions may include, without limitation, temperature, pressure, light, pH, and cell density.

In some embodiments, a culture medium is provided. A “culture medium” or “growth medium” as used herein refers to a mixture of components that supports the growth of cells. In some embodiments, the culture medium may exist in a liquid or solid phase. A culture medium of the present disclosure can contain any nutrients required for growth of microorganisms. In certain embodiments, the culture medium may further include any compound used to reduce the growth rate of, kill, or otherwise inhibit additional contaminating microorganisms, preferably without limiting the growth of a host cell of the present disclosure (e.g., an antibiotic, in the case of a host cell bearing an antibiotic resistance marker of the present disclosure). The growth medium may also contain any compound used to modulate the expression of a nucleic acid, such as one operably linked to an inducible promoter (for example, when using a yeast cell, galactose may be added into the growth medium to activate expression of a recombinant nucleic acid operably linked to a GAL1 or GAL10 promoter). In further embodiments, the culture medium may lack specific nutrients or components to limit the growth of contaminants, select for microorganisms with a particular auxotrophic marker, or induce or repress expression of a nucleic acid responsive to levels of a particular component.

In some embodiments, the methods of the present disclosure may include culturing a host cell under conditions sufficient for the production of a product, e.g., 3-HP. In certain embodiments, culturing a host cell under conditions sufficient for the production of a product entails culturing the cells in a suitable culture medium. Suitable culture media may differ among different microorganisms depending upon the biology of each microorganism. Selection of a culture medium, as well as selection of other parameters required for growth (e.g., temperature, oxygen levels, pressure, etc.), suitable for a given microorganism based on the biology of the microorganism are well known in the art. Examples of suitable culture media may include, without limitation, common commercially prepared media, such as Luria Bertani (LB) broth, Sabouraud Dextrose (SD) broth, or Yeast medium (YM, YPD, YPG, YPAD, etc.) broth. In other embodiments, alternative defined or synthetic culture media may also be used.

Certain aspects of the present disclosure relate to culturing a recombinant host cell of the present disclosure in a culture medium comprising a substrate under conditions suitable for the recombinant host cell to convert the substrate to 3-HP. A variety of substrates are contemplated for use herein. In some embodiments, the substrate is a compound described herein that can be used as a metabolic precursor to generate oxaloacetate.

In some embodiments, the substrate comprises glucose. In some embodiments, the substrate is glucose. In some embodiments, at least about 50%, at least about 55%, at least about 60%, at least about 65%, at least about 70%, at least about 75%, at least about 80%, at least about 85%, at least about 90%, at least about 95%, or 100% of the glucose metabolized by the recombinant host cell is converted to 3-HP.

Other substrates contemplated for use herein include, without limitation, sucrose, fructose, xylose, arabinose, cellobiose, cellulose, alginate, mannitol, laminarin, galactose, and galactan. In some embodiments, at least about 50%, at least about 55%, at least about 60%, at least about 65%, at least about 70%, at least about 75%, at least about 80%, at least about 85%, at least about 90%, at least about 95%, or 100% of the substrate metabolized by the recombinant host cell is converted to 3-HP. A variety of techniques suitable for engineering a recombinant host cell able to metabolize these and other substrates have been described. See, e.g., Enquist-Newman, M. et al. (2014) Nature 505:239-43 (describing S. cerevisiae host cells capable of metabolizing 4-deoxy-L-erythro-5-hexoseulose urinate or mannitol); Wargacki, A. J. et al. (2012) Science 335:308-313 (describing E. coli host cells capable of metabolizing alginate, mannitol, and glucose); and Turner, T. L. et al. (2016) Biotechnol. Bioeng. 113:1075-1083 (describing S. cerevisiae host cells capable of cellobiose and xylose).

In some embodiments, a recombinant host cell of the present disclosure is cultured under semiacrobic or anaerobic conditions (e.g., semiacrobic/anacrobic conditions suitable for the host cell to produce 3-HP). As described herein, production of 3-HP using a recombinant host cell of the present disclosure is thought to be advantageous, e.g., for increasing scale of production, yield, and/or cost efficacy. In some embodiments, anaerobic conditions may refer to conditions in which average oxygen concentration is 20% or less than the average oxygen concentration of tap water or of an average aqueous environment.

### Purification of Products from Host Cells

In some embodiments, the methods of the present disclosure further comprise substantially purifying 3-HP produced by a host cell of the present disclosure, e.g., from a cell culture or cell culture medium.

A variety of methods known in the art may be used to purify a product from a host cell or host cell culture. In some embodiments, one or more products may be purified continuously, e.g., from a continuous culture. In other embodiments, one or more products may be purified separately from fermentation, e.g., from a batch or fed-batch culture. One of skill in the art will appreciate that the specific purification method(s) used may depend upon, inter alia, the host cell, culture conditions, and/or particular product(s).

In some embodiments, purifying 3-HP comprises: separating or filtering the host cells from a cell culture medium, separating the 3-HP from the culture medium (e.g., by solvent extraction), concentration of water (e.g., by evaporation), and crystallization of the 3-HP. Techniques for purifying 3-HP are known in the art; see. e.g., U.S. Pat. Nos. 7,279,598 and 6,852,517; U.S. PG Pub. Nos. US20100021978, US2009032548, and US20110244575; and International Pub. Nos. WO2010011874, WO2013192450, and WO2013192451. In some embodiments, the solvent is an organic solvent, including without limitation alcohols, aldehydes, ethers, and ketones. For descriptions of exemplary purification schemes, see. e.g., WO2013192450.

In some embodiments, the methods of the present disclosure further comprise converting 3-HP (e.g., substantially purified 3-HP) into acrylic acid. Techniques for converting 3-HP into acrylic acid are known: see, e.g., WO2013192451 and WO2013185009. In some embodiments, 3-HP is converted into acrylic acid via a catalyst and heat. In some embodiments, 3-HP is converted into acrylic acid by vaporizing 3-HP in aqueous solution and contacting the vapor with a catalyst or inert surface area. In some embodiments, the aqueous solution containing the 3-HP is obtained from a cell culture medium, e.g., by concentrating the medium (e.g., by removal of water).

### Examples

The present disclosure will be more fully understood by reference to the following examples. They should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Identification of Novel Oxaloacetate Decarboxylases

This study shows the identification of candidate enzymes capable of directly catalyzing the decarboxylation of oxaloacetate to 3-oxoproponanoate using a genomic mining method. Purified candidate enzymes were characterized in functional assays to assess catalytic activity and substrate preference for oxaloacetate compared to pyruvate.

Materials and Methods

### Genomic Enzyme Mining

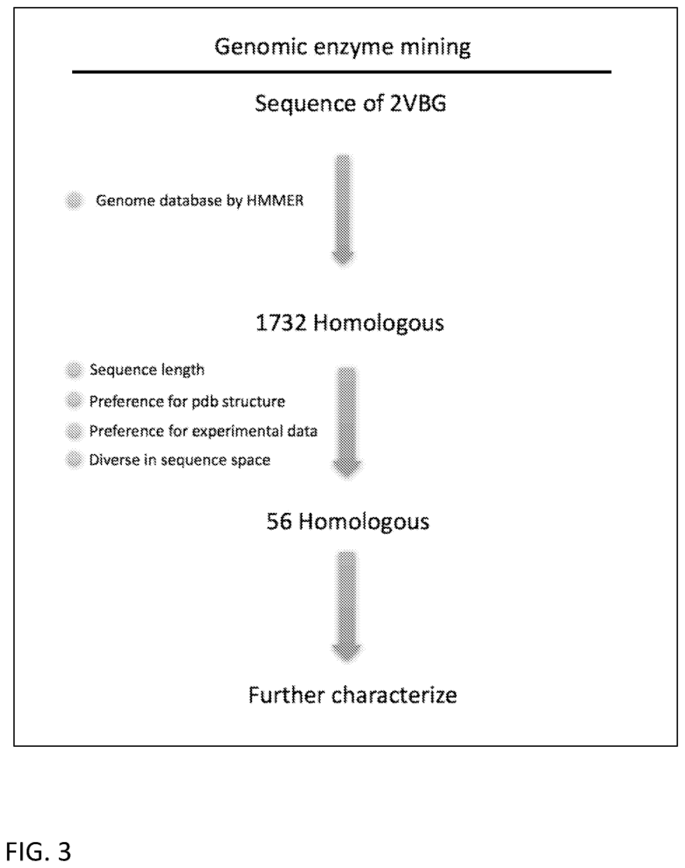

FIG. 3 depicts an overview of the genomic enzyme mining scheme employed to identify candidate oxaloacetate decarboxylase enzymes. Briefly, branched-chain ketoacid decarboxylase from Lactococcus lactis (crystal structure PDB code: 2VBG) was identified to have a relatively broad substrate spectrum (Smit, B. A. et al. (2005) Appl. Environ. Microbiol. 71:303-311). Therefore, its sequence was used as the input to perform genomic database searching via HMMER (Finn, R. D. et al. (2011) Nucleic Acids Res. 39:W29-W37). The target database was set to 15 representative proteomes, and the significance level for E-values was set at 1e-50.

The search resulted in 1,732 significant hits, and the resulting sequences were subsequently filtered using the CD-HIT online server with a 90% identity cutoff. A set of 1,303 homologous gene sequences was then generated. Sequences derived from bacteria were preferred due to the increased likelihood of producing soluble proteins in E. coli. Enzymes with a sequence length less than 200 amino acids or more than 700 amino acids were removed since the average sequence length of ketoacid decarboxylases is about 500 amino acids. To select enzymes for characterization studies, proteins sequences that were experimentally validated and annotated as TPP binding proteins were prioritized. For the purpose of diversifying enzyme candidates, the selected sequences broadly covered the entire enzyme family.

Table 2 shows the final sequence library containing 56 sequences with an average of 15% sequence identity, which were verified by phylogenetic analysis. These candidates were subsequently characterized for activity towards oxaloacetate.

TABLE 2
Protein and gene sequences of candidate oxaloacetate decarboxylase enzymes.

| Enzyme | | |
|---|---|---|
| name or | | |
| UniProt/ | | |
| Genebank ID | Species | Protein Sequence |
| 4COK | Gluconacetobacter | MTYTVGRYLADRLAQIGLKHHFAVAGDYNLVLLDQL |
| | diazotrophicus | LLNTDMQQIYCSNELNCGFSAEGYARANGAAAAIVTF |
| | | SVGALSAFNALGGAYAENLPVILISGAPNANDHGTGHI |
| | | LHHTLGTTDYGYQLEMARHITCAAESIVAAEDAPAKID |
| | | HVIRTALREKKPAYLEIACNVAGAPCVRPGGIDALLSP |
| | | PAPDEASLKAAVDAALAFIEQRGSVTMLVGSRIRAAG |
| | | AQAQAVALADALGCAVTTMAAAKSFFPEDHPGYRGH |
| | | YWGEVSSPGAQQAVEGADGVICLAPVFNDYATVGWS |
| | | AWPKGDNVMLVERHAVTVGGVAYAGIDMRDFLTRL |
| | | AAHTVRRDATARGGAYVTPQTPAAAPTAPLNNAEMA |
| | | RQIGALLTPRTTLTAETGDSWFNAVRAMKLPHGARVEL |
| | | EMQWGHIGWSVPAAFGNALAAPERQHVLMVGDGSFQ |
| | | LTAQEVAQMIRHDLPVIIFLINNHGYTIEVMIHDGPYNN |
| | | VKNWDYAGLMEVFNAGEGNGLGLRARTGGELAAAIE |
| | | QARANRNGPTLIECTLDRDDCTQELVTWGKRVAAAN |
| | | ARPPRAG |
| | | (SEQ ID NO: 1) |
| A0A0F6SDN1_9DELT | Sandaracinus | MADLLAIHRHAVRARLLDERLTQLARAGRIGFHPDAR |
| | amylolyticus | GFEPAIAAAVLAMRAEDAIFPSARDHAAFLVRGLPISR |
| | | YVAHAFGSVEDPMRGHAAPGHLASRELRIAAASGLVS |
| | | NHMTHAAGYAWAAKLRGETCAVLTMFADTAADAGD |
| | | FHSAVNFAGATKAPVIFFCRTDRTRSAHPPTPIDRVAD |
| | | KGIAYGVESLVCSADDAGAVASAMAQAHQRALAGEG |
| | | PTLVEAIRESKSDPIEALEARLSSEGHWDAHRALELRRE |
| | | LMTEIESAVAHAQQVGAPPREAVFEDVYATLPRHLED |
| | | QRTTLLATANHEDR |
| | | (SEQ ID NO: 3) |
| 4K9Q | Polynucleobacter | MRTVKEITFDLLRKLQVTTVVGNPGSTEETFLKDFPSD |
| | necessarius subsp. | FNYVLALQEASVVAIADGLSQSLRKPVIVNIHTGAGLG |
| | Asymbioticus | NAMGCLLTAYQNKTPLIITAGQQTREMLLNEPLLTNIE |
| | | AINMPKPWVKWSYEPARPEDVPGAFMRAYATAMQQP |
| | | QGPVFLSLPLDDWEKLIPEVDVARTVSTRQGPDPDKV |
| | | KEFAQRITASKNPLLIYGSDIARSQAWSDGIAFAERLNA |
| | | PVWAAPFAERTPFPEDHPLFQGALTSGIGSLEKQIQGH |
| | | DLIVVIGAPVFRYYPWIAGQFIPEGSTLLQVSDDPNMTS |
| | | KAVVGDSLVSDSKLFLIEALKLIDQREKNNTPQRSPMT |
| | | KEDRTAMPLRPHAVLEVLKENSPKEIVLVEECPSIVPL |
| | | MQDVFRINQPDTFYTFASGGLGWDLPAAVGLALGEEV |
| | | SGRNRPVVTLMGDGSFQYSVQGIYTGVQQKTHVIYVV |
| | | FQNEEYGILKQFAELEQTPNVPGLDLPGLDIVAQGKAY |
| | | GAKSLKVETLDELKTAYLEALSFKGTSVIVVPITKELKP |
| | | LFG |
| | | (SEQ ID NO: 5) |
| D6ZJY9_MOBCV | Mobiluncus curtisii | MLKQIEGSQAIARAVAACQPNVVAAYPISPQTHIVEAL |
| | | SALVKSGQLEHCEYVNVESEFAAMSACIGSSAVGARS |
| | | YTATASQGLLYMVEAVYNAAGLGFPIVMTVANRAIG |
| | | APINIWNDHSDSMSQRDSGWLQLFAENNQEAADLHV |
| | | QAFRIAEELSVPVMVCMDGFILTHAVEQVDLPESEQVK |
| | | QFLPPYEPRQVLDPDDPLSIGAMVGPEAFTEVRYIAHH |
| | | KMLQALDLIPQVQSEFKSIFGRDSGGLLHTYRCEDAETI |
| | | IVALGSVVGTLKDVVDQRRENGEKIGIMSLVSFRPFPF |
| | | AAIREVLQSAKRWCLEKAFQLGIGGIVSSELRAAMRG |
| | | LPFTCYEVIAGLGGRNITKNSLHAMLDQAVADTIEPLT |
| | | FMDLDMELVQGELEREAATRRSGAFATNLQRERVLRA |
| | | NAKIAEAGPKPKADKVGNPRVASPSIKQDAVPVVPDQ |
| | | AE |
| | | (SEQ ID NO: 7) |
| \|Q1LMD8_CUPMC | Cupriavidus | MIEAVQFVEAARERGFEWYAGVPCSYLTPFINYVVQD |
| | metallidurans | PSLHYVSAANEGDAVAFIAGVTQGARNGVRGITMMQ |
| | | NSGLGNAVSPLTSLTWTFRLPQLLIVTWRGQPGGASDE |
| | | PQHALMGPVTPAMLDTMEIPWELFPTEPDAVGPALDR |
| | | AIAHMDATGRPYALIMQKGSVAPYPLKTQTPPVARAK |
| | | ATPQVSRSGATPLPSRQEALQRVIAHTPADSTVVLAST |
| | | GFCGRELYALDDRPNQLYMVGSMGCLTPFALGLAMA |
| | | RPDLKVVAVDGDGAALMRMGVFATLGAYGPANLTH |
| | | VLLDNNAHDSTGGQATVSHNVSFAGVAAACGYASAIE |
| | | GDDLDMLDRVLASAATATSGPNFVCLQTRAGTPDGLP |
| | | RPSVTPVEVKTRLGRQIGADQGHAGEKHAAA |
| | | (SEQ ID NO: 9) |
| Q9F768 | Bacteroides fragilis | MNTLTSQIEQLQSLAHELLYLGVDGAPIYTDHFRQLNK |
| | | EVLEQSDALYPQRGATPEEEANICLALLMGYNATIYNQ |
| | | GDKEEKKQVVLNRCWDVLDQLPATLLKCQLLTYCYG |
| | | EVFEELAKEAHTIIESWSNRELLKAEKEIAESLNNLEA |
| | | NPYPYSELHE |
| | | (SEQ ID NO: 11) |
| I3BXS7_9GAMM | Thiothrix nivea | MQIQVSELIVKFLQKLGVDTIFGMPGAHILPVYDELYD |
| | DSM 5205 | SGIKTVLVKFIEQGAAFMAGGYARVSGRIGACITTAGP |
| | | GASNLITGIANAYADKLPMIVITGEAPTHIFGRGGLQES |
| | | SGEGGSIDQTALFSGVTRYHKLIERTDYITNVLSQAAR |
| | | QLVADVPGPVVLSIPVNVQKELVDASILENLPTLKPLP |
| | | KLQIAPPVLEQCADMIRKARCPVILAGYGCLQSVRARL |
| | | ELRKFSEHLNIPVATSLKGKGAIDERSALSLGSLGVTSS |
| | | GHAMHYFMQEADLIILLGAGFNERTSYVWKADLTQER |
| | | KIIQVDRNVAQLEKVVKADLAIQSDLGDFLHALNTCC |
| | | VPQGIEPKSCPDLAAFKQKVDQQAAQSGQVIFNQKFD |
| | | LVKSLFARLEPHFAEGIVLVDDNIIYAQNFYRVKDGDL |
| | | FVPNTGVSSLGHAIPAAIGARFVLDKPMFAILGDGGFQ |
| | | MCCMEIMTAVNYNIPLNIVLFNNQTLGLIRKNQHQQY |
| | | EQRFLDCDFQNPDYALLAQSFGINHFHVGNNADLQRV |
| | | FDTADFHHAINLIELMVDREAYPNYSSRR |
| | | (SEQ ID NO: 13) |
| 1JSC | Saccharomyces | MIRQSTLKNFAIKRCFQHIAYRNTPAMRSVALAQRFYS |
| | cerevisiae | SSSRYYSASPLPASKRPEPAPSFNVDPLEQPAEPSKLAK |
| | | KLRAEPDMDTSFVGLTGGQIFNEMMSRQNVDTVFGYP |
| | | GGAILPVYDAIHNSDKFNFVLPKHEQGAGHMAEGYAR |
| | | ASGKPGVVLVTSGPGATNVVTPMADAFADGIPMVVFT |
| | | GQVPTSAIGTDAFQEADVVGISRSCTKWNVMVKSVEE |
| | | LPLRINEAFEIATSGRPGPVLVDLPKDVTAAILRNPIPTK |
| | | TTLPSNALNQLTSRAQDEFVMQSINKAADLINLAKKPV |
| | | LYVGAGILNHADGPRLLKELSDRAQIPVTTTLQGLGSF |
| | | DQEDPKSLDMLGMHGCATANLAVQNADLIIAVGARF |
| | | DDRVTGNISKFAPEARRAAAEGRGGIIHFEVSPKNINK |
| | | VVQTQIAVEGDATTNLGKMMSKIFPVKERSEWFAQIN |
| | | KWKKEYPYAYMEETPGSKIKPQTVIKKLSKVANDTGR |
| | | HVIVTTGVGQHQMWAAQHWTWRNPHTFITSGGLGTM |
| | | GYGLPAAIGAQVAKPESLVIDIDGDASFNMTLTELSSA |
| | | VQAGTPVKILILNNEEQGMVTQWQSLFYEHRYSHTHQ |
| | | LNPDFIKLAEAMGLKGLRVKKQEELDAKLKEFVSTKG |
| | | PVLLEVEVDKKVPVLPMVAGGSGLDEFINFDPEWRQ |
| | | QTELRHKRTGGKH |
| | | (SEQ ID NO: 15) |
| O86938\|PPD_STRVT | Streptomyces | MIGAADLVAGLTGLGVTTVAGVPCSYLTPLINRVISDP |
| | viridochromogenes | ATRYLTVTQEGEAAAVAAGAWLGGGLGCAITQNSGL |
| | | GNMTNPLTSLLHPARIPAVVITTWRGRPGEKDEPQHHL |
| | | MGRITGDLLDLCDMEWSLIPDTTDELHTAFAACRASL |
| | | AHRELPYGFLLPQGVVADEPLNETAPRSATGQVVRYA |
| | | RPGRSAARPTRIAALERLLAELPRDAAVVSTTGKSSRE |
| | | LYTLDDRDQHFYMVGAMGSAATVGLGVALHTPRPVV |
| | | VVDGDGSVLMRLGSLATVGAHAPGNLVHLVLDNGVH |
| | | DSTGGQRTLSSAVDLPAVAAACGYRAVHACTSLDDLS |
| | | DALATALATDGPTLVHLAIRPGSLDGLGRPKVTPAEVA |
| | | RRFRAFVTTPPAGTATPVHAGGVTAR |
| | | (SEQ ID NO: 17) |
| 3L84_3M34 | Campylobacter | MNIQILQEQANTLRFLSADMVQKANSGHPGAPLGLAD |
| | jejuni | ILSVLSYHLKHNPKNPTWLNRDRLVFSGGHASALLYSF |
| | | LHLSGYDLSLEDLKNFRQLHSKTPGHPEISTLGVEIATG |
| | | PLGQGVANAVGFAMAAKKAQNLLGSDLIDHKIYCLC |
| | | GDGDLQEGISYEACSLAGLHKLDNFILRYDSNNISIEGD |
| | | VGLAFNENVKMRFEAQGFEVLSINGHDYEEINKALEQ |
| | | AKKSTKPCLIIAKTTIAKGAGELEGSHKSHGAPLGEEVI |
| | | KKAKEQAGFDPNISFHIPQASKIRFESAVELGDLEEAK |
| | | WKDKLEKSAKKELLERLLNPDFNKIAYPDFKGKDLAT |
| | | RDSNGEILNVLAKNLEGFLGGSADLGPSNKTELHSMG |
| | | DFVEGKNIHFGIREHAMAAINNAFARYGIFLPFSATFFIF |
| | | SEYLKPAARIAALMKIKHFFIFTHDSIGVGEDGPTHQPI |
| | | EQLSTFRAMPNFLTFRPADGVENVKAWQIALNADIPSA |
| | | FVLSRQKLKALNEPVFGDVKNGAYLLKESKEAKFTLL |
| | | ASGSEVWLCLESANELEKQGFACNVVSMPCFELFEKQ |
| | | DKAYQERLLKGEVIGVEAAHSNELYKFCHKVYGIESF |
| | | GESGKDKDVFERFGFSVSKLVNFILSK |
| | | (SEQ ID NO: 19) |
| lupa_A | Streptomyces | MSRVSTAPSGKPTAAHALLSRLRDHGVGKVFGVVGRE |
| | clavuligerus | AASILFDEVEGIDFVLTRHEFTAGVAADVLARITGRPQ |
| | | ACWATLGPGMTNLSTGIATSVLDRSPVIALAAQSESHD |
| | | IFPNDTHQCLDSVAIVAPMSKYAVELQRPHEITDLVDS |
| | | AVNAAMTEPVGPSFISLPVDLLGSSEGIDTTVPNPPANT |
| | | PAKPVGVVADGWQKAADQAAALLAEAKHPVLVVGA |
| | | AAIRSGAVPAIRALAERLNIPVITTYIAKGVLPVGHELN |
| | | YGAVTGYMDGILNFPALQTMFAPVDLVLTVGYDYAE |
| | | DLRPSMWQKGIEKKTVRISPTVNPIPRVYRPDVDVVTD |
| | | VLAFVEHFETATASFGAKQRHDIEPLRARIAEFLADPET |
| | | YEDGMRVHQVIDSMNTVMEEAAEPGEGTIVSDIGFFR |
| | | HYGVLFARADQPFGFLTSAGCSSFGYGIPAAIGAQMAR |
| | | PDQPTFLIAGDGGFHSNSSDLETIARLNLPIVTVVVNND |
| | | TNGLIELYQNIGHHRSHDPAVKFGGVDFVALAEANGV |
| | | DATRATNREELLAALRKGAELGRPFLIEVPVNYDFQPG |
| | | GFGALSI |
| | | (SEQ ID NO: 21) |
| A0A016CS86_BACFG | Fibrobacter | MLSPKFFVETLQTYSMDFFTGVPDSLLKNMCAYITDHI |
| | succinogenes | ESQNNIIAVNEGTALGLAAGYYIATGCIPIVYMQNSGIG |
| | | NTVNPLLSLTDKVVYNIPVLLLIGWRGEPGIKDEPQHIK |
| | | QGMITIPLLDTLGIKNQILNKDPNMAKSQINDAIEYMR |
| | | MTKEAFAFVIQKDTFEEYKLQNTEDSKFDLDREEAIKI |
| | | VCNSLDKGSVIVSTTGMISRELFEYRESIDANHETDFLT |
| | | VGSMGHASQIALGIALRRKNKKVYCFDGDGAVLMHM |
| | | GALTTIGTSRAVNYIHIVFNNGAHDSVGGQPTVGLKVN |
| | | LSKIASACGYNNVISVDSKATLKESLDRFKSINGPVLLE |
| | | VKVRKGARKDLGRPTLTPVKNKELLMNFLEEADESDK |
| | | SDNVFK |
| | | (SEQ ID NO: 23) |
| A0A0F2PQV5_9FIRM | Peptococcaceae | MISTKRFGEELKKLGFDFYSGVPCSFLKNLINYTTNHC |
| | bacterium | NYLAATNEGEAVAVAAGAFLAGKKPVVLMQNSGLTN |
| | BRH_c4b | AVSPLVSLNYLFRLPVLGFVSLRGEPGIPDEPQHQLMG |
| | | RITTQMLDLVEIQWEYLSTDFDEVKKQLLQAYSCIESN |
| | | QPFFFVVKKDTFEKEQLTDSQKRLSKNMFKSERTKAD |
| | | QVPKRFETLRLINSLKDVKTVQLTTTGITGRELYEIEDV |
| | | SNNLYMVGSMGCVSSLGLGLALTKKDKDVVVIEGDG |
| | | ALLMRMGNLATNGYYGPPNMLHILLDNNMHESTGGQ |
| | | STVSYNINFVDIAAACGYTKSIYVHNLVELESHIKDWK |
| | | REKNLTFLYLKIAKGSIEGLGRPKMKPHEVKERLKVFL |
| | | DG |
| | | (SEQ ID NO: 25) |
| D7DTG5_METV3 | Methanococcus | MKTIVILLDGVADRPSKELNYKTPLQYANIPNLDEFAK |
| | voltae | SSLTGLMCPQKIGVPLGTEVAHFLLWGYDISQFPGRGV |
| | | IEALGEGIDLKKDSIYLRATLGHVNYNQKENNFLVLDR |
| | | RTKDINNQEISELLNKISNINIDGYLFTIHHMQGIHSILEI |
| | | SKLENDGNLKTEPNLKKNNLKKNGFELTYEEFCNEKNI |
| | | LKYGNINNSNNCISNKISDSDPFYKDRHVIMVKPVIKLI |
| | | GTYEEYLNALNVSNALNKYLTTCNTLLENDSINISRKN |
| | | ENKSLANFLLTKWAGSYKKLPSFKQKWGLNGVIIANS |
| | | SLFRGLAKLLKMDYYEVKEFDKAIELGLKFKNDNTNN |
| | | NNNSNNNNNNNQNNNINNKKIYDFIHIHTKEPDEAGH |
| | | TKNPINKVRVLEKLDKNLKVVIDEIDKEKENGDENLYII |
| | | TGDHATPSTGGLIHSGELVPIAICGKNVGKDSTKAFNE |
| | | MDVLNGYYRINSTDIMNLVLNYTDKALLYGLRPNGDL |
| | | KKYIPEDNELEFLKKDN |
| | | (SEQ ID NO: 27) |
| 3E9Y | Arabidopsis | MAAATTTTTTSSSISFSTKPSPSSSKSPLPISRFSLPFSLNP |
| | thaliana | NKSSSSSRRRGIKSSSPSSISAVLNTTTNVTTTPSPTKPT |
| | | KPETFISRFAPDQPRKGADILVEALERQGVETVFAYPG |
| | | GASMEIHQALTRSSSIRNVLPRHEQGGVFAAEGYARSS |
| | | GKPGICIATSGPGATNLVSGLADALLDSVPLVAITGQVP |
| | | RRMIGTDAFQETPIVEVTRSITKHNYLVMDVEDIPRIIEE |
| | | AFFLATSGRPGPVLVDVPKDIQQQLAIPNWEQAMRLP |
| | | GYMSRMPKPPEDSHLEQIVRLISESKKPVLYVGGGCLN |
| | | SSDELGRFVELTGIPVASTLMGLGSYPCDDELSLHMLG |
| | | MHGTVYANYAVEHSDLLLAFGVRFDDRWGKLEAFA |
| | | SRAKIVHIDIDSAEIGKNKTPHVSVCGDWLALQGMNK |
| | | VLENRAEELKLDFGVWRNELNVQKQKFPLSFKTFGEA |
| | | IPPQYAIKVLDELTDGKAIISTGVGQHQMQWAAQFYNY |
| | | KKPRQWLSSGGLGAMGFGLPAAIGASVANPDAIVVDI |
| | | DGDGSFIMNVQELATIRVENLPVKVLLLNNQHLGMVM |
| | | QWEDRFYKANRAHTFLGDPAQEDEIFPNMLLFAAACG |
| | | IPAARVTKKADLREAIQTMLDTPGPYLLDVICPHQEHV |
| | | LPMIPSGGTFNDVITEGDGRIKY |
| | | (SEQ ID NO: 29) |
| 2ZKT | Pyrococcus | MVLKRKGLLIILDGLGDRPIKELNGLTPLEYANTPNMD |
| | furiosus | KLAEIGILGQQDPIKPGQPAGSDTAHLSIFGYDPYETYR |
| | | GRGFFEALGVGLDLSKDDLAFRVNFATLENGIITDRRA |
| | | GRISTEEAHELARAIQEEVDIGVDFIFKGATGHRAVLVL |
| | | KGMSRGYKVGDNDPHEAGKPPLKFSYEDEDSKKVAEI |
| | | LEEFVKKAQEVLEKHPINERRRKEGKPIANYLLIRGAG |
| | | TYPNIPMKFTEQWKVKAAGVIAVALVKGVARAVGFD |
| | | VYTPEGATGEYNTNEMAKAKKAVELLKDYDFWLHF |
| | | KPTDAAGHDNKPKLKAELIERADRMIGYILDHVDLEE |
| | | VYIAITGDHSTPCEVMNHSGDPVPLLIAGGGVRTDDTK |
| | | RFGEREAMKGGLGRIRGHDIVPIMMDLMNRSEKFGA |
| | | (SEQ ID NO: 31) |
| A0A124FLS8_9FIRM | Clostridia | MLLVVLDGLGGLPVPELNGRTELEAAATPNLDALAKR |
| | bacterium 62_21 | SSLGLAHPVLPGIAPGSSAGHLALFGYDPLRYVIGRGV |
| | | LEALGIGFDLHPGDVAVRANFATVQDTRNGPWTDRR |
| | | AGRPPTEHTRSICRRLQDAIPEIDGVRVFIEPVKEHRFVI |
| | | VLRGEGLDDRVADTDPQREGMPPLQPQPLAEEARRTA |
| | | MLAGTLVQRIAELVRDEPRTNFALLRGFSRRPRLDPFP |
| | | ERYRARAGAVAVYPMYRGLASLVGMDLLPVAGDTLA |
| | | DEIASLKENWPEYDYFFLHVKGTDSRGEDGDWAGKIK |
| | | IIEEFDAQLPAILDLNPDALVITGDHSTPATYAAHSWHP |
| | | VPFLLYSRWVLPDRDAPGFGEHACARGVLGQFPLLYT |
| | | MNLLLANAGRLGKFSA |
| | | (SEQ ID NO: 33) |
| 4WBX | Pyrococcus | MNKRFPFPVGEPDFIQGDEAIARAAILAGCRFYAGYPIT |
| | furiosus | PASEIFEAMALYMPLVDGVVIQMEDEIASIAAAIGASW |
| | | AGAKAMTATSGPGFSLMQENIGYAVMTETPVVIVDVQ |
| | | RSGPSTGQPTLPAQGDMQATWGTHGDHSLIVLSPSTV |
| | | QEAFDFTIRAFNLSEKYRTPVILLTDAEVGHMRERVYIP |
| | | NPDEIEIINRKLPRNEEEAKLPFGDPHGDGVPPMPIFGK |
| | | GYRTYVTGLTHDEKGRPRTWREVHERLIKRIVEKIEK |
| | | NKKDIFTYETYELEDAEIGWATGIVARSALRAVKMLR |
| | | EEGIKAGLLKIETIWPFDFELIERIAERVDKLYVPEMNL |
| | | GQLYHLIKEGANGKAEVKLISKIGGEVHTPMEIFEFIRR |
| | | EFK |
| | | (SEQ ID NO: 35) |
| C4L9G3_TOLAT | Tolumonas auensis | MTEQWQSLDSLNALWSALLIEELARLGIRDICIAPGSRS |
| | | TPLTLAAAANPAISTHLHFDERGLGFLALGLAQGSQRP |
| | | VAVIVTSGSAVANLLPAVVEARQSGIPLWLLTADRPAE |
| | | LLGCGANQAITQANIFANYPVYQQLFPAPDHDETPSWL |
| | | LASVDQAAFQQQQTPGPVHLNCPFREPLYPVAGQQIPG |
| | | NALRGLTHWLRSAQPWTQYHAVQPICQTHPLWAEVR |
| | | QSKGIIIAGRLSRQQDTGAILKLAQQTGWPLLADIQSQL |
| | | RFHPQAMTYADLALHHPAFREELAQAETLLLFGGRLT |
| | | SKRLQQFADGHNWQHCWQIDAGSERLDSGLAVQQRF |
| | | VTSPELWCQAHQCEPHRIPWHQLPRWDGKLAGLITQQ |
| | | LPEWGEITLCHQLNSQLQGQLFIGNSMPIRLLDMLGTS |
| | | GAQPSHIYTNRGASGIDGLIATAAGIARANTSQPTTLLL |
| | | GDSSALYDLNSLALLRELTAPFVLIIINNDDGGNIFHMLP |
| | | VPEQNQIRERFYQLPHGLDFRASAEQFRLAYAAPTGAI |
| | | SFRQAYQQALSHPGATLLECKVATGEAADWLKNFAL |
| | | QVRSLPA |
| | | (SEQ ID NO: 37) |
| A0A0K1FGX4_9FIRM | Selenomonas noxia | MNANDLIAALGAEFFTGVPDSKLRPLVDCLMDTYGAN |
| | ATCC 43541 | SPSHIIAANEGNAAALAAGYHLAAGKVPLVYLQNSGL |
| | | GNIVNPLLSLLHAEVYGIPCIFVIGWRGEPDLHDEPQHL |
| | | VQGRLTLPLLETIGVKTMVLTEASQPEDVSAWMEQIRP |
| | | HLAAGGQCALLVRKGALTHPKHKYANENPLRREDAIA |
| | | RILDAAQGAVVVATTGKTGRELFELRAARGEDHAHDF |
| | | LTVGSMGHAGAIALGIALHRPSQRVFLEDGDGAALMH |
| | | MGAMATIGAAAPANIVHVLLNNEAHESVGGAPTAAH |
| | | TVDFPAVARAVGYRLVQTAADAAELAQILPAVGRSDA |
| | | LTFLEWTAIGSRADLGRPTTTPTENKEALMRTLRE |
| | | (SEQ ID NO: 39) |
| A0A0R2PY37_9ACTN | Acidimicrobium sp. | MASSEKMRVGEAIIDLLVREYELDTWGIPGVHNIELFR |
| | BACL17 | GLHSSGVRWAPRHEQGAGFMADGWSIATGKPGVCA |
| | | LISGPGLTNAITPIAQAYHDSRAMLVLASTTPTHSLGKK |
| | | FGPLHDLDDQSAVVRTVTAFSETVTDPTQFPQLIERAW |
| | | NVFTSSRPRPVHIAIPTDVLEQFVDPFTRVTTDISKPVA |
| | | QDSDIQRAAQLLAAAKRPMIIAGGGALGTGALISNIAT |
| | | AIDSPIVLTGNAKGEVPSTHPLCVGSAMVlPRVQEEIEQ |
| | | SDVVLVIGSEISDADLYNGGRAQGFSGSVIRIDIDTEQIS |
| | | RRVAPHVSLVADAADSLSRISAELTKAGVALTNSGSAR |
| | | ATNLRMAARSGVRQDLLPWIDAIEQSVPDNTLVAVDS |
| | | TQLAYAAHTVMSCNSPRSWLAPFGFGTLGCALPMAIG |
| | | AAIADTTRPVLAIAGDGGWLFTLAEMAAAIDEGIDMV |
| | | LVLWDNRGYGQIRESFDDWAPRMGVDVSSHDPSAIA |
| | | NGFGWNAIDVTTIEAFRIVLSEAFENRGAHFIRISVS |
| | | (SEQ ID NO: 41) |
| X1WK73_ACYPI | Acyrthosiphon | MQEADFEVNHARNADIPIVGDAKQTLSQMLELLAQSD |
| | pisum | AKQELDSLRDWWQTIDGWRSRKCLEFDRTSDKIKPQA |
| | | VIETIWRLTKGDAYVTSDVGQHQMFAALYYQFDKPRR |
| | | WINSGGLGTMGFGLPAALGVKMALPDETVICVTGDGS |
| | | IQMNIQELSTALQYDLPVLVLNLNNGFLGMVKQWQD |
| | | MIYSGRHSQSYMQSLPDFVRLAEAYGHVGISIAHPAEL |
| | | EEKLQLALDTLAKGRLVFVDVNIDGSEHVYPMQIRGG |
| | | VIVKLDEIARLAGVSRTTASYVINGKARQYRVSDKTVE |
| | | KVMAVVREHNYHPNAVAAGLRAGRTRSIGLVIPDLEN |
| | | TSYTRIANYLERQARQRGYQLLIACSEQQPDNEMRCIE |
| | | HLLQRQVDAIIVSTSLPPEHPFYQRWINDPLPILALDRAL |
| | | DREHFTSVVGADQDDAHALAAELRQLPVKNVLFLGA |
| | | LPELSVSFLREMGFRDAWKDDERMVDYLYCNSFDRT |
| | | AAATLFEKYLEDHPMPDALFTTSFGLLQGVMDITLKR |
| | | DGRLPTDLAIATFGDHELLDFLECPVLAVGQRHRDVA |
| | | ERVLELVLASLDEPRKPKPGLTRIRRNLFRRGQLSRRTK |
| | | (SEQ ID NO: 43) |
| B1HLR4_BURPE | Burkholderia | MKTEDLIGILTDAGVDLAVGVPDSLLKSFCGRLNDPDC |
| | pseudomallei | PLRHLVASSEGGAVGIAIGHHLATGGLAAVYMQNSGI |
| | | GNATNPLVSLADRAVYGIPLVLIVGWRAEISASGAQVH |
| | | DEPQHVTQGRITLPLLDALSIRHLVLERAGGENDALAP |
| | | SIARLIAGARQTSQPVALWRKDAFDDASASRPGAAAP |
| | | HAGRMTREQAIALIVEHADAGTAIVSTTGVASRELYEL |
| | | RDRLGHSHARDFLTVGGMGHASQIAVGIALARPAQKV |
| | | ICIDGDGALLMHMGGLAYCAGAPNLTHVVINNGVHDS |
| | | VGGQPTLAAHLRLSHIAASCGYAFSRSVATPIELESALH |
| | | HASRLDGSAFIEVTCRPGYRSDLGRPRTSPAENKRHFM |
| | | AFLSRNGATHERDDHAQESGIQDAVQCARH |
| | | (SEQ ID NO: 45) |
| X8CA07_MYCXE | Mycobacterium | MLAKHEFSAATMADGYSRCGQKLGVVAATSGGAALN |
| | xenopi 3993 | LVPGLGESLASRVPVLALVGQPATTMDGRGSFQDTSG |
| | | RNGSLDAEALFSAVSVFCRRVLKPADIITALPAAVAAA |
| | | QTGGPAVLLLPKDIQQTQVGINGYAEHGVAPSRSVGD |
| | | PHSIVRALRQVTGPVTIIAGEQVARDDARAELEWLRAV |
| | | LRARVACVPDAKDVAGTPGFGSSSALGVTGVMGHPG |
| | | VADALAKSALCLVVGTRLSVTARTGLDDALAAVRVV |
| | | SIGSAPPYVPCTHVHTDDLRASLRLLTAALSGRGRPTG |
| | | VRVPDAVVRTELTPRRSTVPACAIATR |
| | | (SEQ ID NO: 47) |
| D1Y3P7_9BACT | Pyramidobacter | MQISSFIAQLQRIASSHFLGVPDSQLKALCNYLYKNCGI |
| | piscolens W5455 | SSDHIIAANEGNCTALAAGYYLATGKVPWYMQNSGL |
| | | GNVVNPVASLLNDKWGIPCVFVIGWRGEPGLKDEPQ |
| | | HIFQGAVTLDLLKVMDIASFVVRKDTTEQELAAQMAE |
| | | FQPLLAAGKSVAFVIAKEALTYDEKVSFKNDFTMTREE |
| | | VIRHITAFSGEDPIVSTTGKASRELFEIRVRNGQPHKYD |
| | | FLTVGSMGHSSSIALGIALSKPHTKIWCIDGDGAALMH |
| | | MGALAVIGSQRPRNLVHIVINNGAHESVGGLPTVARSA |
| | | SLAKVAEACGYVNVKTVGTFAELDAALKDARNADEL |
| | | TFIEAKTAIGARADLGRPTTSAMENRDGFMAYLKELR |
| | | (SEQ ID NO: 49) |
| F4RJP4_MELLP | Melampsora larici- | MPAFSLVEIEAKMSFFSDFLNQVKTPSVASKQIYVSKV |
| | populina | LIQITNFDQLDFDFQIKILNQVTLHPSQPKLTQEEKSKLL |
| | | NNTSILRDSIVFFTDTGAARGVGGHAGGPFDTVREVVL |
| | | LLASFASGSDSKIFDHTVSDEAGHRAQSKLPGHPQLGL |
| | | TPGVKFSSWVDWATCGLFSRVSHSPTETVTCFCSDGS |
| | | QHEGSDAEAARLARAQKLNKLLIDNNNVTISGHTSGY |
| | | LKGYKVGKTLEAHALKIWAEGEKYTGCNDVKSKVIR |
| | | INFDLKGSTGFEAIHQSRPGIFIPSVPVEHGNFCAAAGFG |
| | | FEKGKEKMRKLDAVISFGEIVHRALDAGDQLGIEGFDV |
| | | GLVNKSTLNVIDEKPWMNMDIRNLF |
| | | (SEQ ID NO: 51) |
| A0A081BQW3_9BACT | Candidatus | MTTLGNSRVAFRDALMELAERDPRYVLVCSDSGLVIK |
| | Moduliftexus | AQPFIEKFPQRFFDVGIAEQNAVGVAAGLASSGLVPFF |
| | flocculans | ATYAGFITMRACEQVRTFVAYPGLNVKLVGANGGMA |
| | | SGEREGVTHQFFEDVGILRAIPGITVVVPADADQVVAA |
| | | TKAVALKDGPAYIRIGSGRDPMVEGETPPFELGKVRIL |
| | | KTYGHDVAIFAMGFIMNRALEAAAQLNSEGIRAVVVD |
| | | VHTLKPLDVEAITAILQKTSAAVTVEDHNIIGGLGSAIA |
| | | EVSAEEMPTPLRRIGLRDVYPESGHPEPLLDKYHLGVS |
| | | DIISAAKTVLKKKNHPPRRIAFSTRENAEEGFSNGNMG |
| | | EEIYE |
| | | (SEQ ID NO: 53) |
| CAK95977 | Pseudomonas | MKTVHGATYDILRQHGLTTIFGNPGSNELPFLKGFPED |
| | fluorescens | FRYILGLHEGAVVGMADGYALASGQPTFVNLHAAAG |
| | | TGNGMGALTNAWYSHSPLVITAGQQWSMIGVEAML |
| | | ANVDAAQLPKPLVKWSHEPATAQDVPRALSQAIHTAN |
| | | LPPRGPVYVSIPYDDWACEAPSGVEHLARRQVSSAGLP |
| | | SPAQLQHLCERLAAARNPVLVLGPDVDGSAANGLAV |
| | | QLAEKLRMPAWVAPSASRCPFPTRHACFRGVLPAAIA |
| | | GISHNLAGHDLILVVGAPVFRYHQFAPGNYLPAGCELL |
| | | HLTCDPGEAARAPMGDALVGDIALTLEAVLDGVPQSV |
| | | RQMPTALPAAEPVADDGGLLRPETVFDLLNALAPKDA |
| | | IYVKESTSTVGAFWRRVEMREPGSYFFPAAGGLGFGLP |
| | | AAVGVQLASPGRQVTGVIGDGSANYGITALWTAAQYN |
| | | IPVVFIILKNGTYGALRWFADVLDVNDAPGLDVPGLDF |
| | | CAIARGYGVQAVHAATGSAFAQALREALESDRPVLIE |
| | | VPTQTIEP |
| | | (SEQ ID NO: 55) |
| YP_831380 | Arthrobacter sp. | MTTVHAAAYELLRSNRLTTIFGNPGDNELPFLDAMPA |
| | | DFRYILGLHEGVVVGMADGFAQASGQAAFVNLHAAS |
| | | GTGNAMGALTNAWYSHTPLVITAGOQVRPMIGLEAM |
| | | LSNVDAASLPRPLVKWSAEPAQAPDVPRALSQAIHTAT |
| | | SDPKGPVYLSIPYDDWNQDTGNLSEHLSSRSVSRAGNP |
| | | SAEQLDDILSALREAANPALVFGPDVDAARANHHAVR |
| | | LAEKLAAPVWIAPAAPRCPFPTRHPNFRGVLPASIAGIS |
| | | ALLNGHDLIVVIGAPVFRYHQYQPGSYLPENSRLIHITC |
| | | DAGEAARAPMGDALVADIGQTLRALADIIPQSKRPPLR |
| | | PRVIPPVPDSQDDLLAPDAVFEVMNEVAPEDVVYVNE |
| | | SVSTVTALWERVELKHPGSYYFPASGGLGFGMPAAVG |
| | | VQLANDRRRVIAVIGDGSANYGITALWTAAQEKIPVVF |
| | | IILNNGTYGALRAFAKLLNAENAAGLDVPGICFCAIAE |
| | | GYGVEAHRITSLENFKDKLSAALQSDTPTLLEVPTSTTS |
| | | PF |
| | | (SEO ID NO: 57) |
| ZP_06547677 | Pseudomonas | MKTIHSAAYALLRRHGMTTIFGNPGSNELPFLKSFPED |
| | putida CSV86 | FQYVLGLHEGAWGMADGYALASGKPAFVNLHAAA |
| | | GTGNGMGALTNSWYSHSPLVITAGQQVRPMIGVEAM |
| | | LANVTJATQLPKPLVKWSYEPANAQDVPRALSQAIHYA |
| | | NTTPKAPWLSIPYDDWDQPSGPGVEHLIERDVQTAGT |
| | | PDARQLQVLVQQVQDARNPVLVLGPDVDATLSNDHA |
| | | VALADKLRMPVWIAPAASRCPFPTRHPSFRGVLPAAIA |
| | | GISKTLQGHDLIIVVGAPVFRYLQFAPGDYLPVGAQLL |
| | | HITSDPLEATRAPMGHALVGDIRETLRVLAEEVVQQSR |
| | | PYPEALAAPECVTDEPHHLHPETLFDVLDAVAPHDAIY |
| | | VKESTSTVTAFWQRMNLRHPGSYYFPAAGGLGFGLPA |
| | | AVGVQLAQPQRRWALIGDGSANYGITALWTAAQYRI |
| | | PVVFIILKNGTYGALRWFAGVLKAEDSPGLDVPGLDFC |
| | | ALAKGYGVKAVHTDTRDSFEAALRTALDANEPTVIEVP |
| | | TLTIQPH |
| | | (SEQ ID NO: 59) |
| ZP_06846103 | Halotalea | MTSRSSFSPPSASEQRGADIFAEVLQCEGVRYIFGNPGT |
| | alkalilenta | TELPLLDALTDITGIHYVLGLHEASWAMADGYAQAS |
| | | GKPGFVNLHTAGGLGNAMGAILNAKMANTPLVVTAG |
| | | QQDTRHGVTDPLLHGDLTGIARPNVKWAEEIHHPEHIP |
| | | MLLRRALQDCRTGPAGPVFLSLPIDTMERCTSVGAGE |
| | | ASRIERASVANMLHALATALAEVTAGHIALVAGEEVF |
| | | TANASVEAVALAEALGAPVFGASWPGHIPFPTAHPQW |
| | | QGTLPPKASDIRETLGPFDAVLILGGHSLISYPYSEGPAI |
| | | PPHCRLFQLTGDGHQIGRVHETTLGLVGDLQLSLRALL |
| | | PLLARKLQPQNGAVARLRQVATLKRDARRTEAAERSA |
| | | REFDASATTPFVAAFETIRAIGPDVPIVDEAPVTIPHVRA |
| | | CLDSASARQYLFTRSAILGWGMPAAVGVSLGLDRSPV |
| | | VCLVGDGSAMYSPQALWTAAHERLPVTFVVFNNGEY |
| | | NILKNYARAQTNYRSARANRFIGLDISDPAIDFPALASS |
| | | LGVPARRVERAGDIAIAVEDGIRSGRPNLIDVLISSSS |
| | | (SEQ ID NO: 61) |
| ZP_07290467 | Streptomyces sp. | MRTVRESALDVLRARGMTTVFGNPGSTELPMLKQFPD |
| | | DFRYVLGLQEAVVVGMADGFALASGTTGLVNLHTGP |
| | | GTGNAMGAILNARANRTPMVVTAGQQVRAMLTMEA |
| | | LLTNPQSTLLPQPAVKWAYEPPRAADVAPALARAVQV |
| | | AETPPQGPVFVSLPMDDFDVVLGEDEDRAAQRAAART |
| | | VTHAAAPSAEVVRRLAARLSGARSAVLVAGNDVDAS |
| | | GAWDAVVELAERTGLPVWSAPTEGRVAFPKSHPQYR |
| | | GMLPPAIAPLSRCLEGHDLVLVIGAPVFCYYPYVPGAH |
| | | LPENTELWLTRDADEAARAPVGDAVVADLALTVRAL |
| | | LAELPAREAAAPAARTARAESTAEVDGVLTPLAAMTA |
| | | IAQGAPANTLWVNESPSNLGQFHDATRIDTPGSFLFTA |
| | | GGGLGFGLAAAVGAQLGAPDRPWCVIGDGSTHYAV |
| | | QALWTAAAYKVPVTFVVLSNQRYAILQWFAQVEGAQ |
| | | GAPGLDIPGLDIAAVATGYGVRAHRATGFGELSKLVR |
| | | ESALOQDGPVLIDVPVTTELPTL |
| | | (SEQ ID NO: 63) |
| ZP_08570611 | Rheinheimera sp. | MSSINSFTVADYLLTRLHQLGLRKVFQVPGDYVANFM |
| | A13L | DALEQFNGIEAVGDLTELGAGYAADGYARLTGIGAVS |
| | | VQFGVGTFSVLNAIAGSYVERNPVVVITASPSTGNRKTI |
| | | KETGVLFFIHSTGDLLADSKWANVTVAAEVLSDPSDA |
| | | RQKIDKALTLAITFRRPIYLEAWQDVWGLACEKPEGEL |
| | | KALPLISEEGALKAMLADSLKLLNSARQPLVLLGVEIN |
| | | RFGLODAVLDLLKASGLPYSTTSLAKTVISENEGIFVGT |
| | | YADGASFPATVEYTEKADCVLALGVIFTDDYLTMLSK |
| | | QFDQMIVVNNDETSRLGHAYYHOLYLADFILQLTDEIK |
| | | KSSLYPRQNSALPLLPPQPQITPALLQQQLSYONFFDLF |
| | | YGYLLQHQLQDNISLILGESSSLYMSARLYGLPQDSFIA |
| | | DAAWGSLGHETGCVTGIAYASDKRAMAIAGDGGFMM |
| | | MCQCLSTISRHQLNSWFVISNKVYAIEQSFVDICAFAK |
| | | GGHFAPFDLLPTWDYLSLAKAFSVEGYRVQNGEELLQ |
| | | ALEHIMTQKDKPALVEVVIQSQDLAPAMAGLVKSITG |
| | | HTVEQCAIPT |
| | | (SEQ ID NO: 65) |
| YP_001240047 | Bradyrhizobium sp. | MHPDACSIACAAMPTNWGPRTVTKLPLPDPQSRATTH |
| | STM3843 | HRTAHYFLEALIDLGVEYIFANLGTDHVSLIEEIARWDS |
| | | EGRRHPEVILCPHEVVAVHMAMGYAMTTGRGQAVFV |
| | | HVDAGTANACMAIQNAFRYRLPVLLIAGRAPFAIHGEL |
| | | PGGRDTYVHFVQDSFDQGSIVRPYVKWEYTLPSGVVV |
| | | KEALTRAAAFMHSDPPGPVSMMLPREVLAEAWDDDA |
| | | MPAYPPARYGSVRAGGVDPERAQAIADALMTAENPIA |
| | | LTAYLGRSAEAVSVLDRLALVCGIRVVEFNPITMNICQ |
| | | DSPCFAGSDPAALVADADLGLLIDIDVPFIPQLLKSADR |
| | | LRWIQIDIDALKADIPMWGFATDLRIQGDSAVILRQVL |
| | | EIVIARGNDSYMRKVRDRIASWRPAREAAQAKRMAA |
| | | AANKGSPGAINPAYLFARLQALLSEQDIVVNEAVRNAP |
| | | VLQQQLRRTKPMTYVGLAGGGLGFSGGMALGLKLAN |
| | | PSHRVVQIVGDGAFHFAAPDSVYAVSQQYRLPIFSVIL |
| | | DNKGWQAVKASVQRVYPDGVAQQTDSFLSRLATGRQ |
| | | DEQRRLVDIARAFGAHGERVDDPDELDAAIRSCLAAL |
| | | DDGRAAVLHVNITPL |
| | | (SEQ ID NO: 67) |
| YP_001279645 | Psychrobacter sp. | MQHDSITPLSKKTSMLDTTAESVVSQTVQQVVFELMR |
| | | TLNMTTVFGNPGSTELNFLTNFPEDFSYVLGLHEASVV |
| | | GMADGYAQATGNAAFVNLHSAAGVGNALGNIFTAYR |
| | | NHTPLVITAGQQARSLLPFAPYLGAEQAAQFPQPYIKW |
| | | SIEPARAEDVPLAIAQAYLIAMQHPQGPTFVSIPSDDWD |
| | | KPAVLPLLSQSCGHSIPSPDALAELVEVMSTSQNMALV |
| | | VGSDVDRQGGFELAVSVAEACQAPVWEAPNSSRASFP |
| | | ENHPLFAGFLPAIPEKLSEKLLGYDTIVVIGAPAFTLHV |
| | | AGTLSLKKSKIYQLTDDPQYAAQSVATKTLSGNIRDSL |
| | | QALLDKLPTSMTPRSGLDLPVRKPAAEVQGSNPISIEY |
| | | VMATLAKYCPEDVVIVEEAPSHRPAIORYLPITQPKSFY |
| | | TMASGGLGYGLPAAVGVALGTQRRTLCLIGDGSSMYS |
| | | IQAIWTAVQHNLPVTVIVLNNTGYGAMRSFSKIMGSTQ |
| | | VPGLDLPNINFVQLAQSMGCQAQKVTDYSVLDKVFAD |
| | | TMQAAGSYLLEIMVDANTGAVY |
| | | (SEQ ID NO: 69) |
| ZP_01901192 | Roseobacter sp. | MKMTTEEAFVKTLQRHGIEHAFGIIGSAMMPISDLFPQ |
| | AzwK-3b | AGITFWDCAHEGSAGMMSDGYTRATGKMSMMIAQN |
| | | GPGITNFVTAVKTAYWNHTPLLLVTPQAANKTIGQGG |
| | | FQEVEQMKLFEDMVAYQEEVRDPSPRMJAEVLARVISK |
| | | AKNLSGPAQINIPRDYWTQVIDIELPDPIEFERSPGGENS |
| | | VAEAARLISEARNPVILNGAGVVLSEGGIAASQALAER |
| | | LDAPVCVGYQHNDAFPGSHPLFAGPLGYNGSKAAME |
| | | LIKDADVVLCLGTRLNPFSTLPGYGMDYWPKDAKIIQ |
| | | VDINPDRIGLTKKVSVGIIGDAAKVARGILGQLSDSAG |
| | | DEGRDARRARIAETKSKWAQQLSSMDHEDDDPGTSW |
| | | NERAREAKPDWMSPRMAWRAIQSALPREAIISSDIGNN |
| | | CAIGNAYPSFEEGRKYLAPGLFGPCGYGLPAIVGAKIG |
| | | RPDVPWGFAGDGAFGIAVNELTAIGRSEWPGITQIVF |
| | | RNYQWGAEKRNSTLWFDDNFVGTELDDDVSYAGIAK |
| | | ACGLKGVVARTMDELTDALNQAIKDQMENGTTTLIEA |
| | | MINOELGEPFRRDAMKKPVAVAGISPDDMRPOKVA |
| | | (SEQ ID NO: 71) |
| ZP_06549025 | Serratia | MSNAITKVQNANARRGGDVLLEVLESEGVEYVFGNPG |
| | marcescens FGI94 | TTELPFMDALLRKPSIQYVLALQEASAVAMADGYAQA |
| | | AKKPGFLNLHTAGGLGHGMGNLLNAKCSQTPLVVTA |
| | | GQQDSRHTTTDPLLLGDLVGMGKTFAKWSQEVTHVD |
| | | QLPVLVRRAFHDSDAAPKGSVFLSLPMDVMEAMSAIG |
| | | IGAPSTIDRNAVAGSLPLLASKLAAFTPGNVALIAGDEI |
| | | YQSEAANEVVALAEMLAADVYGSTWPNRIPYPTAHPL |
| | | WRGNLSTKATEINRALSQYDAIFALGGKSLITILYTEGQ |
| | | AVPEQCKVFQLSADAGDLGRTYSSELSVVGDIKSSLKV |
| | | LLPELEKATANHRRDYQRRFEKAINEFKLSKESLLGQV |
| | | QEQQSATVITPLVAAFEAARAIGPDVAIVDEAIATSGSL |
| | | RKSLNSHRADQYAFLRGGGLGWGMPAAVGYSLGLGK |
| | | APVVCFVGDGAAMYSPQALWTAAHEKLPVTFIVMNN |
| | | TEYNVLKNFMRSQADYTSAQTDRFIAMDLVNPSVDYQ |
| | | ALGASMGLETRKVIRAGDIAPAVEAALASGKPNVIEIII |
| | | SKS |
| | | (SEQ ID NO: 73) |
| ZP_07033476 | Granulicella | MNIAYETRENKVASGRECLLEILRDEGVTHVFGNPGTT |
| | mallensis ATCC | ELALIDALAGDDDFHFILGLQEAAVVGMADGYAQATG |
| | BAA-1857 | RPSFVNLHTTAGLGNGMGNLTNAFATNVPMVVTAGQ |
| | | QDIRHLAYDPLLSGDLVGLARATVKWAHEVRSLQELP |
| | | IILRRAFRDANTEPRGPVFVSLPMNIIDEIGTVSIPPRSTI |
| | | VQAESGDISQLVRLLVESAGNLCLVVGDEVGRYGATE |
| | | AAVRVAELLGAPVYGSPFHSNVPFPTDHPLWRFTLPPN |
| | | TGEMRKVLGGYDRILLIGDRAFMSYTYSDELPLSPKTQ |
| | | LLQIAVDRHSLGRCHAVELGLYGDPLSLLAAVGDALS |
| | | QERALAPSRDSRLAIARDWRASWEQDLKDECERLAPS |
| | | RPLYPLVAADAVLRGVPPGTVIVDECLATNKYVRQLY |
| | | PVRKPGEYYYFRGAGLGWGMPAAVGVSLGLERQORV |
| | | VCLLGDGAAMYSPQALWSAAHESLPITFVVFNNSEYNI |
| | | LKNFMRSRPGYNAQSGRFVGMEINQPSIDFCALARSM |
| | | GVDAVRLTEPDDITAYMIAAGDREGPSLLEIPIAATAS |
| | | (SEQ ID NO: 75) |
| WP_010764607.1 | Enterococcus | MYTVADYLLDRLKELGIDEVFGVPGDYNLQFLDHITA |
| | haemoperoxidus | RKDLEWIGNANELNAAYMADGYARTKGISALVTTFG |
| | ATCC BAA-382 | VGELSAINGLAGSYAESIPVIEIVGSPTTTVQQNKKLVH |
| | | HTLGDGDFLRFERIHEEVSAAIAHLSTENAPSEIDRVLT |
| | | VAMTEKRPVYINLPIDIAEMKASAPTTPLNHTTDQLTT |
| | | VETAILTKVEDALKQSKNPVVIAGHEILSYHIENQLEQF |
| | | IQKFNLPITVLPFGKGAFNEEDAHYLGTYTGSTTDESM |
| | | KNRVDHADLVLLLGAKLTDSATSGFSFGFTEKQMISIG |
| | | STEVLFYGEKQETVQLDRFVSALSTLSFSRFTDEMPSV |
| | | KRLATPKVRDEKLTQKQFWQMVESFLLQGDTVVGEQ |
| | | GTSFFGLTNVPLKKDMHFIGQPLWGSIGYTFPSALGSQI |
| | | ANKESRHLLFIGDGSLQLTVQELGTAIREKLTPIVFVIN |
| | | NNGYTVEREIHGATEQYNDIPMWDYQKLPFVFGGTDQ |
| | | TVATYKVSTEIELDNAMTRARTDVDRLQWIEVVMDQ |
| | | NDAPVLLKKLAKIFAKQNS |
| | | (SEQ ID NO: 77) |
| WP_002115026.1 | Acinetobacter | MELLSGGEMLVRALADEGVEHVFGYPGGAVLHIYDA |
| | baumannii | LFQQDKINHYLVRHEQAAGHMADAYSRATGKTGVVL |
| | | VTSGPGATNTVTPIATAYMDSIPMVILSGQVASHLIGED |
| | | AFQETDMVGISRPIVKHSFQVRHASEIPAIIKKAFYIAAS |
| | | GRPGPVVVDIPKDATNPAEKFAYEYPEKVKMRSYQPP |
| | | SRGHSGQIRKAIDELLSAKRPVIYTGGGVVQGNASALL |
| | | TELAHLLGYPVTNTLMGLGGFPGDDPQFVGMLGMHG |
| | | TYEANMAMHNADVILAIGARFDDRVTNNPAKFCVNA |
| | | KVIHIDIDPASISKTIMAHIPIVGAVEPVLQEMLTQLKQL |
| | | NVSKPNPEAIAAWWDQINEWRKVHGLKFETPTDGTM |
| | | KPQQVVEALYKATNGDAIITSDVGQHQMFGALYYKY |
| | | KRPRQWINSGGLGTMGVGLPYAMAAKLAFPDQQVVC |
| | | ITGEASIQMCIQELSTCKQYGMNVKILCLNNRALGMV |
| | | KQWQDMNYEGRHSSSYVESLPDFGKLMEAYGHVGIQI |
| | | DHADELESKLAEAMAINDKCVFINVMVDRTEHVYPM |
| | | LIAGQSMKDMWLGKGERT (SEQ ID NO: 79) |
| YP_005756646.1 | Staphylococcus | MKQRIGAYLIDAIHRAGVDKIFGVPGDFNLAFLDDIISN |
| | aureus | PNVDWVGNTNELNASYAADGYARLNGLAALVTTFGV |
| | | GELSAVNGIAGSYAERIPVIAITGAPTRAVEHAGKYVH |
| | | HSLGEGTFDDYRKMFAHITVAQGYITPENATTEIPRLIN |
| | | TAIAERRPVHLHLPIDVAISEIEIPTPFEVTAAKDTDAST |
| | | YIELLTSKLHQSKQPIIITGHEINSFHLHQELEDFVNQTQ |
| | | IPVAQLSLGKGAFNEENPYYMGIYDGKIAEDKIRDYVD |
| | | NSDLILNIGAKLTDSATAGFSYQFNIDDVVMLNHHNIKI |
| | | DDVTNDEISLPSLLKQLSNISHTNNATFPAYHRPTSPDY |
| | | TVGTEPLTQQTYFKMMQNFLKPNDVIIADQGTSFFGA |
| | | YDLALYKNNTFIGQPLWGSIGYTLPATLGSQLADKDR |
| | | RNLLLIGDGSLQLTVQAISTMIRQHIKPVLFVINNDGYT |
| | | VERLIHGMYEPYNEIHMWDYKALPAVFGGKNVEIHDV |
| | | ESSKDLQDTFNAINGHPDVMHFVEVKMSVEDAPKKLI |
| | | DIAKAFSQQNK |
| | | (SEQ ID NO: 81) |
| WP_008347133.1 | Bacillus pumilus | MPQRTAGKEVTALLEEWGVKHIYGMPGDSINELIEELR |
| | SAFR-032 | HESSKIQFIQTRHEEVAALSAAADAKLTGKLGVCLSIA |
| | | GPGAVHLLNGLYDAKADGAPVLAIAGQVASTEVGRD |
| | | AFQEIKLERMFDDVAVFNQQVQTAEALPDLLNQAIKA |
| | | AYTHKGVAVLTVSDDLFSQKIKRSPVYTSPLYVEGDV |
| | | RPKKDQLLKAAQLINNAKKPVILAGKGLRNAKEELLSF |
| | | AEKAAAPIVITLPAKGVVPDRHAYFLGNLGQIGTKPAY |
| | | EAMEECDLLIMLGTSFPYRDYLPEDTPAIQLDIKPDQIG |
| | | KRYPVEVGIVSDSKTGLHELTSYIEYKEQRGFLEACTE |
| | | HMMKWREEMDKEKSIATSPLKPQQVIARLEEAVDDD |
| | | AILSVDVGNVTVWMARHFEMKQQDFIISSWLATMGC |
| | | GLPGAISAKLNEPNRQAIAVCGDGGFTMVMQDFVTAV |
| | | KYKLPIVVVILNNNNLGMIEYEQQVKGNINYGIELEDI |
| | | DFAKFAEACGGKGISVSSHEELAPAFDQALQADKPVII |
| | | DVAVTNEPPLPGKITYTQAAGFSKYLLKKFFEKGELDI |
| | | PPLKKSLKRFF |
| | | (SEQ ID NO: 83) |
| WP_018535238.1 | Streptomyces | MVSRPARVAILEQLRADGVRYMFGNPGTVEQGFLDEL |
| | glaucescens | RNFPDIEYILALQEAGVVGLADGYARATRTPAVLQLHT |
| | | GVGVGNAVGMLYQAKRGHAPLVAIAGEAGLRYDAM |
| | | EAQMAVDLVAMAEPVTKWATRVVDPESTLRVLRRA |
| | | MKVAATPPYGPVLVVLPADVMDRDTSEAAVPTSYVD |
| | | FAATPDPQVLDRAAELLAGAERPIVIAGDGVHFAGAQ |
| | | EELGRLAQTWGAEVWGADWAEVNLSVEHPAYAGQL |
| | | GHMFGDSSRRVTGAADAVLLVGTYALPEVYPALDGV |
| | | FADGAPVVHIDLDTDAIAKNFPVDLGLAADPRRALDG |
| | | LARALERRMSPESRARAGEWFTGRSAQRSYEIAAARE |
| | | QDEAALAPDALPVTAFLQELARQLPEDAVVFDEALTA |
| | | SPDVTRHLPPTRPGHWHQTRGGSLGVGIPGAIAAQLAH |
| | | PDRTVVGFTGDGGSLYTIQALWTAARYDIGATFVICNN |
| | | SSYKLLELNIEEYWKSVDVAAHEQPEMFDLARPAIDFV |
| | | ALSRSLGVPAVRVEKPDQAKAAVEQALGTPGPFLIDLV |
| | | TGRGRED |
| | | (SEQ ID NO: 85) |
| YP_006485164.1 | Pseudomonas | MKTVHSASYEILRRHGLTTVFGNPGSNELPFLKDFPED |
| | aeruginosa | FRYILGLHEGAVVGMADGFALASGRPAFVNLHAAAGT |
| | | GNGMGALTNAWYSHSPLVITAGQQVRSMIGVEAMLA |
| | | NVDAGQLPKPLVKWSHEPACAQDVPRALSQAIQTASL |
| | | PPRAPVYLSIPYDDWAQPAPAGVEHLAARQVSGAALP |
| | | APALLAELGERLSRSRNPVLVLGPDVDGANANGLAVE |
| | | LAEKLRMPAWGAPSASRCPFPTRHACFRGVLPAAIAGI |
| | | SRLLDGHDLILVVGAPVFRYHQFAPGDYLPAGAELVQ |
| | | VTCDPGEAARAPMGDALVGDIALTLEALLEQVRPSAR |
| | | PLPEALPRPPALAEEGGPLRPETVFDVIDALAPRDAIFV |
| | | KESTSTVTAFWQRVEMREPGSYFFPAAGGLGFGLPAA |
| | | VGAQLAQPRRQVIGIIGDGSANYGITALWSAAQYRVP |
| | | AVFIILKNGTYGALRWFAGVLEVPDAPGLDVPGLDFC |
| | | AIARGYGVEALHAATREELEGALKHALAADRPVLIEV |
| | | PTQTIEP |
| | | (SEQ ID NO: 87) |
| YP_005461458.1 | Actinoplanes | MIDLDGTVTVAEYLGLRLRHAGVEHLFGVPGDFNLNL |
| | missouriensis | LDGLAFVEGLRWVGSPNELGAGYAADAYARRRGLSA |
| | | LFTTYGVGELSAINAVAGSAAEDSPVVHVVGSPRTTTV |
| | | AGGALVHHTIADGDFRHFARAYAEVTVAQAMVTATD |
| | | AGAQIDRVLLAALTHRKPVYLSIPQDLALHRIPAAPLR |
| | | EPLTPASDPAAVERFRTAVRDLLTPAVRPIMLVGQLVS |
| | | RYGLSTLVTDMTTRSGIPVAAQLSAKGVIDESVEGNLG |
| | | LYAGSMLDGPAASLIDSADVVLHLGTALTAELTGFFTH |
| | | RRPDARTVQLLSTAALVGTTRFDNVLFPDAMTTLAEV |
| | | LTTFPAPARLAAPTTRAEPTGLAASITPPAPSAVDLTAS |
| | | TATDLTAPTAGDISEMSRVLTQDAFWAGMQAWLPAG |
| | | HALVADTGTSYWGALALRLPGDTVTLGQPIWNSIGWA |
| | | LPAVLGQGLADPDRRPVLVIGDGAAQMTIQELSTIVAA |
| | | GLRPIILLLNNRGYTIERALQSPNAGYNDVADWNWRA |
| | | VVAAFAGPDTDYHHAATGTELAKALTAASESNRPVFI |
| | | EVELDAFDTPPLLRRLAERATAPS |
| | | (SEQ ID NO: 89) |
| YP_006991301.1 | Carnobacterium | MYTVGNYLLDRLTELGIRDIFGVPGDYNLKFLDHVMT |
| | maltaromaticum | HKELNWIGNANELNAAYAADGYARTKGIAALVTTFG |
| | LMA28 | VGELSAANGTAGSYAEKVPVVQIVGTPTTAVQNSHKL |
| | | VHHTLGDGRFDHFEKMQTEINGAIAHLTADNALAEID |
| | | RVLRIAVTERCPVYINLAIDVAEVVAEKPLKPLMEESK |
| | | KVEEETTLVLNKIEKALQDSKNPVVLIGNEIASFHLESA |
| | | LADFVKKFNLPVTVLPFGKGGFDEEDAHFIGVYTGAPT |
| | | AESIKERVEKADLILIIGAKLTDSATAGFSYDFEDRQVIS |
| | | VGSDEVSFYGEIMKPVAFAQFVNGLNSLNYLGYTGEIK |
| | | QVERVADIEAKASNLTQNNFWKFVEKYLSNGDTLVAE |
| | | QGTSFFGASLVPLKSKMKFIGQPLWGSIGYTFPAMLGS |
| | | QIANPASRHLLFIGDGSLQLTIQELGMTFREKLTPIVFVI |
| | | NNDGYTVEREIHGPNELYNDIPMWDYQNLPYVFGGN |
| | | KGNVATYKVTTEEELVAAMSQARQDTTRLQWIEVVM |
| | | GKQDSPDLLVQLGKVFAKQNS (SEQ ID NO: 91) |
| NP_594083.1 | Schizosaccharomyces | MSSEKVLVGEYLFTRLLQLGIKSILGVPGDFNLALLDLI |
| | pombe | EKVGDETFRWVGNENELNGAYAADAYARVKGISAIV |
| | | TTFGVGELSALNGFAGAYSERIPVVHIVGVPNTKAQAT |
| | | RPLLHHTLGNGDFKVFQRMSSELSADVAFLDSGDSAG |
| | | RLIDNLLETCVRTSRPVYLAVPSDAGYFYTDASPLKTP |
| | | LVFPVPENNKEIEHEVVSEILELIEKSKNPSILVDACVSR |
| | | FHIQQETQDFIDATHFPTYVTPMGKTAINESSPYFDGVY |
| | | IGSLTEPSIKERAESTDLLLIIGGLRSDFNSGTFTYATPAS |
| | | QTIEFHSDYTKIRSGVYEGISMKHLLPKLTAAIDKKSVQ |
| | | AKARPVHFEPPKAVAAEGYAEGTITHKWFWPTFASFL |
| | | RESDVVTTETGTSNFGILDCIFPKGCQNLSQVLWGSIG |
| | | WSVGAMFGATLGIKDSDAPHRRSILIVGDGSLHLTVQE |
| | | ISATIRNGLTPIIFVINNKGYTIERLIHGLHAVYNDINTE |
| | | WDYQNLLKGYGAKNSRSYNIHSEKELLDLFKDEEFGK |
| | | ADVIQLVEVHMPVLDAPRVLIEQAKLTASLNKQ |
| | | (SEQ ID NO: 93) |
| WP_003075272.1 | Comamonas | MPANTAPNAQAAEVFTVRHAVINMLRELGMTRIFGNP |
| | testosteroni | GSTELPLFRDYPEDFSYILGLQETVVVGMADGYAQAT |
| | | RNASFVNLHSAAGVGHAMANIFTAFKNRTPMVITAGQ |
| | | QTRSLLQFDPFLHSNQAAELPKPYVKWSCEPARAEDV |
| | | PQALARAYYIAMQEPRGPVFVSIPADDWDVPCEPITLR |
| | | KVGFETRPDPRLLDSIGQALEGARAPAFVVGAAVDRS |
| | | QAFEAVQALAERHQARVYVAPMSGRCGFPEDHALFG |
| | | GFLPAMRERIVDRLSGHDVVFVIGAPAFTYHVEGHGPF |
| | | IAEGTQLFQLIEDPAIAAWAPVGDAAVGNIRMGVQELL |
| | | ARPLTHPRPALQPRPAIPAPAAPEPGRLMTDAFLMHTL |
| | | AQVRSRDSIIVEEAPGSRSIIQAHLPIYAAETFFTMCSGG |
| | | LGHSLPASVGIALARPDKKVIGVIGDGSAMYAIQALWS |
| | | AAHLKLPVTYIIVKNRRYAALQDFSRVFGYREGEKVE |
| | | GTDLPDIDFVALAKGQGCDGVRVTDAAQLSQVLRDAL |
| | | RSPRATLVEVEVA |
| | | (SEQ ID NO: 95) |
| WP_020634527.1 | Amycolatopsis | MNVAELVGRTLAELGVGAAFGVVGSGNFVVTNGLRA |
| | orientalis | GGVRFVAARHEGGAASMADAYARMSGRVSVLSLHQ |
| | HCCB10007 | GCGLTNALTGITEAAKSRTPMIVLTGDTAASAVLSNFR |
| | | IGQDALATAVGAVPERVHSAPTAVADTVRAYRTAVQ |
| | | QRRTVLLNLPLDVQAQEAPEAVEIPKVRGPAPIRPDAG |
| | | MVAKLADLLAEARRPVFIAGRGARASAVPLRELAEISG |
| | | ALLATSAVAHGLFHDDPFSLGISGGFSSPRTADLIVDAD |
| | | LVIGWGCALNMWTTRHGTLLGPAARLVQVDVEQAAL |
| | | GAHRPIDLGVVGDVAGTAVDVHAELDKRGHQRSREA |
| | | PTGTRWNDVPYNDLSGDGRIDPRTLSRRLDEILPAERM |
| | | VSIDSGNFMGYPSAYLSVPDENGFCFTQAFQSIGLGLG |
| | | TAIGAALARPDRLPVLGVGDGGFHMAVSELETAVRLR |
| | | IPLVIVVYNDAAYGAEIHHFGDADMTTVRFPDTDIAAI |
| | | GRGFGCDGVTVRSVGDLAAVKEWLGGPRDAPLVIDA |
| | | KIADDGGSWWLAEAFRH (SEQ ID NO: 97) |
| IOVM | Enterobacter sp. | MRTPYCVADYLLDRLTDCGADHLFGVPGDYNLQFLD |
| | | HVIDSPDICWVGCANELNASYAADGYARCKGFAALLT |
| | | TFGVGELSAMNGIAGSYAEHVPVLHIVGAPGTAAQQR |
| | | GELLHHTLGDGEFRHFYHMSEPITVAQAVLTEQNACY |
| | | EIDRVLTTMLRERRPGYLMLPADVAKKAATPPVNALT |
| | | HKQAHADSACLKAFRDAAENKLAMSKRTALLADFLV |
| | | LRHGLKHALQKWVKEVPMAHATMLMGKGIFDERQA |
| | | GFYGTYSGSASTGAVKEAIEGADTVLCVGTRFTDTLTA |
| | | GFTHQLTPAQTIEVQPHAARVGDVWFTGIPMNQAIETL |
| | | VELCKQHVHAGLMSSSSGAIPFPQPDGSLTQENFWRTL |
| | | QTFIRPGDIILADQGTSAFGAIDLRLPADVNFIVQPLWG |
| | | SIGYTLAAAFGAQTACPNRRVIVLTGDGAAQLTIQELG |
| | | SMLRDKQHPIILVLNNEGYTVERAIHGAEQRYNDIALW |
| | | NWTHIPQALSLDPQSECWRVSEAEQLADVLEKVAHHE |
| | | RLSLIEVMLPKADIPPLLGALTKALEACNNA |
| | | (SEQ ID NO: 99) |
| 2Q5Q | Azospirillum | MKLAEALLRALKDRGAQAMFGIPGDFALPFFKVAEET |
| | brasilense Sp24 | QILPLHTLSHEPAVGFAADAAARYSSTLGVAAVTYGA |
| | | GAFNMVNAVAGAYAEKSPVVVISGAPGTTEGNAGLLL |
| | | HHQGRTLDTQFQVFKEITVAQARLDDPAKAPAEIARV |
| | | LGAARAQSRPVYLEIPRNMVNAEVEPVGDDPAWPVD |
| | | RDALAACADEVLAAMRSATSPVLMVCVEVRRYGLEA |
| | | KVAELAQRLGVPVVTTFMGRGLLADAPTPPLGTYIGV |
| | | AGDAEITRLVEESDGLFLLGAILSDTNFAVSQRKIDLRK |
| | | TIHAFDRAVTLGYHTYADIPLAGLVDALLERLPPSDRT |
| | | TRGKEPHAYPTGLQADGEPIAPMDIARAVNDRVRAGQ |
| | | EPLLIAADMGDCLFTAMDMIDAGLMAPGYYAGMGFG |
| | | VPAGIGAQCVSGGKRILTVVGDGAFQMTGWELGNCR |
| | | RLGIDPIVILFNNASWEMLRTFQPESAFNDLDDWRFAD |
| | | MAAGMGGDGVRVRTRAELKAALDKAFATRGRFQLIE |
| | | AMIPRGVLSDTLARFVQGQKRLHAAPRE (SEQ ID NO: |
| | | 101) |
| 2VBG | Lactococcus lactis | MYTVGDYLLDRLHELGIEEIFGVPGDYNLQFLDQIISRE |
| | | DMKWIGNANELNASYMADGYARTKKAAAFLTTFGV |
| | | GELSAINGLAGSYAENLPVVEIVGSPTSKVQNDGKFVH |
| | | HTLADGDFKHFMKMHEPVTAARTLLTAENATYEIDRV |
| | | LSQLLKERKPVYINLPVDVAAAKAEKPALSLEKESSTT |
| | | NTTEQVILSKIEESLKNAQKPVVIAGHEVISFGLEKTVT |
| | | QFVSETKLPITTLNFGKSAVDESLPSFLGIYNGKLSEISL |
| | | KNFVESADFILMLGVKLTDSSTGAFTHHLDENKMISLN |
| | | IDEGIIFNKVVEDFDFRAVVSSLSELKGIEYEGQYIDKQ |
| | | YEEFIPSSAPLSQDRLWQAVESLTQSNETIVAEQGTSFF |
| | | GASTIFLKSNSRFIGQPLWGSIGYTFPAALGSQIADKES |
| | | RHLLFIGDGSLQLTVQELGLSIREKLNPICFIINNDGYTV |
| | | EREIHGPTQSYNDIPMWNYSKLPETFGATEDRVVSKIV |
| | | RTENEFVSVMKEAQADVNRMYWIELVLEKEDAPKLL |
| | | KKMGKLFAEQNK |
| | | (SEQ ID NO: 103) |
| 2VBI | Acetobacter syzygii | MTYTVGMYLAERLVQIGLKHHFAVAGDYNLVLLDQL |
| | 9H-2 | LLNKDMKQIYCCNELNCGFSAEGYARSNGAAAAVVT |
| | | FSVGAISAMNALGGAYAENLPVILISGAPNSNDQGTGH |
| | | ILHHTIGKTDYSYQLEMARQVTCAAESITDAHSAPAKI |
| | | DHVIRTALRERKPAYLDIACNIASEPCVRPGPVSSLLSE |
| | | PEIDHTSLKAAVDATVALLEKSASPVMLLGSKLRAAN |
| | | ALAATETLADKLQCAVTIMAAAKGFFPEDHAGFRGLY |
| | | WGEVSNPGVQELVETSDALLCIAPVFNDYSTVGWSAW |
| | | PKGPNVILAEPDRVTVDGRAYDGFTLRAFLQALAEKA |
| | | PARPASAQKSSVPTCSLTATSDEAGLTNDEIVRHTNALL |
| | | TSNTTLVAETGDSWFNAMRMTLPRGARVELEMQWGH |
| | | IGWSVPSAFGNAMGSQDRQHVVMVGDGSFQLTAQEV |
| | | AQMWYELPVIIFLINNRGYVIEIAIHDGPYNYIKNWDY |
| | | AGLMEVFNAGEGHGLGLKATTPKELTEAIARAKANTR |
| | | GPTLIECQIDRTDCTDMLVQWGRKVASTNARKTTLA |
| | | (SEQ ID NO: 105) |
| 3FZN | Agrobacterium | MASVHGTTYELLRRQGIDTVFGNPGSNELPFLKDFPED |
| | radiobacter | FRYILALQEACVVGIADGYAQASRKPAFINLHSAAGTG |
| | | NAMGALSNAWNSHSPLIWAGQQTRAMIGVEALLTNV |
| | | DAANLPRPLWWSYEPASAAEWHAMSRAIHMASMA |
| | | PQGPVYLSVPYDDWDKDADPQSHHLFDRHVSSSVRLN |
| | | DQDLDILVKALNSASNPAIVLGPDVDAANANADCVML |
| | | AERLKAPVWVAPSAPRCPFPTRHPCFRGLMPAGIAAIS |
| | | QLLEGHDVVLVIGAPVFRYHQYDPGQYLKPGTRLISVT |
| | | CDPLEAARAPMGDAIVADIGAMASALANLVEESSRQL |
| | | PTAAPEPAKVDQDAGRLHPETVFDTLNDMAPENAIYL |
| | | NESTSTTAQMWQRLNMRNPGSYYFCAAGGLGFALPA |
| | | AIGVQLAEPERQVIAVIGDGSANYSISALWTAAQYNIPT |
| | | IFVIMNNGTYGALRWFAGVLEAENVPGLDVPGIDFRA |
| | | LAKGYGVQALKADNLEQLKGSLQEALSAKGPVLIEVS |
| | | TVSPVK |
| | | (SEQ ID NO: 107) |
| IZPD | Zymomonas | MSYTVGTYLAERLVQIGLKHHFAVAGDYNLVLLDNLL |
| | mobilis subsp. | LNKNMEQVYCCNELNCGFSAEGYARAKGAAAAVVT |
| | mobilis | YSVGALSAFDAIGGAYAENLPVILISGAPNNNDHAAGH |
| | | VLHHALGKTDYHYQLEMAKNITAAAEAIYTPEEAPAK |
| | | IDHVIKTALREKKPVYLEIACNIASMPCAAPGPASALFN |
| | | DEASDEASLNAAVDETLKFIANRDKVAVLVGSKLRAA |
| | | GAEEAAVKFTDALGGAVATMAAAKSFFPEENALYIGT |
| | | SWGEVSYPGVEKTMKEADAVIALAPVFNDYSTTGWT |
| | | DIPDPKKLVLAEPRSVVVNGIRFPSVHLKDYLTRLAQK |
| | | VSKKTGSLDFFKSLNAGELKKAAPADPSAPLVNAEIAR |
| | | QVEALLTPNTTVIAETGDSWFNAQRMKLPNGARVEYE |
| | | MQWGHIGWSVPAAFGYAVGAPERRNILMVGDGSFQL |
| | | TAQEVAQMWLKLPVIIFLINNYGYTIEVMIHDGPYNNI |
| | | KNWDYAGLMEVFNGNGGYDSGAAKGLKAKTGGELA |
| | | EAIKVALANTDGPTLIECFIGREDCTEELVKWGKRVAA |
| | | ANSRKPVNKW |
| | | (SEQ ID NO: 109) |
| 1OZF | Klebsiella | MDKQYPVRQWAHGADLVVSQLEAQGVRQVFGIPGAK |
| | pneumoniae subsp. | IDKVFDSLLDSSIRIIPVRHEANAAFMAAAVGRITGKAG |
| | Pneumoniae | VALVTSGPGCSNLITGMATANSEGDPVVALGGAVKRA |
| | | DKAKQVHQSMDTVAMFSPVTKYAIEVTAPDALAEVV |
| | | SNAFRAAEQGRPGSAFVSLPQDVVDGPVSGKVLPASG |
| | | APQMGAAPDDAIDQVAKLIAQAKNPIFLLGLMASQPE |
| | | NSKALRRLLETSHIPVTSTYQAAGAVNQDNFSRFAGRV |
| | | GLFNNQAGDRLLQLADLVICIGYSPVEYEPAMWNSGN |
| | | ATLVHIDVLPAYEERNYTPDVELVGDIAGTLNKLAQNI |
| | | DHRLVLSPQAAEILRDRQHQRELLDRRGAQLNQFALH |
| | | PLRIVRAMQDIVNSDVTLTVDMGSFHIWIARYLYTFRA |
| | | RQVMISNGQQTMGVALPWAIGAWLVNPERKVVSVSG |
| | | DGGFLQSSMELETAVRLKANVLHLIWVDNGYNMVAI |
| | | QEEKKYQRLSGVEFGPMDFKAYAESFGAKGFAVESAE |
| | | ALEPTLRAAMDVDGPAVVAIPVDYRDNPLLMGQLHLS |
| | | QIL |
| | | (SEQ ID NO: 111) |
| YP_006485164.1 | Pseudomonas | MKTVHSASYEILRRHGLTTVFGNPGSNELPFLKDFPED |
| | aeruginosa | FRYILGLHEGAWGMADGFALASGRPAFVNLHAAAGT |
| | | GNGMGALTNAWYSHSPLVITAGQQVRSMIGVEAMLA |
| | | NVDAGQLPKPLVKWSHEPACAQDVPRALSQAIQTASL |
| | | PPRAPVYLSIPYDDWAQPAPAGVEHLAARQVSGAALP |
| | | APALLAELGERLSRSRNPVLVLGPDVDGANANGLAVE |
| | | LAEKLRMPAWGAPSASRCPFPTRHACFRGVLPAAIAGI |
| | | SRLLDGHDLILWGAPVFRYHQFAPGDYLPAGAELVQ |
| | | VTCDPGEAARAPMGDALVGDIALTLEALLEQVRPSAR |
| | | PLPEALPRPPALAEEGGPLRPETVFDVIDALAPRDAIFV |
| | | KESTSTVTAFWQRVEMREPGSYFFPAAGGLGFGLPAA |
| | | VGAQLAQPRRQVIGIIGDGSANYGITALWSAAQYRVP |
| | | AVFIILKNGTYGALRWFAGVLEVPDAPGLDVPGLDFC |
| | | AIARGYGVEALHAATREELEGALKHALAADRPVLIEV |
| | | PTQTIEP (SEQ ID NO: 112) |
| YP_005461458.1 | Actinoplanes | MIDLDGTVTVAEYLGLRLRHAGVEHLFGVPGDFNLNL |
| | missouriensis | LDGLAFVEGLRWVGSPNELGAGYAADAYARRRGLSA |
| | | LFTTYGVGELSAINAVAGSAAEDSPVVHVVGSPRTTTV |
| | | AGGALVHHTIADGDFRHFARAYAEVTVAQAMVTATD |
| | | AGAQIDRVLLAALTHRKPVYLSIPQDLALHRIPAAPLR |
| | | EPLTPASDPAAVERFRTAVRDLLTPAVRPIMLVGQLVS |
| | | RYGLSTLVTDMTTRSGIPVAAQLSAKGVIDESVEGNLG |
| | | LYAGSMLDGPAASLIDSADVVLHLGTALTAELTGFFTH |
| | | RRPDARTVQLLSTAALVGTTRFDNVLFPDAMTTLAEV |
| | | LTTFPAPARLAAPTTRAEPTGLAASITPPAPSAVDLTAS |
| | | TATDLTAPTAGDISEMSRVLTQDAFWAGMQAWLPAG |
| | | HALVADTGTSYWGALALRLPGDTVFLGQPIWNSIGWA |
| | | LPAVLGQGLADPDRRPVLVIGDGAAQMTIQELSTIVAA |
| | | GLRPIILLLNNRGYTIERALQSPNAGYNDVADWNWRA |
| | | VVAAFAGPDTDYHHAATGTELAKALTAASESNRPVFI |
| | | EVELDAFDTPPLLRRLAERATAPS (SEQ ID NO: 113) |
| YP_006991301.1 | Carnobacterium | MYTVGNYLLDRLTELGIRDIFGVPGDYNLKFLDHVMT |
| | maltaromaticum | HKELNWIGNANELNAAYAADGYARTKGIAALVTTFG |
| | LMA28 | VGELSAANGTAGSYAEKVPVVQIVGTPTTAVQNSHKL |
| | | VHHTLGDGRFDHFEKMQTEINGAIAHLTADNALAEID |
| | | RVLRIAVTERCPVYINLAIDVAEVVAEKPLKPLMEESK |
| | | KVEEETTLVLNKIEKALQDSKNPVVLIGNEIASFHLESA |
| | | LADFVKKFNLPVTVLPFGKGGFDEEDAHFIGVYTGAPT |
| | | AESIKERVEKADLILIIGAKLTDSATAGFSYDFEDRQVIS |
| | | VGSDEVSFYGEIMKPVAFAQFVNGLNSLNYLGYTGEIK |
| | | QVERVADIEAKASNLTQNNFWKFVEKYLSNGDTLVAE |
| | | QGTSFFGASLVPLKSKMKFIGQPLWGSIGYTFPAMLGS |
| | | QIANPASRHLLFIGDGSLQLTIQELGMTFREKLTPIVFVI |
| | | NNDGYTVEREIHGPNELYNDIPMWDYQNLPYVFGGN |
| | | KGNVATYKVTTEEELVAAMSQARQDTTRLQWIEVVM |
| | | GKQDSPDLLVQLGKVFAKQNS (SEQ ID NO: 114) |
| WP_003075272.1 | Comamonas | MPANTAPNAQAAEVFTVRHAVINMLRELGMTRIFGNP |
| | testosteroni | GSTELPLFRDYPEDFSYILGLQETVVVGMADGYAQAT |
| | | RNASFVNLHSAAGVGHAMANIFTAFKNRTPMVITAGQ |
| | | QTRSLLQFDPFLHSNQAAELPKPYVKWSCEPARAEDV |
| | | PQALARAYYIAMQEPRGPVFVSIPADDWDVPCEPITLR |
| | | KVGFETRPDPRLLDSIGQALEGARAPAFVVGAAVDRS |
| | | QAFEAVQALAERHQARVYVAPMSGRCGFPEDHALFG |
| | | GFLPAMRERIVDRLSGHDVVFVIGAPAFTYHVEGHGPF |
| | | IAEGTQLFQLIEDPAIAAWAPVGDAAVGNIRMGVQELL |
| | | ARPLTHPRPALQPRPAIPAPAAPEPGRLMTDAFLMHTL |
| | | AQVRSRDSIIVEEAPGSRSIIQAHLPIYAAETFFTMCSGG |
| | | LGHSLPASVGIALARPDKKVIGVIGDGSAMYAIQALWS |
| | | AAHLKLPVTYIIVKNRRYAALQDFSRVFGYREGEKVE |
| | | GTDLPDIDFVALAKGQGCDGVRVTDAAQLSQVLRDAL |
| | | RSPRATLVEVEVA (SEQ ID NO: 115) |
| WP_020634527.1 | Amycolatopsis | MNVAELVGRTLAELGVGAAFGWGSGNFVVTNGLRA |
| | orientalis | GGVRFVAARHEGGAASMADAYARMSGRVSVLSLHQ |
| | HCCB10007 | GCGLTNALTGITEAAKSRTPMIVLTGDTAASAVLSNFR |
| | | IGQDALATAVGAVPERVHSAPTAVADTVRAYRTAVQ |
| | | QRRTVLLNLPLDVQAQEAPEAVEIPKVRGPAPIRPDAG |
| | | MVAKLADLLAEARRPVFIAGRGARASAVPLRELAEISG |
| | | ALLATSAVAHGLFHDDPFSLGISGGFSSPRTADLIVDAD |
| | | LVIGWGCALNMWTTRHGTLLGPAARLVQVDVEQAAL |
| | | GAHRPIDLGVVGDVAGTAVDVHAELDKRGHQRSREA |
| | | PTGTRWNDVPYNDLSGDGRIDPRTLSRRLDEILPAERM |
| | | VSIDSGNFMGYPSAYLSVPDENGFCFTQAFQSIGLGLG |
| | | TAIGAALARPDRLPVLGVGDGGFHMAVSELETAVRLR |
| | | IPLVIVVYNDAAYGAEIHHFGDADMTTVRFPDTDIAAI |
| | | GRGFGCDGVTVRSVGDLAAVKEWLGGPRDAPLVIDA |
| | | KIADDGGSWWLAEAFRH (SEQ ID NO: 116) |
| 1OVM | Enterobacter sp. | MRTPYCVADYLLDRLTDCGADHLFGVPGDYNLQFLD |
| | | HVIDSPDICWVGCANELNASYAADGYARCKGFAALLT |
| | | TFGVGELSAMNGIAGSYAEHVPVLHIVGAPGTAAQQR |
| | | GELLHHTLGDGEFRHFYHMSEPITVAQAVLTEQNACY |
| | | EIDRVLTTMLRERRPGYLMLPADVAKKAATPPVNALT |
| | | HKQAHADSACLKAFRDAAENKLAMSKRTALLADFLV |
| | | LRHGLKHALQKWVKEVPMAHATMLMGKGIFDERQA |
| | | GFYGTYSGSASTGAVKEAIEGADTVLCVGTRFTDTLTA |
| | | GFTHQLTPAQTIEVQPHAARVGDVWFTGIPMNQAIETL |
| | | VELCKQHVHAGLMSSSSGAIPFPQPDGSLTQENFWRTL |
| | | QTFIRPGDIILADQGTSAFGAIDLRLPADVNFIVQPLWG |
| | | SIGYTLAAAFGAQTACPNRRVIVLTGDGAAQLTIQELG |
| | | SMLRDKQHPIILVLNNEGYTVERAIHGAEQRYNDIALW |
| | | NWTHIPQALSLDPQSECWRVSEAEQLADVLEKVAHHE |
| | | RLSLIEVMLPKADIPPLLGALTKALEACNNA (SEQ ID |
| | | NO: 117) |
| 2Q5Q | Azospirillum | MKLAEALLRALKDRGAQAMFGIPGDFALPFFKVAEET |
| | brasilense Sp24 | QILPLHTLSHEPAVGFAADAAARYSSTLGVAAVTYGA |
| | | GAFNMVNAVAGAYAEKSPVVVISGAPGTTEGNAGLLL |
| | | HHQGRTLDTQFQVFKEITVAQARLDDPAKAPAEIARV |
| | | LGAARAQSRPVYLEIPRNMVNAEVEPVGDDPAWPVD |
| | | RDALAACADEVLAAMRSATSPVLMVCVEVRRYGLEA |
| | | KVAELAQRLGVPVVTTFMGRGLLADAPTPPLGTYIGV |
| | | AGDAEITRLVEESDGLFLLGAILSDTNFAVSQRKIDLRK |
| | | TIHAFDRAVTLGYHTYADIPLAGLVDALLERLPPSDRT |
| | | TRGKEPHAYPTGLQADGEPIAPMDIARAVNDRVRAGQ |
| | | EPLLIAADMGDCLFTAMDMIDAGLMAPGYYAGMGFG |
| | | VPAGIGAQCVSGGKRILTVVGDGAFQMTGWELGNCR |
| | | RLGIDPIVILFNNASWEMLRTFQPESAFNDLDDWRFAD |
| | | MAAGMGGDGVRVRTRAELKAALDKAFATRGRFQLIE |
| | | AMIPRGVLSDTLARFVQGQKRLHAAPRE (SEQ ID |
| | | NO: 118) |
| 2VBG | Lactococcus lactis | MNVAELVGRTLAELGVGAAFGVVGSGNFVVTNGLRA |
| | | GGVRFVAARHEGGAASMADAYARMSGRVSVLSLHQ |
| | | GCGLTNALTGITEAAKSRTPMIVLTGDTAASAVLSNFR |
| | | IGQDALATAVGAVPERVHSAPTAVADTVRAYRTAVQ |
| | | QRRTVLLNLPLDVQAQEAPEAVEIPKVRGPAPIRPDAG |
| | | MVAKLADLLAEARRPVFIAGRGARASAVPLRELAEISG |
| | | ALLATSAVAHGLFHDDPFSLGISGGFSSPRTADLIVDAD |
| | | LVIGWGCALNMWTTRHGTLLGPAARLVQVDVEQAAL |
| | | GAHRPIDLGVVGDVAGTAVDVHAELDKRGHQRSREA |
| | | PTGTRWNDVPYNDLSGDGRIDPRTLSRRLDEILPAERM |
| | | VSIDSGNFMGYPSAYLSVPDENGFCFTQAFQSIGLGLG |
| | | TAIGAALARPDRLPVLGVGDGGFHMAVSELETAVRLR |
| | | IPLVIVVYNDAAYGAEIHHFGDADMTTVRFPDTDIAAI |
| | | GRGFGCDGVTVRSVGDLAAVKEWLGGPRDAPLVIDA |
| | | KIADDGGSWWLAEAFRH (SEQ ID NO: 119) |
| 2VBI | Acetobacter syzygii | MTYTVGMYLAERLVQIGLKHHFAVAGDYNLVLLDQL |
| | 9H-2 | LLNKDMKQIYCCNELNCGFSAEGYARSNGAAAAVVT |
| | | FSVGAISAMNALGGAYAENLPVILISGAPNSNDQGTGH |
| | | ILHHTIGKTDYSYQLEMARQVTCAAESITDAHSAPAKI |
| | | DHVIRTALRERKPAYLDIACNIASEPCVRPGPVSSLLSE |
| | | PEIDHTSLKAAVDATVALLEKSASPVMLLGSKLRAAN |
| | | ALAATETLADKLQCAVTIMAAAKGFFPEDHAGFRGLY |
| | | WGEVSNPGVQELVETSDALLCIAPVFNDYSTVGWSAW |
| | | PKGPNVILAEPDRVTVDGRAYDGFTLRAFLQALAEKA |
| | | PARPASAQKSSVPTCSLTATSDEAGLTNDEIVRHINALL |
| | | TSNTTLVAETGDSWFNAMRMTLPRGARVELEMQWGH |
| | | IGWSVPSAFGNAMGSQDRQHVVMVGDGSFQLTAQEV |
| | | AQMVRYELPVIIFLINNRGYVIEIAIHDGPYNYIKNWDY |
| | | AGLMEVFNAGEGHGLGLKATTPKELTEAIARAKANTR |
| | | GPTLIECQIDRTDCTDMLVQWGRKVASTNARKTTLAL |
| | | E (SEQ ID NO: 120) |
| 3FZN | Agrobacterium | MASVHGTTYELLRRQGIDTVFGNPGSNELPFLKDFPED |
| | radiobacter | FRYILALQEACVVGIADGYAQASRKPAFINLHSAAGTG |
| | | NAMGALSNAWNSHSPLIVTAGQQTRAMIGVEALLTNV |
| | | DAANLPRPLVKWSYEPASAAEVPHAMSRAIHMASMA |
| | | PQGPVYLSVPYDDWDKDADPQSHHLFDRHVSSSVRLN |
| | | DQDLDILVKALNSASNPAIVLGPDVDAANANADCVML |
| | | AERLKAPVWVAPSAPRCPFPTRHPCFRGLMPAGIAAIS |
| | | QLLEGHDVVLVIGAPVFRYHQYDPGQYLKPGTRLISVT |
| | | CDPLEAARAPMGDAIVADIGAMASALANLVEESSRQL |
| | | PTAAPEPAKVDQDAGRLHPETVFDTLNDMAPENAIYL |
| | | NESTSTTAQMWQRLNMRNPGSYYFCAAGGLGFALPA |
| | | AIGVQLAEPERQVIAVIGDGSANYSISALWTAAQYNIPT |
| | | IFVIMNNGTYGALRWFAGVLEAENVPGLDVPGIDFRA |
| | | LAKGYGVQALKADNLEQLKGSLQEALSAKGPVLIEVS |
| | | TVSPVKHHHHHH (SEQ ID NO: 121) |

| Enzyme | |
|---|---|
| name or | |
| UniProt/ | |
| Genebank ID | Gene sequence |
| 4COK | ATGACGTATACCGTGGGCCGCTATCTGGCTGACCGTTTAG |
| | CCCAAATTGGTCTTAAACATCACTTTGCCGTGGCAGGCGA |
| | CTACAACTTGGTTCTGTTAGACCAGCTGCTGCTGAATACC |
| | GACATGCAACAGATTTACTGCAGTAATGAACTTAACTGTG |
| | GGTTCAGTGCCGAAGGCTATGCGCGCGCCAACGGCGCGG |
| | CTGCAGCCATTGTCACCTTTTCCGTCGGCGCTCTGAGCGC |
| | CTTCAACGCCTTGGGCGGCGCATACGCGGAAAACTTGCC |
| | GGTCATCCTGATCTCTGGCGCACCGAACGCGAATGACCAC |
| | GGGACCGGCCATATCTTGCACCATACGCTGGGCACCACA |
| | GATTATGGCTACCAACTGGAAATGGCACGCCATATTACAT |
| | GTGCGGCGGAATCAATTGTCGCTGCAGAGGATGCGCCAG |
| | CGAAAATTGATCACGTGATTCGCACCGCGCTGCGCGAAA |
| | AAAAACCAGCATACCTGGAAATTGCGTGTAATGTGGCTG |
| | GCGCTCCATGCGTTCGCCCGGGCGGTATTGATGCGCTTCT |
| | GTCGCCGCCCGCCCCGGATGAAGCCAGCCTGAAGGCGGC |
| | CGTTGACGCCGCCCTGGCCTTCATTGAACAACGCGGCTCA |
| | GTGACGATGCTCGTTGGTAGTCGTATCCGTGCAGCCGGAG |
| | CCCAGGCTCAGGCGGTCGCCCTCGCGGATGCTCTGGGCTG |
| | CGCGGTGACGACGATGGCGGCAGCGAAATCTTTTTTTCCA |
| | GAAGATCATCCGGGTTATCGTGGTCACTACTGGGGTGAG |
| | GTGTCATCCCCGGGTGCCCAACAGGCCGTGGAGGGCGCT |
| | GACGGTGTGATTTGTTTGGCCCCGGTTTTCAATGACTATG |
| | CCACTGTGGGCTGGAGCGCGTGGCCGAAAGGGGATAACG |
| | TCATGCTTGTGGAACGTCACGCGGTTACCGTAGGTGGTGT |
| | TGCGTATGCCGGCATCGATATGCGAGACTTTCTGACACGT |
| | CTGGCGGCTCACACCGTACGCCGTGATGCCACCGCACGC |
| | GGCGGGGCATATGTAACCCCGCAGACGCCGGCAGCGGCT |
| | CCGACTGCCCCTCTGAACAACGCGGAGATGGCGCGCCAG |
| | ATCGGCGCGCTACTGACGCCGCGGACAACTTTGACCGCG |
| | GAAACCGGCGACAGCTGGTTCAATGCGGTCCGTATGAAA |
| | CTGCCGCACGGCGCGCGGGTCGAACTGGAAATGCAATGG |
| | GGGCACATCGGTTGGAGCGTGCCGGCGGCGTTTGGTAAC |
| | GCGCTGGCGGCGCCGGAACGCCAGCACGTCCTGATGGTG |
| | GGTGACGGCTCATTTCAGCTGACTGCACAGGAAGTGGCC |
| | CAGATGATTCGTCATGACTTACCGGTGATAATCTTTCTGA |
| | TCAACAACCACGGCTATACTATAGAAGTGATGATCCATG |
| | ACGGGCCGTATAACAACGTGAAGAACTGGGATTACGCGG |
| | GCCTGATGGAAGTCTTCAATGCGGGGGAAGGTAACGGCC |
| | TCGGTCTTCGTGCCCGCACTGGGGGCGAACTGGCGGCGG |
| | CTATTGAACAGGCCCGCGCCAACCGTAACGGCCCGACCC |
| | TGATCGAATGTACCCTGGACCGCGATGACTGCACGCAGG |
| | AACTGGTGACCTGGGGCAAACGTGTTGCAGCTGCCAACG |
| | CGCGCCCTCCTCGTGCAGGA |
| | (SEQ ID NO: 2) |
| A0A0F6SDN1_9DELT | ATGGCCGATCTGCTGGCGATTCACCGACATGCCGTGCGTG |
| | CCCGTCTGCTGGATGAGCGTTTAACGCAACTTGCCCGCGC |
| | TGGCCGCATCGGGTTCCACCCTGATGCACGTGGTTTCGAG |
| | CCGGCTATTGCGGCTGCCGTACTGGCTATGCGCGCGGAAG |
| | ATGCTATTTTCCCGTCCGCGCGAGATCACGCAGCGTTCTT |
| | GGTTCGCGGATTGCCGATTAGCCGGTATGTGGCCCATGCG |
| | TTTGGCAGTGTTGAGGATCCTATGCGTGGCCACGCTGCCC |
| | CCGGGCACTTAGCGTCACGCGAACTGCGCATTGCCGCGG |
| | CCAGCGGTCTGGTCAGCAACCATATGACTCACGCCGCCG |
| | GTTACGCGTGGGCAGCTAAACTTCGCGGGGAAACGTGCG |
| | CGGTTTTGACCATGTTTGCAGACACCGCTGCGGACGCTGG |
| | TGACTTTCATTCAGCGGTAAACTTTGCGGGTGCCACCAAG |
| | GCGCCGGTTATCTTTTTTTGCCGTACAGATCGGACCCGTA |
| | GTGCACATCCGCCGACGCCGATTGACCGTGTGGCCGATA |
| | AGGGCATTGCATACGGTGTGGAGAGCTTGGTTTGTTCGGC |
| | CGATGATGCCGGTGCGGTGGCTAGCGCCATGGCACAGGC |
| | ACACCAGCGCGCTCTGGCCGGCGAAGGTCCTACGCTGGT |
| | GGAAGCGATTCGTGAATCCAAAAGCGATCCCATCGAGGC |
| | CCTGGAGGCTCGCCTGTCTAGCGAAGGTCACTGGGATGC |
| | GCACCGTGCGCTGGAACTGCGCCGCGAGCTGATGACTGA |
| | GATCGAGTCTGCCGTGGCGCATGCCCAGCAGGTTGGTGCT |
| | CCCCCACGCGAAGCCGTGTTCGAAGATGTCTATGCAACCT |
| | TGCCGCGTCACCTGGAAGACCAGCGTACGACATTACTGG |
| | CCACCGCCAACCACGAAGATCGG |
| | (SEQ ID NO: 4) |
| 4K9Q | ATGCGCACCGTTAAAGAGATCACATTCGATCTGTTGCGGA |
| | AACTGCAAGTTACCACCGTGGTGGGCAACCCAGGCTCCA |
| | CCGAGGAAACGTTTCTGAAAGATTTTCCGTCGGACTTTAA |
| | CTATGTACTGGCCCTCCAGGAAGCGAGCGTCGTCGCGATC |
| | GCGGACGGCTTATCCCAGAGTCTTCGTAAGCCCGTGATCG |
| | TTAACATTCACACGGGGGCAGGCTTGGGCAATGCTATGG |
| | GGTGCTTGTTGACAGCCTATCAGAATAAAACCCCCCTTAT |
| | TATAACCGCGGGGCAACAAACCCGCGAAATGCTGCTCAA |
| | CGAACCGTTATTAACCAACATAGAAGCGATCAATATGCC |
| | GAAACCGTGGGTGAAGTGGAGCTATGAACCGGCACGGCC |
| | GGAGGACGTCCCGGGCGCATTCATGCGCGCGTATGCGAC |
| | GGCTATGCAACAGCCCCAGGGTCCGGTTTTTCTGAGCCTT |
| | CCGCTTGACGATTGGGAAAAACTTATCCCTGAAGTAGATG |
| | TCGCCCGCACAGTGTCTACCCGTCAAGGTCCGGATCCGGA |
| | CAAGGTCAAAGAATTTGCGCAACGCATTACCGCATCAAA |
| | AAATCCGCTGCTCATTTATGGCAGCGATATTGCGCGCTCG |
| | CAAGCGTGGAGCGATGGTATCGCATTCGCAGAACGCCTA |
| | AACGCACCGGTCTGGGCGGCTCCCTTCGCGGAACGGACC |
| | CCATTTCCTGAAGATCATCCCCTTTTTCAGGGTGCCCTGA |
| | CCTCGGGTATCGGAAGCCTGGAAAAGCAAATCCAGGGTC |
| | ATGATTTAATCGTGGTCATCGGTGCCCCGGTGTTTCGCTA |
| | CTACCCTTGGATCGCGGGGCAATTTATTCCGGAGGGCTCA |
| | ACCCTCCTTCAGGTGTCGGATGATCCTAATATGACCAGCA |
| | AAGCGGTAGTTGGTGATTCCTTGGTTAGCGATTCGAAATT |
| | GTTCCTGATCGAAGCACTTAAACTGATCGATCAGCGCGAA |
| | AAAAACAATACGCCACAGCGCAGCCCGATGACCAAAGAG |
| | GACCGTACCGCCATGCCACTCCGTCCCCATGCTGTTCTCG |
| | AAGTGCTGAAAGAAAATTCACCGAAAGAGATAGTACTGG |
| | TCGAAGAGTGTCCATCCATCGTTCCTCTGATGCAGGACGT |
| | TTTCCGCATTAACCAACCGGATACCTTCTACACCTTTGCA |
| | AGTGGCGGCTTGGGTTGGGACCTGCCGGCCGCAGTAGGG |
| | CTGGCCCTGGGCGAGGAAGTTAGCGGCCGCAACCGGCCT |
| | GTGGTTACGCTTATGGGCGATGGATCCTTCCAATATAGCG |
| | TTCAAGGTATTTACACGGGAGTGCAGCAAAAAACCCATG |
| | TAATTTACGTGGTGTTCCAGAACGAAGAATATGGGATCTT |
| | AAAGCAGTTTGCAGAACTTGAACAGACTCCGAACGTGCC |
| | CGGACTGGATCTGCCGGGGCTGGACATTGTGGCTCAGGG |
| | TAAAGCGTATGGCGCAAAAAGCCTTAAAGTGGAAACACT |
| | TGATGAATTAAAAACCGCCTATCTGGAAGCGCTGAGCTTT |
| | AAGGGTACGTCTGTCATTGTCGTGCCGATCACCAAGGAAT |
| | TAAAACCACTTTTCGGA |
| | (SEQ ID NO: 6) |
| D6ZJY9_MOBCV | ATGCTGAAACAGATTGAAGGCTCTCAGGCAATAGCACGT |
| | GCCGTTGCTGCGTGCCAGCCAAACGTGGTCGCAGCCTATC |
| | CGATCTCACCGCAGACCCATATTGTGGAAGCACTTTCTGC |
| | GCTGGTAAAAAGTGGCCAGCTGGAACACTGCGAGTACGT |
| | GAACGTAGAATCCGAATTCGCAGCCATGTCTGCCTGCATT |
| | GGCTCGTCCGCAGTTGGCGCGCGCTCATATACTGCGACGG |
| | CATCACAGGGCTTGCTGTATATGGTTGAAGCGGTCTACAA |
| | CGCCGCTGGCCTGGGCTTCCCGATTGTCATGACGGTGGCG |
| | AACCGTGCAATTGGAGCTCCGATCAATATCTGGAATGACC |
| | ACAGTGATTCGATGTCGCAGCGCGACTCTGGCTGGCTGCA |
| | GCTGTTCGCCGAGAACAACCAGGAAGCCGCAGACTTACA |
| | TGTGCAGGCATTTCGTATCGCTGAGGAGTTGAGCGTCCCG |
| | GTTATGGTGTGCATGGATGGTTTCATTCTAACGCATGCCG |
| | TTGAACAGGTCGACCTCCCGGAATCTGAACAAGTGAAAC |
| | AGTTTCTCCCTCCCTACGAACCACGTCAAGTTCTGGACCC |
| | GGACGATCCGTTATCTATTGGCGCTATGGTTGGTCCGGAA |
| | GCGTTTACCGAGGTGCGCTATATTGCTCATCATAAAATGC |
| | TGCAGGCTCTGGATCTGATCCCACAAGTGCAGTCCGAATT |
| | TAAATCAATATTTGGCCGGGACTCTGGGGGACTGCTGCAT |
| | ACGTATCGGTGCGAAGATGCGGAAACTATTATTGTGGCCC |
| | TGGGTTCCGTTGTAGGTACCCTGAAAGATGTCGTGGACCA |
| | ACGTCGCGAGAATGGCGAGAAAATCGGCATCATGAGCTT |
| | AGTGAGCTTCCGCCCCTTCCCATTTGCTGCCATCCGCGAG |
| | GTCCTGCAGTCAGCGAAACGCGTGGTTTGCCTGGAGAAA |
| | GCGTTTCAATTGGGTATTGGGGGGATTGTATCTTCTGAGC |
| | TGCGGGCGGCCATGCGTGGTTTGCCGTTCACTTGTTACGA |
| | AGTAATCGCCGGTTTGGGTGGCCGCAACATTACTAAAAA |
| | CAGTCTACATGCTATGCTTGATCAGGCCGTCGCTGATACG |
| | ATCGAGCCGCTAACCTTTATGGATCTGGATATGGAGCTGG |
| | TGCAGGGCGAGCTCGAACGGGAAGCAGCGACGAGACGCT |
| | CTGGCGCTTTCGCCACCAACCTGCAACGCGAACGTGTCCT |
| | GCGTGCGAACGCTAAAATTGCAGAAGCAGGTCCGAAACC |
| | AAAAGCAGATAAAGTAGGTAACCCGCGGGTTGCGTCTCC |
| | GTCAATCAAGCAGGATGCGGTGCCTGTAGTCCCTGACCA |
| | GGCTGAA |
| | (SEQ ID NO: 8) |
| \|Q1LMD8_CUPMC | ATGATTGAGGCTGTTCAGTTTGTCGAGGCGGCACGGGAA |
| | CGTGGCTTTGAATGGTACGCGGGGGTTCCCTGCAGTTATT |
| | TGACTCCGTTCATTAATTATGTAGTTCAGGATCCGTCGCT |
| | GCACTACGTCAGTGCCGCGAACGAGGGAGATGCTGTTGC |
| | ATTCATCGCGGGCGTCACCCAAGGTGCTCGCAACGGCGTC |
| | CGTGGTATCACCATGATGCAAAATTCCGGTCTGGGTAACG |
| | CCGTGTCCCCGCTGACCAGCCTGACCTGGACCTTCCGCCT |
| | GCCGCAGCTGTTGATAGTAACGTGGCGTGGTCAGCCGGG |
| | CGGCGCCTCAGACGAACCACAACATGCGCTGATGGGCCC |
| | TGTGACCCCGGCGATGCTGGACACCATGGAGATCCCGTG |
| | GGAACTGTTTCCGACAGAACCGGATGCAGTGGGGCCAGC |
| | CCTCGATCGCGCCATCGCACACATGGACGCCACGGGCCG |
| | TCCTTACGCGCTGATCATGCAGAAGGGCTCGGTGGCTCCA |
| | TACCCGCTGAAGACACAGACTCCGCCGGTTGCACGCGCG |
| | AAGGCGACCCCACAGGTTAGTCGCTCAGGTGCCACGCCA |
| | TTACCATCGCGTCAAGAAGCCCTTCAGCGGGTTATCGCCC |
| | ATACCCCGGCTGATTCAACTGTGGTTCTGGCATCTACTGG |
| | CTTTTGCGGTCGAGAACTGTATGCGTTGGATGACCGCCCG |
| | AACCAATTATATATGGTGGGTTCCATGGGTTGTCTGACGC |
| | CATTCGCACTGGGGTTGGCAATGGCGCGTCCGGATCTCAA |
| | AGTGGTTGCAGTAGATGGCGATGGCGCGGCCCTAATGCG |
| | CATGGGGGTGTTCGCGACTCTGGGGGCGTATGGGCCGGC |
| | TAACCTCACCCACGTTTTATTAGACAACAACGCACACGAT |
| | TCAACCGGCGGCCAGGCCACCGTAAGCCATAATGTTTCTT |
| | TTGCGGGGGTCGCAGCGGCGTGCGGCTACGCCTCTGCAAT |
| | CGAAGGTGACGACTTGGATATGCTGGACCGTGTGTTAGC |
| | GTCCGCCGCAACAGCGACTTCCGGGCCGAACTTCGTGTGC |
| | TTACAAACTCGTGCAGGTACGCCGGACGGCTTACCACGA |
| | CCATCTGTGACCCCGGTTGAAGTGAAAACGCGCCTTGGTC |
| | GGCAAATTGGCGCCGACCAGGGCCACGCAGGCGAAAAAC |
| | ACGCCGCGGCC |
| | (SEQ ID NO: 10) |
| Q9F768 | ATGAATACCCTGACCTCTCAGATTGAACAACTGCAAAGCC |
| | TGGCCCACGAACTGCTGTATCTGGGTGTGGACGGTGCCCC |
| | TATCTATACCGACCATTTTCGTCAGCTGAACAAGGAAGTC |
| | CTGGAACAAAGCGATGCGCTCTATCCACAGAGGGGCGCT |
| | ACCCCGGAAGAAGAGGCCAACATTTGCCTGGCACTGCTT |
| | ATGGGTTATAATGCAACGATTTACAATCAGGGCGATAAG |
| | GAAGAGAAAAAACAAGTGGTCCTGAATCGCTGTTGGGAT |
| | GTGCTGGATCAGCTCCCGGCAACCCTCCTGAAGTGTCAGC |
| | TTCTCACGTACTGCTATGGCGAAGTTTTTGAAGAAGAGTT |
| | AGCGAAAGAAGCCCACACAATCATAGAGTCATGGAGTAA |
| | CCGCGAACTGCTGAAAGCAGAAAAAGAAATCGCGGAATC |
| | GCTGAATAACCTCGAGGCGAATCCGTACCCGTATTCCGAA |
| | CTGCACGAA |
| | (SEQ ID NO: 12) |
| I3BXS7_9GAMM | ATGCAAATCCAGGTTAGCGAGCTGATTGTAAAGTTCTTGC |
| | AGAAATTAGGTGTCGATACAATTTTTGGCATGCCAGGCGC |
| | CCACATCCTGCCCGTGTATGATGAATTATACGACAGCGGC |
| | ATAAAAACCGTTCTCGTTAAGCACGAACAGGGCGCCGCG |
| | TTCATGGCGGGTGGCTACGCCCGGGTTTCTGGTCGAATTG |
| | GTGCGTGTATCACTACCGCTGGCCCGGGGGCCTCGAATCT |
| | AATCACCGGTATCGCTAACGCGTATGCGGATAAATTGCCG |
| | ATGATTGTTATCACCGGCGAGGCCCCTACCCACATTTTCG |
| | GCCGAGGCGGCTTACAGGAATCTTCCGGTGAAGGTGGCT |
| | CAATCGACCAAACCGCACTCTTCAGCGGGGTGACCCGAT |
| | ACCACAAACTGATTGAACGTACCGATTACATTACCAATGT |
| | CCTCTCCCAGGCCGCCCGGCAGCTTGTAGCCGATGTACCA |
| | GGACCCGTTGTCCTCTCGATTCCAGTTAACGTGCAAAAAG |
| | AGCTTGTCGACGCAAGTATTTTAGAAAACTTACCTACGCT |
| | TAAACCGCTGCCGAAACTGCAGATCGCGCCGCCGGTGCT |
| | GGAGCAGTGTGCGGATATGATCCGCAAGGCTCGTTGTCC |
| | AGTCATCCTGGCGGGGTATGGCTGTCTGCAGTCGGTGCGC |
| | GCTAGATTAGAGCTGCGTAAATTCAGCGAACACCTGAAT |
| | ATTCCAGTGGCGACGAGTCTTAAAGGGAAGGGAGCGATT |
| | GATGAACGTTCGGCACTCAGCCTGGGGTCGCTGGGCGTG |
| | ACGAGTAGCGGACATGCTATGCACTATTTTATGCAAGAG |
| | GCGGATCTCATCATTCTGCTAGGGGCGGGCTTTAATGAAC |
| | GTACGTCTTATGTTTGGAAGGCAGACTTAACCCAAGAGCG |
| | TAAAATCATTCAGGTCGATCGTAATGTTGCTCAGCTAGAA |
| | AAAGTGGTTAAGGCCGATTTGGCAATTCAGTCTGATCTGG |
| | GCGATTTTTTACACGCGCTGAACACCTGTTGTGTGCCCCA |
| | GGGTATTGAACCGAAATCATGTCCGGATCTGGCAGCCTTT |
| | AAACAGAAAGTGGATCAGCAGGCGGCCCAGAGTGGCCAG |
| | GTGATCTTCAACCAGAAATTTGATTTAGTTAAGTCGTTGT |
| | TTGCACGACTGGAACCTCATTTTGCCGAAGGTATCGTATT |
| | GGTGGATGACAATATCATCTATGCGCAAAACTTCTACCGC |
| | GTGAAAGACGGGGACCTGTTTGTACCGAACACTGGGGTG |
| | AGCAGCCTGGGACATGCGATTCCCGCCGCCATTGGTGCGC |
| | GCTTCGTCTTGGATAAACCGATGTTTGCGATTCTTGGCGA |
| | TGGTGGCTTCCAAATGTGTTGTATGGAAATAATGACCGCT |
| | GTGAATTATAATATTCCGCTCAACATCGTGCTCTTTAACA |
| | ATCAGACCCTGGGACTGATACGTAAAAACCAACATCAAC |
| | AGTATGAACAGCGTTTCCTGGATTGTGATTTCCAGAACCC |
| | AGACTATGCCCTACTGGCGCAAAGCTTTGGCATTAACCAC |
| | TTTCATGTGGGTAACAACGCCGATCTGCAGCGCGTTTTTG |
| | ACACGGCGGATTTTCATCATGCTATCAACCTGATTGAGCT |
| | CATGGTTGATCGCGAAGCTTATCCAAACTATTCAAGCCGT |
| | CGC |
| | (SEQ ID NO: 14) |
| 1JSC | ATGATCCGTCAGTCTACCCTGAAAAACTTTGCTATCAAAC |
| | GCTGCTTTCAGCATATTGCCTATCGTAACACTCCGGCCAT |
| | GCGTTCGGTAGCGCTAGCACAGCGCTTCTATTCCTCTTCT |
| | AGCAGATACTATTCGGCATCTCCGCTGCCGGCCAGTAAAC |
| | GCCCCGAACCAGCTCCGTCGTTCAACGTTGATCCACTGGA |
| | ACAGCCAGCGGAACCTTCTAAGCTGGCGAAAAAACTTCG |
| | CGCGGAACCGGATATGGATACTTCATTCGTAGGTCTGACA |
| | GGAGGCCAGATCTTTAATGAGATGATGAGTCGTCAAAAC |
| | GTCGACACGGTATTCGGCTACCCGGGCGGAGCCATCCTGC |
| | CGGTATATGATGCGATTCATAACTCGGATAAATTCAACTT |
| | TGTGTTGCCGAAACATGAACAGGGCGCGGGCCACATGGC |
| | AGAGGGATATGCGCGTGCAAGCGGCAAACCGGGTGTCGT |
| | GCTGGTAACATCAGGCCCGGGTGCAACAAATGTTGTCAC |
| | ACCTATGGCGGATGCTTTTGCCGACGGTATCCCGATGGTA |
| | GTGTTCACCGGCCAAGTGCCAACCAGCGCGATTGGAACA |
| | GACGCTTTCCAGGAAGCTGATGTGGTCGGCATCTCCCGCA |
| | GTTGTACAAAGTGGAACGTGATGGTGAAGAGCGTAGAAG |
| | AGTTGCCTCTGCGTATCAACGAAGCGTTCGAGATTGCGAC |
| | CAGTGGGCGCCCGGGGCCCGTCTTAGTCGACTTACCTAAG |
| | GACGTAACCGCCGCGATCCTGCGCAATCCTATTCCGACCA |
| | AAACTACGTTACCCAGTAACGCGCTGAACCAGCTTACCA |
| | GCCGCGCTCAGGACGAATTCGTCATGCAGTCCATCAATAA |
| | AGCTGCGGACCTTATTAACCTGGCTAAAAAGCCTGTGCTC |
| | TATGTTGGTGCCGGTATTCTCAATCACGCCGATGGACCGC |
| | GTCTGCTGAAAGAGCTGAGCGACCGCGCTCAGATCCCCG |
| | TGACCACTACGCTTCAAGGCCTTGGCTCCTTTGATCAGGA |
| | AGATCCTAAAAGCTTAGATATGTTAGGAATGCACGGATG |
| | CGCCACGGCGAACCTGGCGGTGCAGAATGCGGATCTGAT |
| | TATTGCCGTCGGCGCCCGTTTTGACGACCGTGTGACCGGC |
| | AACATTAGCAAATTTGCTCCTGAAGCTCGTCGTGCTGCTG |
| | CGGAAGGACGTGGAGGAATTATTCATTTTGAAGTAAGTC |
| | CAAAAAATATTAACAAAGTCGTACAGACCCAGATTGCGG |
| | TCGAGGGTGATGCGACCACCAATCTGGGGAAGATGATGA |
| | GCAAAATCTTCCCTGTAAAAGAACGTAGTGAGTGGTTCGC |
| | CCAGATAAATAAGTGGAAAAAAGAATATCCATATGCCTA |
| | TATGGAGGAAACGCCAGGTAGTAAAATTAAACCGCAAAC |
| | TGTGATCAAAAAACTGTCAAAAGTCGCAAACGATACGGG |
| | TCGTCATGTAATCGTAACTACGGGCGTGGGTCAGCATCAG |
| | ATGTGGGCGGCGCAGCATTGGACCTGGCGTAACCCGCAT |
| | ACCTTTATTACGAGCGGCGGATTGGGGACCATGGGCTATG |
| | GGTTGCCGGCGGCGATTGGCGCCCAGGTGGCCAAGCCAG |
| | AGTCACTGGTCATCGATATTGACGGTGACGCGAGCTTCAA |
| | CATGACGCTGACGGAGTTGTCCTCAGCGGTTCAGGCCGGT |
| | ACTCCGGTGAAAATCCTGATTCTGAACAATGAGGAACAG |
| | GGTATGGTTACGCAGTGGCAAAGCTTATTCTACGAGCACC |
| | GATATTCCCACACGCATCAGCTGAACCCTGACTTCATTAA |
| | ACTTGCTGAAGCAATGGGGCTGAAGGGCCTGCGCGTGAA |
| | AAAGCAGGAAGAACTTGATGCTAAACTGAAAGAATTCGT |
| | CTCGACGAAGGGACCAGTACTTTTAGAAGTGGAGGTGGA |
| | TAAAAAAGTTCCAGTCTTACCTATGGTCGCTGGCGGTAGC |
| | GGCCTGGATGAATTTATTAATTTCGATCCGGAGGTCGAAC |
| | GTCAGCAAACTGAATTGCGCCATAAACGGACAGGAGGTA |
| | AACAC |
| | (SEQ ID NO: 16) |
| O86938\|PPD_STRVT | ATGATTGGGGCTGCCGATCTGGTCGCTGGTCTGACCGGTC |
| | TGGGTGTGACCACAGTGGCCGGTGTACCGTGCAGTTATTT |
| | AACTCCGTTAATCAACCGAGTAATCAGTGACCCGGCAAC |
| | GAGATATTTGACGGTGACGCAGGAAGGAGAAGCAGCGGC |
| | AGTTGCAGCAGGGGCCTGGTTGGGTGGTGGTCTGGGCTG |
| | CGCGATTACCCAAAACAGCGGTCTTGGCAACATGACCAA |
| | CCCTCTCACCTCTTTACTTCACCCTGCCCGTATCCCGGCGG |
| | TAGTTATCACCACCTGGCGCGGCCGCCCGGGTGAGAAAG |
| | ATGAGCCCCAGCACCACCTAATGGGCCGCATTACTGGTG |
| | ATCTCCTGGACCTGTGTGATATGGAGTGGTCGCTGATTCC |
| | GGATACGACCGACGAACTGCACACAGCGTTTGCTGCTTGC |
| | CGTGCTTCCCTGGCGCACCGTGAGCTGCCTTATGGTTTTCT |
| | GCTTCCGCAGGGTGTGGTGGCCGATGAGCCACTGAACGA |
| | AACGGCTCCGCGTTCGGCCACCGGGCAGGTCGTCCGCTAT |
| | GCGCGTCCAGGCCGGTCTGCTGCCCGGCCTACGCGCATTG |
| | CCGCCCTGGAACGCCTACTCGCCGAGTTACCGCGTGACGC |
| | AGCAGTGGTATCTACCACCGGCAAAAGCTCCCGAGAGCT |
| | GTACACTTTGGACGATCGTGATCAACATTTCTATATGGTC |
| | GGTGCGATGGGCTCTGCCGCGACCGTTGGACTGGGAGTC |
| | GCGTTGCATACCCCCCGTCCGGTCGTTGTTGTTGATGGTG |
| | ACGGCTCCGTCTTGATGCGCCTCGGTTCGCTGGCAACCGT |
| | GGGGGCCCATGCCCCCGGCAACCTGGTGCATCTTGTGCTG |
| | GATAACGGTGTCCACGATAGCACGGGTGGCCAACGCACG |
| | TTGAGCAGCGCGGTGGATCTCCCAGCTGTCGCCGCCGCGT |
| | GCGGCTATCGCGCTGTGCACGCCTGCACCTCTCTGGATGA |
| | TCTCAGTGATGCATTGGCGACCGCGTTAGCGACGGATGGT |
| | CCGACCTTAGTGCACCTGGCGATTCGCCCGGGAAGCCTGG |
| | ATGGTCTGGGCCGCCCGAAAGTCACGCCCGCTGAAGTGG |
| | CCCGTCGTTTTCGTGCGTTCGTGACCACCCCCCCAGCCGG |
| | TACAGCTACGCCTGTTCACGCTGGTGGTGTGACAGCCCGG |
| | (SEQ ID NO: 18) |
| 3L84_3M34 | ATGAACATTCAAATTTTGCAAGAACAAGCGAACACTCTG |
| | CGTTTCTTGAGTGCGGACATGGTCCAGAAAGCCAATAGC |
| | GGCCACCCTGGCGCACCCCTGGGCCTGGCGGATATCCTCT |
| | CTGTGCTCAGTTATCATCTTAAACACAACCCAAAAAACCC |
| | GACCTGGCTTAACCGCGACCGCTTAGTGTTTTCCGGCGGT |
| | CACGCCTCCGCACTGTTGTATTCTTTCCTTCATCTGAGCGG |
| | CTACGACTTAAGTCTGGAAGACCTCAAGAACTTCCGCCAG |
| | CTGCACTCGAAGACCCCGGGGCACCCCGAAATTTCCACCC |
| | TGGGCGTAGAAATTGCCACGGGTCCTCTGGGCCAGGGGG |
| | TGGCGAATGCAGTGGGATTTGCGATGGCGGCAAAAAAAG |
| | CGCAAAATCTGCTGGGCAGTGACCTGATTGATCACAAAA |
| | TCTACTGTCTGTGCGGTGACGGCGATCTGCAGGAGGGTAT |
| | TTCATATGAGGCGTGTTCTCTGGCGGGCCTGCACAAATTA |
| | GATAATTTTATCCTGATATATGATAGTAACAACATTAGCA |
| | TTGAGGGTGACGTCGGTCTGGCGTTCAATGAAAACGTTAA |
| | GATGCGTTTTGAAGCGCAGGGGTTCGAAGTGCTGAGCATT |
| | AATGGTCACGATTATGAAGAAATTAACAAAGCCCTGGAA |
| | CAGGCCAAGAAATCTACCAAACCATGCTTGATTATCGCA |
| | AAAACAACCATTGCGAAAGGCGCGGGTGAACTTGAAGGT |
| | AGCCACAAAAGCCACGGCGCCCCACTGGGTGAAGAAGTG |
| | ATCAAAAAAGCGAAAGAACAGGCTGGCTTTGATCCCAAC |
| | ATCTCTTTTCATATTCCGCAGGCTTCGAAAATCCGCTTTGA |
| | AAGCGCCGTTGAACTGGGGGACCTGGAAGAAGCGAAATG |
| | GAAGGACAAACTTGAAAAATCCGCAAAAAAAGAACTGCT |
| | CGAACGCCTGCTGAACCCAGATTTTAACAAGATTGCGTAT |
| | CCCGATTTCAAAGGCAAAGACCTGGCCACGCGAGACAGT |
| | AACGGGGAGATTTTAAATGTTCTGGCCAAAAATCTGGAG |
| | GGTTTCCTGGGCGGCTCCGCTGACCTGGGTCCTTCGAACA |
| | AGACGGAGCTACACTCAATGGGTGACTTTGTTGAGGGCA |
| | AGAACATTCACTTTGGTATTCGTGAACATGCCATGGCGGC |
| | TATTAACAATGCCTTTGCGCGCTATGGAATCTTTCTGCCCT |
| | TTTCAGCGACGTTCTTCATCTTCAGCGAATATCTTAAACC |
| | GGCGGCGCGCATCGCCGCGCTGATGAAGATCAAACATTT |
| | TTTCATTTTTACGCACGACAGCATCGGAGTAGGAGAAGAC |
| | GGCCCGACGCACCAGCCTATAGAACAATTAAGTACCTTTC |
| | GCGCCATGCCGAATTTCCTCACTTTTCGTCCGGCGGATGG |
| | GGTAGAAAACGTAAAAGCTTGGCAGATTGCACTCAATGC |
| | CGACATTCCATCTGCGTTCGTCCTCTCACGTCAGAAGCTG |
| | AAGGCCTTGAACGAGCCTGTTTTTGGTGACGTGAAGAAC |
| | GGAGCATACCTGCTGAAAGAATCTAAAGAAGCCAAGTTT |
| | ACCCTGCTTGCTTCTGGCTCGGAGGTGTGGCTGTGCTTAG |
| | AAAGCGCAAACGAACTTGAAAAACAAGGCTTTGCCTGCA |
| | ACGTCGTGAGTATGCCGTGTTTTGAGCTGTTCGAAAAGCA |
| | GGATAAAGCTTACCAGGAACGCCTGCTTAAAGGAGAAGT |
| | AATTGGCGTGGAGGCGGCACACTCTAATGAACTGTACAA |
| | ATTTTGCCATAAAGTGTATGGGATCGAAAGCTTTGGCGAG |
| | AGTGGCAAAGACAAAGACGTTTTTGAACGTTTCGGCTTTT |
| | CGGTGTCCAAACTTGTGAATTTTATTCTGTCCAAA |
| | (SEQ ID NO: 20) |
| lupa_A | ATGAGCCGTGTCTCTACAGCGCCTTCGGGTAAACCTACGG |
| | CAGCTCACGCACTTTTAAGTCGCCTGCGTGACCATGGGGT |
| | AGGCAAGGTTTTCGGTGTGGTGGGCCGTGAAGCCGCCTC |
| | GATCCTGTTCGATGAAGTCGAAGGTATCGATTTCGTCCTG |
| | ACCCGCCATGAGTTTACCGCAGGCGTAGCCGCGGACGTG |
| | TTAGCACGTATCACCGGGCGTCCACAAGCCTGCTGGGCTA |
| | CCCTGGGACCGGGAATGACCAATCTGAGCACCGGGATTG |
| | CAACGTCAGTATTAGACCGTTCGCCGGTTATTGCGCTCGC |
| | AGCTCAGAGTGAATCACACGATATTTTCCCAAACGACACC |
| | CACCAATGTTTAGACTCAGTGGCGATTGTGGCACCGATGA |
| | GCAAATATGCGGTTGAGCTGCAGCGCCCACACGAAATTA |
| | CGGATTTGGTCGATAGTGCCGTTAATGCCGCGATGACTGA |
| | ACCCGTGGGCCCCAGCTTTATTAGCCTACCAGTCGATCTG |
| | CTGGGGTCGAGCGAAGGGATTGACACAACAGTGCCGAAC |
| | CCGCCGGCGAATACCCCGGCTAAACCGGTGGGCGTGGTA |
| | GCTGATGGCTGGCAGAAAGCGGCAGATCAAGCTGCTGCG |
| | CTTTTGGCAGAGGCCAAACATCCAGTATTAGTGGTGGGTG |
| | CAGCGGCGATCCGTAGCGGAGCTGTTCCTGCAATTAGAG |
| | CTTTGGCAGAACGTTTGAACATCCCCGTCATCACCACCTA |
| | TATCGCTAAAGGTGTCCTGCCGGTTGGTCATGAACTGAAT |
| | TACGGTGCTGTCACCGGCTATATGGATGGCATCCTGAACT |
| | TCCCAGCGCTGCAAACCATGTTTGCTCCGGTGGATTTAGT |
| | ACTGACCGTGGGTTATGATTATGCAGAAGATCTGCGACCT |
| | TCGATGTGGCAAAAAGGTATCGAAAAAAAGACAGTTCGA |
| | ATTTCGCCGACTGTGAACCCCATCCCTCGGGTCTATCGTC |
| | CGGACGTGGACGTCGTGACCGACGTGCTGGCTTTTGTGGA |
| | ACACTTTGAAACCGCGACCGCGTCCTTCGGTGCGAAACA |
| | GCGACACGACATCGAACCCTTGCGTGCACGTATTGCAGA |
| | ATTCTTGGCGGACCCGGAAACCTATGAGGATGGAATGCG |
| | AGTCCATCAGGTAATCGATTCTATGAACACCGTCATGGAA |
| | GAGGCGGCAGAGCCAGGCGAAGGCACCATTGTTAGTGAT |
| | ATTGGGTTCTTCCGCCACTATGGTGTCTTGTTTGCTCGTGC |
| | GGACCAACCCTTTGGGTTCCTGACCTCTGCGGGTTGTTCA |
| | TCTTTTGGATACGGTATTCCAGCGGCTATCGGAGCACAGA |
| | TGGCCCGTCCGGATCAACCTACATTTTTAATTGCAGGCGA |
| | TGGCGGTTTTCACTCTAATTCG AGCGACCTGGAAACCATT |
| | GCTCGCCTTAACCTGCCGATCGTGACGGTTGTCGTGAACA |
| | ATGACACGAACGGCCTGATTGAACTGTACCAGAATATCG |
| | GTCATCATCGCAGTCATGATCCAGCCGTAAAGTTCGGGGG |
| | TGTCGATTTTGTGGCGCTGGCGGAAGCAAACGGCGTTGAT |
| | GCGACCCGGGCAACCAATCGTGAGGAGCTGCTTGCGGCG |
| | TTGCGTAAAGGCGCAGAACTGGGTCGTCCGTTCCTGATCG |
| | AAGTACCGGTAAACTATGACTTTCAGCCGGGTGGCTTTGG |
| | CGCTCTGTCTATT |
| | (SEQ ID NO. 22) |
| A0A016CS86_BACFG | ATGCTGAGCCCCAAATTCTTTGTCGAAACCCTGCAAACCT |
| | ATTCCATGGACTTTTTTACGGGCGTGCCCGATTCGCTGTT |
| | GAAAAACATGTGCGCCTATATAACTGATCATATTGAATCA |
| | CAGAACAACATTATCGCAGTTAATGAAGGCACTGCGCTT |
| | GGGCTGGCGGCGGGTTACTACATCGCAACCGGTTGCATCC |
| | CGATTGTATATATGCAGAACAGTGGGATTGGTAACACTGT |
| | AAATCCTCTTTTGAGTTTGACGGACAAAGTTGTGTACAAC |
| | ATCCCGGTGCTTCTCCTTATTGGCTGGCGCGGCGAGCCGG |
| | GCATTAAGGATGAACCGCAGCATATCAAACAGGGGATGA |
| | TCACCATCCCGTTGCTGGATACACTAGGCATTAAAAACCA |
| | AATTCTCAATAAGGACCCAAACATGGCCAAATCACAAAT |
| | TAACGATGCCATCGAGTACATGCGGATGACGAAAGAGGC |
| | ATTCGCCTTTGTAATTCAGAAAGACACTTTCGAGGAATAC |
| | AAACTGCAAAACACCGAAGACAGCAAGTTCGACCTGGAC |
| | CGCGAAGAGGCGATTAAAATCGTGTGTAATTCCTTAGAC |
| | AAAGGCTCCGTGATTGTGAGTACGACCGGCATGATCTCGC |
| | GTGAATTATTCGAGTACCGCGAAAGCATCGATGCTAACC |
| | ATGAAACTGACTTCCTCACAGTCGGTTCCATGGGTCACGC |
| | CAGTCAAATCGCTCTGGGCATCGCACTGCGCCGTAAAAA |
| | CAAAAAAGTCTACTGTTTCGATGGCGATGGAGCCGTCTTA |
| | ATGCATATGGGCGCCTTAACGACAATTGGCACGAGCCGC |
| | GCTGTCAACTACATCCACATTGTGTTCAACAATGGGGCAC |
| | ACGATAGCGTAGGGGGCCAGCCGACGGTTGGCCTCAAAG |
| | TAAACCTGAGTAAAATTGCAAGCGCGTGCGGTTACAACA |
| | ATGTAATCTCCGTGGATTCTAAGGCAACATTGAAAGAAA |
| | GCCTCGATCGTTTTAAATCAATAAATGGTCCGGTATTGCT |
| | CGAAGTTAAGGTACGCAAAGGCGCGCGTAAAGACCTGGG |
| | TCGCCCGACCTTAACACCGGTTAAAAACAAGGAACTGCT |
| | GATGAACTTTCTGGAAGAAGCTGATGAAAGCGATAAAAG |
| | CGATAATGTTTTCAAA |
| | (SEQ ID NO: 24) |
| A0A0F2PQV5_9FIRM | ATGATTAGCACTAAACGCTTTGGTGAAGAACTAAAAAAA |
| | CTGGGCTTTGATTTCTATTCCGGCGTTCCTTGCAGCTTCCT |
| | GAAAAACCTAATCAATTACACCACGAATCACTGTAACTA |
| | CCTGGCCGCTACCAACGAGGGAGAGGCAGTCGCGGTTGC |
| | CGCGGGTGCGTTCCTGGCCGGCAAAAAACCGGTTGTGCT |
| | GATGCAAAACTCCGGGTTGACGAATGCCGTCTCTCCCCTT |
| | GTAAGCCTGAACTATCTCTTCCGCTTACCGGTGCTGGGTT |
| | TTGTCTCCCTTCGCGGTGAACCTGGTATCCCAGACGAGCC |
| | GCAACACCAGCTCATGGGCCGTATTACCACCCAAATGCTT |
| | GATCTGGTTGAAATTCAGTGGGAGTATCTCTCCACAGATT |
| | TTGATGAGGTGAAAAAACAGCTGTTACAGGCATACAGCT |
| | GTATTGAATCAAATCAACCGTTCTTTTTCGTGGTAAAAAA |
| | AGATACCTTTGAAAAAGAACAGTTAACCGACTCTCAGAA |
| | ACGTCTGAGCAAAAACATGTTTAAATCGGAACGCACCAA |
| | AGCGGATCAGGTGCCCAAAAGATTTGAAACCCTGCGGCT |
| | AATAAACTCCCTGAAAGATGTGAAGACCGTGCAGCTCAC |
| | TACGACGGGCATTACCGGCCGTGAACTATACGAAATTGA |
| | AGATCATCAGCAATAACCTATATATGGTAGGTAGTATGGG |
| | CTGTGTCAGTTCGCTGGGCCTGGGACTGGCGCTGACTAAA |
| | AAAGACAAAGATGTGGTTGTTATCGAAGGTGATGGCGCC |
| | CTGCTGATGCGGATGGGTAACCTTGCGACGAACGGTTACT |
| | ACGGTCCGCCGAATATGCTGCACATTTTGCTGGATAATAA |
| | TATGCATGAATCCACTGGAGGTCAGAGTACCGTTAGCTAC |
| | AACATCAATTTCGTTGACATTGCTGCCGCGTGCGGTTATA |
| | CTAAATCCATCTATGTGCATAACCTGGTGGAACTCGAGTC |
| | GCATATCAAAGATTGGAAACGGGAGAAAAATCTCACGTT |
| | TCTCTATCTGAAAATCGCCAAGGGTAGCATTGAAGGACTG |
| | GGCCGTCCAAAAATGAAACCTCACGAGGTGAAAGAACGT |
| | TTAAAAGTATTCTTGGATGGT |
| | (SEQ ID NO: 26) |
| D7DTG5_METV3 | ATGAAAACCATCGTTATTCTGCTCGATGGGGTTGCGGATC |
| | GTCCTTCCAAAGAACTGAATTATAAAACTCCGCTTCAATA |
| | CGCGAACATCCCGAATCTCGACGAATTCGCTAAGTCTTCC |
| | TTAACGGGCCTCATGTGTCCCCAGAAAATTGGGGTTCCAC |
| | TGGGCACGGAAGTCGCTCATTTCTTGCTGTGGGGCTACGA |
| | TATTAGTCAGTTCCCCGGACGGGGGGTGATCGAAGCGCT |
| | GGGTGAAGGCATTGACCTGAAAAAAGATTCGATTTACCT |
| | GCGCGCTACCCTCGGTCATGTGAACTATAATCAGAAGGA |
| | GAACAACTTCCTTGTGTTGGATCGTCGGACCAAAGACATT |
| | AACAATCAAGAGATCTCAGAGCTGCTCAACAAAATTTCC |
| | AACATTAACATTGATGGTTATCTGTTTACCATTCATCACA |
| | TGCAGGGTATCCACAGTATTCTGGAAATTTCTAAGCTGGA |
| | GAATGACGGTAATCTGAAAACCGAACCGAACTTGAAGAA |
| | AAACAATCTGAAAAAAAATGGCTTCGAACTGACCTATGA |
| | AGAATTTTGCAACGAGAAAAATATTCTGAAGTATGGCAA |
| | TATTAACAACATCAATAATTGCATCTCTAACAAAATTTCG |
| | GATTCAGACCCGTTTTACAAGGATCGCCACGTGATAATGG |
| | TTAAACCAGTAATTAAACTGATTGGTACCTACGAAGAATA |
| | TCTGAACGCCCTGAATGTAAGCAACGCGCTGAATAAATA |
| | TCTGACAACGTGTAACACCCTGCTGGAAAATGACAGCAT |
| | CAATATTTCACGTAAAAATGAGAATAAATCTCTGGCAAAT |
| | TTTCTGCTGACTAAATGGGCGGGCAGCTATAAAAAGCTGC |
| | CTAGCTTTAAACAGAAATGGGGCTTAAATGGTGTGATTAT |
| | TGCTAACAGTTCTCTGTTCCGTGGTCTGGCCAAACTCCTC |
| | AAAATGGACTATTATGAGGTGAAAGAGTTCGACAAGGCA |
| | ATTGAACTGGGGCTGAAGTTCAAGAACGATAACACGAAC |
| | AATAATAACAACTCCAACAATAACAACAACAACAATCAG |
| | AACAACAATATCAACAATAAGAAGATCTACGACTTTATC |
| | CATATCCATACGAAAGAACCTGATGAGGCCGGGCATACC |
| | AAGAATCCGATCAACAAGGTACGCGTGCTGGAAAAACTC |
| | GATAAAAATTTAAAAGTAGTTATTGATGAGATCGATAAA |
| | GAGAAGGAAAACGGCGATGAAAACCTTTACATTATTACC |
| | GGTGACCACGCGACACCATCGACGGGCGGTCTGATCCAT |
| | TCGGGCGAACTGGTTCCAATTGCAATTTGTGGCAAGAACG |
| | TTGGTAAAGACTCTACGAAGGCGTTTAACGAAATGGACG |
| | TACTGAACGGCTATTACCGGATCAATTCAACCGATATCAT |
| | GAACCTGGTGCTTAACTATACGGATAAAGCCCTCCTGTAT |
| | GGACTCCGTCCAAACGGGGATCTTAAGAAATATATTCCTG |
| | AAGACAATGAACTGGAATTCCTCAAAAAAGATAAC |
| | (SEQ ID NO: 28) |
| 3E9Y | ATGGCGGCTGCTACCACCACTACCACAACATCTTCGTCTA |
| | TATCCTTTTCTACTAAACCGAGCCCTTCTTCTTCCAAAAGT |
| | CCACTGCCCATTTCACGCTTCTCCTTACCGTTTAGCCTGAA |
| | CCCCAACAAGAGCTCGAGCAGCTCACGCCGCCGCGGTAT |
| | TAAATCATCGAGCCCGTCTAGCATATCCGCGGTTCTCAAC |
| | ACCACTACCAACGTTACGACCACTCCTAGCCCGACCAAAC |
| | CCACTAAACCGGAAACCTTTATTTCGCGATTCGCTCCGGA |
| | CCAGCCTCGTAAAGGTGCGGATATTCTTGTGGAAGCGCTG |
| | GAACGCCAGGGCGTGGAAACCGTGTTTGCTTACCCGGGT |
| | GGCGCTTCCATGGAGATACATCAGGCCTTGACACGGAGTT |
| | CATCTATCCGAAATGTTCTGCCGCGTCATGAACAGGGCGG |
| | TGTATTTGCAGCGGAAGGGTACGCGCGCTCCTCTGGCAAA |
| | CCAGGCATCTGCATTGCGACCTCAGGCCCCGGTGCTACCA |
| | ATCTCGTTAGCGGCCTGGCAGATGCGTTACTGGATAGCGT |
| | GCCGTTAGTCGCGATTACCGGTCAGGTGCCACGTCGTATG |
| | ATCGGCACTGATGCGTTCCAGGAAACACCTATAGTAGAG |
| | GTGACCCGTTCAATCACGAAACATAACTATTTGGTGATGG |
| | ATGTAGAGGACATCCCGCGCATTATTGAAGAAGCGTTTTT |
| | TCTAGCCACTTCTGGTCGCCCAGGCCCGGTCCTGGTAGAT |
| | GTGCCCAAAGATATCCAACAGCAGCTGGCGATCCCGAAT |
| | TGGGAGCAGGCAATGCGCCTCCCCGGGTACATGTCGCGA |
| | ATGCCGAAACCGCCGGAAGATTCTCATTTAGAACAGATT |
| | GTGCGTTTAATTTCGGAATCGAAAAAACCGGTTCTGTATG |
| | TTGGCGGTGGCTGCTTGAATTCATCAGATGAACTGGGTCG |
| | TTTCGTAGAACTCACCGGCATTCCGGTAGCGTCAACCCTG |
| | ATGGGCCTGGGTTCCTATCCGTGCGATGACGAGCTCTCGC |
| | TGCATATGCTCGGAATGCACGGTACCGTGTACGCCAATTA |
| | CGCTGTGGAACACAGTGACCTTCTGCTGGCGTTTGGTGTA |
| | CGTTTTGATGATCGTGTCACCGGCAAGCTGGAGGCGTTCG |
| | CGTCGCGCGCGAAAATTGTCCACATTGATATTGATTCTGC |
| | GGAGATTGGGAAAAACAAAACCCCGCACGTCTCCGTGTG |
| | CGGGGACGTTAAGCTCGCACTTCAGGGCATGAATAAAGT |
| | TCTGGAAAACCGTGCAGAAGAACTGAAACTGGATTTCGG |
| | CGTGTGGCGTAACGAACTTAATGTACAGAAGCAGAAATT |
| | TCCGCTGTCTTTTAAAACGTTTGGTGAAGCAATCCCGCCC |
| | CAGTACGCCATCAAAGTCCTTGACGAATTAACCGACGGT |
| | AAGGCAATCATAAGCACCGGTGTGGGTCAACATCAGATG |
| | TGGGCGGCTCAATTTTATAATTATAAAAAACCTAGACAGT |
| | GGCTCTCGTCAGGCGGCCTGGGTGCCATGGGCTTTGGACT |
| | GCCTGCCGCAATCGGCGCAAGTGTAGCGAACCCGGACGC |
| | TATCGTGGTGGATATCGACGGCGATGGTAGTTTTATTATG |
| | AACGTCCAGGAGCTGGCCACCATCCGCGTAGAGAACCTG |
| | CCCGTAAAAGTTTTATTGTTAAACAACCAGCATTTAGGTA |
| | TGGTGATGCAATGGGAAGATCGTTTCTACAAGGCCAATC |
| | GCGCGCACACCTTTTTAGGCGATCCTGCGCAGGAAGATG |
| | AGATTTTTCCTAACATGCTGCTTTTCGCCGCAGCTTGCGG |
| | CATCCCCGCCGCGCGAGTAACCAAGAAAGCAGATCTCCG |
| | TGAAGCCATCCAGACTATGCTCGATACCCCCGGTCCGTAT |
| | CTGCTTGACGTGATTTGTCCGCATCAAGAACACGTTCTTC |
| | CGATGATTCCGAGCGGCGGCACCTTTAATGATGTGATCAC |
| | GGAAGGGGACGGTCGCATTAAATAT |
| | (SEQ ID NO: 30) |
| 2ZKT | ATGGTTCTGAAACGTAAAGGGCTGCTGATTATCTTGGATG |
| | GTCTGGGTGATCGTCCGATCAAAGAATTAAACGGCTTAAC |
| | TCCGTTGGAATATGCCAACACCCCAAATATGGATAAACTG |
| | GCGGAAATCGGCATTCTAGGCCAGCAGGATCCGATCAAA |
| | CCAGGCCAGCCGGCCGGCTCTGACACTGCGCACCTGTCA |
| | ATCTTTGGCTATGATCCCTATGAAACTTACCGTGGGCGGG |
| | GCTTTTTTGAAGCATTAGGGGTGGGCCTTGATCTGAGTAA |
| | AGACGATCTGGCCTTTCGTGTGAATTTTGCCACGCTCGAA |
| | AATGGGATTATTACGGATCGTCGCGCAGGCCGTATTAGCA |
| | CAGAGGAAGCGCACGAACTGGCGCGGGCGATTCAGGAGG |
| | AAGTGGACATTGGGGTTGACTTCATTTTCAAAGGCGCGAC |
| | CGGCCATCGTGCAGTGCTCGTTTTAAAAGGTATGTCTCGT |
| | GGTTATAAAGTGGGTGATAACGATCCGCATGAAGCTGGT |
| | AAACCGCCGTTAAAGTTTTCATATGAAGACGAGGATTCA |
| | AAGAAAGTAGCCGAAATTCTCGAAGAATTCGTGAAAAAA |
| | GCGCAGGAAGTTCTTGAAAAACACCCAATTAATGAAAGA |
| | CGCCGCAAGGAGGGCAAACCGATCGCGAACTATTTGCTG |
| | ATTCGCGGGGCTGGGACGTATCCGAACATACCGATGAAA |
| | TTCACCGAGCAGTGGAAAGTGAAGGCGGCCGGCGTAATT |
| | GCAGTGGCGCTGGTTAAAGGCGTAGCACGTGCAGTCGGC |
| | TTCGACGTATATACCCCTGAAGGGGCGACCGGAGAGTAC |
| | AACACGAACGAAATGGCCAAAGCAAAAAAAGCAGTAGA |
| | ACTGCTAAAAGATTATGATTTTGTGTTCTTACACTTCAAA |
| | CCGACTGATGCCGCGGGGCACGACAACAAACCGAAGCTG |
| | AAAGCGGAATTGATTGAACGCGCCGATCGCATGATTGGG |
| | TATATCTTGGATCATGTTGACTTAGAAGAAGTTGTAATCG |
| | CTATCACCGGCGATCATTCGACGCCATGCGAGGTAATGA |
| | ATCATAGCGGGGACCCTGTCCCACTTTTGATTGCGGGTGG |
| | CGGCGTGCGCACGGACGATACCAAACGTTTCGGCGAGCG |
| | CGAGGCAATGAAAGGCGGCCTTGGCCGCATCCGTGGCCA |
| | CGATATTGTTCCTATCATGATGGATCTAATGAATCGTTCG |
| | GAAAAATTTGGTGCG |
| | (SEQ ID NO: 32) |
| A0A124FLS8_9FIRM | ATGCTGCTGGTTGTTCTGGATGGTCTGGGCGGCCTTCCGG |
| | TGCCTGAACTGAATGGGCGTACGGAACTTGAGGCGGCCG |
| | CGACACCGAACTTAGATGCGCTGGCGAAGCGCTCTTCCCT |
| | GGGCCTGGCACATCCGGTGCTGCCGGGCATAGCGCCTGG |
| | TTCTTCTGCTGGGCATCTGGCTCTTTTCGGTTACGATCCGT |
| | TGCGTTATGTCATTGGCCGCGGCGTCCTGGAGGCCCTGGG |
| | CATTGGTTTCGACCTCCATCCCGGTGATGTGGCCGTCCGT |
| | GCTAATTTCGCAACCGTCCAAGACACGCGGAACGGTCCA |
| | GTCGTGACGGATCGACGTGCGGGCCGTCCGCCGACGGAA |
| | CATACTCGTAGTATCTGTCGTCGCCTGCAGGACGCAATTC |
| | CGGAGATTGACGGTGTACGTGTCTTCATTGAGCCGGTTAA |
| | AGAACATAGATTCGTGATTGTGCTGCGAGGCGAAGGTCT |
| | GGATGATCGCGTCGCCGACACGGATCCCCAACGTGAAGG |
| | GATGCCTCCGTTACAACCGCAACCGCTTGCTGAAGAAGCT |
| | CGTCGCACAGCGATGCTGGCGGGAACCCTGGTGCAACGG |
| | ATTGCTGAGTTAGTCCGCGATGAGCCTCGTACTAATTTTG |
| | CTCTGCTGCGCGGGTTCTCTCGCCGTCCTCGCCTGGACCC |
| | GTTCCCAGAACGTTATCGTGCCCGCGCAGGAGCAGTGGC |
| | AGTCTATCCGATGTATCGCGGTCTGGCATCCCTGGTCGGT |
| | ATGGATCTGCTGCCAGTCGCCGGGGATACGCTTGCCGACG |
| | AAATTGCGAGCCTCAAGGAAAACTGGCCTGAGTATGATT |
| | ACTTCTTTCTGCACGTTAAAGGCACGGACAGTCGCGGTGA |
| | AGATGGTGATTGGGCAGGCAAAATCAAGATTATTGAGGA |
| | ATTTGACGCCCAGCTGCCTGCAATTCTAGATTTAAATCCC |
| | GATGCGTTGGTGATTACAGGCGATCACAGTACGCCTGCTA |
| | CGTACGCGGCCCATAGCTGGCATCCTGTGCCTTTTCTGTT |
| | GTACAGCCGCTGGGTCCTGCCGGATCGCGATGCGCCAGG |
| | TTTCGGCGAACACGCATGCGCCCGTGGAGTGCTGGGTCA |
| | GTTCCCGCTGTTGTATACGATGAATCTTTTGTTGGCCAAT |
| | GCTGGGCGTCTCGGCAAATTCAGCGCC |
| | (SEQ ID NO: 34) |
| 4WBX | ATGAATAAACGGTTTCCGTTCCCGGTGGGAGAACCTGATT |
| | TTATTCAGGGTGATGAGGCTATCGCTCGTGCAGCCATTTT |
| | AGCCGGATGTCGTTTTTATGCGGGATACCCGATCACGCCC |
| | GCGTCGGAAATCTTCGAAGCGATGGCACTATATATGCCGC |
| | TGGTCGATGGCGTAGTTATCCAGATGGAAGATGAGATTG |
| | CGTCGATCGCGGCCGCCATCGGGGCAAGTTGGGCTGGTG |
| | CTAAGGCGATGACCGCTACCTCTGGGCCCGGATTCAGCCT |
| | GATGCAAGAAAACATTGGTTACGCGGTTATGACAGAAAC |
| | GCCTGTGGTTATAGTCGACGTGCAGCGTAGCGGTCCAAGC |
| | ACGGGACAACCGACCCTGCCTGCGCAAGGCGATATTATG |
| | CAGGCGATTTGGGGCACGCATGGCGACCACAGCCTGATA |
| | GTTCTGTCACCGTCGACGGTCCAGGAGGCGTTCGATTTTA |
| | CGATTCGTGCGTTCAACCTGTCCGAAAAGTACCGTACCCC |
| | GGTCATCCTGCTCACCGATGCCGAAGTGGGACATATGCG |
| | GGAACGTGTTTATATCCCGAACCCAGATGAAATCGAAATT |
| | ATTAATCGTAAGCTGCCGCGCAACGAAGAGGAAGCAAAA |
| | TTACCGTTCGGTGATCCGCACGGCGATGGGGTTCCCCCCA |
| | TGCCTATTTTCGGGAAAGGTTACAGGACGTATGTGACCGG |
| | CCTGACCCATGATGAAAAAGGTCGCCCACGCACAGTCGA |
| | TCGTGAAGTGCATGAACGCCTGATTAAACGTATAGTTGAA |
| | AAAATAGAAAAGAACAAGAAAGATATCTTTACGTACGAA |
| | ACGTATGAGCTGGAAGATGCCGAAATTGGAGTGGTTGCA |
| | ACGGGTATTGTGGCCCGTTCGGCCTTACGTGCTGTCAAAA |
| | TGCTGCGCGAAGAGGGCATCAAAGCGGGCCTGTTGAAAA |
| | TTGAAACTATTTGGCCGTTTGACTTCGAATTAATCGAGCG |
| | TATTGCGGAACGCGTGGATAAACTGTATGTACCGGAAAT |
| | GAACTTAGGGCAGCTGTATCACCTGATTAAGGAAGGCGC |
| | GAACGGCAAAGCGGAAGTTAAATTAATCAGCAAGATCGG |
| | TGGAGAAGTGCATACCCCGATGGAGATCTTTGAATTTATT |
| | CGTCGCGAATTCAAA |
| | (SEQ ID NO. 36) |
| C4L9G3_TOLAT | ATGACCGAACAGTGGCAGTCCCTCGATTCTCTGAATGCCT |
| | TGTGGTCTGCGCTGTTGATTGAAGAGCTCGCACGCCTGGG |
| | GATTCGGGATATTTGTATTGCCCCAGGCAGCCGCTCAACC |
| | CCTCTTACTCTGGCCGCCGCTGCTAACCCGGCGATCTCAA |
| | CTCATTTGCATTTTGACGAACGCGGGTTAGGTTTTCTTGCC |
| | CTGGGGTTGGCGCAGGGGAGCCAGCGTCCGGTCGCGGTT |
| | ATCGTGACGTCTGGAAGCGCGGTCGCAAACCTGCTGCCC |
| | GCTGTCGTCGAAGCACGCCAGAGTGGCATTCCGCTTTGGT |
| | TACTGACGGCGGATCGCCCAGCAGAATTGCTCGGTTGCG |
| | GCGCCAATCAGGCGATCACGCAGGCAAACATATTTGCGA |
| | ACTATCCAGTGTATCAGCAACTGTTTCCTGCTCCGGATCA |
| | TGATATTACTCCTAGCTGGCTGCTGGCGAGTGTGGACCAG |
| | GCAGCTTTCCAGCAGCAACAGACGCCGGGACCCGTACAT |
| | CTGAACTGTCCGTTCCGAGAACCACTGTACCCGGTCGCGG |
| | GCCAGCAGATTCCGGGTAATGCACTGCGCGGTCTGACCC |
| | ACTGGTTACGCTCTGCGCAACCGTGGACACAGTATCATGC |
| | GGTCCAACCTATCTGCCAAACCCACCCGCTTTGGGCAGAA |
| | GTGCGCCAGAGCAAAGGCATTATTATTGCGGGCCGACTG |
| | TCACGTCAGCAAGATACCGGTGCCATCCTGAAACTGGCTC |
| | AACAGACCGGCTGGCCGCTGTTGGCTGATATTCAGTCGCA |
| | GCTGCGTTTTCATCCGCAGGCCATGACGTACGCGGATCTG |
| | GCACTCCATCATCCGGCGTTTCGTGAAGAACTAGCGCAGG |
| | CAGAAACCCTCTTACTGTTTGGTGGTCGACTGACTTCGAA |
| | ACGCCTGCAACAATTTGCAGATGGCCACAATTGGCAGCA |
| | TTGCTGGCAGATTGACGCCGGGTCAGAGCGGCTGGACTC |
| | GGGTCTTGCGGTCCAACAGCGTTTTGTGACTTCTCCAGAA |
| | CTGTGGTGCCAGGCGCATCAGTGTGAGCCGCATCGTATCC |
| | CGTGGCACCAACTGCCACGGTGGGACGGTAAACTGGCAG |
| | GTCTGATTACCCAGCAGCTGCCGGAGTGGGGTGAGATTA |
| | CACTATGCCATCAGCTGAACTCACAGTTACAAGGCCAGTT |
| | ATTCATCGGGAATTCGATGCCAATCCGCCTGCTGGATATG |
| | CTCGGCACCAGCGGCGCGCAGCCATCGCATATTTACACTA |
| | ACCGGGGCGCAAGTGGCATTGACGGGCTAATCGCCACGG |
| | CCGCGGGTATCGCCCGTGCGAATACAAGCCAGCCGACGA |
| | CCCTGCTTCTGGGGGACAGCAGCGCCCTGTACGACTTGAA |
| | CAGCCTGGCACTATTACGCGAACTGACCGCTCCGTTCGTA |
| | CTGATCATAATCAATAATGACGGCGGCAATATCTTTCATA |
| | TGCTGCCGGTTCCAGAGCAGAATCAGATTCGCGAACGGTT |
| | CTATCAGCTGCCGCATGGCCTGGACTTTCGCGCTAGTGCC |
| | GAACAATTCCGATTAGCGTATGCCGCGCCCACCGGAGCC |
| | ATCTCCTTTCGTCAAGCGTACCAACAAGCCCTGAGCCATC |
| | CGGGGGCGACACTGCTGGAGTGCAAAGTTGCCACGGGCG |
| | AAGCCGCAGATTGGCTCAAAAATTTTGCGCTCCAAGTCCG |
| | CAGTCTTCCGGCG |
| | (SEQ ID NO: 38) |
| A0A0K1FGX4_9FIRM | ATGAATGCTAACGATCTCATTGCGGCACTGGGTGCCGAAT |
| | TCTTCACTGGCGTTCCCGATTCTAAATTGCGCCCGTTGGTT |
| | GATTGCCTGATGGATACCTATGGCGCTAATTCACCAAGCC |
| | ACATCATTGCGGCCAACGAGGGGAATGCCGCGGCTCTGG |
| | CCGCTGGCTACCACTTAGCTGCAGGTAAAGTTCCTCTGGT |
| | TTACCTGCAGAACAGTGGGTTGGGTAATATCGTCAATCCG |
| | TTGTTATCATTACTGCATGCGGAAGTATATGGCATTCCGT |
| | GCATCTTCGTGATTGGTTGGCGCGGTGAACCTGACTTACA |
| | TGACGAACCGCAACACCTGGTCCAGGGTCGTTTGACCCTT |
| | CCGTTACTGGAAACCATTGGCGTGAAAACAATGGTACTG |
| | ACCGAAGCGAGCCAGCCGGAAGATGTCTCCGCCTGGATG |
| | GAACAAATTCGTCCGCATCTGGCAGCGGGGGGCCAGTGC |
| | GCCTTGCTGGTGCGCAAGGGCGCGCTGACTCATCCGAAA |
| | CACAAATATGCAAACGAAAACCCCCTGCGTCGCGAGGAT |
| | GCAATCGCACGGATCCTCGATGCAGCGCAGGGCGCTGTT |
| | GTTGTGGCCACCACCGGCAAAACCGGTCGTGAACTGTTTG |
| | AACTGCGCGCCGCCCGCGGCGAAGACCATGCCCATGATT |
| | TCCTGACCGTGGGTAGTATGGGTCACGCCGGTGCAATCGC |
| | ACTGGGTATTGCCCTGCACCGGCCGTCCCAACGCGTATTT |
| | TTACTGGATGGGGATGGCGCGGCCCTGATGCATATGGGT |
| | GCGATGGCAACCATTGGTGCAGCGGCACCCGCCAACATC |
| | GTGCACGTCCTGCTGAATAACGAAGCGCATGAATCTGTG |
| | GGCGGCGCACCAACCGCAGCTCACACCGTCGATTTTCCGG |
| | CGGTAGCCCGCGCCGTGGGCTACCGTTTAGTACAGACTGC |
| | GGCGGATGCCGCAGAACTGGCGCAGATTCTGCCAGCAGT |
| | GGGCCGCAGCGACGCCCTGACGTTCTTGGAAGTTCGTACT |
| | GCTATTGGTTCACGCGCAGACCTGGGTCGTCCTACTACTA |
| | CCCCAACCGAAAACAAAGAGGCACTTATGCGTACGCTGC |
| | GCGAA |
| | (SEQ ID NO: 40) |
| A0A0R2PY37_9ACTN | ATGGCGAGCTCTGAGAAAATGCGCGTAGGCGAAGCGATT |
| | ATAGATCTGCTGGTGCGCGAATATGAACTAGATACCGTGT |
| | TCGGGATTCCCGGAGTGCACAACATTGAGCTGTTTAGAGG |
| | CTTACATAGCTCTGGTGTGCGCGTCGTTGCGCCTCGCCAT |
| | GAACAAGGTGCAGGCTTTATGGCGGACGGCTGGAGCATT |
| | GCTACAGGCAAACCTGGTGTCTGCGCCTTGATAAGTGGGC |
| | CGGGCTTAACCAATGCAATAACCCCGATAGCGCAAGCGT |
| | ACCACGATAGTCGCGCGATGTTAGTCCTGGCGAGTACTAC |
| | GCCGACGCACAGCCTGGGCAAAAAATTTGGCCCATTACA |
| | CGATCTTGACGATCAGTCCGCCGTGGTGCGTACCGTGACT |
| | GCTTTTTCAGAGACTGTTACAGATCCTACGCAGTTCCCAC |
| | AGCTGATTGAACGGGCGTGGAATGTTTTCACATCATCTCG |
| | TCCGCGTCCAGTTCATATCGCAATCCCGACCGACGTGCTG |
| | GAGCAGTTTGTGGATCCGTTTACGCGAGTGACCACCGATA |
| | TTTCGAAACCAGTGGCCCAGGACTCCGATATTCAAAGAG |
| | CGGCGCAGCTCCTAGCAGCGGCCAAACGTCCCATGATCA |
| | TTGCGGGCGGAGGCGCTCTGGGCACAGGTGCATTGATCTC |
| | GAACATTGCCACAGCTATTGATAGCCCGATCGTGTTGACC |
| | GGTAATGCGAAGGGTGAGGTACCGAGTACCCACCCGTTA |
| | TGTGTCGGCTCTGCTATGGTTATTCCACGCGTGCAGGAAG |
| | AAATCGAACAAAGTGATGTCGTTTTGGTGATTGGCAGCG |
| | AAATCTCTGATGCAGACCTGTACAACGGTGGTCGCGCCCA |
| | GGGATTTTCTGGTAGCGTTATCCGCATCGACATTGATACC |
| | GAGCAGATTAGTCGTCGAGTGGCCCCGCACGTCAGCCTG |
| | GTGGCTGATGCGGCGGATTCCTTGTCACGTATTTCTGCCG |
| | AACTGACAAAGGCCGGTGTGGCGCTGACGAATTCTGGCA |
| | GCGCACGTGCGACGAATTTACGTATGGCAGCCCGTAGCG |
| | GCGTGCGACAAGACCTGCTGCCGTGGATCGATGCCATTG |
| | AACAATCCGTGCCGGACAACACGCTGGTGGCGGTAGATT |
| | CAACCCAGCTGGCGTATGCGGCGCATACAGTCATGAGTT |
| | GTAATTCTCCGCGTTCTTGGTTAGCGCCATTCGGCTTTGGT |
| | ACGCTTGGTTGTGCCCTTCCAATGGCGATCGGCGCCGCAA |
| | TCGCGGATACGACCCGTCCAGTCCTGGCCATTGCGGGCGA |
| | TGGTGGTTGGCTGTTTACCTTAGCCGAAATGGCGGCAGCA |
| | ATCGACGAAGGCATTGATATGGTTCTTGTACTGTGGGATA |
| | ATCGCGGCTATGGACAAATCCGTGAAAGCTTCGACGATG |
| | TGCGAGCACCCCGTATGGGTGTAGATGTTTCAAGCCATGA |
| | CCCTTCCGCAATAGCCAACGGCTTCGGTTGGAACGCGATT |
| | GACGTGACCACCATTGAGGCGTTCCGAATTGTTCTGTCGG |
| | AAGCGTTTGAGAACCGTGGTGCTCACTTTATTCGTATTTC |
| | CGTGAGC |
| | (SEQ ID NO. 42) |
| X1WK73_ACYPI | ATGCAGGAAGCGGATTTTGAAGTGAATCATGCGCGTAAC |
| | GCGGACATTCCGATCGTCGGAGACGCGAAACAGACTCTG |
| | TCGCAGATGCTGGAACTCCTGGCGCAATCAGACGCTAAA |
| | CAGGAGCTTGACTCCCTGCGCGACTGGTGGCAGACCATTG |
| | ATGGATGGCGGAGTCGCAAATGCCTGGAATTTGATCGTA |
| | CGTCAGATAAGATCAAACCACAAGCGGTTATTGAGACGA |
| | TTTGGCGCCTGACCAAAGGCGATGCCTACGTGACTTCCGA |
| | TGTCGGCCAACACCAGATGTTCGCGGCACTGTACTACCAG |
| | TTTGATAAGCCGAGACGTTGGATTAACAGTGGTGGCCTTG |
| | GCACGATGGGTTTTGGGCTCCCGGCGGCGCTGGGTGTTAA |
| | AATGGCACTTCCCGATGAGACAGTAATCTGCGTTACGGGC |
| | GACGGTTCGATTCAGATGAATATCCAGGAACTGTCTACTG |
| | CGTTACAGTACGATTTGCCGGTACTGGTGCTGAACTTGAA |
| | CAACGGTTTTCTTGGCATGGTTAAACAATGGCAGGATATG |
| | ATCTATAGCGGCCGCCATAGCCAGAGCTACATGCAATCCC |
| | TTCCGGATTTCGTACGCCTGGCAGAAGCGTACGGGCATGT |
| | CGGGATAAGCATCGCGCACCCGGCTGAACTGGAAGAAAA |
| | ATTACAGCTGGCCTTAGATACGCTGGCAAAGGGGCGCCTT |
| | GTGTTTGTTGATGTCAATATTGACGGGAGTGAACATGTAT |
| | ATCCCATGCAAATCCGTGGTGGTGTTATTGTGAAGCTCGA |
| | TGAGATCGCACGCCTGGCAGGAGTATCTCGTACCACAGC |
| | CTCGTACGTCATTAATGGAAAGGCACGTCAGTACCGAGTC |
| | TCCGATAAAACGGTCGAAAAGGTGATGGCGGTGGTGCGC |
| | GAACATAACTATCATCCTAATGCTGTGGCTGCTGGTTTGC |
| | GGGCAGGACGTACTCGTAGCATTGGATTAGTAATCCCGG |
| | ATCTGGAAAACACATCATACACGCGCATTGCGAACTATCT |
| | GGAACGCCAGGCGCGCCAGCGCGGCTATCAGCTGTTAAT |
| | CGCTTGCAGCGAGGACCAGCCAGATAATGAAATGCGCTG |
| | CATCGAACACTTGCTGCAACGACAGGTGGACGCCATTATT |
| | GTCTCTACTTCCCTGCCCCCGGAACATCCGTTCTACCAAC |
| | GCTGGATCAACGATCCACTCCCGATCATCGCGCTGGATCG |
| | TGCGCTGGACCGCGAGCATTTTACGAGCGTAGTAGGGGC |
| | CGATCAGGACGATGCCCATGCCCTAGCCGCCGAACTTCGT |
| | CAGCTTCCGGTCAAAAACGTGCTGTTTCTGGGCGCCCTGC |
| | CGGAACTGAGCGTGTCGTTTTTGCGTGAAATGGGCTTCCG |
| | TGACGCCTGGAAAGATGATGAACGAATGGTCGATTACCT |
| | GTATTGTAACAGCTTCGATCGTACGGCCGCAGCTACCCTG |
| | TTTGAGAAATATCTCGAAGATCACCCGATGCCGGATGCGT |
| | TGTTCACTACCTCCTTCGGTTTGCTGCAGGGTGTGATGGA |
| | TATTACACTAAAACGCGACGGCCGCTTGCCGACCGATCTG |
| | GCGATCGCGACCTTTGGGGACCATGAATTATTGGACTTCT |
| | TGGAATGTCCGGTCCTGGCTGTGGGCCAACGCCACCGGG |
| | ATGTGGCGGAACGCGTCCTGGAACTGGTGCTGGCCAGCC |
| | TGGATGAACCGCGCAAACCGAAACCAGGTCTGACGCGCA |
| | TCCGTCGCAACCTGTTTCGGCGCGGCCAGCTTAGCCGTCG |
| | GACCAAA |
| | (SEQ ID NO: 44) |
| B1HLR4_BURPE | ATGAAAACCGAAGACCTGATAGGCATCCTGACGGATGCT |
| | GGTGTAGATCTCGCAGTCGGAGTCCCGGACAGCTTACTGA |
| | AAAGTTTTTGTGGTCGTCTGAATGACCCGGACTGCCCGCT |
| | ACGGCACCTGGTAGCATCATCAGAGGGTGGTGCCGTAGG |
| | GATTGCGATTGGTCACCATCTCGCCACCGGGGGCCTGGCC |
| | GCGGTATATATGCAAAACTCAGGTATCGGTAACGCCATC |
| | AACCCTCTTGTTTCGCTGGCAGACCGCGCTGTGTACGGCA |
| | TTCCGCTGGTTCTTATCGTGGGATGGCGTGCGGAAATCTC |
| | TGCCAGTGGCGCACAGGTACACGACGAGCCACAACACGT |
| | GACGCAGGGACGCATTACCTTACCGCTGCTGGACGCGCT |
| | GTCGATTCGCCACTTGGTTCTGGAACGCGCGGGAGGCGA |
| | AAATGACGCTCTGGCCCCCTCTATTGCGCGCTTGATTGCG |
| | GGCGCGCGTCAAACTAGCCAGCCGGTTGCTCTGGTGGTGC |
| | GTAAGGATGCGTTCGATGATGCTTCTGCAAGTCGTCCTGG |
| | CGCCGCTGCTCCACACGCAGGTCGCATGACCCGTGAACA |
| | AGCGATTGCCCTGATTGTTGAGCATGCGGACGCAGGTACC |
| | GCCATTGTAAGTACCACTGGCGTGGCATCGCGCGAACTTT |
| | ACGAATTACGCGACCGTTTAGGTCATTCCCATGCCCGCGA |
| | TTTTCTGACCGTCGGCGGCATGGGTCATGCCTCTCAGATC |
| | GCAGTGGGAATTGCGCTGGCACGCCCCGCGCAGAAAGTC |
| | ATTTGCATTGATGGTGATGGCGCACTGTTGATGCACATGG |
| | GTGGTCTGGCATATTGTGCGGGCGCCCCAAACCTGACACA |
| | CGTGGTGATTAATAACGGAGTTCATGATAGTGTCGGAGG |
| | CCAGCCGACCCTGGCTGCCCATTTGCGCCTGTCACACATC |
| | GCGGCAAGCTGCGGCTACGCATTTTCACGCAGCGTAGCA |
| | ACGCCTATAGAACTTGAATCAGCGCTGCACCACGCTAGC |
| | AGACTGGATGGCTCAGCGTTCATTGAAGTGACCTGTCGTC |
| | CGGGCTATCGCAGCGATCTGGGCCGTCCTCGTACGTCCCC |
| | GGCCGAAAATAAACGCCACTTTATGGCGTTCTTAAGCCGC |
| | AACGGGGCCACCCATGAGCGTGATGACCACGCACAGGAA |
| | TCGGGTATTCAAGACGCAGTGCAGTGCGCACGTCAT |
| | (SEQ ID NO: 46) |
| X8CA07_MYCXE | ATGCTGGCGAAACATGAGTTCTCCGCAGCGACCATGGCG |
| | GATGGTTACAGCCGTTGCGGTCAAAAACTGGGCGTAGTT |
| | GCGGCGACGAGCGGCGGTGCGGCACTGAACTTGGTCCCA |
| | GGCTTAGGTGAAAGCTTAGCGTCACGAGTGCCGGTGTTG |
| | GCGCTGGTGGGCCAGCCGGCGACCACCATGGATGGGAGA |
| | GGCTCCTTCCAGGACACGAGTGGCCGCAATGGCAGCTTG |
| | GACGCTGAAGCATTGTTCTCTGCCGTGTCCGTGTTTTGCC |
| | GTCGTGTACTTAAACCAGCTGACATTATTACTGCATTACC |
| | AGCAGCAGTTGCTGCGGCCCAGACCGGTGGTCCTGCAGT |
| | CCTGCTGCTTCCGAAAGACATTCAACAGACTCAAGTGGGC |
| | ATCAACGGTTACGCAGAACATGGCGTCGCGCCGAGTCGC |
| | TCAGTAGGCGATCCGCATTCAATTGTGCGTGCCCTTCGTC |
| | AGGTGACTGGGCCGGTGACTATAATTGCCGGGGAACAAG |
| | TGGCCCGTGATGATGCGCGCGCGGAACTTGAATGGTTGC |
| | GAGCTGTATTAAGAGCACGTGTTGCTTGTGTACCTGATGC |
| | AAAAGATGTTGCGGGGACGCCAGGCTTCGGTTCCTCTTCC |
| | GCGCTGGGCGTCACTGGTGTGATGGGTCATCCGGGCGTG |
| | GCTGACGCGCTGGCTAAAAGCGCCCTGTGTTTAGTTGTCG |
| | GTACGCGTTTGTCGGTCACAGCACGTACGGGCCTGGATGA |
| | TGCGCTGGCCGCTGTCCGCGTTGTGAGCATCGGTTCCGCG |
| | CCGCCGTACGTGCCATGTACGCATGTGCATACTGATGACC |
| | TGCGTGCTTCCTTACGACTGCTCACCGCGGCGTTATCAGG |
| | TCGCGGTCGTCCGACCGGGGTACGTGTTCCTGATGCGGTG |
| | GTGCGCACGGAACTGACTCCTCGTCGTAGCACCGTTCCGG |
| | CATGTGCCATTGCGACGCGT |
| | (SEQ ID NO: 48) |
| D1Y3P7_9BACT | ATGCAGATTTCGTCCTTCATTGCGCAGTTACAGCGCATCG |
| | CAAGCTCACATTTTTTAGGAGTGCCGGACAGCCAGCTCAA |
| | AGCTTTGTGTAATTATCTGTACAAAAACTGTGGCATCTCA |
| | AGTGACCACATCATTGCCGCGAACGAAGGCAACTGTACT |
| | GCGCTGGCTGCGGGGTATTACCTGGCTACGGGCAAGGTG |
| | CCGGTTGTTTACATGCAGAACAGCGGGTTAGGGAATGTTG |
| | TGAATCCGGTTGCGTCCTTGCTGAATGACAAAGTGTACGG |
| | GATCCCGTGTGTGTTTGTCATTGGCTGGCGGGGCGAGCCC |
| | GGCCTCAAGGACGAACCTCAACACATCTTCCAGGGCGCG |
| | GTGACTCTGGATCTGCTTAAAGTAATGGATATCGCGAGCT |
| | TCGTTGTCCGTAAAGATACCACGGAACAGGAATTAGCGG |
| | CCCAGATGGCTGAGTTTCAACCGCTGCTGGCGGCCGGCA |
| | AATCGGTTGCCTTCGTCATTGCAAAAGAAGCCCTGACGTA |
| | CGATGAGAAAGTAAGTTTTAAAAACGACTTCACTATGACT |
| | CGCGAAGAAGTGATTCGTCATATCACAGCGTTTTCCGGCG |
| | AAGACCCTATCGTGAGCACCACCGGAAAAGCTAGCCGCG |
| | AATTATTCGAAATTCGAGTCCGTAACGGTCAGCCCCACAA |
| | ATACGATTTCCTGACTGTGGGCTCTATGGGCCATAGCAGT |
| | TCTATTGCGCTGGGTATTGCACTATCGAAGCCCCACACGA |
| | AAATATGGTGTATCGATGGCGACGGTGCCGCCCTGATGC |
| | ATATGGGGGCCCTGGCGGTGATTGGTAGCCAACGTCCGC |
| | GCAATTTAGTCCATATTGTTATTAATAATGGTGCCCATGA |
| | GAGCGTTGGTGGTCTTCCGACCGTGGCACGGTCTGCGAGT |
| | CTGGCGAAAGTCGCAGAAGCCTGTGGTTATGTTAACGTA |
| | AAAACGGTGGGTACCTTTGCAGAGTTAGATGCAGCTTTAA |
| | AAGACGCCCGTAACGCCGATGAACTGACTTTTATAGAAG |
| | CCAAAACCGCGATCGGAGCCCGCGCGGATCTCGGTCGCC |
| | CAACCACCTCCGCTATGGAAAACCGTGACGGATTTATGGC |
| | CTATCTGAAGGAGCTGCGT |
| | (SEQ ID NO: 50) |
| F4RJP4_MELLP | ATGCCGGCATTCTCCCTGGTAGAGATAGAAGCGAAAATG |
| | TCCTTTTTTTCTGATTTTCTGAATCAAGTCAAGACGCCGAG |
| | TGTCGCCTCAAAGCAAATTTATGTTAGCAAAGTGCTTATT |
| | CAGATTACTAACTTTGATCAGCTGGATTTTGACTTTCAAA |
| | TCAAGATCCTCAACCAGGTTACTCTGCATCCATCCCAGCC |
| | AAAATTGACCCAGGAGGAAAAATCAAAACTCTTGAACAA |
| | CACGAGTATCCTGCGCGATAGTATCGTCTTCTTCACGGAT |
| | ACGGGTGCAGCACGTGGTGTAGGTGGTCACGCGGGCGGA |
| | CCATTTGATACCGTACGCGAGGTTGTGCTCCTGTTGGCTA |
| | GCTTTTGCCAGTGGGAGCGACAGCAAAATCTTTGATCATAC |
| | TGTGTCAGATGAAGCGGGCCATCGTGCCCAATCAAAGCT |
| | GCCGGGTCATCCGCAACTGGGTCTTACGCCGGGCGTGAA |
| | ATTCAGCAGCGTGGTCGTAGATTGGGCGACCTGCGGTCTG |
| | TTCAGCCGTGTGTCACACAGCCCAACGGAAACCGTGTTTT |
| | GCTTTTGCAGCGATGGTAGTCAGCACGAAGGCAGCGATG |
| | CGGAAGCCGCAAGACTGGCCCGTGCGCAGAAGCTTAACA |
| | TTAAATTATTGATCGATAACAACAATGTAACTATCTCTGG |
| | GCACACCAGCGGTTACCTTAAAGGATACAAAGTCGGTAA |
| | AACGCTGGAAGCACATGCCTTAAAAATAGTACGTGCAGA |
| | AGGTGAAAAATATACCGGCTGCAA CGATGTGAAATCTAA |
| | GGTGATACGGATCAACTTTGACCTCAAAGGTTCTACCGGC |
| | TTCGAGGCGATTCATCAGTCCCGCCCGGGTATTTTTCATTC |
| | CGTCGGTAATCGTGGAACATGGCAATTTTTGCGCAGCAGC |
| | GGGTTTCGGATTTGAAAAAGGCAAAGAAAAGATGCGTAA |
| | GCTGGACGCTGTTATTTCTTTTGGCGAGATTGTTCATCGTG |
| | CCTTGGACGCCGGCGATCAACTGGGCATAGAGGGGTTTG |
| | ATGTCGGCCTCGTAAACAAAAGTACCCTGAATGTGATTGA |
| | TGAAAAGCCGTGGATGAACATGGATATCCGCAACCTGTT |
| | (SEQ ID NO: 52) |
| A0A081BQW3_9BACT | ATGACCACGCTGGGAAACTCCCGCGTGGCGTTTCGCGATG |
| | CCTTAATGGAGCTGGCAGAACGCGACCCGCGGTACGTAC |
| | TGGTGTGTTCGGATTCTGGCCTGGTGATTAAGGCCCAACC |
| | TTTCATCGAGAAATTCCCCCAGCGCTTTTTTGATGTTGGA |
| | ATCGCGGAGCAGAACGCGGTTGGCGTGGCCGCGGGTCTG |
| | GCATCCAGCGGGTTGGTACCTTTTTTTGCGACCTACGCCG |
| | GTTTTATCACGATGCGTGCTTGTGAACAGGTACGCACCTT |
| | CGTCGCTTATCCGGGTCTGAACGTCAAACTGGTCGGCGCC |
| | AACGGCGGCATGGCGTCTGGGGAACGCGAAGGGGTCACG |
| | CACCAGTTTTTCGAGGATGTCGGTATACTGCGTGCAATTC |
| | CTGGCATTACAGTCGTCGTACCTGCCGATGCCGATCAGGT |
| | AGTAGCGGCAACCAAAGCGGTAGCATTAAAAGATGGCCC |
| | GGCCTATATACGTATCGGAAGCGGGCGTGACCCGATGGT |
| | TGAGGGGGAAACCCCGCCTTTTGAACTTGGCAAAGTTCGT |
| | ATTCTGAAAACCTACGGGCATGACGTAGCTATCTTCGCCA |
| | TGGGTTTTATAATGAACCGCGCGCTTGAGGCAGCGGCGC |
| | AACTGAACAGTGAAGGCATTCGGGCAGTTGTAGTAGACG |
| | TGCACACCCTGAAACCCCTGGATGTGGAGGCAATTACCG |
| | CGATCCTCCAGAAAACTTCTGCAGCGGTAACCGTGGAGG |
| | ATCATAACATCATTGGCGGCCTCGGGAGCGCGATAGCCG |
| | AGGTGTCGGCGGAGGAAATGCCGACCCCCCTGCGCCGTA |
| | TTGGTCTGCGCGATGTTTATCCGGAAAGTGGTCACCCGGA |
| | GCCTCTGCTGGATAAATACCACTTGGGCGTTAGCGACATC |
| | ATCAGCGCCGCCAAGACGGTGCTGAAAAAAAAGAATCAC |
| | CCGCCCCGCCGTATCGCCTTCAGCACCCGGGAAAATGCCG |
| | AGGAGGGTTTCAGTAACGGCAATATGGGCGAGGAAATTT |
| | ATGAAG |
| | (SEQ ID NO: 54) |
| CAK95977 | ATGAAGACGGTCCACGGTGCAACCTACGACATCCTGCGC |
| | CAGCATGGTCTGACGACGATTTTTGGTAATCCGGGTGATA |
| | ACGAACTGCCGTTTCTGAAAGGTTTCCCGGAAGACTTTCG |
| | TTATATTCTGGGCCTGCATGAAGGTGCCGTGGTTGGCATG |
| | GCAGATGGTTACGCGCTGGCCAGTGGTCAGCCGACCTTTG |
| | TGAACCTGCATGCGGCGGCGGGCACCGGTAACGGCATGG |
| | GTGCACTGACGAATGCTTGGTATAGTCACTCCCCGCTGGT |
| | TATTACGGCGGGTCAGCAAGTCCGCTCTATGATCGGCGTG |
| | GAAGCTATGCTGGCGAACGTGGACGCTGCACAGCTGCCG |
| | AAACCGCTGGTTAAGTGGTCACATGAACCGGCAACCGCT |
| | CAGGATGTGCCGCGTGCGCTGTCGCAAGCCATTCACACG |
| | GCAAATCTGCCGCCGCGCGGTCCGGTGTATGTTTCAATCC |
| | CGTACGATGACTGGGCCTGCGAAGCACCGTCGGGTGTTG |
| | AACATCTGGCGCGTCGCCAGGTCAGCTCTGCCGGCCTGCC |
| | GAGCCCGGCACAGCTGCAACACCTGTGTGAACGTCTGGC |
| | CGCAGCTCGTAACCCGGTCCTGGTGCTGGGTCCGGATGTG |
| | GATGGTTCTGCGGCCAATGGCCTGGCTGTTCAGCTGGCGG |
| | AAAAGCTGCGTATGCCGGCTTGGGTGGCACCGTCAGCCTC |
| | GCGCTGCCCGTTCCCGACCCGTCACGCCTGTTTTCGCGGT |
| | GTTCTGCCGGCAGCTATTGCCGGTATCAGCCATAACCTGG |
| | CAGGCCACGATCTGATTCTGGTCGTGGGTGCGCCGGTGTT |
| | CCGTTATCATCAGTTTGCGCCGGGTAATTACCTGCCGGCG |
| | GGTTGCGAACTGCTGCACCTGACCTGTGATCCGGGTGAAG |
| | CAGCCCGCGCTCCGATGGGTGACGCGCTGGTTGGCGATAT |
| | CGCCCTGACCCTGGAAGCAGTGCTGGATGGCGTTCCGCA |
| | GAGCGTCCGTCAAATGCCGACGGCACTGCCGGCAGCTGA |
| | ACCGGTGGCAGATGACGGTGGTCTGCTGCGTCCGGAAAC |
| | CGTTTTCGACCTGCTGAACGCGCTGGCCCCGAAAGATGCC |
| | ATTTATGTTAAGGAAAGCACCTCTACGGTCGGTGCATTCT |
| | GGCGTCGCGTGGAAATGCGTGAACCGGGCTCCTACTTTTT |
| | CCCGGCGGCCGGCGGTCTGGGTTTTGGTCTGCCGGCAGCT |
| | GTTGGTGTCCAGCTGGCCAGTCCGGGTCGCCAAGTGATTG |
| | GCGTTATCGGCGATGGTTCCGCTAACTATGGTATTACCGC |
| | ACTGTGGACGGCGGCCCAGTACAACATCCCGGTTGTCTTC |
| | ATTATCCTGAAAAATGGCACCTATGGTGCTCTGCGTTGGT |
| | TTGCGGATGTCCTGGACGTGAATGATGCGCCGGGTCTGGA |
| | CGTGCCGGGCCTGGATTTCTGCGCAATCGCTCGCGGCTAC |
| | GGTGTTCAGGCAGTCCATGCAGCTACCGGCAGCGCATTTG |
| | CCCAAGCACTGCGTGAAGCGCTGGAATCTGATCGCCCGG |
| | TGCTGATTGAAGTTCCGACCCAGACGATCGAACCG |
| | (SEQ ID NO: 56) |
| YP_831380 | ATGACGACGGTCCATGCCGCCGCCTATGAACTGCTGCGTA |
| | GCAATCGCCTGACGACGATCTTTGGTAATCCGGGTGATAA |
| | TGAACTGCCGTTTCTGGATGCAATGCCGGCTGACTTCCGC |
| | TATATTCTGGGCCTGCATGAGGGTGTGGTTGTCGGCATGG |
| | CGGATGGTTTTGCGCAGGCCAGCGGTCAAGCGGCCTTCGT |
| | TAACCTGCATGCAGCTTCTGGCACCGGTAACGCGATGGGC |
| | GCCCTGACGAATGCATGGTACAGTCACACCCCGCTGGTG |
| | ATTACGGCGGGCCAGCAAGTTCGTCCGATGATCGGTCTGG |
| | AAGCGATGCTGAGCAATGTTGATGCAGCCTCTCTGCCGCG |
| | CCCGCTGGTCAAATGGTCTGCCGAACCGGCACAGGCTCC |
| | GGATGTTCCGCGTGCGCTGAGCCAAGCCATTCATACCGCA |
| | ACGTCTGACCCGAAGGGTCCGGTGTATCTGAGTATCCCGT |
| | ACGATGACTGGAACCAGGATACCGGTAATCTGTCCGAAC |
| | ACCTGAGCAGCCGTAGCGTGAGCCGTGCGGGTAACCCGT |
| | CAGCTGAACAACTGGATGACATTCTGTCGGCACTGCGTGA |
| | AGCAGCTAACCCGGCGCTGGTTTTTGGTCCGGATGTGGAT |
| | GCGGCCCGCGCTAATCATCACGCGGTGCGTCTGGCCGAA |
| | AAACTGGCAGCTCCGGTTTGGATCGCACCGGCGGCACCG |
| | CGTTGCCCGTTTCCGACCCGCCATCCGAACTTCCGTGGCG |
| | TTCTGCCGGCAAGTATTGCTGGCATCTCCGCCCTGCTGAA |
| | TGGTCATGATCTGATTGTGGTTATCGGTGCACCGGTGTTC |
| | CGTTATCACCAGTACCAACCGGGCAGTTATCTGCCGGAAA |
| | ATTCCCGCCTGATTCACATCACCTGTGATGCAGGTGAAGC |
| | AGCTCGTGCCCCGATGGGTGATGCGCTGGTTGCCGACATT |
| | GGTCAGACGCTGCGCGCGCTGGCCGACATTATCCCGCAA |
| | AGCAAACGTCCGCCGCTGCGCCCGCGTGTCATCCCGCCGG |
| | TGCCGGATTCACAGGATGACCTGCTGGCACCGGACGCTGT |
| | CTTTGAAGTGATGAACGAAGTCGCGCCGGAAGATGTCGT |
| | GTATGTGAATGAATCAGTTTCGACCGTCACGGCCCTGTGG |
| | GAACGTGTGGAACTGAAGCATCCGGGTTCATATTACTTTC |
| | CGGCGTCGGGCGGTCTGGGTTTCGGTATGCCGGCGGCCGT |
| | GGGTGTTCAGCTGGCCAACGATCGTCGCCGTGTGATTGCA |
| | GTTATCGGCGACGGTAGCGCAAATTATGGCATTACCGCTC |
| | TGTGGACGGCAGCTCAGGAAAAAATCCCGGTTGTCTTTAT |
| | TATCCTGAACAATGGCACCTACGGTGCGCTGCGCGCATTC |
| | GCTAAGCTGCTGAACGCCGAAAATGCGGCCGGCCTGGAT |
| | GTGCCGGGCATTTGCTTTTGTGCGATCGCCGAAGGCTATG |
| | GTGTGGAAGCGCACCGTATTACCAGCCTGGAAAACTTCA |
| | AAGATAAGCTGTCAGCAGCTCTGCAATCGGACACCCCGA |
| | CGCTGCTGGAAGTGCCGACCAGCACCACGTCTCCGTTT |
| | (SEQ ID NO: 58) |
| ZP_06547677 | ATGAAGACCATCCACTCTGCCGCCTATGCCCTGCTGCGTC |
| | GCCACGGTATGACCACCATTTTCGGTAATCCGGGTAGCAA |
| | TGAACTGCCGTTTCTGAAAAGTTTCCCGGAAGACTTTCAG |
| | TATGTTCTGGGCCTGCATGAAGGTGCCGTGGTTGGCATGG |
| | CAGATGGTTACGCCCTGGCAAGCGGCAAGCCGGCATTCG |
| | TGAACCTGCATGCGGCGGCGGGCACCGGTAACGGCATGG |
| | GTGCCCTGACCAATTCTTGGTATAGCCACTCTCCGCTGGT |
| | GATTACGGCAGGCCAGCAAGTTCGTCCGATGATCGGTGTC |
| | GAAGCGATGCTGGCCAATGTGGACGCGACCCAGCTGCCG |
| | AAACCGCTGGTTAAGTGGAGCTATGAACCGGCTAACGCG |
| | CAGGATGTTCCGCGCGCACTGTCGCAAGCTATTCATTACG |
| | CGAATACCACGCCGAAAGCCCCGGTGTATCTGAGCATCC |
| | CGTACGATGACTGGGATCAGCCGTCTGGTCCGGGCGTCG |
| | AACACCTGATTGAACGTGACGTGCAAACGGCTGGCACCC |
| | CGGATGCACGTCAGCTGCAAGTTCTGGTCCAGCAAGTTCA |
| | GGATGCACGTAACCCGGTGCTGGTTCTGGGTCCGGATGTG |
| | GATGCGACCCTGAGCAATGACCATGCCGTGGCACTGGCT |
| | GATAAACTGCGTATGCCGGTTTGGATCGCACCGGCTGCGA |
| | GTCGCTGCCCGTTCCCGACGCGTCATCCGTCCTTTCGTGG |
| | TGTGCTGCCGGCCGCAATTGCAGGTATCAGCAAGACCCTG |
| | CAAGGTCACGATCTGATTATCGTCGTGGGTGCGCCGGTTT |
| | TCCGTTATCTGCAATTTGCGCCGGGTGACTACCTGCCGGT |
| | GGGTGCACAACTGCTGCATATTACGTCAGATCCGCTGGAA |
| | GCAACCCGTGCTCCGATGGGCCACGCCCTGGTTGGTGATA |
| | TCCGTGAAACCCTGCGCGTCCTGGCAGAAGAAGTTGTCCA |
| | GCAATCGCGCCCGTATCCGGAAGCGCTGGCTGCACCGGA |
| | ATGTGTGACGGACGAACCGCATCACCTGCATCCGGAAAC |
| | CCTGTTCGATGTCCTGGACGCAGTGGCACCGCACGATGCT |
| | ATTTACGTGAAAGAAAGTACCTCCACGGTTACCGCCTTTT |
| | GGCAGCGTATGAACCTGCGCCATCCGGGCAGCTATTACTT |
| | CCCGGCCGCAGGCGGTCTGGGTTTTGGTCTGCCGGCTGCG |
| | GTCGGTGTGCAGCTGGCACAGCCGCAACGTCGCGTGGTT |
| | GCTCTGATTGGCGATGGTTCTGCGAACTATGGTATCACGG |
| | CACTGTGGACCGCCGCACAGTACCGTATTCCGGTCGTGTT |
| | CATTATCCTGAAAAATGGCACCTATGGTGCCCTGCGCTGG |
| | TTTGCAGGTGTCCTGAAGGCTGAAGATAGTCCGGGCCTGG |
| | ACGTGCCGGGTCTGGATTTCTGCGCAATCGCTAAAGGCTA |
| | CGGTGTTAAGGCGGTCCATACGGATACCCGTGACTCCTTT |
| | GAAGCTGCACTGCGTACGGCGCTGGATGCAAACGAACCG |
| | ACCGTGATTGAAGTTCCGACGCTGACCATCCAGCCGCAC |
| | (SEQ ID NO: 60) |
| ZP_06846103 | ATGACCAGCCGTAGCTCGTTTAGCCCGCCGTCAGCGTCAG |
| | AACAGCGTGGTGCGGATATTTTTGCCGAAGTCCTGCAATG |
| | TGAAGGTGTCCGCTATATTTTTGGCAATCCGGGCACCACG |
| | GAACTGCCGCTGCTGGATGCACTGACCGACATTACGGGT |
| | ATCCATTATGTGCTGGGCCTGCACGAAGCGTCAGTGGTTG |
| | CGATGGCCGATGGTTACGCACAGGCTTCGGGCAAACCGG |
| | GTTTCGTTAACCTGCATACCGCCGGCGGTCTGGGTAATGC |
| | GATGGGTGCCATTCTGAACGCAAAGATGGCTAATACCCC |
| | GCTGGTCGTGACGGCGGGTCAGCAAGATACCCGTCATGG |
| | CGTTACCGATCCGCTGCTGCACGGCGACCTGACCGGTATC |
| | GCACGTCCGAATGTCAAATGGGCCGAAGAAATTCATCAC |
| | CCGGAACATATCCCGATGCTGCTGCGTCGTGCGCTGCAAG |
| | ATTGCCGCACGGGTCCGGCTGGTCCGGTGTTTCTGAGTCT |
| | GCCGATTGACACGATGGAACGTTGTACGTCCGTGGGTGC |
| | AGGTGAAGCCAGCCGTATCGAACGCGCGAGCGTGGCTAA |
| | CATGCTGCATGCGCTGGCCACCGCACTGGCTGAAGTGAC |
| | GGCCGGTCACATTGCGCTGGTCGCCGGTGAAGAAGTGTTC |
| | ACCGCGAATGCCAGTGTTGAAGCAGTCGCTCTGGCGGAA |
| | GCACTGGGCGCACCGGTTTTTGGTGCTTCCTGGCCGGGTC |
| | ATATTCCGTTCCCGACCGCACACCCGCAGTGGCAGGGTAC |
| | GCTGCCGCCGAAGGCGAGCGATATCCGTGAAACCCTGGG |
| | CCCGTTTGACGCCGTGCTGATTCTGGGCGGTCATAGTCTG |
| | ATCTCCTATCCGTACTCAGAAGGTCCGGCAATTCCGCCGC |
| | ACTGCCGCCTGTTCCAGCTGACCGGCGATGGTCATCAAAT |
| | CGGCCGTGTTCACGAAACCACGCTGGGCCTGGTGGGCGA |
| | TCTGCAACTGAGTCTGCGCGCGCTGCTGCCGCTGCTGGCC |
| | CGTAAACTGCAACCGCAAAACGGTGCAGTCGCTCGTCTG |
| | CGCCAAGTGGCAACCCTGAAGCGTGATGCTCGTCGCACG |
| | GAAGCGGCCGAACGTTCAGCCCGCGAATTTGACGCGTCG |
| | GCCACCACGCCGTTTGTTGCAGCTTTCGAAACCATTCGCG |
| | CAATCGGCCCGGATGTGCCGATTGTTGACGAAGCGCCGG |
| | TTACGATCCCGCATGTCCGTGCCTGCCTGGATAGCGCATC |
| | TGCTCGCCAGTACCTGTTTACCCGTTCTGCAATTCTGGGTT |
| | GGGGTATGCCGGCGGCCGTCGGTGTGAGTCTGGGTCTGG |
| | ATCGTTCCCCGGTTGTCTGTCTGGTGGGCGACGGTTCAGC |
| | GATGTACTCGCCGCAGGCACTGTGGACCGCAGCTCACGA |
| | ACGCCTGCCGGTTACGTTTGTGGTTTTCAACAATGGTGAA |
| | TATAACGCCCTGAAAAATTTTGCGCGTGCCCAAACCAACT |
| | ACCGTAGCGCACGCGCTAATCGTTTTATTGGCCTGGATAT |
| | CTCTGACCCGGCGATTGATTTCCCGGCGCTGGCCAGCTCT |
| | CTGGGTGTGCCGGCACGTCGCGTTGAACGTGCTGGTGATA |
| | TTGCAATCGCTGTCGAAGACGGCATCCGCAGCGGTCGTCC |
| | GAACCTGATTGATGTGCTGATCAGTTCCTCATCG |
| | (SEQ ID NO: 62) |
| ZP_07290467 | ATGCGTACGGTGCGTGAATCGGCTCTGGACGTGCTGCGTG |
| | CGCGTGGTATGACGACGGTTTTTGGTAATCCGGGCTCAAC |
| | GGAACTGCCGATGCTGAAACAGTTTCCGGATGACTTCCGC |
| | TATGTTCTGGGTCTGCAAGAAGCTGTGGTTGTCGGTATGG |
| | CAGATGGCTTTGCCCTGGCAAGTGGCACCACGGGTCTGGT |
| | GAATCTGCATACCGGTCCGGGCACGGGTAACGCGATGGG |
| | CGCAATTCTGAACGCTCGTGCGAATCGTACCCCGATGGTG |
| | GTTACGGCGGGCCAGCAAGTGCGTGCCATGCTGACGATG |
| | GAAGCACTGCTGACCAATCCGCAGAGTACGCTGCTGCCG |
| | CAACCGGCTGTCAAGTGGGCGTACGAACCGCCGCGCGCG |
| | GCCGATGTGGCACCGGCACTGGCTCGTGCGGTCCAGGTG |
| | GCAGAAACCCCGCCGCAAGGTCCGGTTTTTGTCTCCCTGC |
| | CGATGGATGACTTCGATGTCGTGCTGGGCGAAGATGAAG |
| | ACCGTGCAGCTCAGCGTGCGGCGGCACGTACCGTTACGC |
| | ACGCTGCGGCCCCGAGCGCGGAAGTTGTCCGTCGCCTGG |
| | CAGCTCGTCTGAGTGGTGCTCGTTCCGCGGTGCTGGTTGC |
| | GGGTAATGATGTGGACGCCTCTGGCGCATGGGATGCTGT |
| | GGTTGAACTGGCCGAACGTACCGGTCTGCCGGTCTGGAGT |
| | GCACCGACGGAAGGTCGTGTGGCATTTCCGAAATCCCATC |
| | CGCAGTATCGTGGTATGCTGCCGCCGGCAATTGCACCGCT |
| | GAGCCGTTGCCTGGAAGGTCACGATCTGGTCCTGGTGATC |
| | GGTGCGCCGGTGTTCTGTTATTACCCGTACGTTCCGGGTG |
| | CCCATCTGCCGGAAAACACCGAACTGGTTCACCTGACGC |
| | GCGATGCAGACGAAGCAGCCCGTGCCCCGGTTGGTGATG |
| | CAGTCGTGGCCGACCTGGCACTGACCGTGCGCGCTCTGCT |
| | GGCGGAACTGCCGGCGCGTGAAGCAGCTGCGCCGGCCGC |
| | ACGTACCGCTCGCGCGGAATCTACGGCCGAAGTCGATGG |
| | TGTGCTGACCCCGCTGGCTGCAATGACGGCAATTGCACAG |
| | GGCGCTCCGGCAAACACCCTGTGGGTTAATGAAAGCCCG |
| | TCTAACCTGGGTCAATTTCATGATGCAACCCGTATCGACA |
| | CGCCGGGCAGCTTTCTGTTCACCGCCGGCGGTGGCCTGGG |
| | TTTCGGTCTGGCCGCAGCTGTGGGTGCCCAGCTGGGCGCA |
| | CCGGATCGTCCGGTTGTCTGCGTTATTGGCGACGGTTCAA |
| | CCCACTATGCAGTCCAGGCACTGTGGACCGCGGCGGCGT |
| | ACAAAGTTCCGGTCACCTTTGTGGTTCTGTCGAATCAGCG |
| | CTATGCAATCCTGCAATGGTTCGCGCAAGTGGAAGGCGCT |
| | CAAGGTGCGCCGGGCCTGGATATTCCGGGTCTGGACATC |
| | GCTGCGGTTGCAACGGGTTACGGTGTCCGTGCCCATCGTG |
| | CAACCGGCTTTGGTGAACTGTCAAAGCTGGTGCGTGAATC |
| | GGCGCTGCAACAAGATGGCCCGGTTCTGATCGACGTGCC |
| | GGTTACCACGGAACTGCCGACCCTG |
| | (SEQ ID NO: 64) |
| ZP_08570611 | ATGTCATCAATCAACTCGTTCACCGTCGCCGACTACCTGC |
| | TGACCCGTCTGCATCAACTGGGCCTGCGTAAGGTTTTTCA |
| | AGTGCCGGGCGATTATGTCGCTAACTTTATGGACGCGCTG |
| | GAACAGTTCAATGGCATTGAAGCCGTGGGTGATCTGACC |
| | GAACTGGGTGCAGGTTATGCGGCCGACGGTTACGCACGT |
| | CTGACCGGTATCGGTGCAGTGTCTGTTCAGTTTGGCGTGG |
| | GTACGTTTTCTGTTCTGAACGCAATTGCTGGCAGTTACGT |
| | TGAACGTAATCCGGTGGTTGTCATCACCGCGTCGCCGAGC |
| | ACGGGTAACCGCAAAACCATTAAGGAAACGGGCGTGCTG |
| | TTTCATCACTCCACCGGTGATCTGCTGGCTGACTCAAAAG |
| | TGTTCGCGAATGTCACGGTGGCAGCTGAAGTTCTGTCTGA |
| | TCCGAGTGACGCGCGCCAGAAAATTGATAAGGCCCTGAC |
| | CCTGGCAATTACGTTTCGTCGCCCGATCTATCTGGAAGCC |
| | TGGCAGGATGTTTGGGGCCTGGCATGCGAAAAACCGGAA |
| | GGTGAACTGAAGGCCCTGCCGCTGATCAGCGAAGAAGGC |
| | GCGCTGAAAGCCATGCTGGCAGATTCTCTGAAGCTGCTGA |
| | ACAGTGCACGTCAGCCGCTGGTTCTGCTGGGTGTCGAAAT |
| | TAATCGCTTCGGTCTGCAAGATGCTGTTCTGGACCTGCTG |
| | AAAGCGTCTGGTCTGCCGTATTCCACCACGTCACTGGCCA |
| | AGACCGTTATTAGTGAAAACGAAGGCATCTTTGTCGGCAC |
| | CTATGCGGATGGTGCGTCCTTCCCGGCAACGGTGGAATAC |
| | ATCGAAAAAGCCGATTGTGTCCTGGCACTGGGTGTGATTT |
| | TTACCGATGACTACCTGACGATGCTGTCAAAACAGTTCGA |
| | TCAAATGATCGTGGTTAACAATGACGAAACCTCGCGTCTG |
| | GGCCATGCTTATTACCACCAGCTGTATCTGGCGGATTTTA |
| | TTCTGCAACTGACGGACGAAATTAAAAAATCTAGCCTGTA |
| | CCCGCGTCAGAACAGCGCACTGCCGCTGCTGCCGCCGCA |
| | ACCGCAGATTACCCCGGCGCTGCTGCAACAACAGCTGAG |
| | TTATCAGAACTTTTTCGACCTGTTTTATGGTTACCTGCTGC |
| | AACATCAGCTGCAAGACAATATTTCCCTGATCCTGGGCGA |
| | AAGTTCCTCACTGTATATGTCAGCTCGTCTGTACGGTCTG |
| | CCGCAGGATTCTTTCATCGCAGACGCAGCATGGGGCAGTC |
| | TGGGTCACGAAACCGGCTGCGTTACGGGTATCGCGTATGC |
| | CAGCGATAAACGTGCAATGGCTATTGCGGGTGACGGCGG |
| | TTTTATGATGATGTGCCAGTGTCTGAGCACCATTAGCCGC |
| | CATCAACTGAACTCCGTCGTGTTCGTTATTTCAAATAAAG |
| | TCTACGCCATCGAACAGTCCTTTGTGGATATTTGTGCCTTC |
| | GCAAAGGGCGGTCACTTTGCGCCGTTCGATCTGCTGCCGA |
| | CCTGGGACTATCTGTCGCTGGCTAAAGCGTTTAGCGTGGA |
| | AGGCTACCGCGTTCAGAACGGTGAAGAACTGCTGCAAGC |
| | GCTGGAACATATCATGACCCAGAAAGATAAGCCGGCCCT |
| | GGTGGAAGTTGTCATTCAGTCGCAGGATCTGGCACCGGC |
| | AATGGCTGGCCTGGTCAAAAGCATCACCGGTCACACGGT |
| | GGAACAGTGCGCCATTCCGACC |
| | (SEQ ID NO: 66) |
| YP_001240047 | |
| YP_001279645 | |
| ZP_01901192 | |
| ZP_06549025 | |
| ZP_07033476 | |
| WP_010764607.1 | |
| WP_002115026.1 | |
| YP_005756646.1 | |
| WP_008347133.1 | |
| WP_018535238.1 | |
| YP_006485164.1 | |
| YP_005461458.1 | |
| YP_006991301.1 | |
| NP_594083.1 | |
| WP_003075272.1 | |
| WP_020634527.1 | |
| IOVM | ATGCGTACCCCGTACTGCGTTGCTGACTACCTGCTGGACC |
| | GTCTGACCGATTGCGGCGCGGACCACCTGTTTGGCGTGCC |
| | GGGCGACTACAACCTGCAATTTCTGGACCATGTCATTGAT |
| | TCTCCGGACATCTGCTGGGTGGGCTGTGCCAACGAACTGA |
| | ATGCAAGTTATGCGGCCGATGGCTACGCACGTTGCAAAG |
| | GTTTTGCAGCTCTGCTGACCACGTTCGGCGTGGGTGAACT |
| | GTCCGCGATGAATGGCATTGCCGGCAGCTATGCGGAACA |
| | TGTGCCGGTTCTGCACATCGTTGGCGCGCCGGGCACCGCG |
| | GCGCAGCAACGTGGTGAACTGCTGCATCACACGCTGGGC |
| | GATGGTGAATTTCGCCATTTCTACCACATGTCCGAACCGA |
| | TTACCGTTGCCCAAGCAGTCCTGACGGAACAGAACGCCT |
| | GCTATGAAATCGACCGTGTGCTGACCACGATGCTGCGCG |
| | AACGTCGTCCGGGCTATCTGATGCTGCCGGCTGATGTTGC |
| | GAAAAAGGCAGCTACCCCGCCGGTCAACGCACTGACGCA |
| | TAAACAGGCTCACGCGGATTCCGCTTGTCTGAAGGCGTTT |
| | CGTGACGCGGCCGAAAATAAACTGGCCATGTCAAAGCGT |
| | ACCGCCCTGCTGGCAGACTTCCTGGTGCTGCGTCATGGCC |
| | TGAAACACGCGCTGCAAAAATGGGTTAAGGAAGTCCCGA |
| | TGGCCCATGCAACCATGCTGATGGGCAAGGGTATTTTTGA |
| | TGAACGCCAGGCCGGCTTCTATGGCACCTACTCAGGCTCG |
| | GCCAGCACGGGTGCAGTGAAAGAAGCTATCGAAGGCGCG |
| | GATACCGTGCTGTGCGTTGGTACGCGTTTTACCGACACGC |
| | TGACCGCCGGTTTCACGCATCAGCTGACCCCGGCACAAAC |
| | GATTGAAGTTCAGCCGCACGCAGCTCGCGTCGGTGATGTG |
| | TGGTTTACCGGTATTCCGATGAACCAAGCGATCGAAACGC |
| | TGGTTGAACTGTGTAAACAGCATGTCCACGCTGGCCTGAT |
| | GAGCAGCAGCAGCGGTGCCATTCCGTTCCCGCAACCGGA |
| | TGGCTCTCTGACCCAGGAAAATTTTTGGCGTACGCTGCAA |
| | ACCTTCATTCGTCCGGGCGATATTATCCTGGCGGACCAGG |
| | GCACCTCTGCTTTTGGTGCGATCGATCTGCGTCTGCCGGC |
| | CGACGTGAACTTCATTGTTCAACCGCTGTGGGGCAGTATC |
| | GGTTATACCCTGGCGGCGGCGTTTGGCGCCCAGACGGCAT |
| | GTCCGAATCGTCGCGTCATTGTGCTGACCGGCGATGGTGC |
| | TGCGCAGCTGACGATCCAAGAACTGGGTAGCATGCTGCG |
| | CGACAAACAACATCCGATTATCCTGGTGCTGAACAATGA |
| | AGGCTATACCGTTGAACGTGCCATTCATGGTGCAGAACA |
| | GCGCTACAACGATATTGCACTGTGGAATTGGACCCACATC |
| | CCGCAAGCGCTGTCTCTGGACCCGCAGAGTGAATGCTGG |
| | CGTGTGTCGGAAGCTGAACAGCTGGCGGATGTCCTGGAA |
| | AAAGTGGCGCATCACGAACGCCTGAGCCTGATTGAAGTT |
| | ATGCTGCCGAAAGCTGATATCCCGCCGCTGCTGGGTGCGC |
| | TGACCAAGGCTCTGGAAGCGTGTAACAATGCC |
| | (SEQ ID NO: 100) |
| 2Q5Q | |
| 2VBG | ATGTACACCGTTGGCGACTACCTGCTGGACCGTCTGCATG |
| | AACTGGGCATCGAAGAAATCTTTGGCGTGCCGGGTGACT |
| | ATAACCTGCAATTTCTGGATCAGATTATCAGCCGTGAAGA |
| | CATGAAATGGATTGGTAACGCTAATGAACTGAACGCATC |
| | TTATATGGCTGATGGTTACGCACGTACCAAAAAGGCGGC |
| | GGCGTTTCTGACCACGTTCGGCGTTGGTGAACTGAGCGCA |
| | ATTAACGGCCTGGCCGGTTCTTATGCAGAAAATCTGCCGG |
| | TGGTTGAAATCGTTGGCTCACCGACGTCGAAAGTCCAGA |
| | ATGATGGCAAGTTTGTGCATCACACCCTGGCCGATGGCGA |
| | CTTTAAACATTTCATGAAGATGCACGAACCGGTGACGGCT |
| | GCGCGTACCCTGCTGACGGCGGAAAACGCCACCTATGAA |
| | ATTGATCGTGTGCTGAGCCAGCTGCTGAAAGAACGCAAG |
| | CCGGTTTACATCAATCTGCCGGTTGATGTCGCCGCAGCTA |
| | AAGCTGAAAAGCCGGCGCTGTCTCTGGAAAAAGAAAGCT |
| | CTACCACGAACACCACGGAACAGGTTATTCTGAGCAAAA |
| | TCGAAGAATCTCTGAAAAATGCCCAAAAGCCGGTCGTGA |
| | TTGCAGGCCATGAAGTGATCTCATTTGGTCTGGAAAAAAC |
| | CGTCACGCAGTTCGTGTCGGAAACCAAGCTGCCGATTACC |
| | ACGCTGAACTTTGGTAAAAGTGCCGTGGATGAAAGCCTG |
| | CCGTCTTTCCTGGGCATTTATAACGGTAAACTGAGTGAAA |
| | TCTCCCTGAAGAATTTTGTCGAAAGCGCCGATTTCATTCT |
| | GATGCTGGGCGTGAAACTGACCGACAGTTCCACGGGTGC |
| | ATTTACCCATCACCTGGATGAAAACAAGATGATCAGTCTG |
| | AACATCGACGAAGGCATCATCTTCAACAAGGTTGTCGAA |
| | GATTTCGACTTCCGTGCGGTGGTTTCATCGCTGTCCGAAC |
| | TGAAGGGCATTGAATATGAAGGCCAGTACATCGATAAGC |
| | AATACGAAGAATTTATCCCGAGCAGCGCACCGCTGAGCC |
| | AGGACCGTCTGTGGCAAGCAGTTGAATCACTGACGCAGT |
| | CGAACGAAACCATTGTCGCTGAACAAGGCACCAGCTTTTT |
| | CGGTGCGTCCACCATCTTTCTGAAAAGTAATTCCCGTTTC |
| | ATTGGTCAGCCGCTGTGGGGCAGCATCGGTTATACCTTTC |
| | CGGCGGCACTGGGCTCACAAATTGCGGATAAAGAATCGC |
| | GCCATCTGCTGTTCATCGGCGACGGTAGCCTGCAACTGAC |
| | CGTTCAAGAACTGGGTCTGTCTATTCGTGAAAAACTGAAC |
| | CCGATCTGCTTTATTATCAACAATGATGGCTACACGGTGG |
| | AACGCGAAATTCACGGTCCGACCCAGTCATATAACGACA |
| | TCCCGATGTGGAATTACTCGAAACTGCCGGAAACGTTTGG |
| | CGCCACCGAAGATCGTGTCGTGAGTAAGATTGTGCGCAC |
| | CGAAAACGAATTTGTGTCCGTTATGAAAGAAGCACAGGC |
| | TGATGTTAATCGCATGTATTGGATCGAACTGGTCCTGGAA |
| | AAAGAAGACGCTCCGAAGCTGCTGAAAAAGATGGGCAAA |
| | CTGTTTGCGGAACAGAACAAG |
| | (SEQ ID NO: 104) |
| 2VBI | ATGACCTATACGGTGGGCATGTACCTGGCTGAACGCCTGG |
| | TGCAGATTGGCCTGAAACATCACTTTGCGGTGGCTGGCGA |
| | TTACAACCTGGTGCTGCTGGATCAACTGCTGCTGAACAAA |
| | GACATGAAACAGATTTATTGCTGTAACGAACTGAATTGCG |
| | GCTTTAGCGCAGAAGGTTACGCTCGCTCTAATGGTGCGGC |
| | GGCGGCAGTGGTTACCTTCAGTGTGGGTGCCATTTCCGCA |
| | ATGAACGCTCTGGGCGGTGCTTACGCGGAAAATCTGCCG |
| | GTTATTCTGATCTCAGGCGCGCCGAACTCGAATGATCAGG |
| | GCACGGGTCATATCCTGCATCACACCATTGGTAAAACGG |
| | ATTATAGCTACCAACTGGAAATGGCACGTCAGGTCACCTG |
| | TGCGGCCGAATCAATCACGGATGCGCATTCGGCCCCGGC |
| | AAAAATCGACCACGTTATTCGTACCGCACTGCGTGAACGT |
| | AAACCGGCATATCTGGATATCGCGTGCAACATTGCAAGC |
| | GAACCGTGTGTGCGTCCGGGTCCGGTTAGCTCTCTGCTGA |
| | GTGAACCGGAAATTGATCATACCTCCCTGAAAGCAGCTGT |
| | GGACGCGACGGTTGCCCTGCTGGAAAAATCAGCCTCGCC |
| | GGTGATGCTGCTGGGCTCAAAACTGCGTGCAGCAAACGC |
| | ACTGGCAGCTACCGAAACGCTGGCAGATAAACTGCAGTG |
| | CGCTGTGACCATCATGGCGGCGGCAAAAGGCTTTTTCCCG |
| | GAAGATCACGCCGGCTTCCGTGGTCTGTATTGGGGCGAA |
| | GTTTCAAATCCGGGTGTCCAGGAACTGGTGGAAACCTCG |
| | GATGCACTGCTGTGTATCGCTCCGGTTTTTAACGACTACA |
| | GCACGGTCGGCTGGTCTGCGTGGCCGAAAGGTCCGAATG |
| | TGATTCTGGCCGAACCGGACCGTGTTACCGTCGATGGTCG |
| | TGCGTATGATGGTTTTACGCTGCGTGCTTTCCTGCAAGCT |
| | CTGGCAGAAAAAGCACCGGCACGTCCGGCTAGTGCACAG |
| | AAAAGTTCCGTTCCGACCTGCAGTCTGACCGCGACGTCCG |
| | ATGAAGCCGGCCTGACGAACGACGAAATCGTTCGCCACA |
| | TTAACGCGCTGCTGACCAGCAATACCACGCTGGTCGCGG |
| | AACGGGCGATTCTTGGTTCAATGCCATGCGTATGACCCT |
| | GCCGCGTGGTGCACGCGTCGAACTGGAAATGCAGTGGGG |
| | CCATATTGGTTGGAGCGTGCCGTCTGCATTTGGCAATGCT |
| | ATGGGTAGTCAGGATCGTCAACACGTCGTGATGGTGGGC |
| | GACGGTTCCTTCCAGCTGACCGCGCAAGAAGTTGCCCAG |
| | ATGGTCCGTTATGAACTGCCGGTGATTATCTTTCTGATCA |
| | ACAATCGCGGCTACGTTATTGAAATCGCCATTCATGATGG |
| | TCCGTACAACTACATCAAAAACTGGGACTATGCCGGTCTG |
| | ATGGAAGTTTTTAACGCAGGCGAAGGTCACGGCCTGGGT |
| | CTGAAAGCGACCACGCCGAAAGAACTGACCGAAGCCATT |
| | GCACGTGCTAAAGCGAATACCCGCGGCCCGACGCTGATC |
| | GAATGCCAAATTGATCGTACCGACTGTACGGATATGCTGG |
| | TCCAGTGGGGTCGCAAAGTGGCGTCTACCAACGCACGCA |
| | AAACGACGCTGGCG (SEQ ID NO: 106) |
| 3FZN | ATGGCGAGCGTGCATGGCACCACGTATGAACTGCTGCGT |
| | CGCCAGGGTATCGATACCGTGTTCGGCAACCCGGGTTCAA |
| | ATGAACTGCCGTTTCTGAAAGATTTCCCGGAAGACTTTCG |
| | TTATATCCTGGCACTGCAAGAAGCGTGCGTGGTTGGCATT |
| | GCAGACGGTTACGCGCAAGCCTCGCGCAAACCGGCGTTT |
| | ATTAACCTGCATAGCGCGGCCGGCACCGGTAATGCAATG |
| | GGCGCTCTGAGCAACGCGTGGAACAGCCACAGCCCGCTG |
| | ATCGTGACCGCGGGCCAGCAAACGCGTGCCATGATTGGT |
| | GTGGAAGCACTGCTGACGAACGTTGATGCAGCTAATCTG |
| | CCGCGCCCGCTGGTCAAATGGTCCTATGAACCGGCATCAG |
| | CGGCCGAAGTGCCGCATGCAATGTCTCGTGCCATCCACAT |
| | GGCAAGTATGGCCCCGCAGGGTCCGGTCTATCTGTCTGTG |
| | CCGTACGATGACTGGGATAAAGACGCCGATCCGCAGAGT |
| | CATCACCTGTTTGATCGTCATGTTAGCTCTAGTGTCCGCCT |
| | GAACGACCAGGATCTGGATATCCTGGTTAAAGCACTGAA |
| | CTCTGCTAGTAATCCGGCGATTGTGCTGGGTCCGGATGTT |
| | GACGCAGCTAACGCAAATGCTGATTGCGTGATGCTGGCT |
| | GAACGTCTGAAAGCGCCGGTTTGGGTCGCACCGTCGGCTC |
| | CGCGTTGCCCGTTCCCGACCCGTCACCCGTGTTTTCGTGG |
| | TCTGATGCCGGCCGGTATTGCAGCAATCAGCCAGCTGCTG |
| | GAAGGCCATGATGTCGTGCTGGTCATCGGTGCACCGGTGT |
| | TCCGCTATCACCAGTACGACCCGGGCCAATATCTGAAACC |
| | GGGTACCCGTCTGATTTCTGTTACGTGTGATCCGCTGGAA |
| | GCAGCTCGCGCGCCGATGGGCGATGCAATCGTGGCAGAC |
| | ATTGGTGCGATGGCCAGTGCACTGGCTAACCTGGTTGAAG |
| | AATCCTCACGTCAGCTGCCGACCGCGGCCCCGGAACCGG |
| | CTAAAGTTGATCAAGACGCAGGTCGTCTGCACCCGGAAA |
| | CCGTCTTTGATACGCTGAATGACATGGCCCCGGAAAACGC |
| | AATTTACCTGAATGAATCCACGTCAACCACGGCCCAGATG |
| | TGGCAACGTCTGAACATGCGCAATCCGGGTTCTTATTACT |
| | TCTGTGCAGCTGGCGGTCTGGGTTTTGCACTGCCGGCGGC |
| | AATCGGTGTGCAGCTGGCGGAACCGGAACGTCAAGTGAT |
| | TGCCGTTATCGGCGATGGTAGCGCCAACTATTCGATTAGC |
| | GCACTGTGGACCGCAGCTCAGTACAATATTCCGACGATCT |
| | TCGTTATTATGAACAATGGCACCTATGGTGCCCTGCGTTG |
| | GTTTGCAGGTGTGCTGGAAGCTGAAAACGTTCCGGGCCTG |
| | GATGTCCCGGGTATCGACTTCCGTGCACTGGCAAAAGGCT |
| | ACGGTGTTCAGGCACTGAAAGCTGATAATCTGGAACAGC |
| | TGAAAGGCTCGCTGCAAGAAGCGCTGAGCGCCAAAGGTC |
| | CGGTGCTGATTGAAGTCTCTACCGTGAGTCCGGTTAAA |
| | (SEQ ID NO: 108) |
| IZPD | ATGAGCTATACCGTGGGCACGTACCTGGCTGAACGTCTGG |
| | TTCAAATTGGCCTGAAACATCACTTTGCCGTGGCCGGTGA |
| | TTATAATCTGGTTCTGCTGGACAACCTGCTGCTGAATAAA |
| | AACATGGAACAGGTGTACTGCTGTAATGAACTGAACTGC |
| | GGCTTCAGTGCGGAAGGTTATGCTCGCGCGAAGGGTGCG |
| | GCGGCGGCGGTGGTTACCTACAGTGTTGGTGCCCTGTCCG |
| | CATTTGATGCTATCGGCGGTGCCTATGCAGAAAATCTGCC |
| | GGTTATTCTGATCTCCGGCGCCCCGAACAATAACGATCAT |
| | GCGGCGGGTCATGTCCTGCATCACGCACTGGGTAAAACC |
| | GACTATCATTACCAGCTGGAAATGGCAAAAAACATTACC |
| | GCAGCTGCGGAAGCGATCTATACGCCGGAAGAAGCTCCG |
| | GCGAAAATTGATCACGTTATCAAAACCGCGCTGCGTGAG |
| | AAAAAACCGGTCTACCTGGAAATTGCGTGCAATATCGCCT |
| | CAATGCCGTGTGCAGCACCGGGTCCGGCATCGGCACTGTT |
| | TAATGATGAAGCAAGCGACGAAGCTTCTCTGAACGCTGC |
| | GGTGGATGAAACCCTGAAATTCATTGCGAACCGTGACAA |
| | AGTTGCAGTCCTGGTGGGCAGCAAACTGCGTGCCGCAGG |
| | TGCAGAAGAAGCTGCGGTCAAATTTACCGATGCACTGGG |
| | CGGTGCTGTGGCAACGATGGCCGCAGCTAAAAGCTTTTTC |
| | CCGGAAGAAAATGCCCTGTATATCGGCACCTCATGGGGT |
| | GAAGTGTCGTACCCGGGTGTTGAAAAAACGATGAAAGAA |
| | GCCGATGCAGTCATTGCTCTGGCGCCGGTGTTCAATGACT |
| | ATAGCACCACGGGCTGGACCGATATCCCGGACCCGAAAA |
| | AACTGGTTCTGGCGGAACCGCGTAGCGTCGTGGTTAACG |
| | GTATTCGCTTTCCGTCTGTGCATCTGAAAGATTACCTGAC |
| | CCGTCTGGCCCAAAAAGTTAGCAAGAAAACCGGCTCTCT |
| | GGACTTTTTCAAAAGTCTGAATGCGGGTGAACTGAAAAA |
| | AGCAGCACCGGCCGATCCGTCCGCACCGCTGGTCAATGC |
| | GGAAATTGCACGTCAGGTGGAAGCACTGCTGACCCCGAA |
| | CACCACGGTGATCGCCGAAACGGGCGACTCTTGGTTCAAT |
| | GCACAACGTATGAAACTGCCGAACGGTGCGCGCGTTGAA |
| | TATGAAATGCAGTGGGGCCATATTGGTTGGAGCGTTCCGG |
| | CAGCTTTTGGCTACGCAGTCGGTGCTCCGGAACGTCGCAA |
| | CATCCTGATGGTGGGCGATGGTTCGTTCCAGCTGACCGCA |
| | CAAGAAGTTGCTCAGATGGTCCGTCTGAAACTGCCGGTCA |
| | TCATCTTTCTGATCAACAACTACGGCTACACGATTGAAGT |
| | GATGATCCACGATGGTCCGTATAATAACATCAAAAATTG |
| | GGACTACGCCGGCCTGATGGAAGTGTTTAATGGTAACGG |
| | CGGTTATGATAGTGGCGCGGCCAAAGGTCTGAAAGCGAA |
| | AACCGGCGGTGAACTGGCCGAAGCAATTAAAGTTGCTCT |
| | GGCGAACACCGATGGCCCGACGCTGATTGAATGCTTCATC |
| | GGTCGCGAAGACTGTACCGAAGAACTGGTTAAATGGGGC |
| | AAACGTGTCGCAGCTGCGAATAGCCGCAAACCGGTGAAC |
| | AAAGTCGTG (SEQ ID NO: 110) |
| 1OZF | |
| YP_006485164.1 | |
| YP_005461458.1 | |
| YP_006991301.1 | |
| WP_003075272.1 | |
| WP_020634527.1 | |
| 1OVM | |
| 2Q5Q | |
| 2VBG | |
| 2VBI | |
| 3FZN | |

### Protein Production and Enzyme Purification

Overnight cultures of BLR cells suspended in a 2 mL volume were transformed with a pet29b+ plasmid (encoding polypeptides of interest with a C-terminal His-tag) and grown in Terrific Broth with 50 μg/ml kanamycin. Cultures were diluted 1:1,000 in 500 ml of Terrific Broth with 1 mM MgSO4, 1% glucose and 50 μg/ml antibiotic and then grown at 37° C. for 24 hours. Cultures were pelleted down at 4,700 RPM for 10 minutes and resuspended in auto-induction media (LB broth, 1 mM MgSO4, 0.1 mM TPP, 1×NPS and 1×5052) for induction at 18° C. for 20 hours. At the end of induction, cells were centrifuged, the supernatant was removed and cells were resuspended in 40 mL lysis buffer (100 mM HEPES, pH 7.5, 100 mM NaCl, 10% glycerol, 0.1 mM TPP, 1 mM MgSO4, 10 mM Imidazole, 1 mM TCEP) and 1 mM phenylmethylsulphonyl fluoride. The cell lysate suspension was sonicated for 2 min and followed by centrifugation at 4,700 RPM. The supernatant was loaded onto a gravity flow column with 500 uL Cobalt beads and was washed with 15 mL of wash buffer five times. Proteins were eluted with 1,000 mL of elution buffer (100 mM HEPES, pH 7.5, 100 mM NaCl, 10% glycerol, 0.1 mM TPP, 1 mM MgSO4, 200 mM Imidazole and 1 mM TCEP). Protein concentrations were determined using a Synergy H1 spectrophotometer (Biotek) by measuring absorbance at 280 nm using calculated extinction coefficients.

### Enzyme Activity Assay and Kinetic Characterization

All substrates were dissolved in MilliQ H₂O and the pH was adjusted to 7.2 as necessary. Activity for oxaloacetate, pyruvate, and 2-ketoisovalerate was measured at a 1 mM substrate concentration. The assay was performed in a 96-well half-area plate. Each reaction contained reaction buffer (100 mM HEPES, 100 mM NaCl, 10% glycerol, pH 7.2), ADH (Sigma-Aldrich, A7011, 100 U/mL for pyruvate, 600 U/mL for oxaloacetate, and 600 U/mL for 2-ketoisovalerate), and a final concentration of 0.5 mM NADPH, 0.1 mM TPP, and 1 mM MgSO₄. A range of substrate concentrations (0.1 mM-5 mM) were uSEQ to perform steady-state kinetics measurement over a period of one hour. Absorbance readings were taken at one minute intervals at 340 nm at 21° C. for 60 minutes using the Synergy H1 spectrophotometer (Biotek). Kinetic parameters (k_{cat} and K_{M}) were determined by fitting initial velocity versus substrate concentration data to the Michaelis-Menten equation.

Results

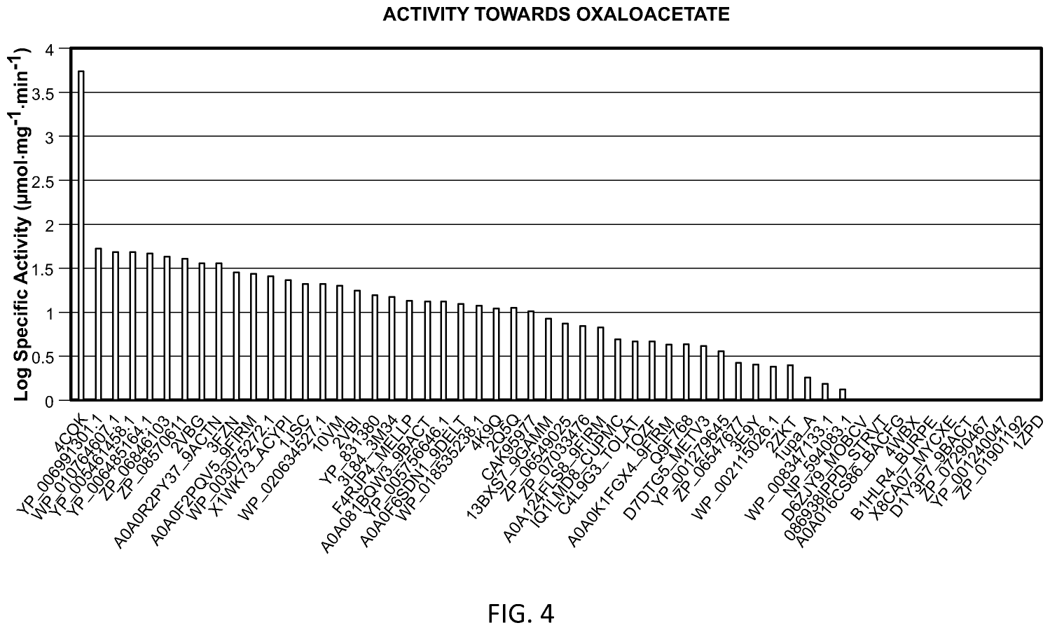

FIG. 4 and Table 3 show the activity of 56 candidate oxaloacetate decarboxylases towards the substrates oxaloacetate, pyruvate, and 2-ketoisovalerate.

TABLE 3
Activity of oxaloacetate decarboxylases

| | | Activity (μmol · mg⁻¹ · min⁻¹) | | |
|---|---|---|---|---|
| Enzyme name or | | | 2-keto | |
| UniProt/Genbank ID | Species | Oxaloacetate | isovalerate | Pyruvate |
| 4COK | Gluconacetobacter diazotrophicus | 5533.300 | 14.118 | 19333.333 |
| A0A0F6SDN1_9DELT | Sandaracinus amylolyticus | 12.307 | 15.578 | 490.212 |
| 4K9Q | Polynucleobacter necessarius subsp. | 10.981 | 55.816 | 0.000 |
| | Asymbioticus | | | |
| D6ZJY9_MOBCV | Mobiluncus curtisii | 0.000 | 15.337 | 32.277 |
| \|Q1LMD8_CUPMC | Cupriavidus metallidurans | 4.712 | 6.326 | 0.000 |
| Q9F768 | Bacteroides fragilis | 4.259 | 0.000 | 0.000 |
| I3BXS7_9GAMM | Thiothrix nivea DSM 5205 | 8.059 | 21.794 | 0.000 |
| 1JSC | Saccharomyces cerevisiae | 21.015 | 22.577 | 0.000 |
| O86938\|PPD_STRVT | Streptomyces viridochromogenes | 0.000 | 3.627 | 0.000 |
| 3L84_3M34 | Campylobacter jejuni | 14.554 | 0.000 | 30.758 |
| 1upa_A | Streptomyces clavuligerus | 1.733 | 17.287 | 1.499 |
| A0A016CS86_BACFG | Fibrobacter succinogenes | 0.000 | 14.840 | 0.000 |
| A0A0F2PQV5_9FIRM | Peptococcaceae bacterium BRH_c4b | 26.972 | 0.000 | 24.122 |
| D7DTG5_METV3 | Methanococcus voltae | 3.983 | 9.969 | 27.183 |
| 3E9Y | Arabidopsis thaliana | 2.499 | 0.000 | 0.000 |
| 2ZKT | Pyrococcus furiosus | 2.385 | 5.429 | 18.603 |
| A0A124FLS8_9FIRM | Clostridia bacterium 62_21 | 6.465 | 57.886 | 79.706 |
| 4WBX | Pyrococcus furiosus | 0.000 | 2424.874 | 69.184 |
| C4L9G3_TOLAT | Tolumonas auensis | 4.623 | 15.720 | 72.346 |
| A0A0K1FGX4_9FIRM | Selenomonas noxia ATCC 43541 | 4.326 | 8.736 | 154.754 |
| A0A0R2PY37_9ACTN | Acidimicrobium sp. BACL17 | 34.977 | 23.241 | 617.232 |
| X1WK73_ACYPI | Acyrthosiphon pisum | 23.275 | 61.946 | 1162.672 |
| B1HLR4_BURPE | Burkholderia pseudomallei | 0.000 | 13.333 | 13.333 |
| X8CA07_MYCXE | Mycobacterium xenopi 3993 | 0.000 | 33.333 | 26.600 |
| D1Y3P7_9BACT | Pyramidobacter piscolens W5455 | 0.000 | 0.000 | 26.700 |
| F4RJP4_MELLP | Melampsora laricipopulina | 13.333 | 24.444 | 26.600 |
| A0A081BQW3_9BACT | Candidatus Moduliflexus flocculans | 13.333 | 42.222 | 66.667 |
| CAK95977 | Pseudomonas fluorescens | 10.22193433 | 0 | 0 |
| YP_831380 | Arthrobacter sp. | 15.81263828 | 0 | 0 |
| ZP_06547677 | Pseudomonas putida CSV86 | 2.636659175 | 708.837523* | 1648.5245* |
| ZP_06846103 | Halotalea alkalilenta | 42.16910984 | 17.5671744* | 1195.18032* |
| ZP_07290467 | Streptomyces sp. | 0 | 83.3824552* | 267.885245* |
| ZP_08570611 | Rheinheimera sp. A13L | 39.1977264 | 0 | 0 |
| YP_001240047 | Bradyrhizobium sp. STM 3843 | 0 | 0 | 0 |
| YP_001279645 | Psychrobacter sp. | 3.556735997 | 0 | 0 |
| ZP_01901192 | Roseobacter sp. AzwK-3b | 0 | 0 | 0 |
| ZP_06549025 | Serratia marcescens FGI94 | 7.392211819 | 139902.1428 | 9.954203568 |
| ZP_07033476 | Granulicella mallensis | 7.065903742 | 811.4324283 | 1174.57377 |
| | ATCC BAA-1857 | | | |
| WP_010764607.1 | Enterococcus haemoperoxidus | 48.42956916 | 63422.30474 | 1689.737705 |
| | ATCC BAA-382 | | | |
| WP_002115026.1 | Acinetobacter baumannii | 2.410507246 | 0 | 30.67169555 |
| YP_005756646.1 | Staphylococcus aureus | 13.01208771 | 792778.8092 | 15900.58689 |
| WP_008347133.1 | Bacillus pumilus SAFR-032 | 1.544738956 | 0 | 0 |
| WP_018535238.1 | Streptomyces glaucescens | 11.67518701 | 93.58311535 | 35.54345178 |
| YP_006485164.1 | Pseudomonas aeruginosa | 44.89076789 | 242.8363761 | 113.7848268 |
| YP_005461458.1 | Actinoplanes missouriensis | 47.6189372 | 70.38233411 | 370.9180328 |
| YP_006991301.1 | Carnobacterium maltaromaticum LMA28 | 52.96875 | 195862.9999 | 2055.147506 |
| NP_594083.1 | Schizosaccharomyces pombe | 1.312105291 | 0 | 8424.567708 |
| WP_003075272.1 | Comamonas testosteroni | 24.95980669 | 623.2146098 | 147.6722275 |
| WP_020634527.1 | Amycolatopsis orientalis | 20.61304942 | 4.067348776 | 11.61476828 |
| | HCCB10007 | | | |
| 1OVM | Enterobacter sp. | 18.7477487 | 8954.54365* | 158.667580* |
| 2Q5Q | Azospirillum brasilense Sp24 | 10.86768802 | 0 | 23.95798121 |
| 2VBG | Lactococcus lactis | 35.41517071 | 67191.9 | 1257 |
| 2VBI | Acetobacter syzygii 9H-2 | 16.99543089 | 36.2215268* | 201944.262* |
| 3FZN | Agrobacterium radiobacter | 27 | 1987.26023* | 370.918032* |
| 1ZPD | Zymomonas mobilis | 0 | 18.1191493* | 453344.262* |
| | subsp. mobilis | | | |
| 1OZF | Klebsiella pneumoniae | 4.537374205 | 419.706428* | 391.524590* |
| | subsp. Pneumoniae | | | |

*Indicates values calculated based on published data (Mak, W. S. et al. (2015) Nat. Commun. 6: 10005).

Functional characterization indicated that 45 of the 56 diverse enzyme candidates identified from the genomic database described earlier showed activity towards oxaloacetate. Among these active homologues, pyruvate decarboxylase from Gluconoacetobacter diazotrophicus (PDB code: 4COK; see van Zyl, L. J. et al. (2014) BMC Struct. Biol. 14:21) was found to be most active. As shown in Table 3, 4COK exhibited more than 100-fold higher activity towards oxaloacetate than any other decarboxylase tested.

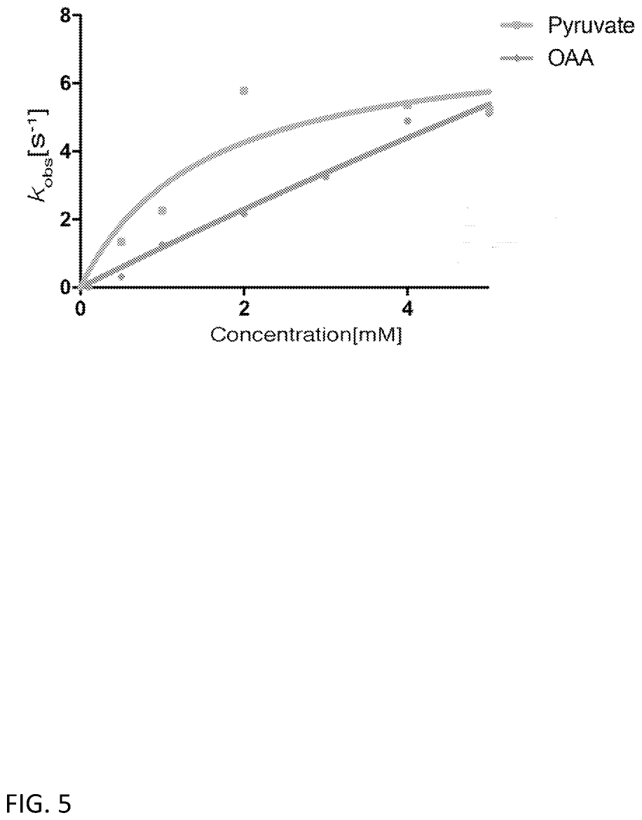

As shown in Table 4 and FIG. 5. 4COK exhibited a catalytic efficiency (k_{cat}/K_{M}) of approximately 2296.4 M⁻¹s⁻¹ for oxaloacetate and approximately 5532.1 M⁻¹s⁻¹ for pyruvate.

TABLE 4
Kinetic constants of 4COK for pyruvate and oxaloacetate

| | Pyruvate | Oxaloacetate |
|---|---|---|
| k_{cat} (s−1) | 8.254 ± 1.87 | n.d. |
| K_{M} (mM) | 1.49 ± 0.43 | n.d. |
| k_{cat}/K_{M} (M⁻¹s⁻¹) | 5532.1 ± 39.4 | 2296.4 ± 116 |

These findings indicated that pyruvate decarboxylase from Gluconoacetobacter diazotrophicus catalyzed the decarboxylation of oxaloacetate to 3-oxopropanoate, acting as an efficient oxaloacetate decarboxylase (OAADC). The direct conversion of oxaloacetate to 3-oxopropanoate using an OAADC enables a novel and advantageous metabolic pathway to produce 3-HP.

### Example 2: Identification of Additional Oxaloacetate Decarboxylases, Alcohol Dehydrogenases, and Phosphoenolpyruvate Carboxykinases

Materials and Methods

### Genome Mining

A second round of genome mining was conducted as described in Example 1, except using the 4COK sequence as the input. Genes encoding candidate OAADCs were synthesized and expressed in E. coli for further characterization. OAADC activity was assayed as described in Example 1.

### Alcohol Dehydrogenase (ADH) Activity

Candidate ADHs were expressed in E. coli, and soluble expression levels were analyzed. 3-HP dehydrogenase (3-HPDH) activity of each was tested based on the reverse reaction, from 3-HP to 3-oxopropanoate. The assay was performed in a 96-well half-area plate. Each reaction contained a final concentration of 1 mM NADP⁺/NAD⁺ in reaction buffer (100 mM Hepes, 100 mM NaCl, 10% glycerol, pH 7.2) and ADHs. A range of substrates from 0.1 mM-5 mM was used to perform steady-state kinetics measurement over a period of an hour. Absorbance readings were taken every 1 min at OD 340 at 21° C. for 60 min. using the Synergy™ H1 Hybrid Multi-Mode Microplate Reader (Biotek). Kinetic parameters (k_{cat} and K_{M}) were determined by fitting initial velocity versus substrate concentration data to the Michaelis-Menten equation.

### Phosphoenolpyruvate Carboxykinase (PEPCK) Activity

5 genes encoding candidate PEPCKs were synthesized and cloned into expression vectors. After obtaining solubly expressed proteins, they were used for activity characterization. Each enzyme was assayed in the phosphoenolpyruvate carboxylation direction in a solution containing 100 mM PBS buffer (pH 6.5), 0.20 mM NADH, 1.25 mM ADP, 2.5 mM PEP, 50 mM KHCO₃, 2 mM MnCl₂, and 4 units malate dehydrogenase.

Results

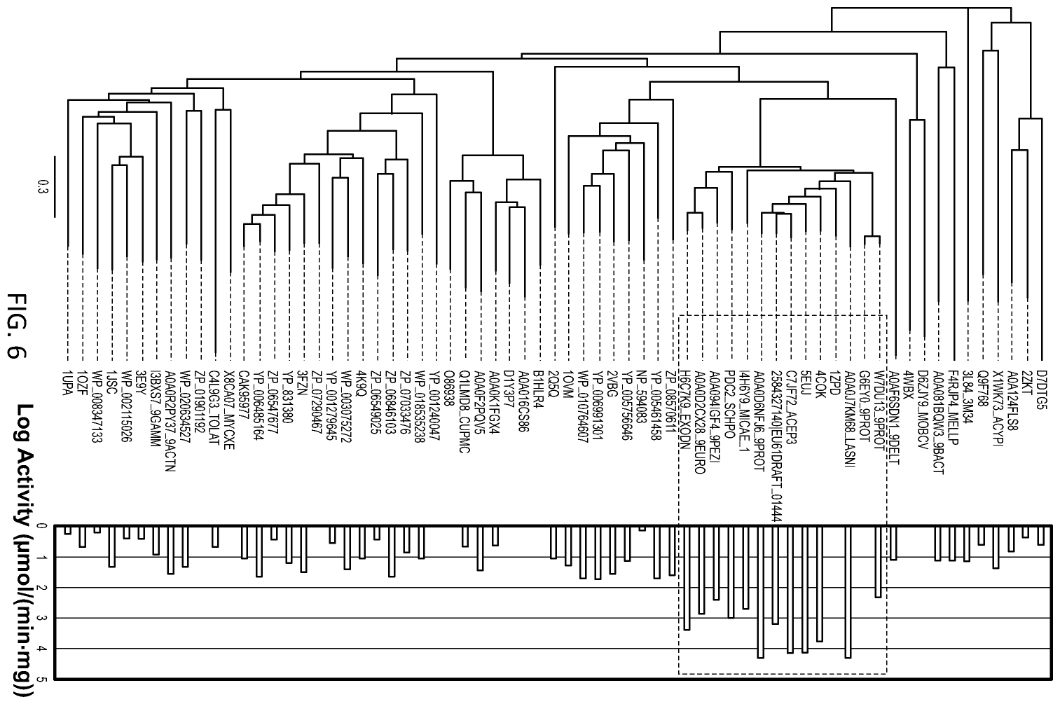

A second round of genome mining was performed to explore the sequence space around the enzyme 4COK, which found to be highly active in the first round of mining described in Example 1. These analyses identified many proteins with measurable OAADC activity. In particular, a highly active enzyme cluster was identified, including the most active, newly identified OAADCs A0A0J7KM68, C7JF72_ACEP3, 5EUJ, and A0A0D6NFJ6_9PROT (FIG. 6). The sequences of the enzymes in the clade highlighted in FIG. 6 are provided in Table 5.

TABLE 5
Candidate sequences in clade with highest OAADC specific activity.

| Enzyme name | Amino acid sequence |
|---|---|
| G6EYP0 9PROT | MEYTVGQYLATRLAQLGLNHFAVAGDYNLTLLDEMAKAKDLEQVYCCNEL |
| | NCGFAGEGYARARIMGASVVTFSVGAFSAFNAVGGAFAENLPLLLISGAPNNN |
| | DYGSGHILHHTMGYSDYRYQMEMAKKITCEAVSVAHADEAPCLIDHAIRSAIR |
| | NRKPAYIEISCNVANQPCTEPGPISSITNSLISDDESLKAAAKACVEALEKAKNPV |
| | VIIGGKIRSAGCAVSKQVAELTKKLGCAVATMAQAKGLSPEEEAEYVGTFWGD |
| | ISSPGVEDLVRDSDCRIYIGAVFNDYSTVGWTCKLVSDNDILISSHHTRVGKKEF |
| | SGVYLKDFIPVLASSVKKNTTSLEQFKAKKLPAKETPVADGNAALTTVELCRQI |
| | QGAINKDTTLFLETGDSWFHGMHFNLPNGARVESEMQWGHIGWSIPSMFGYAV |
| | SEPNRRNHMVGDGSFQLTAQEVCQMIRRNMPWIHLINNSGYTIEVKIHDGPYNRI |
| | KNWDYAGLIDVFNAEDGKGLGLKAKNGAELEKAMKTALAHKDGPTLIEVDID |
| | AQDCSPDLVVWGKKVAKANGRAPRKAGGSG (SEQ ID NO: 137) |
| W7DU13 9PROT | MKYTVGQYLATRLAQLGLNHVFAVAGDYNLTLLDEMAKVEDLEQVYCCNEL |
| | NCGFAGEGYARSRVMGASVVTFSVGAFSAFNAVGGAFAENLPLLLISGAPNNN |
| | DYGSGHILHHTMGYSDYRYQMDMAKQITCEAVSVAHADEAPCLIDHAIRSALR |
| | NRKPAYIEISCNVANQPCTEPGPISSITNSLISDDESLKAAAKACLDALEKAKSPV |
| | VIIGGKIRSAGCAVSKKVAELTKKLGCAVATMAQAKGLSPEEEAEYVGTFWGEI |
| | SSPGVEELVRESDCRIYIGAVFNDYSTVGWTCKLVGENDILISSHHTRVGHKEFS |
| | GVYLKDFIPVLTSCVKKNTTSLDQFKAKKIPVKQVPVADGKAPLTTVELCRQIQ |
| | GAINKDTTIYLETGDSWFHGMHFKLPNGARVESEMQWGHIGWSIPSMFGYAVS |
| | EPNRRNHMVGDGSFQLTAQEVCQMIRRNIPHHLINNSGYTIEVKIHDGPYNRIKN |
| | WDYAGLINVFNAEDGKGLGLKAKNGAELEKAMQTALAHKDGPTLIEVDIDAQ |
| | DCSPDLVVWGKKVAKANGRAPRKFQTFGGSG (SEQ ID NO: 138) |
| I4H6Y9 MICAE_1 | MSNYNVGTYLAERLVQIGVKHHFVVPGDYNLVLLDQFLKNQNLLQVGCCNEL |
| | NCGFAAEGYARANGLGVAVVTYSVGALSALNAIGGAYAENLPVILVSGAPNTN |
| | DYSTGHLLHHTMGTQDLTYVLEIARKLTCAAVSITSAEDAPEQIDHVIRTALREQ |
| | KPAYIEIACNIAAAPCASPGPVSAIINEVPSDAETLAAAVSAAAEFLDSKQKPVLL |
| | IGSQLRAAKAEQEAIELAEALGCSVAVMAAAKSFFPEEHPQYVGTYWGEISSPG |
| | TSAIVDWSDAVVCLGAVFNDYSTVGWTAMPSGPTVLNANKDSVKFDGYHFSGI |
| | HLRDFLSCLARKVEKRDATMAEFARFRSTSVPVEPARSEAKLSRIEMLRQIGPLV |
| | TAKTTVFAETGDSWFNGMKLQLPTGARFEIEMQWGHIGWSIPAAFGYALGAPE |
| | RQIICMIGDGSFQLTAQEVAQMIRQKLPHIFLWNHGYTIEVEIHDGPYNNIKNW |
| | DYAGLIKVFNAEDGAGQGLLATTAGELAQAIEVALENREGPTLIECVIDRDDAT |
| | ADLISWGRAVAVANARPHRGGSG (SEQ ID NO: 139) |
| A0A094IGF4 9PEZI | MATFTVGDYLAERLAQIGIRHHFVVPGDYNLILLDKLQSHPDLSELGCANELNC |
| | SLAAEGYARAQGVAACIVTYSVGAFSAFNGTGSAYAENLPLILVSGSPNTNDSA |
| | KFHLLHHTLGTNDFTYQFEMAKKITCCAVAVGRAQDAPRLIDQAIRAALLAKK |
| | PAYIEIPTNLSGAMCVRPGPISAVVEPVLSDKASLTAAVDRAVQYLCGKQKPAIL |
| | VGPKLRRAGAEMALLQVAEAIGCAVAVQPAAKGFFPEDHKQFAGVFWGQVST |
| | LAADSILNWADTILCVGTIFTDYSTVGWTALPNVPLMIAEMDHVMFPGATFGR |
| | VRLNDFLSGLAKTVGRNESTMVEYGYIRPDPPLVHAAAPDELLNRKETAROVQ |
| | MLLTPETTVFVDTGDSWFNGIRMKLPRGASFEIEMQWGHIGWSIPAAFGYAMG |
| | KPERKVITMVGDGSFQMTAQEVSQMVRYKVPHIFLINNKGYTIEVEIHDGLYNR |
| | IKNWDYALLVRAFNSNDGQAIGFRASTGRELAEAIEKAKAHKDGPTLIECVIDQ |
| | DDCSRELITWGHYVAAANARPPVQTGGSG (SEQ ID NO: 140) |
| A0A0D2CX28 | MSWTVGSYLAERLAQIGIEHHFWPGDYNLVLLDKLQAHPKLSEIGCANELNCS |
| 9EURO | FAAEGYARAKGVAAAVVTFSVGAFSAFNGVGGAYAENLPVILISGAPNTSDSG |
| | AFHLLHHTLGTHDFGYQLEMAKKITCAAVAIRRAQDAPRLIDHAIRSAMSAKKP |
| | AYIEIPTNLSIANCPAPGPISAVIAPERSDEITLAMAVNAALDWLKSKQKPVLLAG |
| | PKLRAAGAEAAFLQLADALGCAVAVLPGAKSFFPEDHKQFVGVYWGQVSTMG |
| | ADAIVDWSDGIFGAGVVFTDYSTVGWTALPPDSITLTADLDHMSFTGAEFNRV |
| | QLAELLSALAERATRNSSTMVEYAHLRPDVLFPHIEEPKLPLHRNEIARQIQQLL |
| | QPKTTLFVETGDSWFNGVQMRLPRSCRFEIEMQWGHIGWSVPASFGYAVGSPE |
| | RQHLMVGDGSFQMTVQEVSQMVRARLPIHFLMNNRGYTIEVEIHDGLYNRIKN |
| | WNYASLIEAFNAEDGHAKGIKASNPEQLAQAIKLATSNSDGPTLIECVIDQDDCT |
| | RELITWGHYVASANARPPAHKGGSG (SEQ ID NO: 141) |
| H6C7K9 EXODN | MRCMSVPSMTFSRHTLRSCATSSDRMTGAPRKPFITSIKRQHQOPWHSICPNVTI |
| | IMSWTVGSYLAERLSQIGIEHHFVVPGDYNLVLLDQLQAHPKLSEIGCANELNC |
| | SFAAEGYARAKGVAAAVVTFSVGAFSAFNGLGGAYAENLPVILISGSPNTNDAG |
| | AFHLLHHTLGTHDFEYQRQIAEKITCAAVAVRRAQDAPRLIDHAIRSALLAKKP |
| | SYIEIPTSNVTCPAPGPISAVIAPEPSDEPTLAAAVHAATNWLKAKQKPILLAG |
| | PKLRAAGGEAGFLQLAEAIGCAVAVMPGAKSFFPEDHKQFVGVYWGQASTMG |
| | ADAIYDWADGIFGAGLWTDYSTVGWTAIPSESITLNADLDNMSFPGATFNRVR |
| | LADLLSALAKEATPNPSTMVEYARLRPDILPPHHEQPKLPLHRVEIAROIQELLH |
| | PKTTLFAETGDSWFNAMQMNLPRDCRFEIEMQWGHIGWSVPASFGYAVGAPE |
| | RQVLLMIGDGSFQMTAQEVSQMWSKPHIFLMNNGGYTIEVEIHDGLYNRIKN |
| | WNYAAMMEVFNAGDGHAKGIKASNPEQLAQAIKLAKSNSEGPTLIECHDQDD |
| | CTKELITWGHYVATANGRPPAHTGGSG (SEQ ID NO: 142) |
| PDC2 SCHPO | MTKDAESTMTVGTYLAQRLWIGIKNHFVVPGDYNLRLLDFLEWPGLSEIGCC |
| | NELNCAFAAEGYARSNGIACAVVTYSVGALTAFDGIGGAYAENLPVILVSGSPN |
| | TNDLSSGHLLHHTLGTHDFEYQMEIAKKLTCAAVAIKRAEDAPVMIDHAIRQAI |
| | LQHKPVYIEIPTNMANQPCPVPGPISAVISPEISDKESLEKATDIAAELISKKEKPIL |
| | LAGPKLRAAGAESAFVKLAEALNCAAFIMPAAKGFYSEEHKNYAGVYWGEVS |
| | SSETTKAVYESSDLVIGAGVLFNDYSTVGWAAPNPNILLNSDYTSVSIPGYVFS |
| | RVYMAEFLELLAKKVSKKPATLEAYNKARPQTVVPKAAEPKAALNRVEVMRQ |
| | IQGLVDSNTTLYAETGDSWFNGLQMKLPAGAKFEVEMQWGHIGWSVPSAMGY |
| | AVAAPERRTIVMVGDGSFQLTGQEISQMIRHKLPVLIFLLNNRGYTIEIQIHDGPY |
| | NRIQNWDFAAFCESLNGETGKAKGLHAKTGEELTSAIKVALQNKEGPTLIECAI |
| | DTDDCTQELVDWGKAVRSANARPPTADNGGSG (SEQ ID NO: 143) |
| 1ZPD | MSYWGTYLAERLVQIGLKHHFAVAGDYNLVLLDNLLLNKNMEQVYCCNELN |
| | CGFSAEGYARAKGAAAAVVTYSVALSAFDAIGGAYAENLPVILISGAPNNND |
| | HAAGHVLHHALGKTDYHYQLEMAKNITAAAEAIYTPEEAPAKIDHVIKTALRE |
| | KKPVYLEIACNIASMPCAAPGPASALFNDEASDEASLNAAVDETLKFIANRDKV |
| | AVLVGSKLRAAGAEEAAVKFTDALGGAVATMAAAKSFFPEENALYIGTSWGE |
| | VSYPGVEKTMKEADAVIALAPVFNDYSTTGWTDIPDPKKLVLAEPRSVVVNGIR |
| | FPSVHLKDYLTRLAQKVSKKTGSLDFFKSLNAGELKKAAPADPSAPLVNAEIAR |
| | QVEALLTPNTTVIAETGDSWFNAQRMKLPNGARVEYEMQWGHIGWSVPAAFG |
| | YAVGAPERRNILMVGDGSFQLTAQEVAQMVRLKLPVIIFLINNYGYTIEVMIHD |
| | GPYNNIKNWDYAGLMEVFNGNGGYDSGAAKGLKAKTGGELAEAIKVALANT |
| | DGPTLIECFIGREDCTEELVKWGKRVAAANSRKPVNKW (SEQ ID NO: 144) |
| 4COK | MTYTVGRYLADRLAQIGLKHHFAVAGDYNLVLLDQLLLNTDMQQIYCSNELN |
| | CGFSAEGYARANGAAAAIVTFSVGALSAFNALGGAYAENLPVILISGAPNANDH |
| | GTGHILHHTLGTTDYGYQLEMARHITCAAESIVAAEDAPAKIDHVIRTALREKK |
| | PAYLEIACNVAGAPCVRPGGIDALLSPPAPDEASLKAAVDAALAFIEQRGSVTM |
| | LVGSRIRAAGAQAQAVALADALGCAVTTMAAAKSFFPEDHPGYRGHYWGEVS |
| | SPGAQQAVEGADGVICLAPVFNDYATVGWSAWPKGDNVMLVERHAVTVGGV |
| | AYAGIDMRDFLTRLAAHTVRRDATARGGAYVTPQTPAAAPTAPLNNAEMARQI |
| | GALLTPRTTLTAETGDSWFNAVRMKLPHGARVELEMQWGHIGWSVPAAFGNA |
| | LAAPERQHVLMVGDGSFQLTAQEVAQMIRHDLPVIIFLINNHGYTIEVMIHDGP |
| | YNNVKNWDYAGLMEVFNAGEGNGLGLRARTGGELAAAIEQARANRNGPTLIE |
| | CTLDRDDCTQELVTWGKRVAAANARPPRAG (SEQ ID NO: 1) |
| A0A0J7KM68 | MSYTVGQYLADRLVQIGLKDHFAIAGDYNLVLLDQFLKNKNWNQIYDCNELN |
| LASNI | CGFAAEGYARANGAAACVVTYTVGAISAMNSALAGAYAENLPVLCISGAPNC |
| | NDYGSGRILHHTIGKPEFTQQLDMVKHWCAAESVVQASEAPAKIDHVIRTMLL |
| | EQRPAYIDIACNISGLECPRPGPIEDLLPQYAADNKSLTSAIDAIAKKIEASQKVTL |
| | YVGPKVRPGKAKEASVKLADALGCAVTVGPASMSFFPAKHPGFRGTYWGIVST |
| | GDANKVVEEAETLIVLGPNWNDYATVGWKAWPKGPRVVTIDEKAAQVDGQV |
| | FSGLSMKALVEGLAKKVSKKPATAEGTKAPHFEYPVAKPDAKLTNAEMARQIN |
| | AILDDNTTLHAETGDSWFNVKNMNWPNGLRIESEMQYGHIGWSIPSGFGGAIGS |
| | PERKHIIMCGDGSFQLTCQEVSQMIRYKLPVTIFLIDNHGYGIEIAIHDGPYNYIQ |
| | NWNFTKLMEVFNGEGEECPYSHNKNGKSGLGLKATTPAELADAIKQAEANKE |
| | GPTLIQVVIDQDDCTKDLLTWGKEVAKTNARSPVVTDKAGGSG (SEQ ID |
| | NO: 145) |
| 5EUJ | MYTVGMYLAERLAQIGLKHHFAVAGDYNLVLLDQLLLNKDVMEQVYCCNELN |
| | CGFSAEGYARARGAAAAIVTFSVGAISAMNAIGGAYAENLPVILISGSPNTNDY |
| | GTGHILHHTIGTTDYNYQLEMVKHVTCAAESIVSAEEAPAKIDHVIRTALRERKP |
| | AYLEIACNVAGAECVRPGPINSLLRELEVDQTSVTAAVDAAVEWLQDRQNVV |
| | MLVGSKLRAAAAEKQAVALADRLGCAVTIMAAAKGFFPEDHPNFRGLYWGEV |
| | SSEGAQELVENADAILCLAPVFNDYATVGWNSWPKGDNVMVMDTDRVTFAG |
| | QSFEGLSLSTFAAALAEKAPSRPATTQGTQAPVLGIEAAEPNAPLTNDEMTRQIQ |
| | SLITSDTTLTAETGDSWFNASRMPIPGGARVELEMQWGHIGWSVPSAFGNAVGS |
| | PERRHIMMVGDGSFQLTAQEVAQMIRYEIPVIITFLINNRGYVIEIAIHDGPYNYIK |
| | NWNYAGLIDVFNDEDGHGLGLKASTGAELEGAIKKALDNRRGPTLIECNIAQD |
| | DCTETLIAWGKRVAATNSRKPQAGGSG (SEQ ID NO: 146) |
| 2584327140 | MAYTVGMYLAERLAQIGLKHHFAVAGDYNLVLLDQLLLNKDMEQIYCCNELN |
| EU61DRAFT | CGFSAEGYARAHGAAAAVVTFSVGAISAMNAIGGAYAENLPVILISGSPNSNDY |
| | GSGHILHHTLGTTDYGYQLEMARHVTCAAESITDAASAPAKIDHVIRTALRERK |
| | PAYLEIACNVSSAECPRPGPVSSLLAEPATDPVSLKAALEASLSALNKAERVVML |
| | VGSKIRAADAQAQAVELADRLGCAVTIMSAAKGFFPEDHPGFRGLYWGEVSSP |
| | GAQELVENADAVLCLAPVFNDYSTVGWNAWPKGDKVLLAEPNRVTVGGQSFE |
| | GFALRDFLKGLTDRAPSKPATAQGTHAPKLEIKPAARDARLTNDEMARQINAM |
| | LTPNTTLAAETGDSWFNAMRMNLPGGARVEVEMQWGHIGWSVPSTFGNAMG |
| | SKDRQHIMMVGDGSFQLTAQEVAQMVRYELPVIIFLVNNKGYVIEIAIHDGPYN |
| | YIKNWDYAGLMEVFNAGEGHGIGLHAKTAGELEDAIKKAQANKRGPTIIECSLE |
| | RTDCTETLIKWGKRVAAANSRKPQAVGGSG (SEQ ID NO: 147) |
| C7JF72 ACEP3 | MTYTYVGMYLAERLSQIGLKHHFAVAGDFNLVLLDQLLVNKEMEQVYCCNELN |
| | CGFSAEGYARAHGAAAAVVTFSVGAISAMNAIAGAYAENLPVILISGSPNSNDY |
| | GTGHILHHTLGTNDYTYQLEMMRHVTCAAESITDAASAPAKIDHVIRTALRERK |
| | PAYVEIACNVSDAECVRPGPVSSLLAELRADDVSLKAAVEASLALLEKSQRVTM |
| | IVGSKVRAAHAQTQTEHLADKLGCAVTIMAAAKSFFPEDHKGFRGLYWGDVSS |
| | PGAQELVEKSDALICVAPVFNDYSTVGWTAWPKGDNVLLAEPNRVTVGGKTY |
| | EGFTLREFLEELAKKAPSPLTAQESKKHTPVIEASKGDARLTNDEMTRQINAM |
| | LTSDTTLVAETGDSWFNATRMDLPRGARVELFMQWGHIGWSVPSAFGNAMGS |
| | QERQHILMVGDGSFQLTAQEMAQMVRYKLPVIIFLVNNRGYVIEIAIHDGPYNY |
| | IKNWDYAGLMEVFNAEDGHGLGLKATTAGELEEAIKKAKTNREGPTIIECQIER |
| | SDCTKTLVEWGKKVAAANSRKPQVSGGSG (SEQ ID NO: 148) |
| A0A0D6NFJ6 | MTYTVGMYLADRLAQIGLKHHFAVAGDYNLVLLDQLLTNKDMQQIYCCNELN |
| 9PROT | CGFSAEGYARAHGAAAAVVTFSVGAISAMNAIGGAYAENLPVILISGSPNSNDY |
| | GSGHILHHTIGSTDYGYQMEMVKHVTCAAESITDAASAPAKIDHVIRTALRESK |
| | PAYLEIACNVSAQECPRPGPVSSLLSEPAPDKTSLDAAVAAAVKLIEGAENTVIL |
| | VGSKLRAARAQAEAEKLADKLECAVTIMAAAKGFFPEDHAGFRGLYWGEVSS |
| | PGTQELVEKADAIICLAPVFNDYSTVGWTAWPKGDKVLLAEPNRVTIKGQTFEG |
| | FALRDFLTALAAKAPARPASAKASSHTPTAFPKADAKAPLTNDEMARQINAML |
| | TSDTTLVAETGDSWFNAMRMTLPRGARVELEMQWGHIGWSVPSSFGNAMGSQ |
| | DRQHVVMVGDGSFQLTAQEVAQMVRYELPVIIFLVNNRGYVIEIAIHDGPYNYI |
| | KNWDYAGLMEVFNAGEGHGLGLHATTAEELEDAIKKAQANRRGPTIIECKIDR |
| | QDCTDTLVQWGKKVASANSRKPQAVGGSG (SEQ ID NO: 166) |

The kinetics of these enzymes were characterized and compared with that of 4COK. As shown in Table 6, four of these enzymes displayed high levels of OAADC activity, similar to or greater than that of 4COK.

TABLE 6
Kinetics of highly active OAADCs.

| | A0A0J7KM68 | C7JF72_ACEP3 | 5EUJ | A0A0D6NFJ6_9PROT | 4COK |
|---|---|---|---|---|---|
| kcat(s⁻¹) | 6.248 | 55.45 | 28.79 | >121 | >55 |
| Km(mM) | 2.389 | 15.53 | 6.667 | >20 | >20 |
| kcat/Km(M⁻¹s⁻¹) | 2615.3 ± 224.2 | 3570.5 ± 252.5 | 4318.3 ± 320.7 | 6045.2 ± 452.5 | 2296.4 ± 116.0 |

To engineer a novel pathway to produce 3-HP, 3-hydroxypropionate dehydrogenase (3-HPDH) and phosphoenolpyruvate carboxykinase (PEPCK) candidates suitable for the novel pathway were also investigated. As shown in FIG. 2B, the final step in the conversion of sugars into 3-HP is the formation of 3-HP from 3-oxopropanoate, which can be catalyzed by a 3-HPDH. 12 candidate ADHs were expressed in E. coli and tested for solubility and 3-HPDH activity. The sequences of the enzymes tested are provided in Table 7.

TABLE 7
Candidate 3-HPDH sequences.

| Enzyme name | Amino acid sequence |
|---|---|
| ADH6_YEAST | MSYPEKFEGIAIQSHEDWKNPKKTKYDPKPFYDHDIDKIEACGVCGSDIHCAAG |
| | HWGNMKMPLVVGHEIVGKVVKLGPKSNSGLKVGQRVGVGAQVFSCLECDRCK |
| | NDNEPYCTKFVTTYSQPYEDGYVSQGGYANYVRVHEHFVVPIPENIPSHLAAPLL |
| | CGGLTVYSPLVRNGCGPGKKVGIVGLGGIGSMGTLISKAMGAETYVISRSSRKRE |
| | DAMKMGADHYIATLEEGDWGEKYFDTFDLIVVCASSLTDIDFNIMPKAMKVGG |
| | RIVSISIPEQHEMLSLKPYGLKAVSISYSALGSIKELNQLLKLVSEKDIKIWVETLPV |
| | GEAGVHEAFERMEKGDVRYRFTLVGYDKEFSD (SEQ ID NO: 149) |
| YQHD_ECOLI | MNNFNLHTFTRILFGKGAIAGLREQIPHDARVLITYGGGSVKKTGVLDQYLDALK |
| | GMDVLEFGGIEPNPAYETLMNAVKLVREQKVTFLLAVGGGSVLDGTKFIAAAA |
| | NYPENIDPWHILQTGGKETKSAIPMGCVLTLPATGSESNAGAVISRKTTGDKQAF |
| | HSAHVQPVFAVLDPVYTYTLPPRQVANGVVDAFVHTYEQYVTKPVDAKIODRF |
| | AEGILLTLIEDGPKALKEPENYDVRANVMWAATQALNGLIGAGVPQDWATHML |
| | GHELTAMHGLDHAQTLAIVLPALWNEKRDTKRAKLLQYAERVWNITEGSDDER |
| | IDAAIAATRNFFEQLGVPTHLSDYGLDGSSIPALLKKLEEHGMTQLGENHDITLD |
| | VSRRIYEAAR (SEQ ID NO: 150) |
| ADH2_YEAST_A1cohol_dehydrogenase_2 | MSIPETQKAIIFYESNGKLEHKDIPVPKPKPNELLINVKYSGVCHTDLHAWHGDW |
| | PLPTKLPLVGGHEGAGVVVGMGENVKGWKIGDYAGIKWLNGSCMACEYCELG |
| | NESNCPHADLSGYTHDGSFQEYATADAVQAAHIPQGTDLAEVAPILCAGITVYK |
| | ALKSANLRAGHWAAISGAAGGLGSLAVQYAKAMGYRVLGIDGGPGKEELFTSL |
| | GGEVFIDFTKEKDIVSAVVKATNGGAHGIINVSVSEAAIEASTRYCRANGTVVLV |
| | GLPAGAKCSSDVFNHVVKSISIVGSYVGNRADTREALDFFARGLVKSPIKVVGLS |
| | SLPEIYEKMEKGQIAGRYWDTSK (SEQ ID NO: 151) |
| YdfG | MIVLVTGATAGFGECITRRFIQQGHKVIATGRRQERLQELKDELGDNLYIAQLDV |
| | RNRAAIEEMLASLPAEWCNIDILVNNAGLALGMEPAHKASVEDWETMIDTNNK |
| | GLVYMTRAVLPGMVERNHGHIINIGSTAGSWPYAGGNVYGATKAFVRQFSLNL |
| | RTDLHGTAVRVTDIEPGLVGGTEFSNVRFKGDDGKAEKTYQNTVALTPEDVSEA |
| | VWWVSTLPAHVNINTLEMMPVTQSYAGLNVHRQ (SEQ ID NO: 152) |
| A9A4M8 | MHTVRIPKVINFGEDALGQTEYPKNALVVTTVPPELSDKWLAKMGIQDYMLYD |
| | KVKPEPSIDDVNTLISEFKEKKPSVLIGLGGGSSMDVVKYAAQDFGVEKILIPTTF |
| | GTGAEMTTYCVLKFDGHCKLLREDRFLADMAVVDSWMDGTPEQVIKNSVCDA |
| | CAQATEGYDSKLGNDLTRTLCKQAFEILYDADIMNDKPENYPYGSMLSGMGFGN |
| | CSTTLGHALSYYFSNEGVPHGYSLSSCTTVAHKHNKSIFYDRFKEAMDKLGFDK |
| | LELKADVSEAADVMTDKGHLDPNPIPISKDDVVKCLEDIKAGNL (SEQ ID |
| | NO: 153) |
| A4YI81 | MTEKVSVVGAGVIGVGWATLFASKGYSVSLYTEKKETLDKGIEKLRNYVQVMK |
| | NNSQITEDVNTVISRVSPTTNLDEAVRGANFVIEAVIEDYDAKKKIFGYLDSVLDK |
| | EVILASSTSGLLITEVQKAMSKHPERAVIAHPWNPPHLLPLVEIVPGEKTSMEVVE |
| | RTKSLMEKLDRIVVVLKKEIPGFIGNRLAFALFREAVYLVDEGVATVEDIDKVMT |
| | AAIGLRWAFMGPFLTYHLGGGEGGLEYFFNRGFGYGANEWMHTLAKYDKFPYT |
| | GVTKAIQQMKEYSFIKGKTFQEISKWRDEKLLKVYKLVWEK (SEQ ID NO: 154) |
| 3OBB | MKQIAFIGLGHMGAPMATNLLKAGYLLNVFDLVQSAVDGLVAAGASAARSARD |
| | AVQGADVVISMLPASQHVEGLYLDDDGLLAHIAPGTLVLECSTIAPTSARKIHAA |
| | ARERGLAMLDAPVSGGTAGAAAGTLTFMVGGDAEALEKARPLFEAMGRNIFHA |
| | GPDGAGQVAKVCNNQLLAVLMIGTAEAMALGVANGLEAKVLAEIMRRSSGGN |
| | WALEVYNPWPGVMENAPASRDYSGGFMAQLMAKDLGLAQEAAQASASSTPM |
| | GSLALSLYRLLLKQGYAERDFSVVQKLFDPTQGQ (SEQ ID NO: 155) |
| 5JE8 | MKKIGFIGLGNMGLPMSKNLVKSGYTVYGVDLNKEAEASFEKEGGIIGLSISKLA |
| | ETCDVVFTSLPSPRAVEAVYFGAEGLFENGHSNVVFIDTSTVSPQLNKQLEEAAK |
| | EKKVDFLAAPVSGGVIGAENRTLTFMVGGSKDVYEKTESIMGVLGANIFHVSEQI |
| | DSGTTVKINNLLIGFYTAGVSEALTLAKKNNMDLDKMFDILNVSYGQSRIYERN |
| | YKSFIAPENYEPGFTVNLLKKDLGFAVDLAKESELHLPVSEMLLNVYDEASQAG |
| | YGENDMAALYKKVSEQLISNQK (SEQ ID NO: 156) |
| Q819E3 | MEHKTLSIGHGIGVMGKSMVYHLMQDGHKVYVYNRTKAKTDSLVQDGANWC |
| | NTPKELVKQVDIVMTMVGYPHDEEVYFGIEGIIEHAKEGTIAIDFTTSTPTLAKR |
| | INEVAKRKNIYTLDAPVSGGDVGAKEAKLAIMVGGEKEIYDRCLPLLEKLGTNIQ |
| | LQGPAGSGQHTKMCNQIAIASNMIGVCEAVAYAKKAGLNPDKVLESISTGAAGS |
| | WSLSNLAPRMLKGDFEPGFYVKHFMKDMKIALEEAERLQLPVPGLSLAKELYEE |
| | LIKDGEENSGTQVLYKKYIRG (SEQ ID NO: 157) |
| Q5FQ06 | MSSPKIGFIGYGAMAQRMGANLRKAGYPVVAYAPSGGKDETEMLPSPRAIAEAA |
| | EIIIFCVPNDAAENESLHGENGALAALTPGKLVLDTSTVSPDQADAFASLAVEHGF |
| | SLLDAPMSGSTPEAETGDLVMLVGGDEAVVKRAQPVLDVIGKLTIHAGPAGSAA |
| | RLKLWNGVMGATLNVIAEGVSYGLAAGLDRDVVFDTLQQVAVVSPHHKRKL |
| | KMGQNREFPSQFPTRLMSKDMGLLLDAGRKVGAFMPGMAVADQALALSNRLH |
| | ANEDYSALIGAMEHSVANLPHK (SEQ ED NO: 158) |
| 2CVZ | MEKVAFIGLGAMGYPMAGHLARRFPTLWNRTFEKALRHQEEFGSEAVPLERV |
| | AEARVIFTCLPTTREVYEVAEALYPYLREGTYWVDATSGEPEASRRLAERLREKG |
| | VTYLDAPVSGGTSGAEAGTLTVMLGGPEEAVERVRPFLAYAKKVVHVGPVGAG |
| | HAVKAINNALLAVNLWAAGEGLLALVKQGVSAEKALEVINASSGRSNATENLIP |
| | QRVLTRAFPKTFALGLLVKDLGIAMGVLDGEKAPSPLLRLAREVYEMAKRELGP |
| | DADHVEALRLLERWGGVEIR (SEQ ID NO: 159) |
| Q05016 | MSQGRKAAERLAKKTVLITGASAGIGKATALEYLEASNGDMKLILAARRLEKLE |
| | ELKKTIDQEFPNAKVHVAQLDITQAEKIKPFIENLPQEFKDIDILVNNAGKALGSD |
| | RVGQIATEDIQDVTDTNVTALINITQAVLPIFQAKNSGDIVNLGSIAGRDAYPTGSI |
| | YCASKFAVGAFTDSLRKELINTKIRVILIAPGLVETEFSLVRYRGNEEQAKNVYKD |
| | TTPLMADDVADLIVYATSRKQNTVIADTLIFPTNQASPHHIFRG |
| | (SEQ ID NO: 160) |

Table 8 shows that 9 out of the 12 candidate 3-HPDHs were expressed in soluble form in E. coli.

TABLE 8
Expression of candidate 3-HPDHs.

| | ADH | | | | | | | | | | | |
|---|---|---|---|---|---|---|---|---|---|---|---|---|
| | YdfG | YMR226C | 2CVZ | Q5FQ06 | Q819E3 | 5JE8 | 3OBB | A4YI81 | A9A4M8 | ADH2_Y | ADH6_Y | YqhD |
| Soluble | No | Yes | Yes | Yes | Yes | Yes | Yes | Yes | Yes | No | Yes | No |
| Expression | | | | | | | | | | | | |

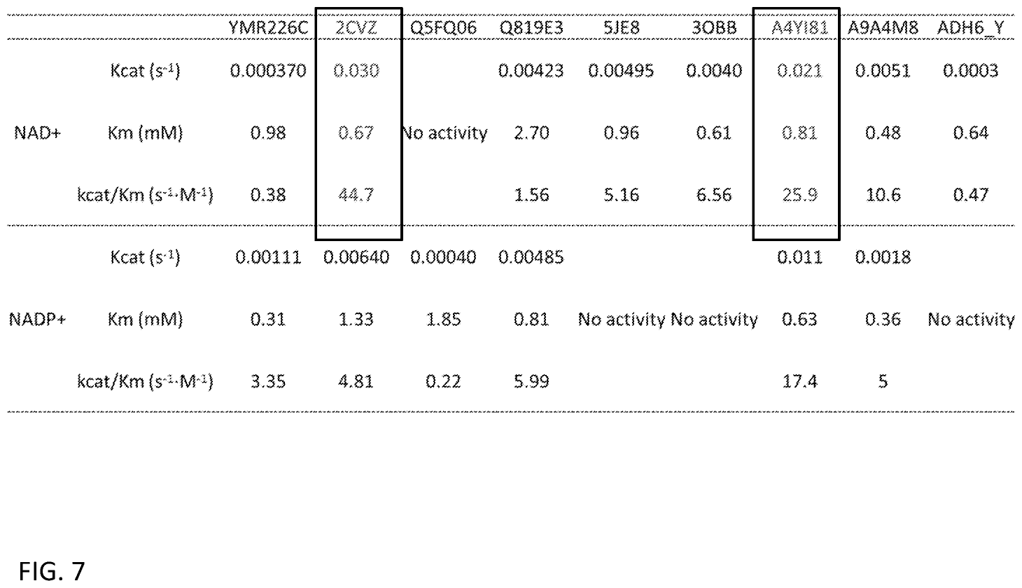

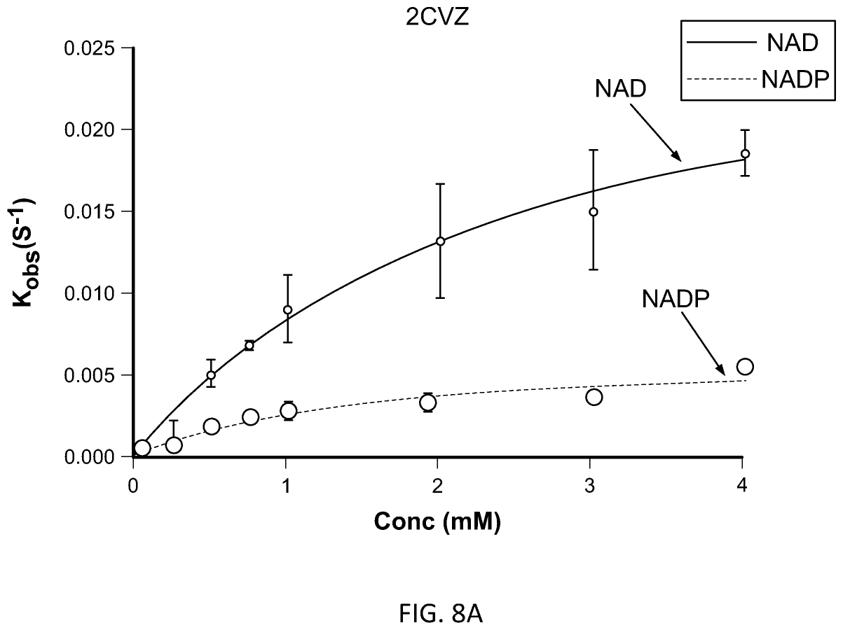

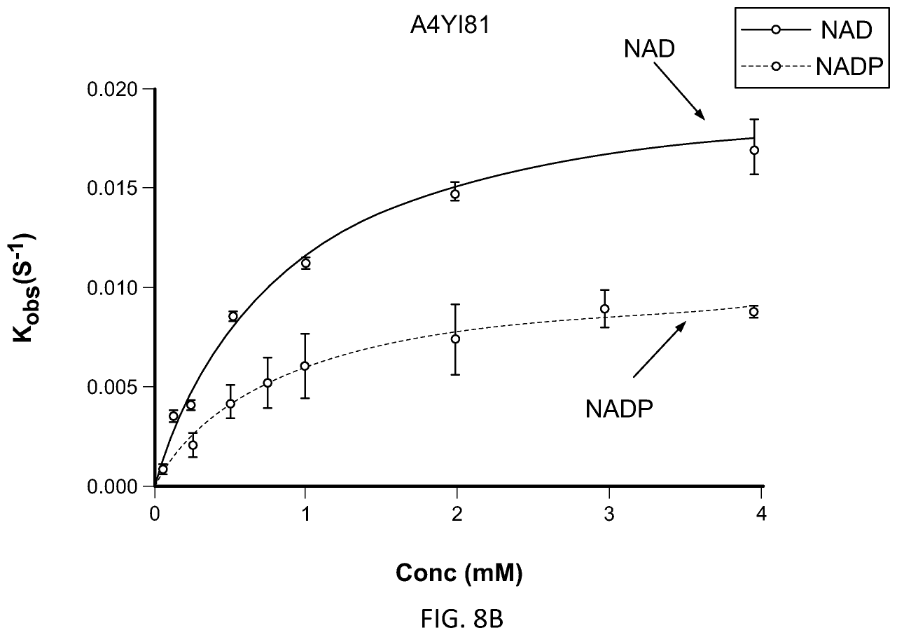

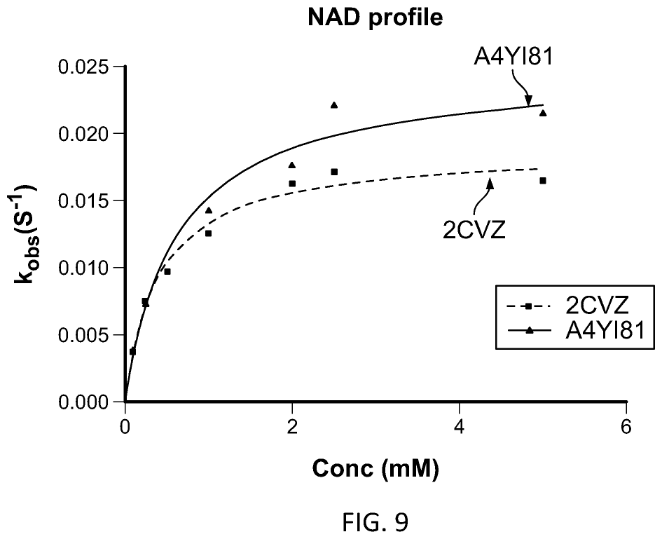

The nine 3-HPDHs from Table 6 that were expressed in soluble form were next characterized for their activity towards 3-HP. As shown in FIG. 7, these results demonstrated that of these enzymes, both 2CVZ and A4YI81 were found to prefer NAD⁺ as the cofactor and have the highest activity against 3-HP. Activity data for these enzymes using NAD+ or NADP+ as a co-factor are shown in FIGS. 8A & 8B. The enzymatic activities of these enzymes using NAD+ are also shown in FIG. 9, demonstrating a Km for NAD+ of 0.42 mM for 2CVZ and 0.65 mM for A4YI81.

The synthetic pathway shown in FIG. 2B also uses a PEPCK to provide oxaloacetate substrate for the OAADC. In order to explore possible active PEPCKs responsible for the conversion of phosphoenolpyruvate to oxaloacetate, 5 PEPCK candidates were synthesized and cloned into an expression vector. The sequences of the enzymes tested are provided in Table 9.

TABLE 9
Candidate PEPCK sequences.

| Enzyme name | Amino acid sequence |
|---|---|
| Q7XAU8 | MASPNGLAKIDTQGKTEVYDGDTAAPVRAQTIDELHLLQRKRSA |
| | PTTPIKDGATSAFAAAISEEDRSQQQLQSISASLTSLARETGPKLVK |
| | GDPSDPAPHKHYQPAAPTIVATDSSLKFTHVLYNLSPAELYEQAF |
| | GQKKSSFITSTGALATLSGAKTGRSPRDKRVVKDEATAQELWWG |
| | KGSPNIEMDERQFVINRERALDYLNSLDKVYVNDQFLNWDPENRI |
| | KVRIITSRAYHALFMHNMCIRPTDEELESFGTPDFTIYNAGEFPAN |
| | RYANYMTSSTSINISLARREMVILGTQYAGEMKKGLFGVMHYLM |
| | PKRGILSLHSGCNMGKDGDVALFFGLSGTGKTTLSTDHNRLLIGD |
| | DEHCWSDNGVSNIEGGCYAKCIDLSQEKEPDIWNAIKFGTVLENV |
| | VFNERTREVDYSDKSITENTRAAYPIEFIPNAKIPCVGPHPKNVILL |
| | ACDAFGVLPPVSKLNLAQTMYHFISGYTALVAGTVDGITEPTATF |
| | SACFGAAFIMYHPTKYAAMLAEKMQKYGATGWLVNTGWSGGR |
| | YGVGKRIRLPHTRKIIDAIHSGELLTANYKKTEVFGLEIPTEINGVP |
| | SEILDPINTWTDKAAYKENLLNLAGLFKKNFEVFASYKIGDDSSLT |
| | DEILAAGPNF (SEQ ID NO: 161) |
| PCKA_Ecoli | MRVNNGLTPQELEAYGISDVHDIVYNPSYDLLYQEELDPSLTGYE |
| | RGVLTNLGAVAVDTGIFTGRSPKDKYIVRDDTTRDTFWWADKGK |
| | GKNDNKPLSPETWQHLKGLVTRQLSGKRLFVVDAFCGANPDTRL |
| | SVRFITEVAWQAHFVKNMFIRPSDEELAGFKPDFIVMNGAKCTNP |
| | QWKEQGLNSENFVAFNLTERMQLIGGTWYGGEMKKGMFSMMN |
| | YLLPLKGIASMHCSANVGEKGDVAVFFGLSGTGKTTLSTDPKRRL |
| | IGDDEHGWDDDGVFNFEGGCYAKTIKLSKEAEPEIYNAIRRDALL |
| | ENVTVREDGTIDFDDGSKTENTRVSYPIYHIDNIVKPVSKAGHATK |
| | VIFLTADAFGVLPPVSRLTADQTQYHFLSGFTAKLAGTERGITEPT |
| | PTFSACFGAAFLSLHPTQYAEVLVKRMQAAGAQAYLVNTGWNG |
| | TGKRISIKDTRAIIDAILNGSLDNAETFTLPMFNLAIPTELPGVDTKI |
| | LDPRNTYASPEQWQEKAETLAKLFIDNFDKYTDTPAGAALVAAG |
| | PKL (SEQ ID NO: 162) |
| PCK from | MTDLNKLVKELNDLGLTDVKEIVYNPSYEQLFEEETKPGLEGFDK |
| Actinobaccilus_succinogenes | GTLTTLGAVAVDTGIFTGRSPKDKYIVCDETTKDTVWWNSEAAK |
| | NDNKPMTQETWKSLRELVAKQLSGKRLFVVEGYCGASEKHRIGV |
| | RMVTEVAWQAHFVKNMFIRPTDEELKNFKADFTVLNGAKCTNP |
| | NWKEQGLNSENFVAFNITEGIQLIGGTWYGGEMKKGMFSMMNY |
| | FLPLCGVASMHCSANVGKDGDVAIFFGLSGTGKTTLSTDPKRQLI |
| | GDDEHGWDESGVFNFEGGCYAKTINLSQENEPDIYGAIRRDALLE |
| | NVVVRADGSVDFDDGSKTENTRVSYPIYHIDNIVRPVSKAGHATK |
| | VIFLTADAFGVLPPVSKLTPEQTEYYFLSGFTAKLAGTERGVTEPT |
| | PTFSACFGAAFLSLHPIQYADVLVERMKASGAEAYLVNTGWNGT |
| | GKRISIKDTRGIIDAILDGSIEKAEMGELPIFNLAIPKALPGVDPAIL |
| | DPRDTYADKAQWQVKAEDLANRFVKNFVKYTANPEAAKLVGA |
| | GPKA (SEQ ID NO: 163) |
| 1J3B | MQRLEALGIHPKKRVFWNTVSPVLVEHTLLRGEGLLAHHGPLVV |
| | DTTPYTGRSPKDKFWREPEVEGEIWWGEVNQPFAPEAFEALYQR |
| | VVQYLSERDLYVQDLYAGADRRYRLAVRVVTESPWHALFARNM |
| | FILPRRFGNDDEVEAFVPGFTVVHAPYFQAVPERDGTRSEVFVGIS |
| | FQRRLYLIVGTKYAGEIKKSIFTVMNYLMPKRGVFPMHASANVG |
| | KEGDVAVFFGLSGTGKTTLSTDPERPLIGDDEHGWSEDGVFNFEG |
| | GCYAKWLSPEHEPLIYKASNQFEAILENVVVNPESRRVQWDDD |
| | SKTENTRSSYPIAHLENVVESGVAGHPRAIFFLSADAYGVLPPIAR |
| | LSPEEAMYYFLSGYTARVAGTERGVTEPRATFSACFGAPFLPMHP |
| | GVYARMLGEKIRKHAPRVYLVNTGWTGGPYGVGYRFPLPVTRA |
| | LLKAALSGALENVPYRRDPVFGFEVPLEAPGVPQELLNPRETWAD |
| | KEAYDQQARKLARLFQENFQKYASGVAKEVAEAGPRTE (SEQ ID |
| | NO: 164) |
| 1YTM | MSLSESLAKYGITGATNIVHNPSHEELFAAETQASLEGFEKGTVTE |
| | MGAVNVMTGVYTGRSPKDKFIVKNEASKEIWWTSDEFKNDNKP |
| | VTEEAWAQLKALAGKELSNKPLYVVDLFCGANENTRLKIRFVME |
| | VAWQAHFVTNMFIRPTEEELKGFEPDFVVLNASKAKVENFKELG |
| | LNSETAVVFNLAEKMQIILNTWYGGEMKKGMFSMMNFYLPLQGI |
| | AAMHCSANTDLEGKNTAIFFGLSGTGKTTLSTDPKRLLIGDDEHG |
| | WDDDGVFNFEGGCYAKVENLSKENEPDIWGAIKRNALLENVTVD |
| | ANGKVDFADKSVTENTRVSYPIFHIKNIVKPVSKAPAAKRVIFLSA |
| | DAFGVLPPVSILSKEQTKYYFLSGFTAKLAGTERGITEPTPTFSSCF |
| | GAAFLTLPPTKYAEVLVKRMEASGAKAYLVNTGWNGTGKRISIK |
| | DTRGIIDAILDGSIDTANTATIPYFNFTVPTELKGVDTKILDPRNTY |
| | ADASEWEVKAKDLAERFQKNFKKFESLGGDLVKAGPOL (SEQ ID |
| | NO: 165) |

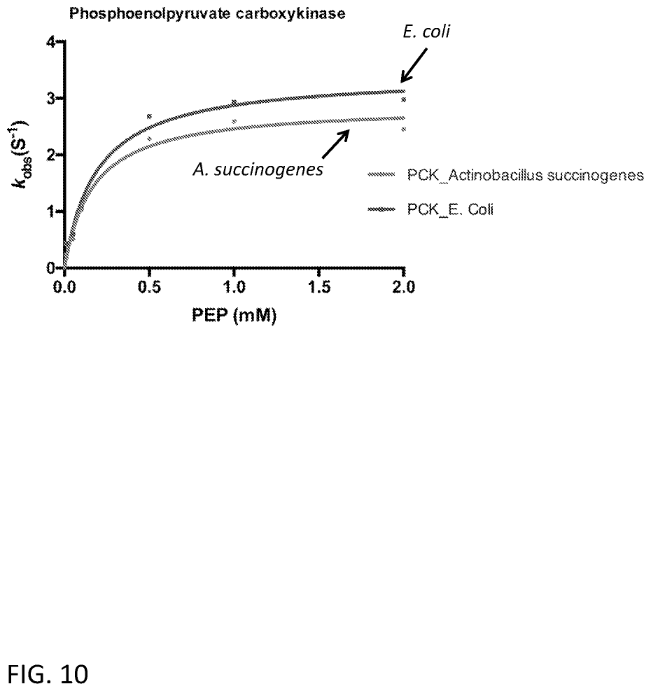

Two highly active PEPCKs were identified from E. coli and A. succinogenes, respectively. The activities of these enzymes using phosphoenolpyruvate (PEP) as a substrate are shown in FIG. 10 and Table 10.

TABLE 10
Kinetics of PEPCK enzymes against PEP.

| | Actinobacillus succinogenes PCK | E. coli PCK |
|---|---|---|
| kcat(s⁻¹) | 2.875 | 3.423 |
| Km(mM) | 0.1692 | 0.1905 |
| kcat/Km(M⁻¹s⁻¹) | 16991.72577 | 17968.50394 |

In summary, these data demonstrate the identification of multiple PEPCK, OAADC, and 3-HPDH enzymes suitable for catalyzing each step of a novel and advantageous metabolic pathway to produce 3-HP.

What is claimed is:

1. A method for producing 3-hydroxypropionate (3-HP), the method comprising:
(a) providing a recombinant host cell, wherein the recombinant host cell comprises a recombinant polynucleotide encoding an oxaloacetate decarboxylase (OAADC) and a polynucleotide encoding a 3-hydroxypropionate dehydrogenase (3-HPDH), and wherein the OAADC has a ratio of activity against pyruvate to activity against oxaloacetate that is less than or equal to about 5:1; and
(b) culturing the recombinant host cell in a culture medium comprising a substrate under conditions suitable for the recombinant host cell to convert the substrate to 3-HP, wherein expression of the OAADC and the 3-HPDH results in increased production of 3-HP, as compared to production by a host cell lacking expression of the OAADC and the 3-HPDH.

2. A method for producing 3-hydroxypropionate (3-HP), the method comprising:
(a) providing a recombinant host cell, wherein the recombinant host cell comprises a recombinant polynucleotide encoding an oxaloacetate decarboxylase (OAADC) and a polynucleotide encoding a 3-hydroxypropionate dehydrogenase (3-HPDH), wherein the OAADC has a specific activity of at least 0.1 μmol/min/mg against oxaloacetate; and
(b) culturing the recombinant host cell in a culture medium comprising a substrate under conditions suitable for the recombinant host cell to convert the substrate to 3-HP, wherein expression of the OAADC and the 3-HPDH results in increased production of 3-HP, as compared to production by a host cell lacking expression of the OAADC and the 3-HPDH.

3. The method of claim 1 or claim 2, wherein the recombinant host cell is a recombinant prokaryotic cell.

4. The method of claim 3, wherein the prokaryotic cell is an Escherichia coli cell.

5. The method of claim 1 or claim 2, wherein the host cell is selected from the group consisting of Acetobacter aceti, Achromobacter, Acidiphilium, Acinetobacter, Actinomadura, Actinoplanes, Aeropyrum pernix, Agrobacterium, Alcaligenes, Ananas comosus (M), Arthrobacter, Bacillus alcalophilus, Bacillus amyloliquefaciens, Bacillus brevis, Bacillus circulans, Bacillus clausii, Bacillus lentus, Bacillus licheniformis, Bacillus macerans, Bacillus stearothermophilus, Bacillus subtilis, Bifidobacterium, Brevibacillus brevis, Burkholderia cepacia, Candida cylindracea, Carica papaya (L), Cellulosimicrobium, Cephalosporium, Chaetomium erraticum, Chaetomium gracile, Clostridium, Clostridium butyricum, Clostridium acetobutylicum, Clostridium thermocellum, Corynebacterium (glutamicum), Corynebacterium efficiens, Escherichia coli, Enterococcus, Erwina chrysanthemi, Gliconobacter, Gluconacetobacter, Haloarcula, Humicola insolens, Kitasatospora setae, Klebsiella, Klebsiella oxytoca, Kocuria, Lactlactis, Lactobacillus, Lactobacillus fermentum, Lactobacillus sake, Lactococcus, Lactococcus lactis, Leuconostoc, Methylocystis, Methanolobus siciliae, Methanogenium organophilum, Methanobacterium bryantii, Microbacterium imperiale, Micrococcus lysodeikticus, Microlunatus, Mucor javanicus, Mycobacterium, Myrothecium, Nitrobacter, Nitrosomonas, Nocardia, Papaya carica, Pediococcus, Pediococcus halophilus, Paracoccus pantotrophus, Propionibacterium, Pseudomonas, Pseudomonas fluorescens, Pseudomonas denitrificans, Pyrococcus, Pyrococcus furiosus, Pyrococcus horikoshii, Rhizobium, Rhizomucor miehei, Rhizomucor pusillus Lindt, Rhizopus, Rhizopus delemar, Rhizopus japonicus, Rhizopus niveus, Rhizopus oryzae, Rhizopus oligosporus, Rhodococcus, Sclerotina libertina, Sphingobacterium multivorum, Sphingobium, Sphingomonas, Streptococcus, Streptococcus thermophilus Y-1, Streptomyces, Streptomyces griseus, Streptomyces lividans, Streptomyces murinus, Streptomyces rubiginosus, Streptomyces violaceoruber, Streptoverticillium mobaraense, Tetragenococcus, Thermus, Thiosphaera pantotropha, Trametes, Vibrio alginolyticus, Xanthomonas, Zymomonas, and Zymomonus mobilis.

6. The method of claim 1 or claim 2, wherein the recombinant host cell is a recombinant fungal cell.

7. A method for producing 3-hydroxypropionate (3-HP), the method comprising:
(a) providing a recombinant host cell, wherein the recombinant host cell comprises a recombinant polynucleotide encoding an oxaloacetate decarboxylase (OAADC) and a polynucleotide encoding a 3-hydroxypropionate dehydrogenase (3-HPDH), and wherein the recombinant host cell is a recombinant fungal cell; and
(b) culturing the recombinant host cell in a culture medium comprising a substrate under conditions suitable for the recombinant host cell to convert the substrate to 3-HP, wherein expression of the OAADC and the 3-HPDH results in increased production of 3-HP, as compared to production by a host cell lacking expression of the OAADC and the 3-HPDH.

8. The method of claim 7, wherein the OAADC has a ratio of activity against pyruvate to activity against oxaloacetate that is less than or equal to about 5:1.

9. The method of claim 7 or claim 8, wherein the OAADC has a specific activity of at least 0.1 μmol/min/mg against oxaloacetate.

10. The method of any one of claims 1-9, wherein the OAADC has a specific activity of at least 10 μmol/min/mg against oxaloacetate.

11. The method of any one of claims 1-10, wherein the OAADC has a specific activity of at least 100 μmol/min/mg against oxaloacetate.

12. The method of any one of claims 1-11, wherein the OAADC has a catalytic efficiency (k_{cat}/K_{M}) for oxaloacetate that is greater than about 2000 M⁻¹s⁻¹.

13. The method of any one of claims 6-12, wherein the recombinant host cell is capable of producing 3-HP at a pH lower than 6.

14. The method of claim 13, wherein the recombinant host cell is capable of producing 3-HP below the pKa of 3-HP.

15. The method of any one of claims 6-14, wherein the fungal cell is a yeast cell.

16. The method of any one of claims 6-14, wherein the fungal cell is of a genus or species selected from the group consisting of Aspergillus, Aspergillus nidulans, Aspargillus niger, Aspargillus oryze, Aspergillus melleus, Aspergillus pulverulentus, Aspergillus saitoi, Aspergillus sojea, Aspergillus terreus, Aspergillus pseudoterreus, Aspergillus usamii, Candida rugosa, Issatchenkia orientalis, Kluyveromyces, Kluyveromyces fragilis, Kluyveromyces lactis, Kluyveromyces marxianas, Penicillium, Penicillium camemberti, Penicillium citrinum, Penicillium emersonii, Penicillium roqueforti, Penicillum lilactinum, Penicillum multicolor, Rhodosporidium toruloides, Saccharomyces cerevisiae, Schizosaccharomyces pombe, Trichoderma, Trichoderma longibrachiatum, Trichoderma reesei, Trichoderma viride, Trichosporon penicillatum, Yarrowia lipolytica, and Zygosaccharomyces rouxii.

17. The method of any one of claims 1-16, wherein the OAADC comprises an amino acid sequence at least 80% identical to SEQ ID NO:1.

18. The method of claim 17, wherein the OAADC comprises the amino acid sequence of SEQ ID NO:1.

19. The method of any one of claims 1-16, wherein the OAADC comprises an amino acid sequence at least 80% identical to a sequence selected from the group consisting of SEQ ID NOs:145, 146, 148, and 166.

20. The method of claim 19, wherein the OAADC comprises an amino acid sequence selected from the group consisting of SEQ ID NOs:145, 146, 148, and 166.

21. The method of any one of claims 1-20, wherein the recombinant polynucleotide is stably integrated into a chromosome of the recombinant host cell.

22. The method of any one of claims 1-20, wherein the recombinant polynucleotide is maintained in the recombinant host cell on an extra-chromosomal plasmid.

23. The method of any one of claims 1-22, wherein the polynucleotide encoding the 3-HPDH is an endogenous polynucleotide.

24. The method of any one of claims 1-22, wherein the polynucleotide encoding the 3-HPDH is a recombinant polynucleotide.

25. The method of any one of claims 1-24, wherein the 3-HPDH comprises an amino acid sequence selected from the group consisting of SEQ ID NOs:122-130.

26. The method of any one of claims 1-24, wherein the 3-HPDH comprises the amino acid sequence of SEQ ID NO:154 or 159.

27. The method of any one of claims 1-26, wherein the recombinant host cell is cultured under anaerobic conditions suitable for the recombinant host cell to convert the substrate to 3-HP.

28. The method of any one of claims 1-27, wherein the substrate comprises glucose.

29. The method of claim 28, wherein at least 95% of the glucose metabolized by the recombinant host cell is converted to 3-HP.

30. The method of claim 29, wherein 100% of the glucose metabolized by the recombinant host cell is converted to 3-HP.

31. The method of any one of claims 1-30, wherein the substrate is selected from the group consisting of sucrose, fructose, xylose, arabinose, cellobiose, cellulose, alginate, mannitol, laminarin, galactose, and galactan.

32. The method of any one of claims 1-31, wherein the recombinant host cell further comprises a recombinant polynucleotide encoding a phosphoenolpyruvate carboxykinase (PEPCK).

33. The method of claim 32, wherein the PEPCK comprises the amino acid sequence of SEQ ID NO:162 or 163.

34. The method of any one of claims 1-33, wherein the recombinant host cell further comprises a modification resulting in decreased production of pyruvate from phosphoenolpyruvate, as compared to a host cell lacking the modification.

35. The method of claim 34, wherein the modification results in decreased pyruvate kinase (PK) activity, as compared to a host cell lacking the modification.

36. The method of claim 34, wherein the modification results in decreased pyruvate kinase (PK) expression, as compared to a host cell lacking the modification.

37. The method of claim 36, wherein the modification comprises an exogenous promoter in operable linkage with an endogenous pyruvate kinase (PK) coding sequence, wherein the exogenous promoter results in decreased endogenous PK coding sequence expression, as compared to expression of the endogenous PK coding sequence in operable linkage with an endogenous PK promoter.

38. The method of claim 37, wherein the exogenous promoter is a MET3, CTR1, or CTR3 promoter.

39. The method of claim 38, wherein the exogenous promoter comprises a polynucleotide sequence selected from the group consisting of SEQ ID NOs:131-133.

40. The method of any one of claims 34-39, wherein the recombinant host cell further comprises a second modification resulting in increased expression or activity of phosphoenolpyruvate carboxykinase (PEPCK), as compared to a host cell lacking the second modification.

41. The method of any one of claims 1-40, further comprising: (c) substantially purifying the 3-HP.

42. The method of any one of claims 1-41, further comprising: (d) converting the 3-HP to acrylic acid.

43. A recombinant host cell comprising a recombinant polynucleotide encoding an oxaloacetate decarboxylase (OAADC), wherein the OAADC has a ratio of activity against pyruvate to activity against oxaloacetate that is less than or equal to about 5:1.

44. A recombinant host cell comprising a recombinant polynucleotide encoding an oxaloacetate decarboxylase (OAADC), wherein the OAADC has a specific activity of at least 0.1 μmol/min/mg against oxaloacetate.

45. The host cell of claim 43 or claim 44, wherein the recombinant host cell is a recombinant prokaryotic cell.

46. The host cell of claim 45, wherein the prokaryotic cell is an Escherichia coli cell.

47. The host cell of claim 43 or claim 44, wherein the host cell is selected from the group consisting of Acetobacter aceti, Achromobacter, Acidiphilium, Acinetobacter, Actinomadura, Actinoplanes, Aeropyrum pernix, Agrobacterium, Alcaligenes, Ananas comosus (M), Arthrobacter, Bacillus alcalophilus, Bacillus amyloliquefaciens, Bacillus brevis, Bacillus circulans, Bacillus clausii, Bacillus lentus, Bacillus licheniformis, Bacillus macerans, Bacillus stearothermophilus, Bacillus subtilis, Bifidobacterium, Brevibacillus brevis, Burkholderia cepacia, Candida cylindracea, Carica papaya (L), Cellulosimicrobium, Cephalosporium, Chaetomium erraticum, Chaetomium gracile, Clostridium, Clostridium butyricum, Clostridium acetobutylicum, Clostridium thermocellum, Corynebacterium (glutamicum), Corynebacterium efficiens, Escherichia coli, Enterococcus, Erwina chrysanthemi, Gliconobacter, Gluconacetobacter, Haloarcula, Humicola insolens, Kitasatospora setae, Klebsiella, Klebsiella oxytoca, Kocuria, Lactlactis, Lactobacillus, Lactobacillus fermentum, Lactobacillus sake, Lactococcus, Lactococcus lactis, Leuconostoc, Methylocystis, Methanolobus siciliae, Methanogenium organophilum, Methanobacterium bryantii, Microbacterium imperiale, Micrococcus lysodeikticus, Microlunatus, Mucor javanicus, Mycobacterium, Myrothecium, Nitrobacter, Nitrosomonas, Nocardia, Papaya carica, Pediococcus, Pediococcus halophilus, Paracoccus pantotrophus, Propionibacterium, Pseudomonas, Pseudomonas fluorescens, Pseudomonas denitrificans, Pyrococcus, Pyrococcus furiosus, Pyrococcus horikoshii, Rhizobium, Rhizomucor miehei, Rhizomucor pusillus Lindt, Rhizopus, Rhizopus delemar, Rhizopus japonicus, Rhizopus niveus, Rhizopus oryzae, Rhizopus oligosporus, Rhodococcus, Sclerotina libertina, Sphingobacterium multivorum, Sphingobium, Sphingomonas, Streptococcus, Streptococcus thermophilus Y-1, Streptomyces, Streptomyces griseus, Streptomyces lividans, Streptomyces murinus, Streptomyces rubiginosus, Streptomyces violaceoruber, Streptoverticillium mobaraense, Tetragenococcus, Thermus, Thiosphaera pantotropha, Trametes, Vibrio alginolyticus, Xanthomonas, Zymomonas, and Zymomonus mobilis.

48. The host cell of claim 43 or claim 44, wherein the recombinant host cell is a recombinant fungal host cell.

49. A recombinant fungal host cell comprising a recombinant polynucleotide encoding an oxaloacetate decarboxylase (OAADC).

50. The host cell of claim 49, wherein the OAADC has a ratio of activity against pyruvate to activity against oxaloacetate that is less than or equal to about 5:1.

51. The host cell of claim 49 or claim 50, wherein the OAADC has a specific activity of at least 0.1 μmol/min/mg against oxaloacetate.

52. The host cell of any one of claims 43-51, wherein the OAADC has a specific activity of at least 10 μmol/min/mg against oxaloacetate.

53. The host cell of any one of claims 43-52, wherein the OAADC has a specific activity of at least 100 μmol/min/mg against oxaloacetate.

54. The host cell of any one of claims 43-53, wherein the OAADC has a catalytic efficiency (k_{cat}/K_{M}) for oxaloacetate that is greater than about 2000 M⁻¹s⁻¹.

55. The host cell of any one of claims 43-54, wherein the host cell further comprises a polynucleotide encoding a 3-hydroxypropionate dehydrogenase (3-HPDH).

56. The host cell of claim 55, wherein the polynucleotide encoding the 3-HPDH is an endogenous polynucleotide.

57. The host cell of claim 55, wherein the polynucleotide encoding the 3-HPDH is a recombinant polynucleotide.

58. The host cell of any one of claims 55-57, wherein the 3-HPDH comprises an amino acid sequence selected from the group consisting of SEQ ID NOs:122-130.

59. The host cell of any one of claims 55-57, wherein the 3-HPDH comprises the amino acid sequence of SEQ ID NO:154 or 159.

60. The host cell of any one of claims 48-59, wherein the recombinant fungal host cell is capable of producing 3-HP at a pH lower than 6.

61. The host cell of claim 60, wherein the recombinant host cell is capable of producing 3-HP below the pKa of 3-HP.

62. The host cell of any one of claims 48-61, wherein the fungal cell is a yeast cell.

63. The host cell of any one of claims 48-61, wherein the fungal cell is of a genus or species selected from the group consisting of Aspergillus, Aspergillus nidulans, Aspargillus niger, Aspargillus oryze, Aspergillus melleus, Aspergillus pulverulentus, Aspergillus saitoi, Aspergillus sojea, Aspergillus terreus, Aspergillus pseudoterreus, Aspergillus usamii, Candida rugosa, Issatchenkia orientalis, Kluyveromyces, Kluyveromyces fragilis, Kluyveromyces lactis, Kluyveromyces marxianas, Penicillium, Penicillium camemberti, Penicillium citrinum, Penicillium emersonii, Penicillium roqueforti, Penicillum lilactinum, Penicillum multicolor, Rhodosporidium toruloides, Saccharomyces cerevisiae, Schizosaccharomyces pombe, Trichoderma, Trichoderma longibrachiatum, Trichoderma reesei, Trichoderma viride, Trichosporon penicillatum, Yarrowia lipolytica, and Zygosaccharomyces rouxii.

64. The host cell of any one of claims 43-63, wherein the OAADC comprises an amino acid sequence at least 80% identical to SEQ ID NO:1.

65. The host cell of claim 64, wherein the OAADC comprises the amino acid sequence of SEQ ID NO:1.

66. The host cell of any one of claims 43-63, wherein the OAADC comprises an amino acid sequence at least 80% identical to a sequence selected from the group consisting of SEQ ID NOs:145, 146, 148, and 166.

67. The host cell of claim 66, wherein the OAADC comprises an amino acid sequence selected from the group consisting of SEQ ID NOs:145, 146, 148, and 166.

68. The host cell of any one of claims 43-67, wherein the recombinant polynucleotide is stably integrated into a chromosome of the recombinant host cell.

69. The host cell of any one of claims 43-67, wherein the recombinant polynucleotide is maintained in the recombinant host cell on an extra-chromosomal plasmid.

70. The host cell of any one of claims 43-69, wherein the recombinant host cell is capable of producing 3-HP under anaerobic conditions.

71. The host cell of any one of claims 43-70, wherein the recombinant host cell further comprises a recombinant polynucleotide encoding a phosphoenolpyruvate carboxykinase (PEPCK).

72. The host cell of claim 71, wherein the PEPCK comprises the amino acid sequence of SEQ ID NO:162 or 163.

73. The host cell of any one of claims 43-72, wherein the recombinant host cell further comprises a modification resulting in decreased production of pyruvate from phosphoenolpyruvate, as compared to a host cell lacking the modification.

74. The host cell of claim 73, wherein the modification results in decreased pyruvate kinase (PK) activity, as compared to a host cell lacking the modification.

75. The host cell of claim 73, wherein the modification results in decreased pyruvate kinase (PK) expression, as compared to a host cell lacking the modification.

76. The host cell of claim 75, wherein the modification comprises an exogenous promoter in operable linkage with an endogenous pyruvate kinase (PK) coding sequence, wherein the exogenous promoter results in decreased endogenous PK coding sequence expression, as compared to expression of the endogenous PK coding sequence in operable linkage with an endogenous PK promoter.

77. The host cell of claim 76, wherein the exogenous promoter is a MET3, CTR1, or CTR3 promoter.

78. The host cell of claim 77, wherein the exogenous promoter comprises a polynucleotide sequence selected from the group consisting of SEQ ID NOs:131-133.

79. The host cell of any one of claims 71-78, wherein the recombinant host cell further comprises a second modification resulting in increased expression or activity of phosphoenolpyruvate carboxykinase (PEPCK), as compared to a host cell lacking the second modification.

80. A vector comprising a polynucleotide that encodes an amino acid sequence at least 80% identical to a sequence selected from the group consisting of SEQ ID NOs:1, 145, 146, 148, and 166.

81. The vector of claim 80, wherein the polynucleotide encodes the amino acid sequence of SEQ ID NO:1.

82. The vector of claim 80, wherein the polynucleotide comprises the polynucleotide sequence of SEQ ID NO:2.

83. The vector of claim 80, wherein the polynucleotide encodes an amino acid sequence selected from the group consisting of SEQ ID NOs:145, 146, 148, and 166.

84. The vector of any one of claims 80-83, wherein the vector further comprises a promoter operably linked to the polynucleotide.

85. The vector of claim 84, wherein the promoter is exogenous with respect to the polynucleotide that encodes the amino acid sequence at least 80% identical to SEQ ID NO:1.

86. The vector of claim 84, wherein the promoter is a T7 promoter.

87. The vector of claim 84, wherein the promoter is a TDH or FBA promoter.

88. The vector of claim 87, wherein the promoter comprises the polynucleotide sequence of SEQ ID NO:135 or 136.

89. The vector of any one of claims 80-88, wherein the vector further comprises a polynucleotide encoding a 3-hydroxypropionate dehydrogenase (3-HPDH).

90. The vector of claim 89, wherein the 3-HPDH comprises an amino acid sequence selected from the group consisting of SEQ ID NOs:122-130.

91. The vector of claim 89, wherein the 3-HPDH comprises the amino acid sequence of SEQ ID NO:154 or 159.

92. The vector of any one of claims 89-91, wherein the polynucleotide that encodes the sequence selected from the group consisting of SEQ ID NOs:1, 145, 146, 148, and 166 and the polynucleotide encoding the 3-hydroxypropionate dehydrogenase (3-HPDH) are arranged in an operon operably linked to the same promoter.

93. The vector of claim 92, wherein the promoter is a T7 or phage promoter.

94. The vector of any one of claims 80-93, wherein the vector further comprises a polynucleotide encoding a phosphoenolpyruvate carboxykinase (PEPCK).

95. The vector of claim 94, wherein the PEPCK comprises the amino acid sequence of SEQ ID NO:162 or 163.

96. The vector of claim 94 or claim 95, wherein the polynucleotide that encodes the sequence selected from the group consisting of SEQ ID NOs:1, 145, 146, 148, and 166: the polynucleotide encoding the 3-hydroxypropionate dehydrogenase (3-HPDH); and the polynucleotide encoding the phosphoenolpyruvate carboxykinase (PEPCK) are arranged in an operon operably linked to the same promoter.

97. The vector of claim 96, wherein the promoter is a T7 or phage promoter.


SEQUENCE LISTING (166 sequences)

SEQ ID NO: 1 - protein, 558 aa, Gluconacetobacter diazotrophicus
MTYTVGRYLADRLAQIGLKHHFAVAGDYNLVLLDQLLLNTDMQQIYCSNELNCGFSAEGYARANGAAAAIVTFSVGALSAFNALGGAYAENLPVILISGAPNANDHGTGHILHHTLGTTDYGYQLEMARHITCAAESIVAAEDAPAKIDHVIRTALREKKPAYLEIACNVAGAPCVRPGGIDALLSPPAPDEASLKAAVDAALAFIEQRGSVTMLVGSRIRAAGAQAQAVALADALGCAVTTMAAAKSFFPEDHPGYRGHYWGEVSSPGAQQAVEGADGVICLAPVFNDYATVGWSAWPKGDNVMLVERHAVTVGGVAYAGIDMRDFLTRLAAHTVRRDATARGGAYVTPQTPAAAPTAPLNNAEMARQIGALLTPRTTLTAETGDSWFNAVRMKLPHGARVELEMQWGHIGWSVPAAFGNALAAPERQHVLMVGDGSFQLTAQEVAQMIRHDLPVIIFLINNHGYTIEVMIHDGPYNNVKNWDYAGLMEVFNAGEGNGLGLRARTGGELAAAIEQARANRNGPTLIECTLDRDDCTQELVTWGKRVAAANARPPRAG

SEQ ID NO: 2 - DNA, 1674 bp, Gluconacetobacter diazotrophicus
atgacgtataccgtgggccgctatctggctgaccgtttagcccaaattggtcttaaacatcactttgccgtggcaggcgactacaacttggttctgttagaccagctgctgctgaataccgacatgcaacagatttactgcagtaatgaacttaactgtgggttcagtgccgaaggctatgcgcgcgccaacggcgcggctgcagccattgtcaccttttccgtcggcgctctgagcgccttcaacgccttgggcggcgcatacgcggaaaacttgccggtcatcctgatctctggcgcaccgaacgcgaatgaccacgggaccggccatatcttgcaccatacgctgggcaccacagattatggctaccaactggaaatggcacgccatattacatgtgcggcggaatcaattgtcgctgcagaggatgcgccagcgaaaattgatcacgtgattcgcaccgcgctgcgcgaaaaaaaaccagcatacctggaaattgcgtgtaatgtggctggcgctccatgcgttcgcccgggcggtattgatgcgcttctgtcgccgcccgccccggatgaagccagcctgaaggcggccgttgacgccgccctggccttcattgaacaacgcggctcagtgacgatgctcgttggtagtcgtatccgtgcagccggagcccaggctcaggcggtcgccctcgcggatgctctgggctgcgcggtgacgacgatggcggcagcgaaatctttttttccagaagatcatccgggttatcgtggtcactactggggtgaggtgtcatccccgggtgcccaacaggccgtggagggcgctgacggtgtgatttgtttggccccggttttcaatgactatgccactgtgggctggagcgcgtggccgaaaggggataacgtcatgcttgtggaacgtcacgcggttaccgtaggtggtgttgcgtatgccggcatcgatatgcgagactttctgacacgtctggcggctcacaccgtacgccgtgatgccaccgcacgcggcggggcatatgtaaccccgcagacgccggcagcggctccgactgcccctctgaacaacgcggagatggcgcgccagatcggcgcgctactgacgccgcggacaactttgaccgcggaaaccggcgacagctggttcaatgcggtccgtatgaaactgccgcacggcgcgcgggtcgaactggaaatgcaatgggggcacatcggttggagcgtgccggcggcgtttggtaacgcgctggcggcgccggaacgccagcacgtcctgatggtgggtgacggctcatttcagctgactgcacaggaagtggcccagatgattcgtcatgacttaccggtgataatctttctgatcaacaaccacggctatactatagaagtgatgatccatgacgggccgtataacaacgtgaagaactgggattacgcgggcctgatggaagtcttcaatgcgggggaaggtaacggcctcggtcttcgtgcccgcactgggggcgaactggcggcggctattgaacaggcccgcgccaaccgtaacggcccgaccctgatcgaatgtaccctggaccgcgatgactgcacgcaggaactggtgacctggggcaaacgtgttgcagctgccaacgcgcgccctcctcgtgcagga

SEQ ID NO: 3 - protein, 311 aa, Sandaracinus amylolyticus
MADLLAIHRHAVRARLLDERLTQLARAGRIGFHPDARGFEPAIAAAVLAMRAEDAIFPSARDHAAFLVRGLPISRYVAHAFGSVEDPMRGHAAPGHLASRELRIAAASGLVSNHMTHAAGYAWAAKLRGETCAVLTMFADTAADAGDFHSAVNFAGATKAPVIFFCRTDRTRSAHPPTPIDRVADKGIAYGVESLVCSADDAGAVASAMAQAHQRALAGEGPTLVEAIRESKSDPIEALEARLSSEGHWDAHRALELRRELMTEIESAVAHAQQVGAPPREAVFEDVYATLPRHLEDQRTTLLATANHEDR

SEQ ID NO: 4 - DNA, 933 bp, Sandaracinus amylolyticus
atggccgatctgctggcgattcaccgacatgccgtgcgtgcccgtctgctggatgagcgtttaacgcaacttgcccgcgctggccgcatcgggttccaccctgatgcacgtggtttcgagccggctattgcggctgccgtactggctatgcgcgcggaagatgctattttcccgtccgcgcgagatcacgcagcgttcttggttcgcggattgccgattagccggtatgtggcccatgcgtttggcagtgttgaggatcctatgcgtggccacgctgcccccgggcacttagcgtcacgcgaactgcgcattgccgcggccagcggtctggtcagcaaccatatgactcacgccgccggttacgcgtgggcagctaaacttcgcggggaaacgtgcgcggttttgaccatgtttgcagacaccgctgcggacgctggtgactttcattcagcggtaaactttgcgggtgccaccaaggcgccggttatctttttttgccgtacagatcggacccgtagtgcacatccgccgacgccgattgaccgtgtggccgataagggcattgcatacggtgtggagagcttggtttgttcggccgatgatgccggtgcggtggctagcgccatggcacaggcacaccagcgcgctctggccggcgaaggtcctacgctggtggaagcgattcgtgaatccaaaagcgatcccatcgaggccctggaggctcgcctgtctagcgaaggtcactgggatgcgcaccgtgcgctggaactgcgccgcgagctgatgactgagatcgagtctgccgtggcgcatgcccagcaggttggtgctcccccacgcgaagccgtgttcgaagatgtctatgcaaccttgccgcgtcacctggaagaccagcgtacgacattactggccaccgccaaccacgaagatcgg

SEQ ID NO: 5 - protein, 533 aa, Polynucleobacter necessarius
MRTVKEITFDLLRKLQVTTVVGNPGSTEETFLKDFPSDFNYVLALQEASVVAIADGLSQSLRKPVIVNIHTGAGLGNAMGCLLTAYQNKTPLIITAGQQTREMLLNEPLLTNIEAINMPKPWVKWSYEPARPEDVPGAFMRAYATAMQQPQGPVFLSLPLDDWEKLIPEVDVARTVSTRQGPDPDKVKEFAQRITASKNPLLIYGSDIARSQAWSDGIAFAERLNAPVWAAPFAERTPFPEDHPLFQGALTSGIGSLEKQIQGHDLIVVIGAPVFRYYPWIAGQFIPEGSTLLQVSDDPNMTSKAVVGDSLVSDSKLFLIEALKLIDQREKNNTPQRSPMTKEDRTAMPLRPHAVLEVLKENSPKEIVLVEECPSIVPLMQDVFRINQPDTFYTFASGGLGWDLPAAVGLALGEEVSGRNRPVVTLMGDGSFQYSVQGIYTGVQQKTHVIYVVFQNEEYGILKQFAELEQTPNVPGLDLPGLDIVAQGKAYGAKSLKVETLDELKTAYLEALSFKGTSVIVVPITKELKPLFG

SEQ ID NO: 6 - DNA, 1599 bp, Polynucleobacter necessarius
atgcgcaccgttaaagagatcacattcgatctgttgcggaaactgcaagttaccaccgtggtgggcaacccaggctccaccgaggaaacgtttctgaaagattttccgtcggactttaactatgtactggccctccaggaagcgagcgtcgtcgcgatcgcggacggcttatcccagagtcttcgtaagcccgtgatcgttaacattcacacgggggcaggcttgggcaatgctatggggtgcttgttgacagcctatcagaataaaaccccccttattataaccgcggggcaacaaacccgcgaaatgctgctcaacgaaccgttattaaccaacatagaagcgatcaatatgccgaaaccgtgggtgaagtggagctatgaaccggcacggccggaggacgtcccgggcgcattcatgcgcgcgtatgcgacggctatgcaacagccccagggtccggtttttctgagccttccgcttgacgattgggaaaaacttatccctgaagtagatgtcgcccgcacagtgtctacccgtcaaggtccggatccggacaaggtcaaagaatttgcgcaacgcattaccgcatcaaaaaatccgctgctcatttatggcagcgatattgcgcgctcgcaagcgtggagcgatggtatcgcattcgcagaacgcctaaacgcaccggtctgggcggctcccttcgcggaacggaccccatttcctgaagatcatcccctttttcagggtgccctgacctcgggtatcggaagcctggaaaagcaaatccagggtcatgatttaatcgtggtcatcggtgccccggtgtttcgctactacccttggatcgcggggcaatttattccggagggctcaaccctccttcaggtgtcggatgatcctaatatgaccagcaaagcggtagttggtgattccttggttagcgattcgaaattgttcctgatcgaagcacttaaactgatcgatcagcgcgaaaaaaacaatacgccacagcgcagcccgatgaccaaagaggaccgtaccgccatgccactccgtccccatgctgttctcgaagtgctgaaagaaaattcaccgaaagagatagtactggtcgaagagtgtccatccatcgttcctctgatgcaggacgttttccgcattaaccaaccggataccttctacacctttgcaagtggcggcttgggttgggacctgccggccgcagtagggctggccctgggcgaggaagttagcggccgcaaccggcctgtggttacgcttatgggcgatggatccttccaatatagcgttcaaggtatttacacgggagtgcagcaaaaaacccatgtaatttacgtggtgttccagaacgaagaatatgggatcttaaagcagtttgcagaacttgaacagactccgaacgtgcccggactggatctgccggggctggacattgtggctcagggtaaagcgtatggcgcaaaaagccttaaagtggaaacacttgatgaattaaaaaccgcctatctggaagcgctgagctttaagggtacgtctgtcattgtcgtgccgatcaccaaggaattaaaaccacttttcgga

SEQ ID NO: 7 - protein, 452 aa, Mobiluncus curtisii
MLKQIEGSQAIARAVAACQPNVVAAYPISPQTHIVEALSALVKSGQLEHCEYVNVESEFAAMSACIGSSAVGARSYTATASQGLLYMVEAVYNAAGLGFPIVMTVANRAIGAPINIWNDHSDSMSQRDSGWLQLFAENNQEAADLHVQAFRIAEELSVPVMVCMDGFILTHAVEQVDLPESEQVKQFLPPYEPRQVLDPDDPLSIGAMVGPEAFTEVRYIAHHKMLQALDLIPQVQSEFKSIFGRDSGGLLHTYRCEDAETIIVALGSVVGTLKDVVDQRRENGEKIGIMSLVSFRPFPFAAIREVLQSAKRVVCLEKAFQLGIGGIVSSELRAAMRGLPFTCYEVIAGLGGRNITKNSLHAMLDQAVADTIEPLTFMDLDMELVQGELEREAATRRSGAFATNLQRERVLRANAKIAEAGPKPKADKVGNPRVASPSIKQDAVPVVPDQAE

SEQ ID NO: 8 - DNA, 1356 bp, Mobiluncus curtisii
atgctgaaacagattgaaggctctcaggcaatagcacgtgccgttgctgcgtgccagccaaacgtggtcgcagcctatccgatctcaccgcagacccatattgtggaagcactttctgcgctggtaaaaagtggccagctggaacactgcgagtacgtgaacgtagaatccgaattcgcagccatgtctgcctgcattggctcgtccgcagttggcgcgcgctcatatactgcgacggcatcacagggcttgctgtatatggttgaagcggtctacaacgccgctggcctgggcttcccgattgtcatgacggtggcgaaccgtgcaattggagctccgatcaatatctggaatgaccacagtgattcgatgtcgcagcgcgactctggctggctgcagctgttcgccgagaacaaccaggaagccgcagacttacatgtgcaggcatttcgtatcgctgaggagttgagcgtcccggttatggtgtgcatggatggtttcattctaacgcatgccgttgaacaggtcgacctcccggaatctgaacaagtgaaacagtttctccctccctacgaaccacgtcaagttctggacccggacgatccgttatctattggcgctatggttggtccggaagcgtttaccgaggtgcgctatattgctcatcataaaatgctgcaggctctggatctgatcccacaagtgcagtccgaatttaaatcaatatttggccgggactctgggggactgctgcatacgtatcggtgcgaagatgcggaaactattattgtggccctgggttccgttgtaggtaccctgaaagatgtcgtggaccaacgtcgcgagaatggcgagaaaatcggcatcatgagcttagtgagcttccgccccttcccatttgctgccatccgcgaggtcctgcagtcagcgaaacgcgtggtttgcctggagaaagcgtttcaattgggtattggggggattgtatcttctgagctgcgggcggccatgcgtggtttgccgttcacttgttacgaagtaatcgccggtttgggtggccgcaacattactaaaaacagtctacatgctatgcttgatcaggccgtcgctgatacgatcgagccgctaacctttatggatctggatatggagctggtgcagggcgagctcgaacgggaagcagcgacgagacgctctggcgctttcgccaccaacctgcaacgcgaacgtgtcctgcgtgcgaacgctaaaattgcagaagcaggtccgaaaccaaaagcagataaagtaggtaacccgcgggttgcgtctccgtcaatcaagcaggatgcggtgcctgtagtccctgaccaggctgaa

SEQ ID NO: 9 - protein, 399 aa, Cupriavidus metallidurans
MIEAVQFVEAARERGFEWYAGVPCSYLTPFINYVVQDPSLHYVSAANEGDAVAFIAGVTQGARNGVRGITMMQNSGLGNAVSPLTSLTWTFRLPQLLIVTWRGQPGGASDEPQHALMGPVTPAMLDTMEIPWELFPTEPDAVGPALDRAIAHMDATGRPYALIMQKGSVAPYPLKTQTPPVARAKATPQVSRSGATPLPSRQEALQRVIAHTPADSTVVLASTGFCGRELYALDDRPNQLYMVGSMGCLTPFALGLAMARPDLKVVAVDGDGAALMRMGVFATLGAYGPANLTHVLLDNNAHDSTGGQATVSHNVSFAGVAAACGYASAIEGDDLDMLDRVLASAATATSGPNFVCLQTRAGTPDGLPRPSVTPVEVKTRLGRQIGADQGHAGEKHAAA

SEQ ID NO: 10 - DNA, 1197 bp, Cupriavidus metallidurans
atgattgaggctgttcagtttgtcgaggcggcacgggaacgtggctttgaatggtacgcgggggttccctgcagttatttgactccgttcattaattatgtagttcaggatccgtcgctgcactacgtcagtgccgcgaacgagggagatgctgttgcattcatcgcgggcgtcacccaaggtgctcgcaacggcgtccgtggtatcaccatgatgcaaaattccggtctgggtaacgccgtgtccccgctgaccagcctgacctggaccttccgcctgccgcagctgttgatagtaacgtggcgtggtcagccgggcggcgcctcagacgaaccacaacatgcgctgatgggccctgtgaccccggcgatgctggacaccatggagatcccgtgggaactgtttccgacagaaccggatgcagtggggccagccctcgatcgcgccatcgcacacatggacgccacgggccgtccttacgcgctgatcatgcagaagggctcggtggctccatacccgctgaagacacagactccgccggttgcacgcgcgaaggcgaccccacaggttagtcgctcaggtgccacgccattaccatcgcgtcaagaagcccttcagcgggttatcgcccataccccggctgattcaactgtggttctggcatctactggcttttgcggtcgagaactgtatgcgttggatgaccgcccgaaccaattatatatggtgggttccatgggttgtctgacgccattcgcactggggttggcaatggcgcgtccggatctcaaagtggttgcagtagatggcgatggcgcggccctaatgcgcatgggggtgttcgcgactctgggggcgtatgggccggctaacctcacccacgttttattagacaacaacgcacacgattcaaccggcggccaggccaccgtaagccataatgtttcttttgcgggggtcgcagcggcgtgcggctacgcctctgcaatcgaaggtgacgacttggatatgctggaccgtgtgttagcgtccgccgcaacagcgacttccgggccgaacttcgtgtgcttacaaactcgtgcaggtacgccggacggcttaccacgaccatctgtgaccccggttgaagtgaaaacgcgccttggtcggcaaattggcgccgaccagggccacgcaggcgaaaaacacgccgcggcc

SEQ ID NO: 11 - protein, 161 aa, Bacteroides fragilis
MNTLTSQIEQLQSLAHELLYLGVDGAPIYTDHFRQLNKEVLEQSDALYPQRGATPEEEANICLALLMGYNATIYNQGDKEEKKQVVLNRCWDVLDQLPATLLKCQLLTYCYGEVFEEELAKEAHTIIESWSNRELLKAEKEIAESLNNLEANPYPYSELHE

SEQ ID NO: 12 - DNA, 483 bp, Bacteroides fragilis
atgaataccctgacctctcagattgaacaactgcaaagcctggcccacgaactgctgtatctgggtgtggacggtgcccctatctataccgaccattttcgtcagctgaacaaggaagtcctggaacaaagcgatgcgctctatccacagaggggcgctaccccggaagaagaggccaacatttgcctggcactgcttatgggttataatgcaacgatttacaatcagggcgataaggaagagaaaaaacaagtggtcctgaatcgctgttgggatgtgctggatcagctcccggcaaccctcctgaagtgtcagcttctcacgtactgctatggcgaagtttttgaagaagagttagcgaaagaagcccacacaatcatagagtcatggagtaaccgcgaactgctgaaagcagaaaaagaaatcgcggaatcgctgaataacctcgaggcgaatccgtacccgtattccgaactgcacgaa

SEQ ID NO: 13 - protein, 557 aa, Thiothrix nivea
MQIQVSELIVKFLQKLGVDTIFGMPGAHILPVYDELYDSGIKTVLVKHEQGAAFMAGGYARVSGRIGACITTAGPGASNLITGIANAYADKLPMIVITGEAPTHIFGRGGLQESSGEGGSIDQTALFSGVTRYHKLIERTDYITNVLSQAARQLVADVPGPVVLSIPVNVQKELVDASILENLPTLKPLPKLQIAPPVLEQCADMIRKARCPVILAGYGCLQSVRARLELRKFSEHLNIPVATSLKGKGAIDERSALSLGSLGVTSSGHAMHYFMQEADLIILLGAGFNERTSYVWKADLTQERKIIQVDRNVAQLEKVVKADLAIQSDLGDFLHALNTCCVPQGIEPKSCPDLAAFKQKVDQQAAQSGQVIFNQKFDLVKSLFARLEPHFAEGIVLVDDNIIYAQNFYRVKDGDLFVPNTGVSSLGHAIPAAIGARFVLDKPMFAILGDGGFQMCCMEIMTAVNYNIPLNIVLFNNQTLGLIRKNQHQQYEQRFLDCDFQNPDYALLAQSFGINHFHVGNNADLQRVFDTADFHHAINLIELMVDREAYPNYSSRR

SEQ ID NO: 14 - DNA, 1671 bp, Thiothrix nivea
atgcaaatccaggttagcgagctgattgtaaagttcttgcagaaattaggtgtcgatacaatttttggcatgccaggcgcccacatcctgcccgtgtatgatgaattatacgacagcggcataaaaaccgttctcgttaagcacgaacagggcgccgcgttcatggcgggtggctacgcccgggtttctggtcgaattggtgcgtgtatcactaccgctggcccgggggcctcgaatctaatcaccggtatcgctaacgcgtatgcggataaattgccgatgattgttatcaccggcgaggcccctacccacattttcggccgaggcggcttacaggaatcttccggtgaaggtggctcaatcgaccaaaccgcactcttcagcggggtgacccgataccacaaactgattgaacgtaccgattacattaccaatgtcctctcccaggccgcccggcagcttgtagccgatgtaccaggacccgttgtcctctcgattccagttaacgtgcaaaaagagcttgtcgacgcaagtattttagaaaacttacctacgcttaaaccgctgccgaaactgcagatcgcgccgccggtgctggagcagtgtgcggatatgatccgcaaggctcgttgtccagtcatcctggcggggtatggctgtctgcagtcggtgcgcgctagattagagctgcgtaaattcagcgaacacctgaatattccagtggcgacgagtcttaaagggaagggagcgattgatgaacgttcggcactcagcctggggtcgctgggcgtgacgagtagcggacatgctatgcactattttatgcaagaggcggatctcatcattctgctaggggcgggctttaatgaacgtacgtcttatgtttggaaggcagacttaacccaagagcgtaaaatcattcaggtcgatcgtaatgttgctcagctagaaaaagtggttaaggccgatttggcaattcagtctgatctgggcgattttttacacgcgctgaacacctgttgtgtgccccagggtattgaaccgaaatcatgtccggatctggcagcctttaaacagaaagtggatcagcaggcggcccagagtggccaggtgatcttcaaccagaaatttgatttagttaagtcgttgtttgcacgactggaacctcattttgccgaaggtatcgtattggtggatgacaatatcatctatgcgcaaaacttctaccgcgtgaaagacggggacctgtttgtaccgaacactggggtgagcagcctgggacatgcgattcccgccgccattggtgcgcgcttcgtcttggataaaccgatgtttgcgattcttggcgatggtggcttccaaatgtgttgtatggaaataatgaccgctgtgaattataatattccgctcaacatcgtgctctttaacaatcagaccctgggactgatacgtaaaaaccaacatcaacagtatgaacagcgtttcctggattgtgatttccagaacccagactatgccctactggcgcaaagctttggcattaaccactttcatgtgggtaacaacgccgatctgcagcgcgtttttgacacggcggattttcatcatgctatcaacctgattgagctcatggttgatcgcgaagcttatccaaactattcaagccgtcgc

SEQ ID NO: 15 - protein, 687 aa, Saccharomyces cerevisiae [sequence omitted]

SEQ ID NO: 16 - DNA, 2061 bp, Saccharomyces cerevisiae
atgatccgtcagtctaccctgaaaaactttgctatcaaacgctgctttcagcatattgcctatcgtaacactccggccatgcgttcggtagcgctagcacagcgcttctattcctcttctagcagatactattcggcatctccgctgccggccagtaaacgccccgaaccagctccgtcgttcaacgttgatccactggaacagccagcggaaccttctaagctggcgaaaaaacttcgcgcggaaccggatatggatacttcattcgtaggtctgacaggaggccagatctttaatgagatgatgagtcgtcaaaacgtcgacacggtattcggctacccgggcggagccatcctgccggtatatgatgcgattcataactcggataaattcaactttgtgttgccgaaacatgaacagggcgcgggccacatggcagagggatatgcgcgtgcaagcggcaaaccgggtgtcgtgctggtaacatcaggcccgggtgcaacaaatgttgtcacacctatggcggatgcttttgccgacggtatcccgatggtagtgttcaccggccaagtgccaaccagcgcgattggaacagacgctttccaggaagctgatgtggtcggcatctcccgcagttgtacaaagtggaacgtgatggtgaagagcgtagaagagttgcctctgcgtatcaacgaagcgttcgagattgcgaccagtgggcgcccggggcccgtcttagtcgacttacctaaggacgtaaccgccgcgatcctgcgcaatcctattccgaccaaaactacgttacccagtaacgcgctgaaccagcttaccagccgcgctcaggacgaattcgtcatgcagtccatcaataaagctgcggaccttattaacctggctaaaaagcctgtgctctatgttggtgccggtattctcaatcacgccgatggaccgcgtctgctgaaagagctgagcgaccgcgctcagatccccgtgaccactacgcttcaaggccttggctcctttgatcaggaagatcctaaaagcttagatatgttaggaatgcacggatgcgccacggcgaacctggcggtgcagaatgcggatctgattattgccgtcggcgcccgttttgacgaccgtgtgaccggcaacattagcaaatttgctcctgaagctcgtcgtgctgctgcggaaggacgtggaggaattattcattttgaagtaagtccaaaaaatattaacaaagtcgtacagacccagattgcggtcgagggtgatgcgaccaccaatctggggaagatgatgagcaaaatcttccctgtaaaagaacgtagtgagtggttcgcccagataaataagtggaaaaaagaatatccatatgcctatatggaggaaacgccaggtagtaaaattaaaccgcaaactgtgatcaaaaaactgtcaaaagtcgcaaacgatacgggtcgtcatgtaatcgtaactacgggcgtgggtcagcatcagatgtgggcggcgcagcattggacctggcgtaacccgcatacctttattacgagcggcggattggggaccatgggctatgggttgccggcggcgattggcgcccaggtggccaagccagagtcactggtcatcgatattgacggtgacgcgagcttcaacatgacgctgacggagttgtcctcagcggttcaggccggtactccggtgaaaatcctgattctgaacaatgaggaacagggtatggttacgcagtggcaaagcttattctacgagcaccgatattcccacacgcatcagctgaaccctgacttcattaaacttgctgaagcaatggggctgaagggcctgcgcgtgaaaaagcaggaagaacttgatgctaaactgaaagaattcgtctcgacgaagggaccagtacttttagaagtggaggtggataaaaaagttccagtcttacctatggtcgctggcggtagcggcctggatgaatttattaatttcgatccggaggtcgaacgtcagcaaactgaattgcgccataaacggacaggaggtaaacac

SEQ ID NO: 17 - protein, 397 aa, Streptomyces viridochromogenes
MIGAADLVAGLTGLGVTTVAGVPCSYLTPLINRVISDPATRYLTVTQEGEAAAVAAGAWLGGGLGCAITQNSGLGNMTNPLTSLLHPARIPAVVITTWRGRPGEKDEPQHHLMGRITGDLLDLCDMEWSLIPDTTDELHTAFAACRASLAHRELPYGFLLPQGVVADEPLNETAPRSATGQVVRYARPGRSAARPTRIAALERLLAELPRDAAVVSTTGKSSRELYTLDDRDQHFYMVGAMGSAATVGLGVALHTPRPVVVVDGDGSVLMRLGSLATVGAHAPGNLVHLVLDNGVHDSTGGQRTLSSAVDLPAVAAACGYRAVHACTSLDDLSDALATALATDGPTLVHLAIRPGSLDGLGRPKVTPAEVARRFRAFVTTPPAGTATPVHAGGVTAR

SEQ ID NO: 18 - DNA, 1191 bp, Streptomyces viridochromogenes
atgattggggctgccgatctggtcgctggtctgaccggtctgggtgtgaccacagtggccggtgtaccgtgcagttatttaactccgttaatcaaccgagtaatcagtgacccggcaacgagatatttgacggtgacgcaggaaggagaagcagcggcagttgcagcaggggcctggttgggtggtggtctgggctgcgcgattacccaaaacagcggtcttggcaacatgaccaaccctctcacctctttacttcaccctgcccgtatcccggcggtagttatcaccacctggcgcggccgcccgggtgagaaagatgagccccagcaccacctaatgggccgcattactggtgatctcctggacctgtgtgatatggagtggtcgctgattccggatacgaccgacgaactgcacacagcgtttgctgcttgccgtgcttccctggcgcaccgtgagctgccttatggttttctgcttccgcagggtgtggtggccgatgagccactgaacgaaacggctccgcgttcggccaccgggcaggtcgtccgctatgcgcgtccaggccggtctgctgcccggcctacgcgcattgccgccctggaacgcctactcgccgagttaccgcgtgacgcagcagtggtatctaccaccggcaaaagctcccgagagctgtacactttggacgatcgtgatcaacatttctatatggtcggtgcgatgggctctgccgcgaccgttggactgggagtcgcgttgcataccccccgtccggtcgttgttgttgatggtgacggctccgtcttgatgcgcctcggttcgctggcaaccgtgggggcccatgcccccggcaacctggtgcatcttgtgctggataacggtgtccacgatagcacgggtggccaacgcacgttgagcagcgcggtggatctcccagctgtcgccgccgcgtgcggctatcgcgctgtgcacgcctgcacctctctggatgatctcagtgatgcattggcgaccgcgttagcgacggatggtccgaccttagtgcacctggcgattcgcccgggaagcctggatggtctgggccgcccgaaagtcacgcccgctgaagtggcccgtcgttttcgtgcgttcgtgaccacccccccagccggtacagctacgcctgttcacgctggtggtgtgacagcccgg

SEQ ID NO: 19 - protein, 632 aa, Campylobacter jejuni
MNIQILQEQANTLRFLSADMVQKANSGHPGAPLGLADILSVLSYHLKHNPKNPTWLNRDRLVFSGGHASALLYSFLHLSGYDLSLEDLKNFRQLHSKTPGHPEISTLGVEIATGPLGQGVANAVGFAMAAKKAQNLLGSDLIDHKIYCLCGDGDLQEGISYEACSLAGLHKLDNFILIYDSNNISIEGDVGLAFNENVKMRFEAQGFEVLSINGHDYEEINKALEQAKKSTKPCLIIAKTTIAKGAGELEGSHKSHGAPLGEEVIKKAKEQAGFDPNISFHIPQASKIRFESAVELGDLEEAKWKDKLEKSAKKELLERLLNPDFNKIAYPDFKGKDLATRDSNGEILNVLAKNLEGFLGGSADLGPSNKTELHSMGDFVEGKNIHFGIREHAMAAINNAFARYGIFLPFSATFFIFSEYLKPAARIAALMKIKHFFIFTHDSIGVGEDGPTHQPIEQLSTFRAMPNFLTFRPADGVENVKAWQIALNADIPSAFVLSRQKLKALNEPVFGDVKNGAYLLKESKEAKFTLLASGSEVWLCLESANELEKQGFACNVVSMPCFELFEKQDKAYQERLLKGEVIGVEAAHSNELYKFCHKVYGIESFGESGKDKDVFERFGFSVSKLVNFILSK

SEQ ID NO: 20 - DNA, 1896 bp, Campylobacter jejuni
atgaacattcaaattttgcaagaacaagcgaacactctgcgtttcttgagtgcggacatggtccagaaagccaatagcggccaccctggcgcacccctgggcctggcggatatcctctctgtgctcagttatcatcttaaacacaacccaaaaaacccgacctggcttaaccgcgaccgcttagtgttttccggcggtcacgcctccgcactgttgtattctttccttcatctgagcggctacgacttaagtctggaagacctcaagaacttccgccagctgcactcgaagaccccggggcaccccgaaatttccaccctgggcgtagaaattgccacgggtcctctgggccagggggtggcgaatgcagtgggatttgcgatggcggcaaaaaaagcgcaaaatctgctgggcagtgacctgattgatcacaaaatctactgtctgtgcggtgacggcgatctgcaggagggtatttcatatgaggcgtgttctctggcgggcctgcacaaattagataattttatcctgatatatgatagtaacaacattagcattgagggtgacgtcggtctggcgttcaatgaaaacgttaagatgcgttttgaagcgcaggggttcgaagtgctgagcattaatggtcacgattatgaagaaattaacaaagccctggaacaggccaagaaatctaccaaaccatgcttgattatcgcaaaaacaaccattgcgaaaggcgcgggtgaacttgaaggtagccacaaaagccacggcgccccactgggtgaagaagtgatcaaaaaagcgaaagaacaggctggctttgatcccaacatctcttttcatattccgcaggcttcgaaaatccgctttgaaagcgccgttgaactgggggacctggaagaagcgaaatggaaggacaaacttgaaaaatccgcaaaaaaagaactgctcgaacgcctgctgaacccagattttaacaagattgcgtatcccgatttcaaaggcaaagacctggccacgcgagacagtaacggggagattttaaatgttctggccaaaaatctggagggtttcctgggcggctccgctgacctgggtccttcgaacaagacggagctacactcaatgggtgactttgttgagggcaagaacattcactttggtattcgtgaacatgccatggcggctattaacaatgcctttgcgcgctatggaatctttctgcccttttcagcgacgttcttcatcttcagcgaatatcttaaaccggcggcgcgcatcgccgcgctgatgaagatcaaacattttttcatttttacgcacgacagcatcggagtaggagaagacggcccgacgcaccagcctatagaacaattaagtacctttcgcgccatgccgaatttcctcacttttcgtccggcggatggggtagaaaacgtaaaagcttggcagattgcactcaatgccgacattccatctgcgttcgtcctctcacgtcagaagctgaaggccttgaacgagcctgtttttggtgacgtgaagaacggagcatacctgctgaaagaatctaaagaagccaagtttaccctgcttgcttctggctcggaggtgtggctgtgcttagaaagcgcaaacgaacttgaaaaacaaggctttgcctgcaacgtcgtgagtatgccgtgttttgagctgttcgaaaagcaggataaagcttaccaggaacgcctgcttaaaggagaagtaattggcgtggaggcggcacactctaatgaactgtacaaattttgccataaagtgtatgggatcgaaagctttggcgagagtggcaaagacaaagacgtttttgaacgtttcggcttttcggtgtccaaacttgtgaattttattctgtccaaa

SEQ ID NO: 21 - protein, 573 aa, Streptomyces clavuligerus
MSRVSTAPSGKPTAAHALLSRLRDHGVGKVFGVVGREAASILFDEVEGIDFVLTRHEFTAGVAADVLARITGRPQACWATLGPGMTNLSTGIATSVLDRSPVIALAAQSESHDIFPNDTHQCLDSVAIVAPMSKYAVELQRPHEITDLVDSAVNAAMTEPVGPSFISLPVDLLGSSEGIDTTVPNPPANTPAKPVGVVADGWQKAADQAAALLAEAKHPVLVVGAAAIRSGAVPAIRALAERLNIPVITTYIAKGVLPVGHELNYGAVTGYMDGILNFPALQTMFAPVDLVLTVGYDYAEDLRPSMWQKGIEKKTVRISPTVNPIPRVYRPDVDVVTDVLAFVEHFETATASFGAKQRHDIEPLRARIAEFLADPETYEDGMRVHQVIDSMNTVMEEAAEPGEGTIVSDIGFFRHYGVLFARADQPFGFLTSAGCSSFGYGIPAAIGAQMARPDQPTFLIAGDGGFHSNSSDLETIARLNLPIVTVVVNNDTNGLIELYQNIGHHRSHDPAVKFGGVDFVALAEANGVDATRATNREELLAALRKGAELGRPFLIEVPVNYDFQPGGFGALSI

SEQ ID NO: 22 - DNA, 1719 bp, Streptomyces clavuligerus
atgagccgtgtctctacagcgccttcgggtaaacctacggcagctcacgcacttttaagtcgcctgcgtgaccatggggtaggcaaggttttcggtgtggtgggccgtgaagccgcctcgatcctgttcgatgaagtcgaaggtatcgatttcgtcctgacccgccatgagtttaccgcaggcgtagccgcggacgtgttagcacgtatcaccgggcgtccacaagcctgctgggctaccctgggaccgggaatgaccaatctgagcaccgggattgcaacgtcagtattagaccgttcgccggttattgcgctcgcagctcagagtgaatcacacgatattttcccaaacgacacccaccaatgtttagactcagtggcgattgtggcaccgatgagcaaatatgcggttgagctgcagcgcccacacgaaattacggatttggtcgatagtgccgttaatgccgcgatgactgaacccgtgggccccagctttattagcctaccagtcgatctgctggggtcgagcgaagggattgacacaacagtgccgaacccgccggcgaataccccggctaaaccggtgggcgtggtagctgatggctggcagaaagcggcagatcaagctgctgcgcttttggcagaggccaaacatccagtattagtggtgggtgcagcggcgatccgtagcggagctgttcctgcaattagagctttggcagaacgtttgaacatccccgtcatcaccacctatatcgctaaaggtgtcctgccggttggtcatgaactgaattacggtgctgtcaccggctatatggatggcatcctgaacttcccagcgctgcaaaccatgtttgctccggtggatttagtactgaccgtgggttatgattatgcagaagatctgcgaccttcgatgtggcaaaaaggtatcgaaaaaaagacagttcgaatttcgccgactgtgaaccccatccctcgggtctatcgtccggacgtggacgtcgtgaccgacgtgctggcttttgtggaacactttgaaaccgcgaccgcgtccttcggtgcgaaacagcgacacgacatcgaacccttgcgtgcacgtattgcagaattcttggcggacccggaaacctatgaggatggaatgcgagtccatcaggtaatcgattctatgaacaccgtcatggaagaggcggcagagccaggcgaaggcaccattgttagtgatattgggttcttccgccactatggtgtcttgtttgctcgtgcggaccaaccctttgggttcctgacctctgcgggttgttcatcttttggatacggtattccagcggctatcggagcacagatggcccgtccggatcaacctacatttttaattgcaggcgatggcggttttcactctaattcgagcgacctggaaaccattgctcgccttaacctgccgatcgtgacggttgtcgtgaacaatgacacgaacggcctgattgaactgtaccagaatatcggtcatcatcgcagtcatgatccagccgtaaagttcgggggtgtcgattttgtggcgctggcggaagcaaacggcgttgatgcgacccgggcaaccaatcgtgaggagctgcttgcggcgttgcgtaaaggcgcagaactgggtcgtccgttcctgatcgaagtaccggtaaactatgactttcagccgggtggctttggcgctctgtctatt

SEQ ID NO: 23 - protein, 387 aa, Fibrobacter succinogenes
MLSPKFFVETLQTYSMDFFTGVPDSLLKNMCAYITDHIESQNNIIAVNEGTALGLAAGYYIATGCIPIVYMQNSGIGNTVNPLLSLTDKVVYNIPVLLLIGWRGEPGIKDEPQHIKQGMITIPLLDTLGIKNQILNKDPNMAKSQINDAIEYMRMTKEAFAFVIQKDTFEEYKLQNTEDSKFDLDREEAIKIVCNSLDKGSVIVSTTGMISRELFEYRESIDANHETDFLTVGSMGHASQIALGIALRRKNKKVYCFDGDGAVLMHMGALTTIGTSRAVNYIHIVFNNGAHDSVGGQPTVGLKVNLSKIASACGYNNVISVDSKATLKESLDRFKSINGPVLLEVKVRKGARKDLGRPTLTPVKNKELLMNFLEEADESDKSDNVFK

SEQ ID NO: 24 - DNA, 1161 bp, Fibrobacter succinogenes
atgctgagccccaaattctttgtcgaaaccctgcaaacctattccatggacttttttacgggcgtgcccgattcgctgttgaaaaacatgtgcgcctatataactgatcatattgaatcacagaacaacattatcgcagttaatgaaggcactgcgcttgggctggcggcgggttactacatcgcaaccggttgcatcccgattgtatatatgcagaacagtgggattggtaacactgtaaatcctcttttgagtttgacggacaaagttgtgtacaacatcccggtgcttctccttattggctggcgcggcgagccgggcattaaggatgaaccgcagcatatcaaacaggggatgatcaccatcccgttgctggatacactaggcattaaaaaccaaattctcaataaggacccaaacatggccaaatcacaaattaacgatgccatcgagtacatgcggatgacgaaagaggcattcgcctttgtaattcagaaagacactttcgaggaatacaaactgcaaaacaccgaagacagcaagttcgacctggaccgcgaagaggcgattaaaatcgtgtgtaattccttagacaaaggctccgtgattgtgagtacgaccggcatgatctcgcgtgaattattcgagtaccgcgaaagcatcgatgctaaccatgaaactgacttcctcacagtcggttccatgggtcacgccagtcaaatcgctctgggcatcgcactgcgccgtaaaaacaaaaaagtctactgtttcgatggcgatggagccgtcttaatgcatatgggcgccttaacgacaattggcacgagccgcgctgtcaactacatccacattgtgttcaacaatggggcacacgatagcgtagggggccagccgacggttggcctcaaagtaaacctgagtaaaattgcaagcgcgtgcggttacaacaatgtaatctccgtggattctaaggcaacattgaaagaaagcctcgatcgttttaaatcaataaatggtccggtattgctcgaagttaaggtacgcaaaggcgcgcgtaaagacctgggtcgcccgaccttaacaccggttaaaaacaaggaactgctgatgaactttctggaagaagctgatgaaagcgataaaagcgataatgttttcaaa

SEQ ID NO: 25 - protein, 376 aa, Peptococcaceae bacterium
MISTKRFGEELKKLGFDFYSGVPCSFLKNLINYTTNHCNYLAATNEGEAVAVAAGAFLAGKKPVVLMQNSGLTNAVSPLVSLNYLFRLPVLGFVSLRGEPGIPDEPQHQLMGRITTQMLDLVEIQWEYLSTDFDEVKKQLLQAYSCIESNQPFFFVVKKDTFEKEQLTDSQKRLSKNMFKSERTKADQVPKRFETLRLINSLKDVKTVQLTTTGITGRELYEIEDVSNNLYMVGSMGCVSSLGLGLALTKKDKDVVVIEGDGALLMRMGNLATNGYYGPPNMLHILLDNNMHESTGGQSTVSYNINFVDIAAACGYTKSIYVHNLVELESHIKDWKREKNLTFLYLKIAKGSIEGLGRPKMKPHEVKERLKVFLDG

SEQ ID NO: 26 - DNA, 1128 bp, Peptococcaceae bacterium
atgattagcactaaacgctttggtgaagaactaaaaaaactgggctttgatttctattccggcgttccttgcagcttcctgaaaaacctaatcaattacaccacgaatcactgtaactacctggccgctaccaacgagggagaggcagtcgcggttgccgcgggtgcgttcctggccggcaaaaaaccggttgtgctgatgcaaaactccgggttgacgaatgccgtctctccccttgtaagcctgaactatctcttccgcttaccggtgctgggttttgtctcccttcgcggtgaacctggtatcccagacgagccgcaacaccagctcatgggccgtattaccacccaaatgcttgatctggttgaaattcagtgggagtatctctccacagattttgatgaggtgaaaaaacagctgttacaggcatacagctgtattgaatcaaatcaaccgttctttttcgtggtaaaaaaagatacctttgaaaaagaacagttaaccgactctcagaaacgtctgagcaaaaacatgtttaaatcggaacgcaccaaagcggatcaggtgcccaaaagatttgaaaccctgcggctaataaactccctgaaagatgtgaagaccgtgcagctcactacgacgggcattaccggccgtgaactatacgaaattgaagatgtcagcaataacctatatatggtaggtagtatgggctgtgtcagttcgctgggcctgggactggcgctgactaaaaaagacaaagatgtggttgttatcgaaggtgatggcgccctgctgatgcggatgggtaaccttgcgacgaacggttactacggtccgccgaatatgctgcacattttgctggataataatatgcatgaatccactggaggtcagagtaccgttagctacaacatcaatttcgttgacattgctgccgcgtgcggttatactaaatccatctatgtgcataacctggtggaactcgagtcgcatatcaaagattggaaacgggagaaaaatctcacgtttctctatctgaaaatcgccaagggtagcattgaaggactgggccgtccaaaaatgaaacctcacgaggtgaaagaacgtttaaaagtattcttggatggt

SEQ ID NO: 27 - protein, 512 aa, Methanococcus voltae
MKTIVILLDGVADRPSKELNYKTPLQYANIPNLDEFAKSSLTGLMCPQKIGVPLGTEVAHFLLWGYDISQFPGRGVIEALGEGIDLKKDSIYLRATLGHVNYNQKENNFLVLDRRTKDINNQEISELLNKISNINIDGYLFTIHHMQGIHSILEISKLENDGNLKTEPNLKKNNLKKNGFELTYEEFCNEKNILKYGNINNINNCISNKISDSDPFYKDRHVIMVKPVIKLIGTYEEYLNALNVSNALNKYLTTCNTLLENDSINISRKNENKSLANFLLTKWAGSYKKLPSFKQKWGLNGVIIANSSLFRGLAKLLKMDYYEVKEFDKAIELGLKFKNDNTNNNNNSNNNNNNNQNNNINNKKIYDFIHIHTKEPDEAGHTKNPINKVRVLEKLDKNLKVVIDEIDKEKENGDENLYIITGDHATPSTGGLIHSGELVPIAICGKNVGKDSTKAFNEMDVLNGYYRINSTDIMNLVLNYTDKALLYGLRPNGDLKKYIPEDNELEFLKKDN

SEQ ID NO: 28 - DNA, 1536 bp, Methanococcus voltae
atgaaaaccatcgttattctgctcgatggggttgcggatcgtccttccaaagaactgaattataaaactccgcttcaatacgcgaacatcccgaatctcgacgaattcgctaagtcttccttaacgggcctcatgtgtccccagaaaattggggttccactgggcacggaagtcgctcatttcttgctgtggggctacgatattagtcagttccccggacggggggtgatcgaagcgctgggtgaaggcattgacctgaaaaaagattcgatttacctgcgcgctaccctcggtcatgtgaactataatcagaaggagaacaacttccttgtgttggatcgtcggaccaaagacattaacaatcaagagatctcagagctgctcaacaaaatttccaacattaacattgatggttatctgtttaccattcatcacatgcagggtatccacagtattctggaaatttctaagctggagaatgacggtaatctgaaaaccgaaccgaacttgaagaaaaacaatctgaaaaaaaatggcttcgaactgacctatgaagaattttgcaacgagaaaaatattctgaagtatggcaatattaacaacatcaataattgcatctctaacaaaatttcggattcagacccgttttacaaggatcgccacgtgataatggttaaaccagtaattaaactgattggtacctacgaagaatatctgaacgccctgaatgtaagcaacgcgctgaataaatatctgacaacgtgtaacaccctgctggaaaatgacagcatcaatatttcacgtaaaaatgagaataaatctctggcaaattttctgctgactaaatgggcgggcagctataaaaagctgcctagctttaaacagaaatggggcttaaatggtgtgattattgctaacagttctctgttccgtggtctggccaaactcctcaaaatggactattatgaggtgaaagagttcgacaaggcaattgaactggggctgaagttcaagaacgataacacgaacaataataacaactccaacaataacaacaacaacaatcagaacaacaatatcaacaataagaagatctacgactttatccatatccatacgaaagaacctgatgaggccgggcataccaagaatccgatcaacaaggtacgcgtgctggaaaaactcgataaaaatttaaaagtagttattgatgagatcgataaagagaaggaaaacggcgatgaaaacctttacattattaccggtgaccacgcgacaccatcgacgggcggtctgatccattcgggcgaactggttccaattgcaatttgtggcaagaacgttggtaaagactctacgaaggcgtttaacgaaatggacgtactgaacggctattaccggatcaattcaaccgatatcatgaacctggtgcttaactatacggataaagccctcctgtatggactccgtccaaacggggatcttaagaaatatattcctgaagacaatgaactggaattcctcaaaaaagataac

SEQ ID NO: 29 - protein, 670 aa, Arabidopsis thaliana
MAAATTTTTTSSSISFSTKPSPSSSKSPLPISRFSLPFSLNPNKSSSSSRRRGIKSSSPSSISAVLNTTTNVTTTPSPTKPTKPETFISRFAPDQPRKGADILVEALERQGVETVFAYPGGASMEIHQALTRSSSIRNVLPRHEQGGVFAAEGYARSSGKPGICIATSGPGATNLVSGLADALLDSVPLVAITGQVPRRMIGTDAFQETPIVEVTRSITKHNYLVMDVEDIPRIIEEAFFLATSGRPGPVLVDVPKDIQQQLAIPNWEQAMRLPGYMSRMPKPPEDSHLEQIVRLISESKKPVLYVGGGCLNSSDELGRFVELTGIPVASTLMGLGSYPCDDELSLHMLGMHGTVYANYAVEHSDLLLAFGVRFDDRVTGKLEAFASRAKIVHIDIDSAEIGKNKTPHVSVCGDVKLALQGMNKVLENRAEELKLDFGVWRNELNVQKQKFPLSFKTFGEAIPPQYAIKVLDELTDGKAIISTGVGQHQMWAAQFYNYKKPRQWLSSGGLGAMGFGLPAAIGASVANPDAIVVDIDGDGSFIMNVQELATIRVENLPVKVLLLNNQHLGMVMQWEDRFYKANRAHTFLGDPAQEDEIFPNMLLFAAACGIPAARVTKKADLREAIQTMLDTPGPYLLDVICPHQEHVLPMIPSGGTFNDVITEGDGRIKY

SEQ ID NO: 30 - DNA, 2010 bp, Arabidopsis thaliana
atggcggctgctaccaccactaccacaacatcttcgtctatatccttttctactaaaccgagcccttcttcttccaaaagtccactgcccatttcacgcttctccttaccgtttagcctgaaccccaacaagagctcgagcagctcacgccgccgcggtattaaatcatcgagcccgtctagcatatccgcggttctcaacaccactaccaacgttacgaccactcctagcccgaccaaacccactaaaccggaaacctttatttcgcgattcgctccggaccagcctcgtaaaggtgcggatattcttgtggaagcgctggaacgccagggcgtggaaaccgtgtttgcttacccgggtggcgcttccatggagatacatcaggccttgacacggagttcatctatccgaaatgttctgccgcgtcatgaacagggcggtgtatttgcagcggaagggtacgcgcgctcctctggcaaaccaggcatctgcattgcgacctcaggccccggtgctaccaatctcgttagcggcctggcagatgcgttactggatagcgtgccgttagtcgcgattaccggtcaggtgccacgtcgtatgatcggcactgatgcgttccaggaaacacctatagtagaggtgacccgttcaatcacgaaacataactatttggtgatggatgtagaggacatcccgcgcattattgaagaagcgttttttctagccacttctggtcgcccaggcccggtcctggtagatgtgcccaaagatatccaacagcagctggcgatcccgaattgggagcaggcaatgcgcctccccgggtacatgtcgcgaatgccgaaaccgccggaagattctcatttagaacagattgtgcgtttaatttcggaatcgaaaaaaccggttctgtatgttggcggtggctgcttgaattcatcagatgaactgggtcgtttcgtagaactcaccggcattccggtagcgtcaaccctgatgggcctgggttcctatccgtgcgatgacgagctctcgctgcatatgctcggaatgcacggtaccgtgtacgccaattacgctgtggaacacagtgaccttctgctggcgtttggtgtacgttttgatgatcgtgtcaccggcaagctggaggcgttcgcgtcgcgcgcgaaaattgtccacattgatattgattctgcggagattgggaaaaacaaaaccccgcacgtctccgtgtgcggggacgttaagctcgcacttcagggcatgaataaagttctggaaaaccgtgcagaagaactgaaactggatttcggcgtgtggcgtaacgaacttaatgtacagaagcagaaatttccgctgtcttttaaaacgtttggtgaagcaatcccgccccagtacgccatcaaagtccttgacgaattaaccgacggtaaggcaatcataagcaccggtgtgggtcaacatcagatgtgggcggctcaattttataattataaaaaacctagacagtggctctcgtcaggcggcctgggtgccatgggctttggactgcctgccgcaatcggcgcaagtgtagcgaacccggacgctatcgtggtggatatcgacggcgatggtagttttattatgaacgtccaggagctggccaccatccgcgtagagaacctgcccgtaaaagttttattgttaaacaaccagcatttaggtatggtgatgcaatgggaagatcgtttctacaaggccaatcgcgcgcacacctttttaggcgatcctgcgcaggaagatgagatttttcctaacatgctgcttttcgccgcagcttgcggcatccccgccgcgcgagtaaccaagaaagcagatctccgtgaagccatccagactatgctcgatacccccggtccgtatctgcttgacgtgatttgtccgcatcaagaacacgttcttccgatgattccgagcggcggcacctttaatgatgtgatcacggaaggggacggtcgcattaaatat

SEQ ID NO: 31 - protein, 412 aa, Pyrococcus furiosus
MVLKRKGLLIILDGLGDRPIKELNGLTPLEYANTPNMDKLAEIGILGQQDPIKPGQPAGSDTAHLSIFGYDPYETYRGRGFFEALGVGLDLSKDDLAFRVNFATLENGIITDRRAGRISTEEAHELARAIQEEVDIGVDFIFKGATGHRAVLVLKGMSRGYKVGDNDPHEAGKPPLKFSYEDEDSKKVAEILEEFVKKAQEVLEKHPINERRRKEGKPIANYLLIRGAGTYPNIPMKFTEQWKVKAAGVIAVALVKGVARAVGFDVYTPEGATGEYNTNEMAKAKKAVELLKDYDFVFLHFKPTDAAGHDNKPKLKAELIERADRMIGYILDHVDLEEVVIAITGDHSTPCEVMNHSGDPVPLLIAGGGVRTDDTKRFGEREAMKGGLGRIRGHDIVPIMMDLMNRSEKFGA

SEQ ID NO: 32 - DNA, 1236 bp, Pyrococcus furiosus
atggttctgaaacgtaaagggctgctgattatcttggatggtctgggtgatcgtccgatcaaagaattaaacggcttaactccgttggaatatgccaacaccccaaatatggataaactggcggaaatcggcattctaggccagcaggatccgatcaaaccaggccagccggccggctctgacactgcgcacctgtcaatctttggctatgatccctatgaaacttaccgtgggcggggcttttttgaagcattaggggtgggccttgatctgagtaaagacgatctggcctttcgtgtgaattttgccacgctcgaaaatgggattattacggatcgtcgcgcaggccgtattagcacagaggaagcgcacgaactggcgcgggcgattcaggaggaagtggacattggggttgacttcattttcaaaggcgcgaccggccatcgtgcagtgctcgttttaaaaggtatgtctcgtggttataaagtgggtgataacgatccgcatgaagctggtaaaccgccgttaaagttttcatatgaagacgaggattcaaagaaagtagccgaaattctcgaagaattcgtgaaaaaagcgcaggaagttcttgaaaaacacccaattaatgaaagacgccgcaaggagggcaaaccgatcgcgaactatttgctgattcgcggggctgggacgtatccgaacataccgatgaaattcaccgagcagtggaaagtgaaggcggccggcgtaattgcagtggcgctggttaaaggcgtagcacgtgcagtcggcttcgacgtatatacccctgaaggggcgaccggagagtacaacacgaacgaaatggccaaagcaaaaaaagcagtagaactgctaaaagattatgattttgtgttcttacacttcaaaccgactgatgccgcggggcacgacaacaaaccgaagctgaaagcggaattgattgaacgcgccgatcgcatgattgggtatatcttggatcatgttgacttagaagaagttgtaatcgctatcaccggcgatcattcgacgccatgcgaggtaatgaatcatagcggggaccctgtcccacttttgattgcgggtggcggcgtgcgcacggacgataccaaacgtttcggcgagcgcgaggcaatgaaaggcggccttggccgcatccgtggccacgatattgttcctatcatgatggatctaatgaatcgttcggaaaaatttggtgcg

SEQ ID NO: 33 - protein, 392 aa, Clostridia bacterium
MLLVVLDGLGGLPVPELNGRTELEAAATPNLDALAKRSSLGLAHPVLPGIAPGSSAGHLALFGYDPLRYVIGRGVLEALGIGFDLHPGDVAVRANFATVQDTRNGPVVTDRRAGRPPTEHTRSICRRLQDAIPEIDGVRVFIEPVKEHRFVIVLRGEGLDDRVADTDPQREGMPPLQPQPLAEEARRTAMLAGTLVQRIAELVRDEPRTNFALLRGFSRRPRLDPFPERYRARAGAVAVYPMYRGLASLVGMDLLPVAGDTLADEIASLKENWPEYDYFFLHVKGTDSRGEDGDWAGKIKIIEEFDAQLPAILDLNPDALVITGDHSTPATYAAHSWHPVPFLLYSRWVLPDRDAPGFGEHACARGVLGQFPLLYTMNLLLANAGRLGKFSA

SEQ ID NO: 34 - DNA, 1176 bp, Clostridia bacterium
atgctgctggttgttctggatggtctgggcggccttccggtgcctgaactgaatgggcgtacggaacttgaggcggccgcgacaccgaacttagatgcgctggcgaagcgctcttccctgggcctggcacatccggtgctgccgggcatagcgcctggttcttctgctgggcatctggctcttttcggttacgatccgttgcgttatgtcattggccgcggcgtcctggaggccctgggcattggtttcgacctccatcccggtgatgtggccgtccgtgctaatttcgcaaccgtccaagacacgcggaacggtccagtcgtgacggatcgacgtgcgggccgtccgccgacggaacatactcgtagtatctgtcgtcgcctgcaggacgcaattccggagattgacggtgtacgtgtcttcattgagccggttaaagaacatagattcgtgattgtgctgcgaggcgaaggtctggatgatcgcgtcgccgacacggatccccaacgtgaagggatgcctccgttacaaccgcaaccgcttgctgaagaagctcgtcgcacagcgatgctggcgggaaccctggtgcaacggattgctgagttagtccgcgatgagcctcgtactaattttgctctgctgcgcgggttctctcgccgtcctcgcctggacccgttcccagaacgttatcgtgcccgcgcaggagcagtggcagtctatccgatgtatcgcggtctggcatccctggtcggtatggatctgctgccagtcgccggggatacgcttgccgacgaaattgcgagcctcaaggaaaactggcctgagtatgattacttctttctgcacgttaaaggcacggacagtcgcggtgaagatggtgattgggcaggcaaaatcaagattattgaggaatttgacgcccagctgcctgcaattctagatttaaatcccgatgcgttggtgattacaggcgatcacagtacgcctgctacgtacgcggcccatagctggcatcctgtgccttttctgttgtacagccgctgggtcctgccggatcgcgatgcgccaggtttcggcgaacacgcatgcgcccgtggagtgctgggtcagttcccgctgttgtatacgatgaatcttttgttggccaatgctgggcgtctcggcaaattcagcgcc

SEQ ID NO: 35 - protein, 387 aa, Pyrococcus furiosus
MNKRFPFPVGEPDFIQGDEAIARAAILAGCRFYAGYPITPASEIFEAMALYMPLVDGVVIQMEDEIASIAAAIGASWAGAKAMTATSGPGFSLMQENIGYAVMTETPVVIVDVQRSGPSTGQPTLPAQGDIMQAIWGTHGDHSLIVLSPSTVQEAFDFTIRAFNLSEKYRTPVILLTDAEVGHMRERVYIPNPDEIEIINRKLPRNEEEAKLPFGDPHGDGVPPMPIFGKGYRTYVTGLTHDEKGRPRTVDREVHERLIKRIVEKIEKNKKDIFTYETYELEDAEIGVVATGIVARSALRAVKMLREEGIKAGLLKIETIWPFDFELIERIAERVDKLYVPEMNLGQLYHLIKEGANGKAEVKLISKIGGEVHTPMEIFEFIRREFK

SEQ ID NO: 36 - DNA, 1161 bp, Pyrococcus furiosus
atgaataaacggtttccgttcccggtgggagaacctgattttattcagggtgatgaggctatcgctcgtgcagccattttagccggatgtcgtttttatgcgggatacccgatcacgcccgcgtcggaaatcttcgaagcgatggcactatatatgccgctggtcgatggcgtagttatccagatggaagatgagattgcgtcgatcgcggccgccatcggggcaagttgggctggtgctaaggcgatgaccgctacctctgggcccggattcagcctgatgcaagaaaacattggttacgcggttatgacagaaacgcctgtggttatagtcgacgtgcagcgtagcggtccaagcacgggacaaccgaccctgcctgcgcaaggcgatattatgcaggcgatttggggcacgcatggcgaccacagcctgatagttctgtcaccgtcgacggtccaggaggcgttcgattttacgattcgtgcgttcaacctgtccgaaaagtaccgtaccccggtcatcctgctcaccgatgccgaagtgggacatatgcgggaacgtgtttatatcccgaacccagatgaaatcgaaattattaatcgtaagctgccgcgcaacgaagaggaagcaaaattaccgttcggtgatccgcacggcgatggggttccccccatgcctattttcgggaaaggttacaggacgtatgtgaccggcctgacccatgatgaaaaaggtcgcccacgcacagtcgatcgtgaagtgcatgaacgcctgattaaacgtatagttgaaaaaatagaaaagaacaagaaagatatctttacgtacgaaacgtatgagctggaagatgccgaaattggagtggttgcaacgggtattgtggcccgttcggccttacgtgctgtcaaaatgctgcgcgaagagggcatcaaagcgggcctgttgaaaattgaaactatttggccgtttgacttcgaattaatcgagcgtattgcggaacgcgtggataaactgtatgtaccggaaatgaacttagggcagctgtatcacctgattaaggaaggcgcgaacggcaaagcggaagttaaattaatcagcaagatcggtggagaagtgcataccccgatggagatctttgaatttattcgtcgcgaattcaaa

SEQ ID NO: 37 - protein, 573 aa, Tolumonas auensis
MTEQWQSLDSLNALWSALLIEELARLGIRDICIAPGSRSTPLTLAAAANPAISTHLHFDERGLGFLALGLAQGSQRPVAVIVTSGSAVANLLPAVVEARQSGIPLWLLTADRPAELLGCGANQAITQANIFANYPVYQQLFPAPDHDITPSWLLASVDQAAFQQQQTPGPVHLNCPFREPLYPVAGQQIPGNALRGLTHWLRSAQPWTQYHAVQPICQTHPLWAEVRQSKGIIIAGRLSRQQDTGAILKLAQQTGWPLLADIQSQLRFHPQAMTYADLALHHPAFREELAQAETLLLFGGRLTSKRLQQFADGHNWQHCWQIDAGSERLDSGLAVQQRFVTSPELWCQAHQCEPHRIPWHQLPRWDGKLAGLITQQLPEWGEITLCHQLNSQLQGQLFIGNSMPIRLLDMLGTSGAQPSHIYTNRGASGIDGLIATAAGIARANTSQPTTLLLGDSSALYDLNSLALLRELTAPFVLIIINNDGGNIFHMLPVPEQNQIRERFYQLPHGLDFRASAEQFRLAYAAPTGAISFRQAYQQALSHPGATLLECKVATGEAADWLKNFALQVRSLPA

SEQ ID NO: 38 - DNA, 1719 bp, Tolumonas auensis
atgaccgaacagtggcagtccctcgattctctgaatgccttgtggtctgcgctgttgattgaagagctcgcacgcctggggattcgggatatttgtattgccccaggcagccgctcaacccctcttactctggccgccgctgctaacccggcgatctcaactcatttgcattttgacgaacgcgggttaggttttcttgccctggggttggcgcaggggagccagcgtccggtcgcggttatcgtgacgtctggaagcgcggtcgcaaacctgctgcccgctgtcgtcgaagcacgccagagtggcattccgctttggttactgacggcggatcgcccagcagaattgctcggttgcggcgccaatcaggcgatcacgcaggcaaacatatttgcgaactatccagtgtatcagcaactgtttcctgctccggatcatgatattactcctagctggctgctggcgagtgtggaccaggcagctttccagcagcaacagacgccgggacccgtacatctgaactgtccgttccgagaaccactgtacccggtcgcgggccagcagattccgggtaatgcactgcgcggtctgacccactggttacgctctgcgcaaccgtggacacagtatcatgcggtccaacctatctgccaaacccacccgctttgggcagaagtgcgccagagcaaaggcattattattgcgggccgactgtcacgtcagcaagataccggtgccatcctgaaactggctcaacagaccggctggccgctgttggctgatattcagtcgcagctgcgttttcatccgcaggccatgacgtacgcggatctggcactccatcatccggcgtttcgtgaagaactagcgcaggcagaaaccctcttactgtttggtggtcgactgacttcgaaacgcctgcaacaatttgcagatggccacaattggcagcattgctggcagattgacgccgggtcagagcggctggactcgggtcttgcggtccaacagcgttttgtgacttctccagaactgtggtgccaggcgcatcagtgtgagccgcatcgtatcccgtggcaccaactgccacggtgggacggtaaactggcaggtctgattacccagcagctgccggagtggggtgagattacactatgccatcagctgaactcacagttacaaggccagttattcatcgggaattcgatgccaatccgcctgctggatatgctcggcaccagcggcgcgcagccatcgcatatttacactaaccggggcgcaagtggcattgacgggctaatcgccacggccgcgggtatcgcccgtgcgaatacaagccagccgacgaccctgcttctgggggacagcagcgccctgtacgacttgaacagcctggcactattacgcgaactgaccgctccgttcgtactgatcataatcaataatgacggcggcaatatctttcatatgctgccggttccagagcagaatcagattcgcgaacggttctatcagctgccgcatggcctggactttcgcgctagtgccgaacaattccgattagcgtatgccgcgcccaccggagccatctcctttcgtcaagcgtaccaacaagccctgagccatccgggggcgacactgctggagtgcaaagttgccacgggcgaagccgcagattggctcaaaaattttgcgctccaagtccgcagtcttccggcg

SEQ ID NO: 39 - protein, 371 aa, Selenomonas noxia
MNANDLIAALGAEFFTGVPDSKLRPLVDCLMDTYGANSPSHIIAANEGNAAALAAGYHLAAGKVPLVYLQNSGLGNIVNPLLSLLHAEVYGIPCIFVIGWRGEPDLHDEPQHLVQGRLTLPLLETIGVKTMVLTEASQPEDVSAWMEQIRPHLAAGGQCALLVRKGALTHPKHKYANENPLRREDAIARILDAAQGAVVVATTGKTGRELFELRAARGEDHAHDFLTVGSMGHAGAIALGIALHRPSQRVFLLDGDGAALMHMGAMATIGAAAPANIVHVLLNNEAHESVGGAPTAAHTVDFPAVARAVGYRLVQTAADAAELAQILPAVGRSDALTFLEVRTAIGSRADLGRPTTTPTENKEALMRTLRE

SEQ ID NO: 40 - DNA, 1113 bp, Selenomonas noxia
atgaatgctaacgatctcattgcggcactgggtgccgaattcttcactggcgttcccgattctaaattgcgcccgttggttgattgcctgatggatacctatggcgctaattcaccaagccacatcattgcggccaacgaggggaatgccgcggctctggccgctggctaccacttagctgcaggtaaagttcctctggtttacctgcagaacagtgggttgggtaatatcgtcaatccgttgttatcattactgcatgcggaagtatatggcattccgtgcatcttcgtgattggttggcgcggtgaacctgacttacatgacgaaccgcaacacctggtccagggtcgtttgacccttccgttactggaaaccattggcgtgaaaacaatggtactgaccgaagcgagccagccggaagatgtctccgcctggatggaacaaattcgtccgcatctggcagcggggggccagtgcgccttgctggtgcgcaagggcgcgctgactcatccgaaacacaaatatgcaaacgaaaaccccctgcgtcgcgaggatgcaatcgcacggatcctcgatgcagcgcagggcgctgttgttgtggccaccaccggcaaaaccggtcgtgaactgtttgaactgcgcgccgcccgcggcgaagaccatgcccatgatttcctgaccgtgggtagtatgggtcacgccggtgcaatcgcactgggtattgccctgcaccggccgtcccaacgcgtatttttactggatggggatggcgcggccctgatgcatatgggtgcgatggcaaccattggtgcagcggcacccgccaacatcgtgcacgtcctgctgaataacgaagcgcatgaatctgtgggcggcgcaccaaccgcagctcacaccgtcgattttccggcggtagcccgcgccgtgggctaccgtttagtacagactgcggcggatgccgcagaactggcgcagattctgccagcagtgggccgcagcgacgccctgacgttcttggaagttcgtactgctattggttcacgcgcagacctgggtcgtcctactactaccccaaccgaaaacaaagaggcacttatgcgtacgctgcgcgaa

SEQ ID NO: 41 - protein, 531 aa, Acidimicrobium sp. BACL17
MASSEKMRVGEAIIDLLVREYELDTVFGIPGVHNIELFRGLHSSGVRVVAPRHEQGAGFMADGWSIATGKPGVCALISGPGLTNAITPIAQAYHDSRAMLVLASTTPTHSLGKKFGPLHDLDDQSAVVRTVTAFSETVTDPTQFPQLIERAWNVFTSSRPRPVHIAIPTDVLEQFVDPFTRVTTDISKPVAQDSDIQRAAQLLAAAKRPMIIAGGGALGTGALISNIATAIDSPIVLTGNAKGEVPSTHPLCVGSAMVIPRVQEEIEQSDVVLVIGSEISDADLYNGGRAQGFSGSVIRIDIDTEQISRRVAPHVSLVADAADSLSRISAELTKAGVALTNSGSARATNLRMAARSGVRQDLLPWIDAIEQSVPDNTLVAVDSTQLAYAAHTVMSCNSPRSWLAPFGFGTLGCALPMAIGAAIADTTRPVLAIAGDGGWLFTLAEMAAAIDEGIDMVLVLWDNRGYGQIRESFDDVRAPRMGVDVSSHDPSAIANGFGWNAIDVTTIEAFRIVLSEAFENRGAHFIRISVS

SEQ ID NO: 42 - DNA, 1593 bp, Acidimicrobium sp. BACL17
atggcgagctctgagaaaatgcgcgtaggcgaagcgattatagatctgctggtgcgcgaatatgaactagataccgtgttcgggattcccggagtgcacaacattgagctgtttagaggcttacatagctctggtgtgcgcgtcgttgcgcctcgccatgaacaaggtgcaggctttatggcggacggctggagcattgctacaggcaaacctggtgtctgcgccttgataagtgggccgggcttaaccaatgcaataaccccgatagcgcaagcgtaccacgatagtcgcgcgatgttagtcctggcgagtactacgccgacgcacagcctgggcaaaaaatttggcccattacacgatcttgacgatcagtccgccgtggtgcgtaccgtgactgctttttcagagactgttacagatcctacgcagttcccacagctgattgaacgggcgtggaatgttttcacatcatctcgtccgcgtccagttcatatcgcaatcccgaccgacgtgctggagcagtttgtggatccgtttacgcgagtgaccaccgatatttcgaaaccagtggcccaggactccgatattcaaagagcggcgcagctcctagcagcggccaaacgtcccatgatcattgcgggcggaggcgctctgggcacaggtgcattgatctcgaacattgccacagctattgatagcccgatcgtgttgaccggtaatgcgaagggtgaggtaccgagtacccacccgttatgtgtcggctctgctatggttattccacgcgtgcaggaagaaatcgaacaaagtgatgtcgttttggtgattggcagcgaaatctctgatgcagacctgtacaacggtggtcgcgcccagggattttctggtagcgttatccgcatcgacattgataccgagcagattagtcgtcgagtggccccgcacgtcagcctggtggctgatgcggcggattccttgtcacgtatttctgccgaactgacaaaggccggtgtggcgctgacgaattctggcagcgcacgtgcgacgaatttacgtatggcagcccgtagcggcgtgcgacaagacctgctgccgtggatcgatgccattgaacaatccgtgccggacaacacgctggtggcggtagattcaacccagctggcgtatgcggcgcatacagtcatgagttgtaattctccgcgttcttggttagcgccattcggctttggtacgcttggttgtgcccttccaatggcgatcggcgccgcaatcgcggatacgacccgtccagtcctggccattgcgggcgatggtggttggctgtttaccttagccgaaatggcggcagcaatcgacgaaggcattgatatggttcttgtactgtgggataatcgcggctatggacaaatccgtgaaagcttcgacgatgtgcgagcaccccgtatgggtgtagatgtttcaagccatgacccttccgcaatagccaacggcttcggttggaacgcgattgacgtgaccaccattgaggcgttccgaattgttctgtcggaagcgtttgagaaccgtggtgctcactttattcgtatttccgtgagc

SEQ ID NO: 43 - protein, 597 aa, Acyrthosiphon pisum
MQEADFEVNHARNADIPIVGDAKQTLSQMLELLAQSDAKQELDSLRDWWQTIDGWRSRKCLEFDRTSDKIKPQAVIETIWRLTKGDAYVTSDVGQHQMFAALYYQFDKPRRWINSGGLGTMGFGLPAALGVKMALPDETVICVTGDGSIQMNIQELSTALQYDLPVLVLNLNNGFLGMVKQWQDMIYSGRHSQSYMQSLPDFVRLAEAYGHVGISIAHPAELEEKLQLALDTLAKGRLVFVDVNIDGSEHVYPMQIRGGVIVKLDEIARLAGVSRTTASYVINGKARQYRVSDKTVEKVMAVVREHNYHPNAVAAGLRAGRTRSIGLVIPDLENTSYTRIANYLERQARQRGYQLLIACSEQQPDNEMRCIEHLLQRQVDAIIVSTSLPPEHPFYQRWINDPLPIIALDRALDREHFTSVVGADQDDAHALAAELRQLPVKNVLFLGALPELSVSFLREMGFRDAWKDDERMVDYLYCNSFDRTAAATLFEKYLEDHPMPDALFTTSFGLLQGVMDITLKRDGRLPTDLAIATFGDHELLDFLECPVLAVGQRHRDVAERVLELVLASLDEPRKPKPGLTRIRRNLFRRGQLSRRTK

SEQ ID NO: 44 - DNA, 1791 bp, Acyrthosiphon pisum
atgcaggaagcggattttgaagtgaatcatgcgcgtaacgcggacattccgatcgtcggagacgcgaaacagactctgtcgcagatgctggaactcctggcgcaatcagacgctaaacaggagcttgactccctgcgcgactggtggcagaccattgatggatggcggagtcgcaaatgcctggaatttgatcgtacgtcagataagatcaaaccacaagcggttattgagacgatttggcgcctgaccaaaggcgatgcctacgtgacttccgatgtcggccaacaccagatgttcgcggcactgtactaccagtttgataagccgagacgttggattaacagtggtggccttggcacgatgggttttgggctcccggcggcgctgggtgttaaaatggcacttcccgatgagacagtaatctgcgttacgggcgacggttcgattcagatgaatatccaggaactgtctactgcgttacagtacgatttgccggtactggtgctgaacttgaacaacggttttcttggcatggttaaacaatggcaggatatgatctatagcggccgccatagccagagctacatgcaatcccttccggatttcgtacgcctggcagaagcgtacgggcatgtcgggataagcatcgcgcacccggctgaactggaagaaaaattacagctggccttagatacgctggcaaaggggcgccttgtgtttgttgatgtcaatattgacgggagtgaacatgtatatcccatgcaaatccgtggtggtgttattgtgaagctcgatgagatcgcacgcctggcaggagtatctcgtaccacagcctcgtacgtcattaatggaaaggcacgtcagtaccgagtctccgataaaacggtcgaaaaggtgatggcggtggtgcgcgaacataactatcatcctaatgctgtggctgctggtttgcgggcaggacgtactcgtagcattggattagtaatcccggatctggaaaacacatcatacacgcgcattgcgaactatctggaacgccaggcgcgccagcgcggctatcagctgttaatcgcttgcagcgaggaccagccagataatgaaatgcgctgcatcgaacacttgctgcaacgacaggtggacgccattattgtctctacttccctgcccccggaacatccgttctaccaacgctggatcaacgatccactcccgatcatcgcgctggatcgtgcgctggaccgcgagcattttacgagcgtagtaggggccgatcaggacgatgcccatgccctagccgccgaacttcgtcagcttccggtcaaaaacgtgctgtttctgggcgccctgccggaactgagcgtgtcgtttttgcgtgaaatgggcttccgtgacgcctggaaagatgatgaacgaatggtcgattacctgtattgtaacagcttcgatcgtacggccgcagctaccctgtttgagaaatatctcgaagatcacccgatgccggatgcgttgttcactacctccttcggtttgctgcagggtgtgatggatattacactaaaacgcgacggccgcttgccgaccgatctggcgatcgcgacctttggggaccatgaattattggacttcttggaatgtccggtcctggctgtgggccaacgccaccgggatgtggcggaacgcgtcctggaactggtgctggccagcctggatgaaccgcgcaaaccgaaaccaggtctgacgcgcatccgtcgcaacctgtttcggcgcggccagcttagccgtcggaccaaa

SEQ ID NO: 45 - protein, 408 aa, Burkholderia pseudomallei
MKTEDLIGILTDAGVDLAVGVPDSLLKSFCGRLNDPDCPLRHLVASSEGGAVGIAIGHHLATGGLAAVYMQNSGIGNAINPLVSLADRAVYGIPLVLIVGWRAEISASGAQVHDEPQHVTQGRITLPLLDALSIRHLVLERAGGENDALAPSIARLIAGARQTSQPVALVVRKDAFDDASASRPGAAAPHAGRMTREQAIALIVEHADAGTAIVSTTGVASRELYELRDRLGHSHARDFLTVGGMGHASQIAVGIALARPAQKVICIDGDGALLMHMGGLAYCAGAPNLTHVVINNGVHDSVGGQPTLAAHLRLSHIAASCGYAFSRSVATPIELESALHHASRLDGSAFIEVTCRPGYRSDLGRPRTSPAENKRHFMAFLSRNGATHERDDHAQESGIQDAVQCARH

SEQ ID NO: 46 - DNA, 1224 bp, Burkholderia pseudomallei
atgaaaaccgaagacctgataggcatcctgacggatgctggtgtagatctcgcagtcggagtcccggacagcttactgaaaagtttttgtggtcgtctgaatgacccggactgcccgctacggcacctggtagcatcatcagagggtggtgccgtagggattgcgattggtcaccatctcgccaccgggggcctggccgcggtatatatgcaaaactcaggtatcggtaacgccatcaaccctcttgtttcgctggcagaccgcgctgtgtacggcattccgctggttcttatcgtgggatggcgtgcggaaatctctgccagtggcgcacaggtacacgacgagccacaacacgtgacgcagggacgcattaccttaccgctgctggacgcgctgtcgattcgccacttggttctggaacgcgcgggaggcgaaaatgacgctctggccccctctattgcgcgcttgattgcgggcgcgcgtcaaactagccagccggttgctctggtggtgcgtaaggatgcgttcgatgatgcttctgcaagtcgtcctggcgccgctgctccacacgcaggtcgcatgacccgtgaacaagcgattgccctgattgttgagcatgcggacgcaggtaccgccattgtaagtaccactggcgtggcatcgcgcgaactttacgaattacgcgaccgtttaggtcattcccatgcccgcgattttctgaccgtcggcggcatgggtcatgcctctcagatcgcagtgggaattgcgctggcacgccccgcgcagaaagtcatttgcattgatggtgatggcgcactgttgatgcacatgggtggtctggcatattgtgcgggcgccccaaacctgacacacgtggtgattaataacggagttcatgatagtgtcggaggccagccgaccctggctgcccatttgcgcctgtcacacatcgcggcaagctgcggctacgcattttcacgcagcgtagcaacgcctatagaacttgaatcagcgctgcaccacgctagcagactggatggctcagcgttcattgaagtgacctgtcgtccgggctatcgcagcgatctgggccgtcctcgtacgtccccggccgaaaataaacgccactttatggcgttcttaagccgcaacggggccacccatgagcgtgatgaccacgcacaggaatcgggtattcaagacgcagtgcagtgcgcacgtcat

SEQ ID NO: 47 - protein, 323 aa, Mycobacterium xenopi
MLAKHEFSAATMADGYSRCGQKLGVVAATSGGAALNLVPGLGESLASRVPVLALVGQPATTMDGRGSFQDTSGRNGSLDAEALFSAVSVFCRRVLKPADIITALPAAVAAAQTGGPAVLLLPKDIQQTQVGINGYAEHGVAPSRSVGDPHSIVRALRQVTGPVTIIAGEQVARDDARAELEWLRAVLRARVACVPDAKDVAGTPGFGSSSALGVTGVMGHPGVADALAKSALCLVVGTRLSVTARTGLDDALAAVRVVSIGSAPPYVPCTHVHTDDLRASLRLLTAALSGRGRPTGVRVPDAVVRTELTPRRSTVPACAIATR

SEQ ID NO: 48 - DNA, 969 bp, Mycobacterium xenopi
atgctggcgaaacatgagttctccgcagcgaccatggcggatggttacagccgttgcggtcaaaaactgggcgtagttgcggcgacgagcggcggtgcggcactgaacttggtcccaggcttaggtgaaagcttagcgtcacgagtgccggtgttggcgctggtgggccagccggcgaccaccatggatgggagaggctccttccaggacacgagtggccgcaatggcagcttggacgctgaagcattgttctctgccgtgtccgtgttttgccgtcgtgtacttaaaccagctgacattattactgcattaccagcagcagttgctgcggcccagaccggtggtcctgcagtcctgctgcttccgaaagacattcaacagactcaagtgggcatcaacggttacgcagaacatggcgtcgcgccgagtcgctcagtaggcgatccgcattcaattgtgcgtgcccttcgtcaggtgactgggccggtgactataattgccggggaacaagtggcccgtgatgatgcgcgcgcggaacttgaatggttgcgagctgtattaagagcacgtgttgcttgtgtacctgatgcaaaagatgttgcggggacgccaggcttcggttcctcttccgcgctgggcgtcactggtgtgatgggtcatccgggcgtggctgacgcgctggctaaaagcgccctgtgtttagttgtcggtacgcgtttgtcggtcacagcacgtacgggcctggatgatgcgctggccgctgtccgcgttgtgagcatcggttccgcgccgccgtacgtgccatgtacgcatgtgcatactgatgacctgcgtgcttccttacgactgctcaccgcggcgttatcaggtcgcggtcgtccgaccggggtacgtgttcctgatgcggtggtgcgcacggaactgactcctcgtcgtagcaccgttccggcatgtgccattgcgacgcgt

SEQ ID NO: 49 - protein, 376 aa, Pyramidobacter piscolens
MQISSFIAQLQRIASSHFLGVPDSQLKALCNYLYKNCGISSDHIIAANEGNCTALAAGYYLATGKVPVVYMQNSGLGNVVNPVASLLNDKVYGIPCVFVIGWRGEPGLKDEPQHIFQGAVTLDLLKVMDIASFVVRKDTTEQELAAQMAEFQPLLAAGKSVAFVIAKEALTYDEKVSFKNDFTMTREEVIRHITAFSGEDPIVSTTGKASRELFEIRVRNGQPHKYDFLTVGSMGHSSSIALGIALSKPHTKIWCIDGDGAALMHMGALAVIGSQRPRNLVHIVINNGAHESVGGLPTVARSASLAKVAEACGYVNVKTVGTFAELDAALKDARNADELTFIEAKTAIGARADLGRPTTSAMENRDGFMAYLKELR

SEQ ID NO: 50 - DNA, 1128 bp, Pyramidobacter piscolens
atgcagatttcgtccttcattgcgcagttacagcgcatcgcaagctcacattttttaggagtgccggacagccagctcaaagctttgtgtaattatctgtacaaaaactgtggcatctcaagtgaccacatcattgccgcgaacgaaggcaactgtactgcgctggctgcggggtattacctggctacgggcaaggtgccggttgtttacatgcagaacagcgggttagggaatgttgtgaatccggttgcgtccttgctgaatgacaaagtgtacgggatcccgtgtgtgtttgtcattggctggcggggcgagcccggcctcaaggacgaacctcaacacatcttccagggcgcggtgactctggatctgcttaaagtaatggatatcgcgagcttcgttgtccgtaaagataccacggaacaggaattagcggcccagatggctgagtttcaaccgctgctggcggccggcaaatcggttgccttcgtcattgcaaaagaagccctgacgtacgatgagaaagtaagttttaaaaacgacttcactatgactcgcgaagaagtgattcgtcatatcacagcgttttccggcgaagaccctatcgtgagcaccaccggaaaagctagccgcgaattattcgaaattcgagtccgtaacggtcagccccacaaatacgatttcctgactgtgggctctatgggccatagcagttctattgcgctgggtattgcactatcgaagccccacacgaaaatatggtgtatcgatggcgacggtgccgccctgatgcatatgggggccctggcggtgattggtagccaacgtccgcgcaatttagtccatattgttattaataatggtgcccatgagagcgttggtggtcttccgaccgtggcacggtctgcgagtctggcgaaagtcgcagaagcctgtggttatgttaacgtaaaaacggtgggtacctttgcagagttagatgcagctttaaaagacgcccgtaacgccgatgaactgacttttatagaagccaaaaccgcgatcggagcccgcgcggatctcggtcgcccaaccacctccgctatggaaaaccgtgacggatttatggcctatctgaaggagctgcgt

SEQ ID NO: 51 - protein, 370 aa, Melampsora larici-populina
MPAFSLVEIEAKMSFFSDFLNQVKTPSVASKQIYVSKVLIQITNFDQLDFDFQIKILNQVTLHPSQPKLTQEEKSKLLNNTSILRDSIVFFTDTGAARGVGGHAGGPFDTVREVVLLLASFASGSDSKIFDHTVSDEAGHRAQSKLPGHPQLGLTPGVKFSSVVVDWATCGLFSRVSHSPTETVFCFCSDGSQHEGSDAEAARLARAQKLNIKLLIDNNNVTISGHTSGYLKGYKVGKTLEAHALKIVRAEGEKYTGCNDVKSKVIRINFDLKGSTGFEAIHQSRPGIFIPSVIVEHGNFCAAAGFGFEKGKEKMRKLDAVISFGEIVHRALDAGDQLGIEGFDVGLVNKSTLNVIDEKPWMNMDIRNLF

SEQ ID NO: 52 - DNA, 1109 bp, Melampsora larici-populina
atgccggcattctccctggtagagatagaagcgaaaatgtcctttttttctgattttctgaatcaagtcaagacgccgagtgtcgcctcaaagcaaatttatgttagcaaagtgcttattcagattactaactttgatcagctggattttgactttcaaatcaagatcctcaaccaggttactctgcatccatcccagccaaaattgacccaggaggaaaaatcaaaactcttgaacaacacgagtatcctgcgcgatagtatcgtcttcttcacggatacgggtgcagcacgtggtgtaggtggtcacgcgggcggaccatttgataccgtacgcgaggttgtgctcctgttggctagctttgccagtgggagcgacagcaaaatctttgatcatactgtgtcagatgaagcgggccatcgtgcccaatcaaagctgccgggtcatccgcaactgggtcttacgccgggcgtgaaattcagcagcgtggtcgtagattgggcgacctgcggtctgttcagccgtgtgtcacacagcccaacggaaaccgtgttttgcttttgcagcgatggtagtcagcacgaaggcagcgatgcggaagccgcaagactggcccgtgcgcagaagcttaacattaaattattgatcgataacaacaatgtaactatctctgggcacaccagcggttaccttaaaggatacaaagtcggtaaaacgctggaagcacatgccttaaaaatagtacgtgcagaaggtgaaaaatataccggctgcaacgatgtgaaatctaaggtgatacggatcaactttgacctcaaaggttctaccggcttcgaggcgattcatcagtcccgcccgggtattttcattccgtcggtaatcgtggaacatggcaatttttgcgcagcagcgggtttcggatttgaaaaaggcaaagaaaagatgcgtaagctggacgctgttatttcttttggcgagattgttcatcgtgccttggacgccggcgatcaactgggcatagaggggtttgatgtcggcctcgtaaacaaaagtaccctgaatgtgattgatgaaaagccgtggatgaacatggatatccgcaacctgtt

SEQ ID NO: 53 - protein, 344 aa, Candidatus Moduliflexus flocculans
MTTLGNSRVAFRDALMELAERDPRYVLVCSDSGLVIKAQPFIEKFPQRFFDVGIAEQNAVGVAAGLASSGLVPFFATYAGFITMRACEQVRTFVAYPGLNVKLVGANGGMASGEREGVTHQFFEDVGILRAIPGITVVVPADADQVVAATKAVALKDGPAYIRIGSGRDPMVEGETPPFELGKVRILKTYGHDVAIFAMGFIMNRALEAAAQLNSEGIRAVVVDVHTLKPLDVEAITAILQKTSAAVTVEDHNIIGGLGSAIAEVSAEEMPTPLRRIGLRDVYPESGHPEPLLDKYHLGVSDIISAAKTVLKKKNHPPRRIAFSTRENAEEGFSNGNMGEEIYE

SEQ ID NO: 54 - DNA, 1033 bp, Candidatus Moduliflexus flocculans
atgaccacgctgggaaactcccgcgtggcgtttcgcgatgccttaatggagctggcagaacgcgacccgcggtacgtactggtgtgttcggattctggcctggtgattaaggcccaacctttcatcgagaaattcccccagcgcttttttgatgttggaatcgcggagcagaacgcggttggcgtggccgcgggtctggcatccagcgggttggtacctttttttgcgacctacgccggttttatcacgatgcgtgcttgtgaacaggtacgcaccttcgtcgcttatccgggtctgaacgtcaaactggtcggcgccaacggcggcatggcgtctggggaacgcgaaggggtcacgcaccagtttttcgaggatgtcggtatactgcgtgcaattcctggcattacagtcgtcgtacctgccgatgccgatcaggtagtagcggcaaccaaagcggtagcattaaaagatggcccggcctatatacgtatcggaagcgggcgtgacccgatggttgagggggaaaccccgccttttgaacttggcaaagttcgtattctgaaaacctacgggcatgacgtagctatcttcgccatgggttttataatgaaccgcgcgcttgaggcagcggcgcaactgaacagtgaaggcattcgggcagttgtagtagacgtgcacaccctgaaacccctggatgtggaggcaattaccgcgatcctccagaaaacttctgcagcggtaaccgtggaggatcataacatcattggcggcctcgggagcgcgatagccgaggtgtcggcggaggaaatgccgacccccctgcgccgtattggtctgcgcgatgtttatccggaaagtggtcacccggagcctctgctggataaataccacttgggcgttagcgacatcatcagcgccgccaagacggtgctgaaaaaaaagaatcacccgccccgccgtatcgccttcagcacccgggaaaatgccgaggagggtttcagtaacggcaatatgggcgaggaaatttatgaag

SEQ ID NO: 55 - protein, 528 aa, Pseudomonas fluorescens
MKTVHGATYDILRQHGLTTIFGNPGSNELPFLKGFPEDFRYILGLHEGAVVGMADGYALASGQPTFVNLHAAAGTGNGMGALTNAWYSHSPLVITAGQQVRSMIGVEAMLANVDAAQLPKPLVKWSHEPATAQDVPRALSQAIHTANLPPRGPVYVSIPYDDWACEAPSGVEHLARRQVSSAGLPSPAQLQHLCERLAAARNPVLVLGPDVDGSAANGLAVQLAEKLRMPAWVAPSASRCPFPTRHACFRGVLPAAIAGISHNLAGHDLILVVGAPVFRYHQFAPGNYLPAGCELLHLTCDPGEAARAPMGDALVGDIALTLEAVLDGVPQSVRQMPTALPAAEPVADDGGLLRPETVFDLLNALAPKDAIYVKESTSTVGAFWRRVEMREPGSYFFPAAGGLGFGLPAAVGVQLASPGRQVIGVIGDGSANYGITALWTAAQYNIPVVFIILKNGTYGALRWFADVLDVNDAPGLDVPGLDFCAIARGYGVQAVHAATGSAFAQALREALESDRPVLIEVPTQTIEP

SEQ ID NO: 56 - DNA, 1584 bp, Pseudomonas fluorescens
atgaagacggtccacggtgcaacctacgacatcctgcgccagcatggtctgacgacgatttttggtaatccgggtgataacgaactgccgtttctgaaaggtttcccggaagactttcgttatattctgggcctgcatgaaggtgccgtggttggcatggcagatggttacgcgctggccagtggtcagccgacctttgtgaacctgcatgcggcggcgggcaccggtaacggcatgggtgcactgacgaatgcttggtatagtcactccccgctggttattacggcgggtcagcaagtccgctctatgatcggcgtggaagctatgctggcgaacgtggacgctgcacagctgccgaaaccgctggttaagtggtcacatgaaccggcaaccgctcaggatgtgccgcgtgcgctgtcgcaagccattcacacggcaaatctgccgccgcgcggtccggtgtatgtttcaatcccgtacgatgactgggcctgcgaagcaccgtcgggtgttgaacatctggcgcgtcgccaggtcagctctgccggcctgccgagcccggcacagctgcaacacctgtgtgaacgtctggccgcagctcgtaacccggtcctggtgctgggtccggatgtggatggttctgcggccaatggcctggctgttcagctggcggaaaagctgcgtatgccggcttgggtggcaccgtcagcctcgcgctgcccgttcccgacccgtcacgcctgttttcgcggtgttctgccggcagctattgccggtatcagccataacctggcaggccacgatctgattctggtcgtgggtgcgccggtgttccgttatcatcagtttgcgccgggtaattacctgccggcgggttgcgaactgctgcacctgacctgtgatccgggtgaagcagcccgcgctccgatgggtgacgcgctggttggcgatatcgccctgaccctggaagcagtgctggatggcgttccgcagagcgtccgtcaaatgccgacggcactgccggcagctgaaccggtggcagatgacggtggtctgctgcgtccggaaaccgttttcgacctgctgaacgcgctggccccgaaagatgccatttatgttaaggaaagcacctctacggtcggtgcattctggcgtcgcgtggaaatgcgtgaaccgggctcctactttttcccggcggccggcggtctgggttttggtctgccggcagctgttggtgtccagctggccagtccgggtcgccaagtgattggcgttatcggcgatggttccgctaactatggtattaccgcactgtggacggcggcccagtacaacatcccggttgtcttcattatcctgaaaaatggcacctatggtgctctgcgttggtttgcggatgtcctggacgtgaatgatgcgccgggtctggacgtgccgggcctggatttctgcgcaatcgctcgcggctacggtgttcaggcagtccatgcagctaccggcagcgcatttgcccaagcactgcgtgaagcgctggaatctgatcgcccggtgctgattgaagttccgacccagacgatcgaaccg

SEQ ID NO: 57 - protein, 529 aa, Unknown
  Arthrobacter sp.
MTTVHAAAYELLRSNRLTTIFGNPGDNELPFLDAMPADFRYILGLHEGVVVGMADGFAQASGQAAFVNLHAASGTGNAMGALTNAWYSHTPLVITAGQQVRPMIGLEAMLSNVDAASLPRPLVKWSAEPAQAPDVPRALSQAIHTATSDPKGPVYLSIPYDDWNQDTGNLSEHLSSRSVSRAGNPSAEQLDDILSALREAANPALVFGPDVDAARANHHAVRLAEKLAAPVWIAPAAPRCPFPTRHPNFRGVLPASIAGISALLNGHDLIVVIGAPVFRYHQYQPGSYLPENSRLIHITCDAGEAARAPMGDALVADIGQTLRALADIIPQSKRPPLRPRVIPPVPDSQDDLLAPDAVFEVMNEVAPEDVVYVNESVSTVTALWERVELKHPGSYYFPASGGLGFGMPAAVGVQLANDRRRVIAVIGDGSANYGITALWTAAQEKIPVVFIILNNGTYGALRAFAKLLNAENAAGLDVPGICFCAIAEGYGVEAHRITSLENFKDKLSAALQSDTPTLLEVPTSTTSPF

SEQ ID NO: 58 - DNA, 1587 bp, Unknown
  Arthrobacter sp.
atgacgacggtccatgccgccgcctatgaactgctgcgtagcaatcgcctgacgacgatctttggtaatccgggtgataatgaactgccgtttctggatgcaatgccggctgacttccgctatattctgggcctgcatgagggtgtggttgtcggcatggcggatggttttgcgcaggccagcggtcaagcggccttcgttaacctgcatgcagcttctggcaccggtaacgcgatgggcgccctgacgaatgcatggtacagtcacaccccgctggtgattacggcgggccagcaagttcgtccgatgatcggtctggaagcgatgctgagcaatgttgatgcagcctctctgccgcgcccgctggtcaaatggtctgccgaaccggcacaggctccggatgttccgcgtgcgctgagccaagccattcataccgcaacgtctgacccgaagggtccggtgtatctgagtatcccgtacgatgactggaaccaggataccggtaatctgtccgaacacctgagcagccgtagcgtgagccgtgcgggtaacccgtcagctgaacaactggatgacattctgtcggcactgcgtgaagcagctaacccggcgctggtttttggtccggatgtggatgcggcccgcgctaatcatcacgcggtgcgtctggccgaaaaactggcagctccggtttggatcgcaccggcggcaccgcgttgcccgtttccgacccgccatccgaacttccgtggcgttctgccggcaagtattgctggcatctccgccctgctgaatggtcatgatctgattgtggttatcggtgcaccggtgttccgttatcaccagtaccaaccgggcagttatctgccggaaaattcccgcctgattcacatcacctgtgatgcaggtgaagcagctcgtgccccgatgggtgatgcgctggttgccgacattggtcagacgctgcgcgcgctggccgacattatcccgcaaagcaaacgtccgccgctgcgcccgcgtgtcatcccgccggtgccggattcacaggatgacctgctggcaccggacgctgtctttgaagtgatgaacgaagtcgcgccggaagatgtcgtgtatgtgaatgaatcagtttcgaccgtcacggccctgtgggaacgtgtggaactgaagcatccgggttcatattactttccggcgtcgggcggtctgggtttcggtatgccggcggccgtgggtgttcagctggccaacgatcgtcgccgtgtgattgcagttatcggcgacggtagcgcaaattatggcattaccgctctgtggacggcagctcaggaaaaaatcccggttgtctttattatcctgaacaatggcacctacggtgcgctgcgcgcattcgctaagctgctgaacgccgaaaatgcggccggcctggatgtgccgggcatttgcttttgtgcgatcgccgaaggctatggtgtggaagcgcaccgtattaccagcctggaaaacttcaaagataagctgtcagcagctctgcaatcggacaccccgacgctgctggaagtgccgaccagcaccacgtctccgttt

SEQ ID NO: 59 - protein, 529 aa, Pseudomonas putida
MKTIHSAAYALLRRHGMTTIFGNPGSNELPFLKSFPEDFQYVLGLHEGAVVGMADGYALASGKPAFVNLHAAAGTGNGMGALTNSWYSHSPLVITAGQQVRPMIGVEAMLANVDATQLPKPLVKWSYEPANAQDVPRALSQAIHYANTTPKAPVYLSIPYDDWDQPSGPGVEHLIERDVQTAGTPDARQLQVLVQQVQDARNPVLVLGPDVDATLSNDHAVALADKLRMPVWIAPAASRCPFPTRHPSFRGVLPAAIAGISKTLQGHDLIIVVGAPVFRYLQFAPGDYLPVGAQLLHITSDPLEATRAPMGHALVGDIRETLRVLAEEVVQQSRPYPEALAAPECVTDEPHHLHPETLFDVLDAVAPHDAIYVKESTSTVTAFWQRMNLRHPGSYYFPAAGGLGFGLPAAVGVQLAQPQRRVVALIGDGSANYGITALWTAAQYRIPVVFIILKNGTYGALRWFAGVLKAEDSPGLDVPGLDFCAIAKGYGVKAVHTDTRDSFEAALRTALDANEPTVIEVPTLTIQPH

SEQ ID NO: 60 - DNA, 1587 bp, Pseudomonas putida
atgaagaccatccactctgccgcctatgccctgctgcgtcgccacggtatgaccaccattttcggtaatccgggtagcaatgaactgccgtttctgaaaagtttcccggaagactttcagtatgttctgggcctgcatgaaggtgccgtggttggcatggcagatggttacgccctggcaagcggcaagccggcattcgtgaacctgcatgcggcggcgggcaccggtaacggcatgggtgccctgaccaattcttggtatagccactctccgctggtgattacggcaggccagcaagttcgtccgatgatcggtgtcgaagcgatgctggccaatgtggacgcgacccagctgccgaaaccgctggttaagtggagctatgaaccggctaacgcgcaggatgttccgcgcgcactgtcgcaagctattcattacgcgaataccacgccgaaagccccggtgtatctgagcatcccgtacgatgactgggatcagccgtctggtccgggcgtcgaacacctgattgaacgtgacgtgcaaacggctggcaccccggatgcacgtcagctgcaagttctggtccagcaagttcaggatgcacgtaacccggtgctggttctgggtccggatgtggatgcgaccctgagcaatgaccatgccgtggcactggctgataaactgcgtatgccggtttggatcgcaccggctgcgagtcgctgcccgttcccgacgcgtcatccgtcctttcgtggtgtgctgccggccgcaattgcaggtatcagcaagaccctgcaaggtcacgatctgattatcgtcgtgggtgcgccggttttccgttatctgcaatttgcgccgggtgactacctgccggtgggtgcacaactgctgcatattacgtcagatccgctggaagcaacccgtgctccgatgggccacgccctggttggtgatatccgtgaaaccctgcgcgtcctggcagaagaagttgtccagcaatcgcgcccgtatccggaagcgctggctgcaccggaatgtgtgacggacgaaccgcatcacctgcatccggaaaccctgttcgatgtcctggacgcagtggcaccgcacgatgctatttacgtgaaagaaagtacctccacggttaccgccttttggcagcgtatgaacctgcgccatccgggcagctattacttcccggccgcaggcggtctgggttttggtctgccggctgcggtcggtgtgcagctggcacagccgcaacgtcgcgtggttgctctgattggcgatggttctgcgaactatggtatcacggcactgtggaccgccgcacagtaccgtattccggtcgtgttcattatcctgaaaaatggcacctatggtgccctgcgctggtttgcaggtgtcctgaaggctgaagatagtccgggcctggacgtgccgggtctggatttctgcgcaatcgctaaaggctacggtgttaaggcggtccatacggatacccgtgactcctttgaagctgcactgcgtacggcgctggatgcaaacgaaccgaccgtgattgaagttccgacgctgaccatccagccgcac

SEQ ID NO: 61 - protein, 566 aa, Halotalea alkalilenta
MTSRSSFSPPSASEQRGADIFAEVLQCEGVRYIFGNPGTTELPLLDALTDITGIHYVLGLHEASVVAMADGYAQASGKPGFVNLHTAGGLGNAMGAILNAKMANTPLVVTAGQQDTRHGVTDPLLHGDLTGIARPNVKWAEEIHHPEHIPMLLRRALQDCRTGPAGPVFLSLPIDTMERCTSVGAGEASRIERASVANMLHALATALAEVTAGHIALVAGEEVFTANASVEAVALAEALGAPVFGASWPGHIPFPTAHPQWQGTLPPKASDIRETLGPFDAVLILGGHSLISYPYSEGPAIPPHCRLFQLTGDGHQIGRVHETTLGLVGDLQLSLRALLPLLARKLQPQNGAVARLRQVATLKRDARRTEAAERSAREFDASATTPFVAAFETIRAIGPDVPIVDEAPVTIPHVRACLDSASARQYLFTRSAILGWGMPAAVGVSLGLDRSPVVCLVGDGSAMYSPQALWTAAHERLPVTFVVFNNGEYNILKNYARAQTNYRSARANRFIGLDISDPAIDFPALASSLGVPARRVERAGDIAIAVEDGIRSGRPNLIDVLISSSS

SEQ ID NO: 62 - DNA, 1698 bp, Halotalea alkalilenta
atgaccagccgtagctcgtttagcccgccgtcagcgtcagaacagcgtggtgcggatatttttgccgaagtcctgcaatgtgaaggtgtccgctatatttttggcaatccgggcaccacggaactgccgctgctggatgcactgaccgacattacgggtatccattatgtgctgggcctgcacgaagcgtcagtggttgcgatggccgatggttacgcacaggcttcgggcaaaccgggtttcgttaacctgcataccgccggcggtctgggtaatgcgatgggtgccattctgaacgcaaagatggctaataccccgctggtcgtgacggcgggtcagcaagatacccgtcatggcgttaccgatccgctgctgcacggcgacctgaccggtatcgcacgtccgaatgtcaaatgggccgaagaaattcatcacccggaacatatcccgatgctgctgcgtcgtgcgctgcaagattgccgcacgggtccggctggtccggtgtttctgagtctgccgattgacacgatggaacgttgtacgtccgtgggtgcaggtgaagccagccgtatcgaacgcgcgagcgtggctaacatgctgcatgcgctggccaccgcactggctgaagtgacggccggtcacattgcgctggtcgccggtgaagaagtgttcaccgcgaatgccagtgttgaagcagtcgctctggcggaagcactgggcgcaccggtttttggtgcttcctggccgggtcatattccgttcccgaccgcacacccgcagtggcagggtacgctgccgccgaaggcgagcgatatccgtgaaaccctgggcccgtttgacgccgtgctgattctgggcggtcatagtctgatctcctatccgtactcagaaggtccggcaattccgccgcactgccgcctgttccagctgaccggcgatggtcatcaaatcggccgtgttcacgaaaccacgctgggcctggtgggcgatctgcaactgagtctgcgcgcgctgctgccgctgctggcccgtaaactgcaaccgcaaaacggtgcagtcgctcgtctgcgccaagtggcaaccctgaagcgtgatgctcgtcgcacggaagcggccgaacgttcagcccgcgaatttgacgcgtcggccaccacgccgtttgttgcagctttcgaaaccattcgcgcaatcggcccggatgtgccgattgttgacgaagcgccggttacgatcccgcatgtccgtgcctgcctggatagcgcatctgctcgccagtacctgtttacccgttctgcaattctgggttggggtatgccggcggccgtcggtgtgagtctgggtctggatcgttccccggttgtctgtctggtgggcgacggttcagcgatgtactcgccgcaggcactgtggaccgcagctcacgaacgcctgccggttacgtttgtggttttcaacaatggtgaatataacgccctgaaaaattttgcgcgtgcccaaaccaactaccgtagcgcacgcgctaatcgttttattggcctggatatctctgacccggcgattgatttcccggcgctggccagctctctgggtgtgccggcacgtcgcgttgaacgtgctggtgatattgcaatcgctgtcgaagacggcatccgcagcggtcgtccgaacctgattgatgtgctgatcagttcctcatcg

SEQ ID NO: 63 - protein, 536 aa, Unknown
  Streptomyces sp.
MRTVRESALDVLRARGMTTVFGNPGSTELPMLKQFPDDFRYVLGLQEAVVVGMADGFALASGTTGLVNLHTGPGTGNAMGAILNARANRTPMVVTAGQQVRAMLTMEALLTNPQSTLLPQPAVKWAYEPPRAADVAPALARAVQVAETPPQGPVFVSLPMDDFDVVLGEDEDRAAQRAAARTVTHAAAPSAEVVRRLAARLSGARSAVLVAGNDVDASGAWDAVVELAERTGLPVWSAPTEGRVAFPKSHPQYRGMLPPAIAPLSRCLEGHDLVLVIGAPVFCYYPYVPGAHLPENTELVHLTRDADEAARAPVGDAVVADLALTVRALLAELPAREAAAPAARTARAESTAEVDGVLTPLAAMTAIAQGAPANTLWVNESPSNLGQFHDATRIDTPGSFLFTAGGGLGFGLAAAVGAQLGAPDRPVVCVIGDGSTHYAVQALWTAAAYKVPVTFVVLSNQRYAILQWFAQVEGAQGAPGLDIPGLDIAAVATGYGVRAHRATGFGELSKLVRESALQQDGPVLIDVPVTTELPTL

SEQ ID NO: 64 - DNA, 1608 bp, Unknown
  Streptomyces sp.
atgcgtacggtgcgtgaatcggctctggacgtgctgcgtgcgcgtggtatgacgacggtttttggtaatccgggctcaacggaactgccgatgctgaaacagtttccggatgacttccgctatgttctgggtctgcaagaagctgtggttgtcggtatggcagatggctttgccctggcaagtggcaccacgggtctggtgaatctgcataccggtccgggcacgggtaacgcgatgggcgcaattctgaacgctcgtgcgaatcgtaccccgatggtggttacggcgggccagcaagtgcgtgccatgctgacgatggaagcactgctgaccaatccgcagagtacgctgctgccgcaaccggctgtcaagtgggcgtacgaaccgccgcgcgcggccgatgtggcaccggcactggctcgtgcggtccaggtggcagaaaccccgccgcaaggtccggtttttgtctccctgccgatggatgacttcgatgtcgtgctgggcgaagatgaagaccgtgcagctcagcgtgcggcggcacgtaccgttacgcacgctgcggccccgagcgcggaagttgtccgtcgcctggcagctcgtctgagtggtgctcgttccgcggtgctggttgcgggtaatgatgtggacgcctctggcgcatgggatgctgtggttgaactggccgaacgtaccggtctgccggtctggagtgcaccgacggaaggtcgtgtggcatttccgaaatcccatccgcagtatcgtggtatgctgccgccggcaattgcaccgctgagccgttgcctggaaggtcacgatctggtcctggtgatcggtgcgccggtgttctgttattacccgtacgttccgggtgcccatctgccggaaaacaccgaactggttcacctgacgcgcgatgcagacgaagcagcccgtgccccggttggtgatgcagtcgtggccgacctggcactgaccgtgcgcgctctgctggcggaactgccggcgcgtgaagcagctgcgccggccgcacgtaccgctcgcgcggaatctacggccgaagtcgatggtgtgctgaccccgctggctgcaatgacggcaattgcacagggcgctccggcaaacaccctgtgggttaatgaaagcccgtctaacctgggtcaatttcatgatgcaacccgtatcgacacgccgggcagctttctgttcaccgccggcggtggcctgggtttcggtctggccgcagctgtgggtgcccagctgggcgcaccggatcgtccggttgtctgcgttattggcgacggttcaacccactatgcagtccaggcactgtggaccgcggcggcgtacaaagttccggtcacctttgtggttctgtcgaatcagcgctatgcaatcctgcaatggttcgcgcaagtggaaggcgctcaaggtgcgccgggcctggatattccgggtctggacatcgctgcggttgcaacgggttacggtgtccgtgcccatcgtgcaaccggctttggtgaactgtcaaagctggtgcgtgaatcggcgctgcaacaagatggcccggttctgatcgacgtgccggttaccacggaactgccgaccctg

SEQ ID NO: 65 - protein, 577 aa, Rheinheimera sp. A13L
MSSINSFTVADYLLTRLHQLGLRKVFQVPGDYVANFMDALEQFNGIEAVGDLTELGAGYAADGYARLTGIGAVSVQFGVGTFSVLNAIAGSYVERNPVVVITASPSTGNRKTIKETGVLFHHSTGDLLADSKVFANVTVAAEVLSDPSDARQKIDKALTLAITFRRPIYLEAWQDVWGLACEKPEGELKALPLISEEGALKAMLADSLKLLNSARQPLVLLGVEINRFGLQDAVLDLLKASGLPYSTTSLAKTVISENEGIFVGTYADGASFPATVEYIEKADCVLALGVIFTDDYLTMLSKQFDQMIVVNNDETSRLGHAYYHQLYLADFILQLTDEIKKSSLYPRQNSALPLLPPQPQITPALLQQQLSYQNFFDLFYGYLLQHQLQDNISLILGESSSLYMSARLYGLPQDSFIADAAWGSLGHETGCVTGIAYASDKRAMAIAGDGGFMMMCQCLSTISRHQLNSVVFVISNKVYAIEQSFVDICAFAKGGHFAPFDLLPTWDYLSLAKAFSVEGYRVQNGEELLQALEHIMTQKDKPALVEVVIQSQDLAPAMAGLVKSITGHTVEQCAIPT

SEQ ID NO: 66 - DNA, 1731 bp, Rheinheimera sp. A13L
atgtcatcaatcaactcgttcaccgtcgccgactacctgctgacccgtctgcatcaactgggcctgcgtaaggtttttcaagtgccgggcgattatgtcgctaactttatggacgcgctggaacagttcaatggcattgaagccgtgggtgatctgaccgaactgggtgcaggttatgcggccgacggttacgcacgtctgaccggtatcggtgcagtgtctgttcagtttggcgtgggtacgttttctgttctgaacgcaattgctggcagttacgttgaacgtaatccggtggttgtcatcaccgcgtcgccgagcacgggtaaccgcaaaaccattaaggaaacgggcgtgctgtttcatcactccaccggtgatctgctggctgactcaaaagtgttcgcgaatgtcacggtggcagctgaagttctgtctgatccgagtgacgcgcgccagaaaattgataaggccctgaccctggcaattacgtttcgtcgcccgatctatctggaagcctggcaggatgtttggggcctggcatgcgaaaaaccggaaggtgaactgaaggccctgccgctgatcagcgaagaaggcgcgctgaaagccatgctggcagattctctgaagctgctgaacagtgcacgtcagccgctggttctgctgggtgtcgaaattaatcgcttcggtctgcaagatgctgttctggacctgctgaaagcgtctggtctgccgtattccaccacgtcactggccaagaccgttattagtgaaaacgaaggcatctttgtcggcacctatgcggatggtgcgtccttcccggcaacggtggaatacatcgaaaaagccgattgtgtcctggcactgggtgtgatttttaccgatgactacctgacgatgctgtcaaaacagttcgatcaaatgatcgtggttaacaatgacgaaacctcgcgtctgggccatgcttattaccaccagctgtatctggcggattttattctgcaactgacggacgaaattaaaaaatctagcctgtacccgcgtcagaacagcgcactgccgctgctgccgccgcaaccgcagattaccccggcgctgctgcaacaacagctgagttatcagaactttttcgacctgttttatggttacctgctgcaacatcagctgcaagacaatatttccctgatcctgggcgaaagttcctcactgtatatgtcagctcgtctgtacggtctgccgcaggattctttcatcgcagacgcagcatggggcagtctgggtcacgaaaccggctgcgttacgggtatcgcgtatgccagcgataaacgtgcaatggctattgcgggtgacggcggttttatgatgatgtgccagtgtctgagcaccattagccgccatcaactgaactccgtcgtgttcgttatttcaaataaagtctacgccatcgaacagtcctttgtggatatttgtgccttcgcaaagggcggtcactttgcgccgttcgatctgctgccgacctgggactatctgtcgctggctaaagcgtttagcgtggaaggctaccgcgttcagaacggtgaagaactgctgcaagcgctggaacatatcatgacccagaaagataagccggccctggtggaagttgtcattcagtcgcaggatctggcaccggcaatggctggcctggtcaaaagcatcaccggtcacacggtggaacagtgcgccattccgacc

SEQ ID NO: 67 - protein, 611 aa, Bradyrhizobium sp. STM 3843
MHPDACSIACAAMPTNWGPRTVTKLPLPDPQSRATTHHRTAHYFLEALIDLGVEYIFANLGTDHVSLIEEIARWDSEGRRHPEVILCPHEVVAVHMAMGYAMTTGRGQAVFVHVDAGTANACMAIQNAFRYRLPVLLIAGRAPFAIHGELPGGRDTYVHFVQDSFDQGSIVRPYVKWEYTLPSGVVVKEALTRAAAFMHSDPPGPVSMMLPREVLAEAWDDDAMPAYPPARYGSVRAGGVDPERAQAIADALMTAENPIALTAYLGRSAEAVSVLDRLALVCGIRVVEFNPITMNICQDSPCFAGSDPAALVADADLGLLIDIDVPFIPQLLKSADRLRWIQIDIDALKADIPMWGFATDLRIQGDSAVILRQVLEIVIARGNDSYMRKVRDRIASWRPAREAAQAKRMAAAANKGSPGAINPAYLFARLQALLSEQDIVVNEAVRNAPVLQQQLRRTKPMTYVGLAGGGLGFSGGMALGLKLANPSHRVVQIVGDGAFHFAAPDSVYAVSQQYRLPIFSVILDNKGWQAVKASVQRVYPDGVAQQTDSFLSRLATGRQDEQRRLVDIARAFGAHGERVDDPDELDAAIRSCLAALDDGRAAVLHVNITPL

SEQ ID NO: 68 - skipped

SEQ ID NO: 69 - protein, 551 aa, Unknown
  Psychrobacter sp.
MQHDSITPLSKKTSMLDTTAESVVSQTVQQVVFELMRTLNMTTVFGNPGSTELNFLTNFPEDFSYVLGLHEASVVGMADGYAQATGNAAFVNLHSAAGVGNALGNIFTAYRNHTPLVITAGQQARSLLPFAPYLGAEQAAQFPQPYIKWSIEPARAEDVPLAIAQAYLIAMQHPQGPTFVSIPSDDWDKPAVLPLLSQSCGHSIPSPDALAELVEVMSTSQNMALVVGSDVDRQGGFELAVSVAEACQAPVWEAPNSSRASFPENHPLFAGFLPAIPEKLSEKLLGYDTIVVIGAPAFTLHVAGTLSLKKSKIYQLTDDPQYAAQSVATKTLSGNIRDSLQALLDKLPTSMTPRSGLDLPVRKPAAEVQGSNPISIEYVMATLAKYCPEDVVIVEEAPSHRPAIQRYLPITQPKSFYTMASGGLGYGLPAAVGVALGTQRRTLCLIGDGSSMYSIQAIWTAVQHNLPVTVIVLNNTGYGAMRSFSKIMGSTQVPGLDLPNINFVQLAQSMGCQAQKVTDYSVLDKVFADTMQAAGSYLLEIMVDANTGAVY

SEQ ID NO: 70 - skipped

SEQ ID NO: 71 - protein, 593 aa, Roseobacter sp. AzwK-3b
MKMTTEEAFVKTLQRHGIEHAFGIIGSAMMPISDLFPQAGITFWDCAHEGSAGMMSDGYTRATGKMSMMIAQNGPGITNFVTAVKTAYWNHTPLLLVTPQAANKTIGQGGFQEVEQMKLFEDMVAYQEEVRDPSRMAEVLARVISKAKNLSGPAQINIPRDYWTQVIDIELPDPIEFERSPGGENSVAEAARLISEARNPVILNGAGVVLSEGGIAASQALAERLDAPVCVGYQHNDAFPGSHPLFAGPLGYNGSKAAMELIKDADVVLCLGTRLNPFSTLPGYGMDYWPKDAKIIQVDINPDRIGLTKKVSVGIIGDAAKVARGILGQLSDSAGDEGRDARRARIAETKSKWAQQLSSMDHEDDDPGTSWNERAREAKPDWMSPRMAWRAIQSALPREAIISSDIGNNCAIGNAYPSFEEGRKYLAPGLFGPCGYGLPAIVGAKIGRPDVPVVGFAGDGAFGIAVNELTAIGRSEWPGITQIVFRNYQWGAEKRNSTLWFDDNFVGTELDDDVSYAGIAKACGLKGVVARTMDELTDALNQAIKDQMENGTTTLIEAMINQELGEPFRRDAMKKPVAVAGISPDDMRPQKVA

SEQ ID NO: 72 - skipped

SEQ ID NO: 73 - protein, 564 aa, Serratia marcescens
MSNAITKVQNANARRGGDVLLEVLESEGVEYVFGNPGTTELPFMDALLRKPSIQYVLALQEASAVAMADGYAQAAKKPGFLNLHTAGGLGHGMGNLLNAKCSQTPLVVTAGQQDSRHTTTDPLLLGDLVGMGKTFAKWSQEVTHVDQLPVLVRRAFHDSDAAPKGSVFLSLPMDVMEAMSAIGIGAPSTIDRNAVAGSLPLLASKLAAFTPGNVALIAGDEIYQSEAANEVVALAEMLAADVYGSTWPNRIPYPTAHPLWRGNLSTKATEINRALSQYDAIFALGGKSLITILYTEGQAVPEQCKVFQLSADAGDLGRTYSSELSVVGDIKSSLKVLLPELEKATANHRRDYQRRFEKAINEFKLSKESLLGQVQEQQSATVITPLVAAFEAARAIGPDVAIVDEAIATSGSLRKSLNSHRADQYAFLRGGGLGWGMPAAVGYSLGLGKAPVVCFVGDGAAMYSPQALWTAAHEKLPVTFIVMNNTEYNVLKNFMRSQADYTSAQTDRFIAMDLVNPSVDYQALGASMGLETRKVIRAGDIAPAVEAALASGKPNVIEIIISKS

SEQ ID NO: 74 - skipped

SEQ ID NO: 75 - protein, 562 aa, Granulicella mallensis
MNIAYETRENKVASGRECLLEILRDEGVTHVFGNPGTTELALIDALAGDDDFHFILGLQEAAVVGMADGYAQATGRPSFVNLHTTAGLGNGMGNLTNAFATNVPMVVTAGQQDIRHLAYDPLLSGDLVGLARATVKWAHEVRSLQELPIILRRAFRDANTEPRGPVFVSLPMNIIDEIGTVSIPPRSTIVQAESGDISQLVRLLVESAGNLCLVVGDEVGRYGATEAAVRVAELLGAPVYGSPFHSNVPFPTDHPLWRFTLPPNTGEMRKVLGGYDRILLIGDRAFMSYTYSDELPLSPKTQLLQIAVDRHSLGRCHAVELGLYGDPLSLLAAVGDALSQERALAPSRDSRLAIARDWRASWEQDLKDECERLAPSRPLYPLVAADAVLRGVPPGTVIVDECLATNKYVRQLYPVRKPGEYYYFRGAGLGWGMPAAVGVSLGLERQQRVVCLLGDGAAMYSPQALWSAAHESLPITFVVFNNSEYNILKNFMRSRPGYNAQSGRFVGMEINQPSIDFCALARSMGVDAVRLTEPDDITAYMIAAGDREGPSLLEIPIAATAS

SEQ ID NO: 76 - skipped

SEQ ID NO: 77 - protein, 548 aa, Enterococcus haemoperoxidus
MYTVADYLLDRLKELGIDEVFGVPGDYNLQFLDHITARKDLEWIGNANELNAAYMADGYARTKGISALVTTFGVGELSAINGLAGSYAESIPVIEIVGSPTTTVQQNKKLVHHTLGDGDFLRFERIHEEVSAAIAHLSTENAPSEIDRVLTVAMTEKRPVYINLPIDIAEMKASAPTTPLNHTTDQLTTVETAILTKVEDALKQSKNPVVIAGHEILSYHIENQLEQFIQKFNLPITVLPFGKGAFNEEDAHYLGTYTGSTTDESMKNRVDHADLVLLLGAKLTDSATSGFSFGFTEKQMISIGSTEVLFYGEKQETVQLDRFVSALSTLSFSRFTDEMPSVKRLATPKVRDEKLTQKQFWQMVESFLLQGDTVVGEQGTSFFGLTNVPLKKDMHFIGQPLWGSIGYTFPSALGSQIANKESRHLLFIGDGSLQLTVQELGTAIREKLTPIVFVINNNGYTVEREIHGATEQYNDIPMWDYQKLPFVFGGTDQTVATYKVSTEIELDNAMTRARTDVDRLQWIEVVMDQNDAPVLLKKLAKIFAKQNS

SEQ ID NO: 78 - skipped

SEQ ID NO: 79 - protein, 574 aa, Acinetobacter baumannii
MELLSGGEMLVRALADEGVEHVFGYPGGAVLHIYDALFQQDKINHYLVRHEQAAGHMADAYSRATGKTGVVLVTSGPGATNTVTPIATAYMDSIPMVILSGQVASHLIGEDAFQETDMVGISRPIVKHSFQVRHASEIPAIIKKAFYIAASGRPGPVVVDIPKDATNPAEKFAYEYPEKVKMRSYQPPSRGHSGQIRKAIDELLSAKRPVIYTGGGVVQGNASALLTELAHLLGYPVTNTLMGLGGFPGDDPQFVGMLGMHGTYEANMAMHNADVILAIGARFDDRVTNNPAKFCVNAKVIHIDIDPASISKTIMAHIPIVGAVEPVLQEMLTQLKQLNVSKPNPEAIAAWWDQINEWRKVHGLKFETPTDGTMKPQQVVEALYKATNGDAIITSDVGQHQMFGALYYKYKRPRQWINSGGLGTMGVGLPYAMAAKLAFPDQQVVCITGEASIQMCIQELSTCKQYGMNVKILCLNNRALGMVKQWQDMNYEGRHSSSYVESLPDFGKLMEAYGHVGIQIDHADELESKLAEAMAINDKCVFINVMVDRTEHVYPMLIAGQSMKDMWLGKGERT

SEQ ID NO: 80 - skipped

SEQ ID NO: 81 - protein, 546 aa, Staphylococcus aureus
MKQRIGAYLIDAIHRAGVDKIFGVPGDFNLAFLDDIISNPNVDWVGNTNELNASYAADGYARLNGLAALVTTFGVGELSAVNGIAGSYAERIPVIAITGAPTRAVEHAGKYVHHSLGEGTFDDYRKMFAHITVAQGYITPENATTEIPRLINTAIAERRPVHLHLPIDVAISEIEIPTPFEVTAAKDTDASTYIELLTSKLHQSKQPIIITGHEINSFHLHQELEDFVNQTQIPVAQLSLGKGAFNEENPYYMGIYDGKIAEDKIRDYVDNSDLILNIGAKLTDSATAGFSYQFNIDDVVMLNHHNIKIDDVTNDEISLPSLLKQLSNISHTNNATFPAYHRPTSPDYTVGTEPLTQQTYFKMMQNFLKPNDVIIADQGTSFFGAYDLALYKNNTFIGQPLWGSIGYTLPATLGSQLADKDRRNLLLIGDGSLQLTVQAISTMIRQHIKPVLFVINNDGYTVERLIHGMYEPYNEIHMWDYKALPAVFGGKNVEIHDVESSKDLQDTFNAINGHPDVMHFVEVKMSVEDAPKKLIDIAKAFSQQNK

SEQ ID NO: 82 - skipped

SEQ ID NO: 83 - protein, 573 aa, Bacillus pumilus
MPQRTAGKEVTALLEEWGVKHIYGMPGDSINELIEELRHESSKIQFIQTRHEEVAALSAAADAKLTGKLGVCLSIAGPGAVHLLNGLYDAKADGAPVLAIAGQVASTEVGRDAFQEIKLERMFDDVAVFNQQVQTAEALPDLLNQAIKAAYTHKGVAVLTVSDDLFSQKIKRSPVYTSPLYVEGDVRPKKDQLLKAAQLINNAKKPVILAGKGLRNAKEELLSFAEKAAAPIVITLPAKGVVPDRHAYFLGNLGQIGTKPAYEAMEECDLLIMLGTSFPYRDYLPEDTPAIQLDIKPDQIGKRYPVEVGIVSDSKTGLHELTSYIEYKEQRGFLEACTEHMMKWREEMDKEKSIATSPLKPQQVIARLEEAVDDDAILSVDVGNVTVWMARHFEMKQQDFIISSWLATMGCGLPGAISAKLNEPNRQAIAVCGDGGFTMVMQDFVTAVKYKLPIVVVILNNNNLGMIEYEQQVKGNINYGIELEDIDFAKFAEACGGKGISVSSHEELAPAFDQALQADKPVIIDVAVTNEPPLPGKITYTQAAGFSKYLLKKFFEKGELDIPPLKKSLKRFF

SEQ ID NO: 84 - skipped

SEQ ID NO: 85 - protein, 559 aa, Streptomyces glaucescens
MVSRPARVAILEQLRADGVRYMFGNPGTVEQGFLDELRNFPDIEYILALQEAGVVGLADGYARATRTPAVLQLHTGVGVGNAVGMLYQAKRGHAPLVAIAGEAGLRYDAMEAQMAVDLVAMAEPVTKWATRVVDPESTLRVLRRAMKVAATPPYGPVLVVLPADVMDRDTSEAAVPTSYVDFAATPDPQVLDRAAELLAGAERPIVIAGDGVHFAGAQEELGRLAQTWGAEVWGADWAEVNLSVEHPAYAGQLGHMFGDSSRRVTGAADAVLLVGTYALPEVYPALDGVFADGAPVVHIDLDTDAIAKNFPVDLGLAADPRRALDGLARALERRMSPESRARAGEWFTGRSAQRSYEIAAAREQDEAALAPDALPVTAFLQELARQLPEDAVVFDEALTASPDVTRHLPPTRPGHWHQTRGGSLGVGIPGAIAAQLAHPDRTVVGFTGDGGSLYTIQALWTAARYDIGATFVICNNSSYKLLELNIEEYWKSVDVAAHEQPEMFDLARPAIDFVALSRSLGVPAVRVEKPDQAKAAVEQALGTPGPFLIDLVTGRGRED

SEQ ID NO: 86 - skipped

SEQ ID NO: 87 - protein, 528 aa, Pseudomonas aeruginosa
MKTVHSASYEILRRHGLTTVFGNPGSNELPFLKDFPEDFRYILGLHEGAVVGMADGFALASGRPAFVNLHAAAGTGNGMGALTNAWYSHSPLVITAGQQVRSMIGVEAMLANVDAGQLPKPLVKWSHEPACAQDVPRALSQAIQTASLPPRAPVYLSIPYDDWAQPAPAGVEHLAARQVSGAALPAPALLAELGERLSRSRNPVLVLGPDVDGANANGLAVELAEKLRMPAWGAPSASRCPFPTRHACFRGVLPAAIAGISRLLDGHDLILVVGAPVFRYHQFAPGDYLPAGAELVQVTCDPGEAARAPMGDALVGDIALTLEALLEQVRPSARPLPEALPRPPALAEEGGPLRPETVFDVIDALAPRDAIFVKESTSTVTAFWQRVEMREPGSYFFPAAGGLGFGLPAAVGAQLAQPRRQVIGIIGDGSANYGITALWSAAQYRVPAVFIILKNGTYGALRWFAGVLEVPDAPGLDVPGLDFCAIARGYGVEALHAATREELEGALKHALAADRPVLIEVPTQTIEP

SEQ ID NO: 88 - skipped

SEQ ID NO: 89 - protein, 584 aa, Actinoplanes missouriensis
MIDLDGTVTVAEYLGLRLRHAGVEHLFGVPGDFNLNLLDGLAFVEGLRWVGSPNELGAGYAADAYARRRGLSALFTTYGVGELSAINAVAGSAAEDSPVVHVVGSPRTTTVAGGALVHHTIADGDFRHFARAYAEVTVAQAMVTATDAGAQIDRVLLAALTHRKPVYLSIPQDLALHRIPAAPLREPLTPASDPAAVERFRTAVRDLLTPAVRPIMLVGQLVSRYGLSTLVTDMTTRSGIPVAAQLSAKGVIDESVEGNLGLYAGSMLDGPAASLIDSADVVLHLGTALTAELTGFFTHRRPDARTVQLLSTAALVGTTRFDNVLFPDAMTTLAEVLTTFPAPARLAAPTTRAEPTGLAASITPPAPSAVDLTASTATDLTAPTAGDISEMSRVLTQDAFWAGMQAWLPAGHALVADTGTSYWGALALRLPGDTVFLGQPIWNSIGWALPAVLGQGLADPDRRPVLVIGDGAAQMTIQELSTIVAAGLRPIILLLNNRGYTIERALQSPNAGYNDVADWNWRAVVAAFAGPDTDYHHAATGTELAKALTAASESNRPVFIEVELDAFDTPPLLRRLAERATAPS

SEQ ID NO: 90 - skipped

SEQ ID NO: 91 - protein, 548 aa, Carnobacterium maltaromaticum
MYTVGNYLLDRLTELGIRDIFGVPGDYNLKFLDHVMTHKELNWIGNANELNAAYAADGYARTKGIAALVTTFGVGELSAANGTAGSYAEKVPVVQIVGTPTTAVQNSHKLVHHTLGDGRFDHFEKMQTEINGAIAHLTADNALAEIDRVLRIAVTERCPVYINLAIDVAEVVAEKPLKPLMEESKKVEEETTLVLNKIEKALQDSKNPVVLIGNEIASFHLESALADFVKKFNLPVTVLPFGKGGFDEEDAHFIGVYTGAPTAESIKERVEKADLILIIGAKLTDSATAGFSYDFEDRQVISVGSDEVSFYGEIMKPVAFAQFVNGLNSLNYLGYTGEIKQVERVADIEAKASNLTQNNFWKFVEKYLSNGDTLVAEQGTSFFGASLVPLKSKMKFIGQPLWGSIGYTFPAMLGSQIANPASRHLLFIGDGSLQLTIQELGMTFREKLTPIVFVINNDGYTVEREIHGPNELYNDIPMWDYQNLPYVFGGNKGNVATYKVTTEEELVAAMSQARQDTTRLQWIEVVMGKQDSPDLLVQLGKVFAKQNS

SEQ ID NO: 92 - skipped

SEQ ID NO: 93 - protein, 570 aa, Schizosaccharomyces pombe
MSSEKVLVGEYLFTRLLQLGIKSILGVPGDFNLALLDLIEKVGDETFRWVGNENELNGAYAADAYARVKGISAIVTTFGVGELSALNGFAGAYSERIPVVHIVGVPNTKAQATRPLLHHTLGNGDFKVFQRMSSELSADVAFLDSGDSAGRLIDNLLETCVRTSRPVYLAVPSDAGYFYTDASPLKTPLVFPVPENNKEIEHEVVSEILELIEKSKNPSILVDACVSRFHIQQETQDFIDATHFPTYVTPMGKTAINESSPYFDGVYIGSLTEPSIKERAESTDLLLIIGGLRSDFNSGTFTYATPASQTIEFHSDYTKIRSGVYEGISMKHLLPKLTAAIDKKSVQAKARPVHFEPPKAVAAEGYAEGTITHKWFWPTFASFLRESDVVTTETGTSNFGILDCIFPKGCQNLSQVLWGSIGWSVGAMFGATLGIKDSDAPHRRSILIVGDGSLHLTVQEISATIRNGLTPIIFVINNKGYTIERLIHGLHAVYNDINTEWDYQNLLKGYGAKNSRSYNIHSEKELLDLFKDEEFGKADVIQLVEVHMPVLDAPRVLIEQAKLTASLNKQ

SEQ ID NO: 94 - skipped

SEQ ID NO: 95 - protein, 538 aa, Comamonas testosteroni
MPANTAPNAQAAEVFTVRHAVINMLRELGMTRIFGNPGSTELPLFRDYPEDFSYILGLQETVVVGMADGYAQATRNASFVNLHSAAGVGHAMANIFTAFKNRTPMVITAGQQTRSLLQFDPFLHSNQAAELPKPYVKWSCEPARAEDVPQALARAYYIAMQEPRGPVFVSIPADDWDVPCEPITLRKVGFETRPDPRLLDSIGQALEGARAPAFVVGAAVDRSQAFEAVQALAERHQARVYVAPMSGRCGFPEDHALFGGFLPAMRERIVDRLSGHDVVFVIGAPAFTYHVEGHGPFIAEGTQLFQLIEDPAIAAWAPVGDAAVGNIRMGVQELLARPLTHPRPALQPRPAIPAPAAPEPGRLMTDAFLMHTLAQVRSRDSIIVEEAPGSRSIIQAHLPIYAAETFFTMCSGGLGHSLPASVGIALARPDKKVIGVIGDGSAMYAIQALWSAAHLKLPVTYIIVKNRRYAALQDFSRVFGYREGEKVEGTDLPDIDFVALAKGQGCDGVRVTDAAQLSQVLRDALRSPRATLVEVEVA

SEQ ID NO: 96 - skipped

SEQ ID NO: 97 - protein, 536 aa, Amycolatopsis orientalis
MNVAELVGRTLAELGVGAAFGVVGSGNFVVTNGLRAGGVRFVAARHEGGAASMADAYARMSGRVSVLSLHQGCGLTNALTGITEAAKSRTPMIVLTGDTAASAVLSNFRIGQDALATAVGAVPERVHSAPTAVADTVRAYRTAVQQRRTVLLNLPLDVQAQEAPEAVEIPKVRGPAPIRPDAGMVAKLADLLAEARRPVFIAGRGARASAVPLRELAEISGALLATSAVAHGLFHDDPFSLGISGGFSSPRTADLIVDADLVIGWGCALNMWTTRHGTLLGPAARLVQVDVEQAALGAHRPIDLGVVGDVAGTAVDVHAELDKRGHQRSREAPTGTRWNDVPYNDLSGDGRIDPRTLSRRLDEILPAERMVSIDSGNFMGYPSAYLSVPDENGFCFTQAFQSIGLGLGTAIGAALARPDRLPVLGVGDGGFHMAVSELETAVRLRIPLVIVVYNDAAYGAEIHHFGDADMTTVRFPDTDIAAIGRGFGCDGVTVRSVGDLAAVKEWLGGPRDAPLVIDAKIADDGGSWWLAEAFRH

SEQ ID NO: 98 - skipped

SEQ ID NO: 99 - protein, 552 aa, Unknown
  Enterobacter sp.
MRTPYCVADYLLDRLTDCGADHLFGVPGDYNLQFLDHVIDSPDICWVGCANELNASYAADGYARCKGFAALLTTFGVGELSAMNGIAGSYAEHVPVLHIVGAPGTAAQQRGELLHHTLGDGEFRHFYHMSEPITVAQAVLTEQNACYEIDRVLTTMLRERRPGYLMLPADVAKKAATPPVNALTHKQAHADSACLKAFRDAAENKLAMSKRTALLADFLVLRHGLKHALQKWVKEVPMAHATMLMGKGIFDERQAGFYGTYSGSASTGAVKEAIEGADTVLCVGTRFTDTLTAGFTHQLTPAQTIEVQPHAARVGDVWFTGIPMNQAIETLVELCKQHVHAGLMSSSSGAIPFPQPDGSLTQENFWRTLQTFIRPGDIILADQGTSAFGAIDLRLPADVNFIVQPLWGSIGYTLAAAFGAQTACPNRRVIVLTGDGAAQLTIQELGSMLRDKQHPIILVLNNEGYTVERAIHGAEQRYNDIALWNWTHIPQALSLDPQSECWRVSEAEQLADVLEKVAHHERLSLIEVMLPKADIPPLLGALTKALEACNNA

SEQ ID NO: 100 - DNA, 1656 bp, Unknown
  Enterobacter sp.
atgcgtaccccgtactgcgttgctgactacctgctggaccgtctgaccgattgcggcgcggaccacctgtttggcgtgccgggcgactacaacctgcaatttctggaccatgtcattgattctccggacatctgctgggtgggctgtgccaacgaactgaatgcaagttatgcggccgatggctacgcacgttgcaaaggttttgcagctctgctgaccacgttcggcgtgggtgaactgtccgcgatgaatggcattgccggcagctatgcggaacatgtgccggttctgcacatcgttggcgcgccgggcaccgcggcgcagcaacgtggtgaactgctgcatcacacgctgggcgatggtgaatttcgccatttctaccacatgtccgaaccgattaccgttgcccaagcagtcctgacggaacagaacgcctgctatgaaatcgaccgtgtgctgaccacgatgctgcgcgaacgtcgtccgggctatctgatgctgccggctgatgttgcgaaaaaggcagctaccccgccggtcaacgcactgacgcataaacaggctcacgcggattccgcttgtctgaaggcgtttcgtgacgcggccgaaaataaactggccatgtcaaagcgtaccgccctgctggcagacttcctggtgctgcgtcatggcctgaaacacgcgctgcaaaaatgggttaaggaagtcccgatggcccatgcaaccatgctgatgggcaagggtatttttgatgaacgccaggccggcttctatggcacctactcaggctcggccagcacgggtgcagtgaaagaagctatcgaaggcgcggataccgtgctgtgcgttggtacgcgttttaccgacacgctgaccgccggtttcacgcatcagctgaccccggcacaaacgattgaagttcagccgcacgcagctcgcgtcggtgatgtgtggtttaccggtattccgatgaaccaagcgatcgaaacgctggttgaactgtgtaaacagcatgtccacgctggcctgatgagcagcagcagcggtgccattccgttcccgcaaccggatggctctctgacccaggaaaatttttggcgtacgctgcaaaccttcattcgtccgggcgatattatcctggcggaccagggcacctctgcttttggtgcgatcgatctgcgtctgccggccgacgtgaacttcattgttcaaccgctgtggggcagtatcggttataccctggcggcggcgtttggcgcccagacggcatgtccgaatcgtcgcgtcattgtgctgaccggcgatggtgctgcgcagctgacgatccaagaactgggtagcatgctgcgcgacaaacaacatccgattatcctggtgctgaacaatgaaggctataccgttgaacgtgccattcatggtgcagaacagcgctacaacgatattgcactgtggaattggacccacatcccgcaagcgctgtctctggacccgcagagtgaatgctggcgtgtgtcggaagctgaacagctggcggatgtcctggaaaaagtggcgcatcacgaacgcctgagcctgattgaagttatgctgccgaaagctgatatcccgccgctgctgggtgcgctgaccaaggctctggaagcgtgtaacaatgcc

SEQ ID NO: 101 - protein, 545 aa, Azospirillum brasilense
MKLAEALLRALKDRGAQAMFGIPGDFALPFFKVAEETQILPLHTLSHEPAVGFAADAAARYSSTLGVAAVTYGAGAFNMVNAVAGAYAEKSPVVVISGAPGTTEGNAGLLLHHQGRTLDTQFQVFKEITVAQARLDDPAKAPAEIARVLGAARAQSRPVYLEIPRNMVNAEVEPVGDDPAWPVDRDALAACADEVLAAMRSATSPVLMVCVEVRRYGLEAKVAELAQRLGVPVVTTFMGRGLLADAPTPPLGTYIGVAGDAEITRLVEESDGLFLLGAILSDTNFAVSQRKIDLRKTIHAFDRAVTLGYHTYADIPLAGLVDALLERLPPSDRTTRGKEPHAYPTGLQADGEPIAPMDIARAVNDRVRAGQEPLLIAADMGDCLFTAMDMIDAGLMAPGYYAGMGFGVPAGIGAQCVSGGKRILTVVGDGAFQMTGWELGNCRRLGIDPIVILFNNASWEMLRTFQPESAFNDLDDWRFADMAAGMGGDGVRVRTRAELKAALDKAFATRGRFQLIEAMIPRGVLSDTLARFVQGQKRLHAAPRE

SEQ ID NO: 102 - skipped

SEQ ID NO: 103 - protein, 547 aa, Lactococcus lactis
MYTVGDYLLDRLHELGIEEIFGVPGDYNLQFLDQIISREDMKWIGNANELNASYMADGYARTKKAAAFLTTFGVGELSAINGLAGSYAENLPVVEIVGSPTSKVQNDGKFVHHTLADGDFKHFMKMHEPVTAARTLLTAENATYEIDRVLSQLLKERKPVYINLPVDVAAAKAEKPALSLEKESSTTNTTEQVILSKIEESLKNAQKPVVIAGHEVISFGLEKTVTQFVSETKLPITTLNFGKSAVDESLPSFLGIYNGKLSEISLKNFVESADFILMLGVKLTDSSTGAFTHHLDENKMISLNIDEGIIFNKVVEDFDFRAVVSSLSELKGIEYEGQYIDKQYEEFIPSSAPLSQDRLWQAVESLTQSNETIVAEQGTSFFGASTIFLKSNSRFIGQPLWGSIGYTFPAALGSQIADKESRHLLFIGDGSLQLTVQELGLSIREKLNPICFIINNDGYTVEREIHGPTQSYNDIPMWNYSKLPETFGATEDRVVSKIVRTENEFVSVMKEAQADVNRMYWIELVLEKEDAPKLLKKMGKLFAEQNK

SEQ ID NO: 104 - DNA, 1641 bp, Lactococcus lactis
atgtacaccgttggcgactacctgctggaccgtctgcatgaactgggcatcgaagaaatctttggcgtgccgggtgactataacctgcaatttctggatcagattatcagccgtgaagacatgaaatggattggtaacgctaatgaactgaacgcatcttatatggctgatggttacgcacgtaccaaaaaggcggcggcgtttctgaccacgttcggcgttggtgaactgagcgcaattaacggcctggccggttcttatgcagaaaatctgccggtggttgaaatcgttggctcaccgacgtcgaaagtccagaatgatggcaagtttgtgcatcacaccctggccgatggcgactttaaacatttcatgaagatgcacgaaccggtgacggctgcgcgtaccctgctgacggcggaaaacgccacctatgaaattgatcgtgtgctgagccagctgctgaaagaacgcaagccggtttacatcaatctgccggttgatgtcgccgcagctaaagctgaaaagccggcgctgtctctggaaaaagaaagctctaccacgaacaccacggaacaggttattctgagcaaaatcgaagaatctctgaaaaatgcccaaaagccggtcgtgattgcaggccatgaagtgatctcatttggtctggaaaaaaccgtcacgcagttcgtgtcggaaaccaagctgccgattaccacgctgaactttggtaaaagtgccgtggatgaaagcctgccgtctttcctgggcatttataacggtaaactgagtgaaatctccctgaagaattttgtcgaaagcgccgatttcattctgatgctgggcgtgaaactgaccgacagttccacgggtgcatttacccatcacctggatgaaaacaagatgatcagtctgaacatcgacgaaggcatcatcttcaacaaggttgtcgaagatttcgacttccgtgcggtggtttcatcgctgtccgaactgaagggcattgaatatgaaggccagtacatcgataagcaatacgaagaatttatcccgagcagcgcaccgctgagccaggaccgtctgtggcaagcagttgaatcactgacgcagtcgaacgaaaccattgtcgctgaacaaggcaccagctttttcggtgcgtccaccatctttctgaaaagtaattcccgtttcattggtcagccgctgtggggcagcatcggttatacctttccggcggcactgggctcacaaattgcggataaagaatcgcgccatctgctgttcatcggcgacggtagcctgcaactgaccgttcaagaactgggtctgtctattcgtgaaaaactgaacccgatctgctttattatcaacaatgatggctacacggtggaacgcgaaattcacggtccgacccagtcatataacgacatcccgatgtggaattactcgaaactgccggaaacgtttggcgccaccgaagatcgtgtcgtgagtaagattgtgcgcaccgaaaacgaatttgtgtccgttatgaaagaagcacaggctgatgttaatcgcatgtattggatcgaactggtcctggaaaaagaagacgctccgaagctgctgaaaaagatgggcaaactgtttgcggaacagaacaag

SEQ ID NO: 105 - protein, 558 aa, Acetobacter syzygii
MTYTVGMYLAERLVQIGLKHHFAVAGDYNLVLLDQLLLNKDMKQIYCCNELNCGFSAEGYARSNGAAAAVVTFSVGAISAMNALGGAYAENLPVILISGAPNSNDQGTGHILHHTIGKTDYSYQLEMARQVTCAAESITDAHSAPAKIDHVIRTALRERKPAYLDIACNIASEPCVRPGPVSSLLSEPEIDHTSLKAAVDATVALLEKSASPVMLLGSKLRAANALAATETLADKLQCAVTIMAAAKGFFPEDHAGFRGLYWGEVSNPGVQELVETSDALLCIAPVFNDYSTVGWSAWPKGPNVILAEPDRVTVDGRAYDGFTLRAFLQALAEKAPARPASAQKSSVPTCSLTATSDEAGLTNDEIVRHINALLTSNTTLVAETGDSWFNAMRMTLPRGARVELEMQWGHIGWSVPSAFGNAMGSQDRQHVVMVGDGSFQLTAQEVAQMVRYELPVIIFLINNRGYVIEIAIHDGPYNYIKNWDYAGLMEVFNAGEGHGLGLKATTPKELTEAIARAKANTRGPTLIECQIDRTDCTDMLVQWGRKVASTNARKTTLA

SEQ ID NO: 106 - DNA, 1674 bp, Acetobacter syzygii
atgacctatacggtgggcatgtacctggctgaacgcctggtgcagattggcctgaaacatcactttgcggtggctggcgattacaacctggtgctgctggatcaactgctgctgaacaaagacatgaaacagatttattgctgtaacgaactgaattgcggctttagcgcagaaggttacgctcgctctaatggtgcggcggcggcagtggttaccttcagtgtgggtgccatttccgcaatgaacgctctgggcggtgcttacgcggaaaatctgccggttattctgatctcaggcgcgccgaactcgaatgatcagggcacgggtcatatcctgcatcacaccattggtaaaacggattatagctaccaactggaaatggcacgtcaggtcacctgtgcggccgaatcaatcacggatgcgcattcggccccggcaaaaatcgaccacgttattcgtaccgcactgcgtgaacgtaaaccggcatatctggatatcgcgtgcaacattgcaagcgaaccgtgtgtgcgtccgggtccggttagctctctgctgagtgaaccggaaattgatcatacctccctgaaagcagctgtggacgcgacggttgccctgctggaaaaatcagcctcgccggtgatgctgctgggctcaaaactgcgtgcagcaaacgcactggcagctaccgaaacgctggcagataaactgcagtgcgctgtgaccatcatggcggcggcaaaaggctttttcccggaagatcacgccggcttccgtggtctgtattggggcgaagtttcaaatccgggtgtccaggaactggtggaaacctcggatgcactgctgtgtatcgctccggtttttaacgactacagcacggtcggctggtctgcgtggccgaaaggtccgaatgtgattctggccgaaccggaccgtgttaccgtcgatggtcgtgcgtatgatggttttacgctgcgtgctttcctgcaagctctggcagaaaaagcaccggcacgtccggctagtgcacagaaaagttccgttccgacctgcagtctgaccgcgacgtccgatgaagccggcctgacgaacgacgaaatcgttcgccacattaacgcgctgctgaccagcaataccacgctggtcgcggaaacgggcgattcttggttcaatgccatgcgtatgaccctgccgcgtggtgcacgcgtcgaactggaaatgcagtggggccatattggttggagcgtgccgtctgcatttggcaatgctatgggtagtcaggatcgtcaacacgtcgtgatggtgggcgacggttccttccagctgaccgcgcaagaagttgcccagatggtccgttatgaactgccggtgattatctttctgatcaacaatcgcggctacgttattgaaatcgccattcatgatggtccgtacaactacatcaaaaactgggactatgccggtctgatggaagtttttaacgcaggcgaaggtcacggcctgggtctgaaagcgaccacgccgaaagaactgaccgaagccattgcacgtgctaaagcgaatacccgcggcccgacgctgatcgaatgccaaattgatcgtaccgactgtacggatatgctggtccagtggggtcgcaaagtggcgtctaccaacgcacgcaaaacgacgctggcg

SEQ ID NO: 107 - protein, 528 aa, Agrobacterium radiobacter [sequence omitted]

SEQ ID NO: 108 - DNA, 1584 bp, Agrobacterium radiobacter
atggcgagcgtgcatggcaccacgtatgaactgctgcgtcgccagggtatcgataccgtgttcggcaacccgggttcaaatgaactgccgtttctgaaagatttcccggaagactttcgttatatcctggcactgcaagaagcgtgcgtggttggcattgcagacggttacgcgcaagcctcgcgcaaaccggcgtttattaacctgcatagcgcggccggcaccggtaatgcaatgggcgctctgagcaacgcgtggaacagccacagcccgctgatcgtgaccgcgggccagcaaacgcgtgccatgattggtgtggaagcactgctgacgaacgttgatgcagctaatctgccgcgcccgctggtcaaatggtcctatgaaccggcatcagcggccgaagtgccgcatgcaatgtctcgtgccatccacatggcaagtatggccccgcagggtccggtctatctgtctgtgccgtacgatgactgggataaagacgccgatccgcagagtcatcacctgtttgatcgtcatgttagctctagtgtccgcctgaacgaccaggatctggatatcctggttaaagcactgaactctgctagtaatccggcgattgtgctgggtccggatgttgacgcagctaacgcaaatgctgattgcgtgatgctggctgaacgtctgaaagcgccggtttgggtcgcaccgtcggctccgcgttgcccgttcccgacccgtcacccgtgttttcgtggtctgatgccggccggtattgcagcaatcagccagctgctggaaggccatgatgtcgtgctggtcatcggtgcaccggtgttccgctatcaccagtacgacccgggccaatatctgaaaccgggtacccgtctgatttctgttacgtgtgatccgctggaagcagctcgcgcgccgatgggcgatgcaatcgtggcagacattggtgcgatggccagtgcactggctaacctggttgaagaatcctcacgtcagctgccgaccgcggccccggaaccggctaaagttgatcaagacgcaggtcgtctgcacccggaaaccgtctttgatacgctgaatgacatggccccggaaaacgcaatttacctgaatgaatccacgtcaaccacggcccagatgtggcaacgtctgaacatgcgcaatccgggttcttattacttctgtgcagctggcggtctgggttttgcactgccggcggcaatcggtgtgcagctggcggaaccggaacgtcaagtgattgccgttatcggcgatggtagcgccaactattcgattagcgcactgtggaccgcagctcagtacaatattccgacgatcttcgttattatgaacaatggcacctatggtgccctgcgttggtttgcaggtgtgctggaagctgaaaacgttccgggcctggatgtcccgggtatcgacttccgtgcactggcaaaaggctacggtgttcaggcactgaaagctgataatctggaacagctgaaaggctcgctgcaagaagcgctgagcgccaaaggtccggtgctgattgaagtctctaccgtgagtccggttaaa

SEQ ID NO: 109 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVDETLKFIANRDKVAVLVGSKLRAAGAEEAAVKFTDALGGAVATMAAAKSFFPEENALYIGTSWGEVSYPGVEKTMKEADAVIALAPVFNDYSTTGWTDIPDPKKLVLAEPRSVVVNGIRFPSVHLKDYLTRLAQKVSKKTGSLDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAAKGLKAKTGGELAEAIKVALANTDGPTLIECFIGREDCTEELVKWGKRVAAANSRKPVNKVV

SEQ ID NO: 110 - DNA, 1704 bp, Zymomonas mobilis
atgagctataccgtgggcacgtacctggctgaacgtctggttcaaattggcctgaaacatcactttgccgtggccggtgattataatctggttctgctggacaacctgctgctgaataaaaacatggaacaggtgtactgctgtaatgaactgaactgcggcttcagtgcggaaggttatgctcgcgcgaagggtgcggcggcggcggtggttacctacagtgttggtgccctgtccgcatttgatgctatcggcggtgcctatgcagaaaatctgccggttattctgatctccggcgccccgaacaataacgatcatgcggcgggtcatgtcctgcatcacgcactgggtaaaaccgactatcattaccagctggaaatggcaaaaaacattaccgcagctgcggaagcgatctatacgccggaagaagctccggcgaaaattgatcacgttatcaaaaccgcgctgcgtgagaaaaaaccggtctacctggaaattgcgtgcaatatcgcctcaatgccgtgtgcagcaccgggtccggcatcggcactgtttaatgatgaagcaagcgacgaagcttctctgaacgctgcggtggatgaaaccctgaaattcattgcgaaccgtgacaaagttgcagtcctggtgggcagcaaactgcgtgccgcaggtgcagaagaagctgcggtcaaatttaccgatgcactgggcggtgctgtggcaacgatggccgcagctaaaagctttttcccggaagaaaatgccctgtatatcggcacctcatggggtgaagtgtcgtacccgggtgttgaaaaaacgatgaaagaagccgatgcagtcattgctctggcgccggtgttcaatgactatagcaccacgggctggaccgatatcccggacccgaaaaaactggttctggcggaaccgcgtagcgtcgtggttaacggtattcgctttccgtctgtgcatctgaaagattacctgacccgtctggcccaaaaagttagcaagaaaaccggctctctggactttttcaaaagtctgaatgcgggtgaactgaaaaaagcagcaccggccgatccgtccgcaccgctggtcaatgcggaaattgcacgtcaggtggaagcactgctgaccccgaacaccacggtgatcgccgaaacgggcgactcttggttcaatgcacaacgtatgaaactgccgaacggtgcgcgcgttgaatatgaaatgcagtggggccatattggttggagcgttccggcagcttttggctacgcagtcggtgctccggaacgtcgcaacatcctgatggtgggcgatggttcgttccagctgaccgcacaagaagttgctcagatggtccgtctgaaactgccggtcatcatctttctgatcaacaactacggctacacgattgaagtgatgatccacgatggtccgtataataacatcaaaaattgggactacgccggcctgatggaagtgtttaatggtaacggcggttatgatagtggcgcggccaaaggtctgaaagcgaaaaccggcggtgaactggccgaagcaattaaagttgctctggcgaacaccgatggcccgacgctgattgaatgcttcatcggtcgcgaagactgtaccgaagaactggttaaatggggcaaacgtgtcgcagctgcgaatagccgcaaaccggtgaacaaagtcgtg

SEQ ID NO: 111 - protein, 559 aa, Klebsiella pneumoniae
MDKQYPVRQWAHGADLVVSQLEAQGVRQVFGIPGAKIDKVFDSLLDSSIRIIPVRHEANAAFMAAAVGRITGKAGVALVTSGPGCSNLITGMATANSEGDPVVALGGAVKRADKAKQVHQSMDTVAMFSPVTKYAIEVTAPDALAEVVSNAFRAAEQGRPGSAFVSLPQDVVDGPVSGKVLPASGAPQMGAAPDDAIDQVAKLIAQAKNPIFLLGLMASQPENSKALRRLLETSHIPVTSTYQAAGAVNQDNFSRFAGRVGLFNNQAGDRLLQLADLVICIGYSPVEYEPAMWNSGNATLVHIDVLPAYEERNYTPDVELVGDIAGTLNKLAQNIDHRLVLSPQAAEILRDRQHQRELLDRRGAQLNQFALHPLRIVRAMQDIVNSDVTLTVDMGSFHIWIARYLYTFRARQVMISNGQQTMGVALPWAIGAWLVNPERKVVSVSGDGGFLQSSMELETAVRLKANVLHLIWVDNGYNMVAIQEEKKYQRLSGVEFGPMDFKAYAESFGAKGFAVESAEALEPTLRAAMDVDGPAVVAIPVDYRDNPLLMGQLHLSQIL

SEQ ID NO: 112 - protein, 528 aa, Pseudomonas aeruginosa; identical to SEQ ID NO: 87

SEQ ID NO: 113 - protein, 584 aa, Actinoplanes missouriensis; identical to SEQ ID NO: 89

SEQ ID NO: 114 - protein, 548 aa, Carnobacterium maltaromaticum; identical to SEQ ID NO: 91

SEQ ID NO: 115 - protein, 538 aa, Comamonas testosteroni; identical to SEQ ID NO: 95

SEQ ID NO: 116 - protein, 536 aa, Amycolatopsis orientalis; identical to SEQ ID NO: 97

SEQ ID NO: 117 - protein, 552 aa, Unknown; identical to SEQ ID NO: 99
  Enterobacter sp.

SEQ ID NO: 118 - protein, 545 aa, Azospirillum brasilense; identical to SEQ ID NO: 101

SEQ ID NO: 119 - protein, 536 aa, Lactococcus lactis; identical to SEQ ID NO: 97

SEQ ID NO: 120 - protein, 560 aa, Acetobacter syzygii
MTYTVGMYLAERLVQIGLKHHFAVAGDYNLVLLDQLLLNKDMKQIYCCNELNCGFSAEGYARSNGAAAAVVTFSVGAISAMNALGGAYAENLPVILISGAPNSNDQGTGHILHHTIGKTDYSYQLEMARQVTCAAESITDAHSAPAKIDHVIRTALRERKPAYLDIACNIASEPCVRPGPVSSLLSEPEIDHTSLKAAVDATVALLEKSASPVMLLGSKLRAANALAATETLADKLQCAVTIMAAAKGFFPEDHAGFRGLYWGEVSNPGVQELVETSDALLCIAPVFNDYSTVGWSAWPKGPNVILAEPDRVTVDGRAYDGFTLRAFLQALAEKAPARPASAQKSSVPTCSLTATSDEAGLTNDEIVRHINALLTSNTTLVAETGDSWFNAMRMTLPRGARVELEMQWGHIGWSVPSAFGNAMGSQDRQHVVMVGDGSFQLTAQEVAQMVRYELPVIIFLINNRGYVIEIAIHDGPYNYIKNWDYAGLMEVFNAGEGHGLGLKATTPKELTEAIARAKANTRGPTLIECQIDRTDCTDMLVQWGRKVASTNARKTTLALE

SEQ ID NO: 121 - protein, 534 aa, Agrobacterium radiobacter
MASVHGTTYELLRRQGIDTVFGNPGSNELPFLKDFPEDFRYILALQEACVVGIADGYAQASRKPAFINLHSAAGTGNAMGALSNAWNSHSPLIVTAGQQTRAMIGVEALLTNVDAANLPRPLVKWSYEPASAAEVPHAMSRAIHMASMAPQGPVYLSVPYDDWDKDADPQSHHLFDRHVSSSVRLNDQDLDILVKALNSASNPAIVLGPDVDAANANADCVMLAERLKAPVWVAPSAPRCPFPTRHPCFRGLMPAGIAAISQLLEGHDVVLVIGAPVFRYHQYDPGQYLKPGTRLISVTCDPLEAARAPMGDAIVADIGAMASALANLVEESSRQLPTAAPEPAKVDQDAGRLHPETVFDTLNDMAPENAIYLNESTSTTAQMWQRLNMRNPGSYYFCAAGGLGFALPAAIGVQLAEPERQVIAVIGDGSANYSISALWTAAQYNIPTIFVIMNNGTYGALRWFAGVLEAENVPGLDVPGIDFRALAKGYGVQALKADNLEQLKGSLQEALSAKGPVLIEVSTVSPVKHHHHHH

SEQ ID NO: 122 - protein, 314 aa, Metallosphaera sedula
MTEKVSVVGAGVIGVGWATLFASKGYSVSLYTEKKETLDKGIEKLRNYVQVMKNNSQITEDVNTVISRVSPTTNLDEAVRGANFVIEAVIEDYDAKKKIFGYLDSVLDKEVILASSTSGLLITEVQKAMSKHPERAVIAHPWNPPHLLPLVEIVPGEKTSMEVVERTKSLMEKLDRIVVVLKKEIPGFIGNRLAFALFREAVYLVDEGVATVEDIDKVMTAAIGLRWAFMGPFLTYHLGGGEGGLEYFFNRGFGYGANEWMHTLAKYDKFPYTGVTKAIQQMKEYSFIKGKTFQEISKWRDEKLLKVYKLVWEK

SEQ ID NO: 123 - protein, 292 aa, Bacillus cereus
MEHKTLSIGFIGIGVMGKSMVYHLMQDGHKVYVYNRTKAKTDSLVQDGANWCNTPKELVKQVDIVMTMVGYPHDVEEVYFGIEGIIEHAKEGTIAIDFTTSTPTLAKRINEVAKRKNIYTLDAPVSGGDVGAKEAKLAIMVGGEKEIYDRCLPLLEKLGTNIQLQGPAGSGQHTKMCNQIAIASNMIGVCEAVAYAKKAGLNPDKVLESISTGAAGSWSLSNLAPRMLKGDFEPGFYVKHFMKDMKIALEEAERLQLPVPGLSLAKELYEELIKDGEENSGTQVLYKKYIRG

SEQ ID NO: 124 - protein, 296 aa, Bacillus cereus
MKKIGFIGLGNMGLPMSKNLVKSGYTVYGVDLNKEAEASFEKEGGIIGLSISKLAETCDVVFTSLPSPRAVEAVYFGAEGLFENGHSNVVFIDTSTVSPQLNKQLEEAAKEKKVDFLAAPVSGGVIGAENRTLTFMVGGSKDVYEKTESIMGVLGANIFHVSEQIDSGTTVKLINNLLIGFYTAGVSEALTLAKKNNMDLDKMFDILNVSYGQSRIYERNYKSFIAPENYEPGFTVNLLKKDLGFAVDLAKESELHLPVSEMLLNVYDEASQAGYGENDMAALYKKVSEQLISNQK

SEQ ID NO: 125 - protein, 298 aa, Pseudomonas aeruginosa
MKQIAFIGLGHMGAPMATNLLKAGYLLNVFDLVQSAVDGLVAAGASAARSARDAVQGADVVISMLPASQHVEGLYLDDDGLLAHIAPGTLVLECSTIAPTSARKIHAAARERGLAMLDAPVSGGTAGAAAGTLTFMVGGDAEALEKARPLFEAMGRNIFHAGPDGAGQVAKVCNNQLLAVLMIGTAEAMALGVANGLEAKVLAEIMRRSSGGNWALEVYNPWPGVMENAPASRDYSGGFMAQLMAKDLGLAQEAAQASASSTPMGSLALSLYRLLLKQGYAERDFSVVQKLFDPTQGQ

SEQ ID NO: 126 - protein, 267 aa, Saccharomyces cerevisiae
MSQGRKAAERLAKKTVLITGASAGIGKATALEYLEASNGDMKLILAARRLEKLEELKKTIDQEFPNAKVHVAQLDITQAEKIKPFIENLPQEFKDIDILVNNAGKALGSDRVGQIATEDIQDVFDTNVTALINITQAVLPIFQAKNSGDIVNLGSIAGRDAYPTGSIYCASKFAVGAFTDSLRKELINTKIRVILIAPGLVETEFSLVRYRGNEEQAKNVYKDTTPLMADDVADLIVYATSRKQNTVIADTLIFPTNQASPHHIFRG

SEQ ID NO: 127 - protein, 290 aa, Gluconobacter oxydans
MSSPKIGFIGYGAMAQRMGANLRKAGYPVVAYAPSGGKDETEMLPSPRAIAEAAEIIIFCVPNDAAENESLHGENGALAALTPGKLVLDTSTVSPDQADAFASLAVEHGFSLLDAPMSGSTPEAETGDLVMLVGGDEAVVKRAQPVLDVIGKLTIHAGPAGSAARLKLVVNGVMGATLNVIAEGVSYGLAAGLDRDVVFDTLQQVAVVSPHHKRKLKMGQNREFPSQFPTRLMSKDMGLLLDAGRKVGAFMPGMAVADQALALSNRLHANEDYSALIGAMEHSVANLPHK

SEQ ID NO: 128 - protein, 314 aa, Nitrosopumilus maritimus
MHTVRIPKVINFGEDALGQTEYPKNALVVTTVPPELSDKWLAKMGIQDYMLYDKVKPEPSIDDVNTLISEFKEKKPSVLIGLGGGSSMDVVKYAAQDFGVEKILIPTTFGTGAEMTTYCVLKFDGKKKLLREDRFLADMAVVDSYFMDGTPEQVIKNSVCDACAQATEGYDSKLGNDLTRTLCKQAFEILYDAIMNDKPENYPYGSMLSGMGFGNCSTTLGHALSYVFSNEGVPHGYSLSSCTTVAHKHNKSIFYDRFKEAMDKLGFDKLELKADVSEAADVVMTDKGHLDPNPIPISKDDVVKCLEDIKAGNL

SEQ ID NO: 129 - protein, 248 aa, Escherichia coli
MIVLVTGATAGFGECITRRFIQQGHKVIATGRRQERLQELKDELGDNLYIAQLDVRNRAAIEEMLASLPAEWCNIDILVNNAGLALGMEPAHKASVEDWETMIDTNNKGLVYMTRAVLPGMVERNHGHIINIGSTAGSWPYAGGNVYGATKAFVRQFSLNLRTDLHGTAVRVTDIEPGLVGGTEFSNVRFKGDDGKAEKTYQNTVALTPEDVSEAVWWVSTLPAHVNINTLEMMPVTQSYAGLNVHRQ

SEQ ID NO: 130 - protein, 289 aa, Thermus thermophilus
MEKVAFIGLGAMGYPMAGHLARRFPTLVWNRTFEKALRHQEEFGSEAVPLERVAEARVIFTCLPTTREVYEVAEALYPYLREGTYWVDATSGEPEASRRLAERLREKGVTYLDAPVSGGTSGAEAGTLTVMLGGPEEAVERVRPFLAYAKKVVHVGPVGAGHAVKAINNALLAVNLWAAGEGLLALVKQGVSAEKALEVINASSGRSNATENLIPQRVLTRAFPKTFALGLLVKDLGIAMGVLDGEKAPSPLLRLAREVYEMAKRELGPDADHVEALRLLERWGGVEIR

SEQ ID NO: 131 - DNA, 500 bp, Artificial Sequence
  Synthetic Cosntruct
tgtgaagatgaatgtattgaatataaaattatttcttgatatccatatatcccataaacaagaaattactacttccggaaaaacgtaaacacagtggaaaatttacgataccaatcacgtgatcaaattacaaggaaagcacgtgacttaaggcttcctaaactagaaattgtggctgtcaggatcaattgaaaatggcgccacactttcttctcttatggttaggagtagaccccgaagacagaggattccggcaatcggagcacagtacaactttatactttcgttcactgcatggagagtgaaatttttcaagctgatgcaattgatataaatataacccatttacaggatatgtccctccaaaggttgatccgtttattgctataatgaatattggttcactattttatgcctcttgatttgtaatccgggcctttgctttttgtacttgaccttagaccttaatccaccccaatagtaactaatcagaacacaaa

SEQ ID NO: 132 - DNA, 400 bp, Artificial Sequence
  Synthetic Cosntruct
attcaactagaaagttgcaagtaaagcaactaactgcgggaccaaacaaatttaaacaaacccgtgaatattgttctaccttatcctattgcttcgaaaaaatgagcaaatattaacgacagtttactactgtcgtagcttttacttcaaatagaaggaaaactgatgaatttgcatacatgagcaattttattagaaattattacctaaaaaggcaagaaagcagagataattttctcatgcccccaactacttacttatatctacaattaaaacttaataatatgctctttggcagtatgaaccttttctttaaataacagagtactgccgcttcaaacgatgtattctacattgactaaacgaaaatactacaagctgtcttactttttaaacaaac

SEQ ID NO: 133 - DNA, 500 bp, Artificial Sequence
  Synthetic Cosntruct
ttgcgtaagatagattcaaaccaagtgatggacctgtcactgcttagtgttgatgaacaaacatatcttcgaggccattccgcaatgaaaaatcaatttctgactagcttgcttggagaggagccatcgataccagagtcagatcctgacaacgaatcgtgtcacatttttgtccgtgcccaagcaccgtttcccttccgagatgaagataccatgcaagtaggtgatgttcgtgttgctaaatggaaagacgtggcgcatggtgtagcagagggagctttacacgtgatataaacagcatgcgcctcattgagcaaattaactactaacggtttccgaaataggtaattgagcaaataagaatttcagcactttatgaagaagggtcaagcgtatataaaggacacctcttactttgaggttgtaagtttgtctctagccttatcaatggtctttattttttctgctaccttgattgggaaataatccaatcttcaata

SEQ ID NO: 134 - DNA, 23 bp, Artificial Sequence
  Synthetic Cosntruct
taatacgactcactatagggaga

SEQ ID NO: 135 - DNA, 485 bp, Artificial Sequence
  Synthetic Cosntruct
ttgatttaacctgatccaaaaggggtatgtctattttttagagagtgtttttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggggggggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttgtatcaaacacattacaaaacacacaaaacacacaaacaaacacaattacaaaaa

SEQ ID NO: 136 - DNA, 500 bp, Artificial Sequence
  Synthetic Cosntruct
tatcgtatttattaatccccttccccccagcgcagatcgtcccgtcgatttctattgtttgggcattatcagcgacgcgacggcgacgcgacggcgataatgggcgacggtcacaagatggaacgagaaaacagtttttttcggataggactcattttccaggtgagaatggggtgaccccggggagaaaccttccgcgagtggagtgcgagtggagtgggaaatgtggccccccccccccttgtgggccatgaggttgacaaataccgtgtggcccggtgatggagtgagaaagagagggaaatgataatgggaaaacaaggagaggcccgtttcccgggatttatataaagaggtgtctctatcccagttgaagtagagatttgttgatgtagttgttccttccaataaatttgttcaatcagtacacagctaatactattattacagctactactaatactactactactattactaccacccccaacacaaacaca

SEQ ID NO: 137 - protein, 567 aa, Commensalibacter intestini
MEYTVGQYLATRLAQLGLNHVFAVAGDYNLTLLDEMAKAKDLEQVYCCNELNCGFAGEGYARARIMGASVVTFSVGAFSAFNAVGGAFAENLPLLLISGAPNNNDYGSGHILHHTMGYSDYRYQMEMAKKITCEAVSVAHADEAPCLIDHAIRSAIRNRKPAYIEISCNVANQPCTEPGPISSITNSLISDDESLKAAAKACVEALEKAKNPVVIIGGKIRSAGCAVSKQVAELTKKLGCAVATMAQAKGLSPEEEAEYVGTFWGDISSPGVEDLVRDSDCRIYIGAVFNDYSTVGWTCKLVSDNDILISSHHTRVGKKEFSGVYLKDFIPVLASSVKKNTTSLEQFKAKKLPAKETPVADGNAALTTVELCRQIQGAINKDTTLFLETGDSWFHGMHFNLPNGARVESEMQWGHIGWSIPSMFGYAVSEPNRRNIIMVGDGSFQLTAQEVCQMIRRNMPVIIILINNSGYTIEVKIHDGPYNRIKNWDYAGLIDVFNAEDGKGLGLKAKNGAELEKAMKTALAHKDGPTLIEVDIDAQDCSPDLVVWGKKVAKANGRAPRKAGGSG

SEQ ID NO: 138 - protein, 570 aa, Commensalibacter sp. MX01
MKYTVGQYLATRLAQLGLNHVFAVAGDYNLTLLDEMAKVEDLEQVYCCNELNCGFAGEGYARSRVMGASVVTFSVGAFSAFNAVGGAFAENLPLLLISGAPNNNDYGSGHILHHTMGYSDYRYQMDMAKQITCEAVSVAHADEAPCLIDHAIRSALRNRKPAYIEISCNVANQPCTEPGPISSITNSLISDDESLKAAAKACLDALEKAKSPVVIIGGKIRSAGCAVSKKVAELTKKLGCAVATMAQAKGLSPEEEAEYVGTFWGEISSPGVEELVRESDCRIYIGAVFNDYSTVGWTCKLVGENDILISSHHTRVGHKEFSGVYLKDFIPVLTSCVKKNTTSLDQFKAKKIPVKQVPVADGKAPLTTVELCRQIQGAINKDTTIYLETGDSWFHGMHFKLPNGARVESEMQWGHIGWSIPSMFGYAVSEPNRRNIIMVGDGSFQLTAQEVCQMIRRNIPIIIILINNSGYTIEVKIHDGPYNRIKNWDYAGLINVFNAEDGKGLGLKAKNGAELEKAMQTALAHKDGPTLIEVDIDAQDCSPDLVVWGKKVAKANGRAPRKFQTFGGSG

SEQ ID NO: 139 - protein, 564 aa, Microcystis aeruginosa
MSNYNVGTYLAERLVQIGVKHHFVVPGDYNLVLLDQFLKNQNLLQVGCCNELNCGFAAEGYARANGLGVAVVTYSVGALSALNAIGGAYAENLPVILVSGAPNTNDYSTGHLLHHTMGTQDLTYVLEIARKLTCAAVSITSAEDAPEQIDHVIRTALREQKPAYIEIACNIAAAPCASPGPVSAIINEVPSDAETLAAAVSAAAEFLDSKQKPVLLIGSQLRAAKAEQEAIELAEALGCSVAVMAAAKSFFPEEHPQYVGTYWGEISSPGTSAIVDWSDAVVCLGAVFNDYSTVGWTAMPSGPTVLNANKDSVKFDGYHFSGIHLRDFLSCLARKVEKRDATMAEFARFRSTSVPVEPARSEAKLSRIEMLRQIGPLVTAKTTVFAETGDSWFNGMKLQLPTGARFEIEMQWGHIGWSIPAAFGYALGAPERQIICMIGDGSFQLTAQEVAQMIRQKLPIIIFLVNNHGYTIEVEIHDGPYNNIKNWDYAGLIKVFNAEDGAGQGLLATTAGELAQAIEVALENREGPTLIECVIDRDDATADLISWGRAVAVANARPHRGGSG

SEQ ID NO: 140 - protein, 565 aa, Pseudogymnoascus sp. VKM F-4519
MATFTVGDYLAERLAQIGIRHHFVVPGDYNLILLDKLQSHPDLSELGCANELNCSLAAEGYARAQGVAACIVTYSVGAFSAFNGTGSAYAENLPLILVSGSPNTNDSAKFHLLHHTLGTNDFTYQFEMAKKITCCAVAVGRAQDAPRLIDQAIRAALLAKKPAYIEIPTNLSGAMCVRPGPISAVVEPVLSDKASLTAAVDRAVQYLCGKQKPAILVGPKLRRAGAEMALLQVAEAIGCAVAVQPAAKGFFPEDHKQFAGVFWGQVSTLAADSILNWADTILCVGTIFTDYSTVGWTALPNVPLMIAEMDHVMFPGATFGRVRLNDFLSGLAKTVGRNESTMVEYGYIRPDPPLVHAAAPDELLNRKETARQVQMLLTPETTVFVDTGDSWFNGIRMKLPRGASFEIEMQWGHIGWSIPAAFGYAMGKPERKVITMVGDGSFQMTAQEVSQMVRYKVPIIIFLINNKGYTIEVEIHDGLYNRIKNWDYALLVRAFNSNDGQAIGFRASTGRELAEAIEKAKAHKDGPTLIECVIDQDDCSRELITWGHYVAAANARPPVQTGGSG

SEQ ID NO: 141 - protein, 565 aa, Pseudogymnoascus sp. VKM F-4519
MSWTVGSYLAERLAQIGIEHHFVVPGDYNLVLLDKLQAHPKLSEIGCANELNCSFAAEGYARAKGVAAAVVTFSVGAFSAFNGVGGAYAENLPVILISGAPNTSDSGAFHLLHHTLGTHDFGYQLEMAKKITCAAVAIRRAQDAPRLIDHAIRSAMSAKKPAYIEIPTNLSIANCPAPGPISAVIAPERSDEITLAMAVNAALDWLKSKQKPVLLAGPKLRAAGAEAAFLQLADALGCAVAVLPGAKSFFPEDHKQFVGVYWGQVSTMGADAIVDWSDGIFGAGVVFTDYSTVGWTALPPDSITLTADLDHMSFTGAEFNRVQLAELLSALAERATRNSSTMVEYAHLRPDVLFPHIEEPKLPLHRNEIARQIQQLLQPKTTLFVETGDSWFNGVQMRLPRSCRFEIEMQWGHIGWSVPASFGYAVGSPERQIILMVGDGSFQMTVQEVSQMVRARLPIIIFLMNNRGYTIEVEIHDGLYNRIKNWNYASLIEAFNAEDGHAKGIKASNPEQLAQAIKLATSNSDGPTLIECVIDQDDCTRELITWGHYVASANARPPAHKGGSG

SEQ ID NO: 142 - protein, 621 aa, Exophiala dermatitidis
MRCMSVPSMTFSRHTLRSCATSSDRMTGAPRKPFITSIKRQHQQPWHSICPNVTIIMSWTVGSYLAERLSQIGIEHHFVVPGDYNLVLLDQLQAHPKLSEIGCANELNCSFAAEGYARAKGVAAAVVTFSVGAFSAFNGLGGAYAENLPVILISGSPNTNDAGAFHLLHHTLGTHDFEYQRQIAEKITCAAVAVRRAQDAPRLIDHAIRSALLAKKPSYIEIPTNLSNVTCPAPGPISAVIAPEPSDEPTLAAAVHAATNWLKAKQKPILLAGPKLRAAGGEAGFLQLAEAIGCAVAVMPGAKSFFPEDHKQFVGVYWGQASTMGADAIVDWADGIFGAGLVFTDYSTVGWTAIPSESITLNADLDNMSFPGATFNRVRLADLLSALAKEATPNPSTMVEYARLRPDILPPHHEQPKLPLHRVEIARQIQELLHPKTTLFAETGDSWFNAMQMNLPRDCRFEIEMQWGHIGWSVPASFGYAVGAPERQVLLMIGDGSFQMTAQEVSQMVRSKVPIIIFLMNNGGYTIEVEIHDGLYNRIKNWNYAAMMEVFNAGDGHAKGIKASNPEQLAQAIKLAKSNSEGPTLIECIIDQDDCTKELITWGHYVATANGRPPAHTGGSG

SEQ ID NO: 143 - protein, 573 aa, Exophiala dermatitidis
MTKDAESTMTVGTYLAQRLVEIGIKNHFVVPGDYNLRLLDFLEYYPGLSEIGCCNELNCAFAAEGYARSNGIACAVVTYSVGALTAFDGIGGAYAENLPVILVSGSPNTNDLSSGHLLHHTLGTHDFEYQMEIAKKLTCAAVAIKRAEDAPVMIDHAIRQAILQHKPVYIEIPTNMANQPCPVPGPISAVISPEISDKESLEKATDIAAELISKKEKPILLAGPKLRAAGAESAFVKLAEALNCAAFIMPAAKGFYSEEHKNYAGVYWGEVSSSETTKAVYESSDLVIGAGVLFNDYSTVGWRAAPNPNILLNSDYTSVSIPGYVFSRVYMAEFLELLAKKVSKKPATLEAYNKARPQTVVPKAAEPKAALNRVEVMRQIQGLVDSNTTLYAETGDSWFNGLQMKLPAGAKFEVEMQWGHIGWSVPSAMGYAVAAPERRTIVMVGDGSFQLTGQEISQMIRHKLPVLIFLLNNRGYTIEIQIHDGPYNRIQNWDFAAFCESLNGETGKAKGLHAKTGEELTSAIKVALQNKEGPTLIECAIDTDDCTQELVDWGKAVRSANARPPTADNGGSG

SEQ ID NO: 144 - protein, 568 aa, Zymomonas mobilis; identical to SEQ ID NO: 109

SEQ ID NO: 145 - protein, 577 aa, Exophiala dermatitidis
MSYTVGQYLADRLVQIGLKDHFAIAGDYNLVLLDQFLKNKNWNQIYDCNELNCGFAAEGYARANGAAACVVTYTVGAISAMNSALAGAYAENLPVLCISGAPNCNDYGSGRILHHTIGKPEFTQQLDMVKHVTCAAESVVQASEAPAKIDHVIRTMLLEQRPAYIDIACNISGLECPRPGPIEDLLPQYAADNKSLTSAIDAIAKKIEASQKVTLYVGPKVRPGKAKEASVKLADALGCAVTVGPASMSFFPAKHPGFRGTYWGIVSTGDANKVVEEAETLIVLGPNWNDYATVGWKAWPKGPRVVTIDEKAAQVDGQVFSGLSMKALVEGLAKKVSKKPATAEGTKAPHFEYPVAKPDAKLTNAEMARQINAILDDNTTLHAETGDSWFNVKNMNWPNGLRIESEMQYGHIGWSIPSGFGGAIGSPERKHIIMCGDGSFQLTCQEVSQMIRYKLPVTIFLIDNHGYGIEIAIHDGPYNYIQNWNFTKLMEVFNGEGEECPYSHNKNGKSGLGLKATTPAELADAIKQAEANKEGPTLIQVVIDQDDCTKDLLTWGKEVAKTNARSPVVTDKAGGSG

SEQ ID NO: 146 - protein, 560 aa, Exophiala dermatitidis
MYTVGMYLAERLAQIGLKHHFAVAGDYNLVLLDQLLLNKDMEQVYCCNELNCGFSAEGYARARGAAAAIVTFSVGAISAMNAIGGAYAENLPVILISGSPNTNDYGTGHILHHTIGTTDYNYQLEMVKHVTCAAESIVSAEEAPAKIDHVIRTALRERKPAYLEIACNVAGAECVRPGPINSLLRELEVDQTSVTAAVDAAVEWLQDRQNVVMLVGSKLRAAAAEKQAVALADRLGCAVTIMAAAKGFFPEDHPNFRGLYWGEVSSEGAQELVENADAILCLAPVFNDYATVGWNSWPKGDNVMVMDTDRVTFAGQSFEGLSLSTFAAALAEKAPSRPATTQGTQAPVLGIEAAEPNAPLTNDEMTRQIQSLITSDTTLTAETGDSWFNASRMPIPGGARVELEMQWGHIGWSVPSAFGNAVGSPERRHIMMVGDGSFQLTAQEVAQMIRYEIPVIIFLINNRGYVIEIAIHDGPYNYIKNWNYAGLIDVFNDEDGHGLGLKASTGAELEGAIKKALDNRRGPTLIECNIAQDDCTETLIAWGKRVAATNSRKPQAGGSG

SEQ ID NO: 147 - protein, 562 aa, Kozakia baliensis
MAYTVGMYLAERLAQIGLKHHFAVAGDYNLVLLDQLLLNKDMEQIYCCNELNCGFSAEGYARAHGAAAAVVTFSVGAISAMNAIGGAYAENLPVILISGSPNSNDYGSGHILHHTLGTTDYGYQLEMARHVTCAAESITDAASAPAKIDHVIRTALRERKPAYLEIACNVSSAECPRPGPVSSLLAEPATDPVSLKAALEASLSALNKAERVVMLVGSKIRAADAQAQAVELADRLGCAVTIMSAAKGFFPEDHPGFRGLYWGEVSSPGAQELVENADAVLCLAPVFNDYSTVGWNAWPKGDKVLLAEPNRVTVGGQSFEGFALRDFLKGLTDRAPSKPATAQGTHAPKLEIKPAARDARLTNDEMARQINAMLTPNTTLAAETGDSWFNAMRMNLPGGARVEVEMQWGHIGWSVPSTFGNAMGSKDRQHIMMVGDGSFQLTAQEVAQMVRYELPVIIFLVNNKGYVIEIAIHDGPYNYIKNWDYAGLMEVFNAGEGHGIGLHAKTAGELEDAIKKAQANKRGPTIIECSLERTDCTETLIKWGKRVAAANSRKPQAVGGSG

SEQ ID NO: 148 - protein, 562 aa, Kozakia baliensis
MTYTVGMYLAERLSQIGLKHHFAVAGDFNLVLLDQLLVNKEMEQVYCCNELNCGFSAEGYARAHGAAAAVVTFSVGAISAMNAIAGAYAENLPVILISGSPNSNDYGTGHILHHTLGTNDYTYQLEMMRHVTCAAESITDAASAPAKIDHVIRTALRERKPAYVEIACNVSDAECVRPGPVSSLLAELRADDVSLKAAVEASLALLEKSQRVTMIVGSKVRAAHAQTQTEHLADKLGCAVTIMAAAKSFFPEDHKGFRGLYWGDVSSPGAQELVEKSDALICVAPVFNDYSTVGWTAWPKGDNVLLAEPNRVTVGGKTYEGFTLREFLEELAKKAPSRPLTAQESKKHTPVIEASKGDARLTNDEMTRQINAMLTSDTTLVAETGDSWFNATRMDLPRGARVELEMQWGHIGWSVPSAFGNAMGSQERQHILMVGDGSFQLTAQEMAQMVRYKLPVIIFLVNNRGYVIEIAIHDGPYNYIKNWDYAGLMEVFNAEDGHGLGLKATTAGELEEAIKKAKTNREGPTIIECQIERSDCTKTLVEWGKKVAAANSRKPQVSGGSG

SEQ ID NO: 149 - protein, 360 aa, Saccharomyces cerevisiae
MSYPEKFEGIAIQSHEDWKNPKKTKYDPKPFYDHDIDIKIEACGVCGSDIHCAAGHWGNMKMPLVVGHEIVGKVVKLGPKSNSGLKVGQRVGVGAQVFSCLECDRCKNDNEPYCTKFVTTYSQPYEDGYVSQGGYANYVRVHEHFVVPIPENIPSHLAAPLLCGGLTVYSPLVRNGCGPGKKVGIVGLGGIGSMGTLISKAMGAETYVISRSSRKREDAMKMGADHYIATLEEGDWGEKYFDTFDLIVVCASSLTDIDFNIMPKAMKVGGRIVSISIPEQHEMLSLKPYGLKAVSISYSALGSIKELNQLLKLVSEKDIKIWVETLPVGEAGVHEAFERMEKGDVRYRFTLVGYDKEFSD

SEQ ID NO: 150 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 151 - protein, 348 aa, Saccharomyces cerevisiae
MSIPETQKAIIFYESNGKLEHKDIPVPKPKPNELLINVKYSGVCHTDLHAWHGDWPLPTKLPLVGGHEGAGVVVGMGENVKGWKIGDYAGIKWLNGSCMACEYCELGNESNCPHADLSGYTHDGSFQEYATADAVQAAHIPQGTDLAEVAPILCAGITVYKALKSANLRAGHWAAISGAAGGLGSLAVQYAKAMGYRVLGIDGGPGKEELFTSLGGEVFIDFTKEKDIVSAVVKATNGGAHGIINVSVSEAAIEASTRYCRANGTVVLVGLPAGAKCSSDVFNHVVKSISIVGSYVGNRADTREALDFFARGLVKSPIKVVGLSSLPEIYEKMEKGQIAGRYVVDTSK

SEQ ID NO: 152 - protein, 248 aa, Escherichia coli; identical to SEQ ID NO: 129

SEQ ID NO: 153 - protein, 314 aa, Nitrosopumilus maritimus; identical to SEQ ID NO: 128

SEQ ID NO: 154 - protein, 314 aa, Metallosphaera sedula; identical to SEQ ID NO: 122

SEQ ID NO: 155 - protein, 298 aa, Pseudomonas aeruginosa; identical to SEQ ID NO: 125

SEQ ID NO: 156 - protein, 296 aa, Bacillus cereus; identical to SEQ ID NO: 124

SEQ ID NO: 157 - protein, 292 aa, Bacillus cereus; identical to SEQ ID NO: 123

SEQ ID NO: 158 - protein, 290 aa, Gluconobacter oxydans; identical to SEQ ID NO: 127

SEQ ID NO: 159 - protein, 289 aa, Thermus thermophilus; identical to SEQ ID NO: 130

SEQ ID NO: 160 - protein, 267 aa, Saccharomyces cerevisiae; identical to SEQ ID NO: 126

SEQ ID NO: 161 - protein, 642 aa, Megathyrsus maximus
MASPNGLAKIDTQGKTEVYDGDTAAPVRAQTIDELHLLQRKRSAPTTPIKDGATSAFAAAISEEDRSQQQLQSISASLTSLARETGPKLVKGDPSDPAPHKHYQPAAPTIVATDSSLKFTHVLYNLSPAELYEQAFGQKKSSFITSTGALATLSGAKTGRSPRDKRVVKDEATAQELWWGKGSPNIEMDERQFVINRERALDYLNSLDKVYVNDQFLNWDPENRIKVRIITSRAYHALFMHNMCIRPTDEELESFGTPDFTIYNAGEFPANRYANYMTSSTSINISLARREMVILGTQYAGEMKKGLFGVMHYLMPKRGILSLHSGCNMGKDGDVALFFGLSGTGKTTLSTDHNRLLIGDDEHCWSDNGVSNIEGGCYAKCIDLSQEKEPDIWNAIKFGTVLENVVFNERTREVDYSDKSITENTRAAYPIEFIPNAKIPCVGPHPKNVILLACDAFGVLPPVSKLNLAQTMYHFISGYTALVAGTVDGITEPTATFSACFGAAFIMYHPTKYAAMLAEKMQKYGATGWLVNTGWSGGRYGVGKRIRLPHTRKIIDAIHSGELLTANYKKTEVFGLEIPTEINGVPSEILDPINTWTDKAAYKENLLNLAGLFKKNFEVFASYKIGDDSSLTDEILAAGPNF

SEQ ID NO: 162 - protein, 540 aa, Escherichia coli
MRVNNGLTPQELEAYGISDVHDIVYNPSYDLLYQEELDPSLTGYERGVLTNLGAVAVDTGIFTGRSPKDKYIVRDDTTRDTFWWADKGKGKNDNKPLSPETWQHLKGLVTRQLSGKRLFVVDAFCGANPDTRLSVRFITEVAWQAHFVKNMFIRPSDEELAGFKPDFIVMNGAKCTNPQWKEQGLNSENFVAFNLTERMQLIGGTWYGGEMKKGMFSMMNYLLPLKGIASMHCSANVGEKGDVAVFFGLSGTGKTTLSTDPKRRLIGDDEHGWDDDGVFNFEGGCYAKTIKLSKEAEPEIYNAIRRDALLENVTVREDGTIDFDDGSKTENTRVSYPIYHIDNIVKPVSKAGHATKVIFLTADAFGVLPPVSRLTADQTQYHFLSGFTAKLAGTERGITEPTPTFSACFGAAFLSLHPTQYAEVLVKRMQAAGAQAYLVNTGWNGTGKRISIKDTRAIIDAILNGSLDNAETFTLPMFNLAIPTELPGVDTKILDPRNTYASPEQWQEKAETLAKLFIDNFDKYTDTPAGAALVAAGPKL

SEQ ID NO: 163 - protein, 538 aa, Actinobaccilus succinogenes
MTDLNKLVKELNDLGLTDVKEIVYNPSYEQLFEEETKPGLEGFDKGTLTTLGAVAVDTGIFTGRSPKDKYIVCDETTKDTVWWNSEAAKNDNKPMTQETWKSLRELVAKQLSGKRLFVVEGYCGASEKHRIGVRMVTEVAWQAHFVKNMFIRPTDEELKNFKADFTVLNGAKCTNPNWKEQGLNSENFVAFNITEGIQLIGGTWYGGEMKKGMFSMMNYFLPLKGVASMHCSANVGKDGDVAIFFGLSGTGKTTLSTDPKRQLIGDDEHGWDESGVFNFEGGCYAKTINLSQENEPDIYGAIRRDALLENVVVRADGSVDFDDGSKTENTRVSYPIYHIDNIVRPVSKAGHATKVIFLTADAFGVLPPVSKLTPEQTEYYFLSGFTAKLAGTERGVTEPTPTFSACFGAAFLSLHPIQYADVLVERMKASGAEAYLVNTGWNGTGKRISIKDTRGIIDAILDGSIEKAEMGELPIFNLAIPKALPGVDPAILDPRDTYADKAQWQVKAEDLANRFVKNFVKYTANPEAAKLVGAGPKA

SEQ ID NO: 164 - protein, 529 aa, Thermus thermophilus
MQRLEALGIHPKKRVFWNTVSPVLVEHTLLRGEGLLAHHGPLVVDTTPYTGRSPKDKFVVREPEVEGEIWWGEVNQPFAPEAFEALYQRVVQYLSERDLYVQDLYAGADRRYRLAVRVVTESPWHALFARNMFILPRRFGNDDEVEAFVPGFTVVHAPYFQAVPERDGTRSEVFVGISFQRRLVLIVGTKYAGEIKKSIFTVMNYLMPKRGVFPMHASANVGKEGDVAVFFGLSGTGKTTLSTDPERPLIGDDEHGWSEDGVFNFEGGCYAKVIRLSPEHEPLIYKASNQFEAILENVVVNPESRRVQWDDDSKTENTRSSYPIAHLENVVESGVAGHPRAIFFLSADAYGVLPPIARLSPEEAMYYFLSGYTARVAGTERGVTEPRATFSACFGAPFLPMHPGVYARMLGEKIRKHAPRVYLVNTGWTGGPYGVGYRFPLPVTRALLKAALSGALENVPYRRDPVFGFEVPLEAPGVPQELLNPRETWADKEAYDQQARKLARLFQENFQKYASGVAKEVAEAGPRTE

SEQ ID NO: 165 - protein, 532 aa, Anaerobiospirillum succiniciproducens
MSLSESLAKYGITGATNIVHNPSHEELFAAETQASLEGFEKGTVTEMGAVNVMTGVYTGRSPKDKFIVKNEASKEIWWTSDEFKNDNKPVTEEAWAQLKALAGKELSNKPLYVVDLFCGANENTRLKIRFVMEVAWQAHFVTNMFIRPTEEELKGFEPDFVVLNASKAKVENFKELGLNSETAVVFNLAEKMQIILNTWYGGEMKKGMFSMMNFYLPLQGIAAMHCSANTDLEGKNTAIFFGLSGTGKTTLSTDPKRLLIGDDEHGWDDDGVFNFEGGCYAKVINLSKENEPDIWGAIKRNALLENVTVDANGKVDFADKSVTENTRVSYPIFHIKNIVKPVSKAPAAKRVIFLSADAFGVLPPVSILSKEQTKYYFLSGFTAKLAGTERGITEPTPTFSSCFGAAFLTLPPTKYAEVLVKRMEASGAKAYLVNTGWNGTGKRISIKDTRGIIDAILDGSIDTANTATIPYFNFTVPTELKGVDTKILDPRNTYADASEWEVKAKDLAERFQKNFKKFESLGGDLVKAGPQL

SEQ ID NO: 166 - protein, 562 aa, Acetobacter indonesiensis
MTYTVGMYLADRLAQIGLKHHFAVAGDYNLVLLDQLLTNKDMQQIYCCNELNCGFSAEGYARAHGAAAAVVTFSVGAISAMNAIGGAYAENLPVILISGSPNSNDYGSGHILHHTIGSTDYGYQMEMVKHVTCAAESITDAASAPAKIDHVIRTALRESKPAYLEIACNVSAQECPRPGPVSSLLSEPAPDKTSLDAAVAAAVKLIEGAENTVILVGSKLRAARAQAEAEKLADKLECAVTIMAAAKGFFPEDHAGFRGLYWGEVSSPGTQELVEKADAIICLAPVFNDYSTVGWTAWPKGDKVLLAEPNRVTIKGQTFEGFALRDFLTALAAKAPARPASAKASSHTPTAFPKADAKAPLTNDEMARQINAMLTSDTTLVAETGDSWFNAMRMTLPRGARVELEMQWGHIGWSVPSSFGNAMGSQDRQHVVMVGDGSFQLTAQEVAQMVRYELPVIIFLVNNRGYVIEIAIHDGPYNYIKNWDYAGLMEVFNAGEGHGLGLHATTAEELEDAIKKAQANRRGPTIIECKIDRQDCTDTLVQWGKKVASANSRKPQAVGGSG